(19) United States
(12) Patent Application Publication
(10) Pub. No.: US 2024/0360508 A1
(43) Pub. Date: Oct. 31, 2024

(54) USE OF CIRCULATING CELL-FREE METHYLATED DNA TO DETECT TISSUE DAMAGE

(71) Applicant: Georgetown University, Washington, DC (US)

(72) Inventors: Anton Wellstein, Washington, DC (US); Megan E. McNamara, Washington, DC (US)

(73) Assignee: Georgetown University, Washington, DC (US)

(21) Appl. No.: 18/291,113

(22) PCT Filed: Jul. 25, 2022
(86) PCT No.: PCT/US2022/038244

§ 371 Date: Jan. 22, 2024

Related U.S. Application Data
(60) Provisional application No. 63/224,873, filed on Jul. 23, 2021.
(60) Provisional application No. 63/324,112, filed on Mar. 27, 2022.

(51) Int. Cl. C12Q 1/6883

(52) U.S. Cl. CPC C12Q 1/6883; C12Q 2600/106; C12Q 2600/154

(57) ABSTRACT

Method of determining if a subject has suffered tissue damage from exposure to a toxic agent. The method comprises sequencing cell-free DNA (cfDNA) in a biospecimen from the subject; determining cellular origin of the cfDNA by identifying methylation patterns in one or more portions of the sequence of the cfDNA that contains methylation sites, in which the cellular origin of the cfDNA is determined when the methylation pattern in the one or more portions is the same as a known cell-type specific methylation patterns; measuring the quantity of the cfDNA of the determined cellular origin, and comparing the measured quantity of the cfDNA of the determined cellular origin with a normal quantity of cfDNA of the determined cellular origin. A greater quantity of the measured cfDNA of the determined cellular origin is indicative that the subject has suffered tissue damage.

### CROSS-REFERENCE TO RELATED APPLICATIONS

This application claims the benefit of U.S. Provisional Application No. 63/224,873, filed on Jul. 23, 2022, and U.S. Provisional Application No. 63/324,112, filed on Mar. 27, 2022, each of which is incorporated herein by reference in its entirety.

### STATEMENT REGARDING FEDERALLY SPONSORED RESEARCH OR DEVELOPMENT

This invention was made with government support under grant numbers 132 CA009686, F30 CA250307, and R01 CA231291 awarded by the National Institutes of Health. The government has certain rights in the invention.

### BACKGROUND OF THE INVENTION

The human body is frequently exposed to agents that can have a damaging effect on tissue. Such agents may be, for instance, pathogenic such as bacteria or viruses; environmental such as sunlight; or therapeutic, such as pharmaceuticals that are associated with side effects.

Another type of therapy that can potentially lead to tissue damage are those used to treat cancer, including surgery, chemotherapy, radiotherapy, targeted therapy, and immunotherapy. Each of these interventions can have a significant systemic effect. For example, radiation therapy uses ionizing radiation to target tumor cells (Haussmann et al., 2020; Xu et al., 2008), but normal tissues are also impacted, leading to tissue damage and remodeling. (Ruysscher et al. 2019; Hubenak et al., 2014). For breast cancer patients, the heart and lungs are the most common organs impacted by radiation toxicities and a linear increase in cardiovascular disease risk of 7.4% per gray mean dose to the heart was reported (Darby et al, 2013; White and Joiner, 2006). In addition, radiation-induced lung injury is a severe complication reported in 5-20% of cases, presenting as radiation pneumonitis or fibrosis (Giuramno et al., 2019; Arroyo-Hernandez et al., 2021).

The ability to distinguish different cell types participating and potentially contributing to toxicities with cell-free DNA (cfDNA) in serially drawn blood samples could significantly impact on therapeutic decision making. Although imaging modalities can be used as an indirect way to gage therapeutic efficacy, these results are often unreliable and difficult to interpret. Imaging results can be clouded by depictions of pseudoprogression making them ineffective or crude instruments to monitor for concurrent changes necessary to guide therapy decisions. In light of the risk of tissue damage from radiation therapy or from exposure to other toxic agents, a means to effectively evaluate the tissue damage and monitor the effects of therapies is essential.

### SUMMARY OF INVENTION

Some of the main aspects of the present invention are summarized below. Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

The invention provides novel methods for detecting tissue damage from exposure to toxic agents.

In one aspect, the present invention relates to a method of determining if a subject has suffered tissue damage from exposure to a toxic agent. In some embodiments, the method comprises (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cfDNA is determined when the methylation pattern in the one or more portions is the same as a known cell-type specific methylation patterns; (c) measuring the quantity of the cfDNA of the determined cellular origin, and (d) comparing the measured quantity of the cfDNA of the determined cellular origin with a normal quantity of cfDNA of the determined cellular origin. An increase in the measured quantity of the cfDNA of the determined cellular origin over the normal quantity of cfDNA of the determined cellular origin is indicative that the subject has suffered or suffers tissue damage from the exposure. In other embodiments, the method comprises, at two or more time points, (a) sequencing cfDNA in a biospecimen from the subject; determining cellular origin of the cfDNA by identifying the methylation patterns in one or more portions of the sequence of the cfDNA that contains methylation sites, in which the cellular origin of the cfDNA is determined when the methylation pattern in the one or more portions is the same as a known cell-type specific methylation patterns; and (c) measuring the quantity of the cfDNA of the determined cellular origin. An increase in the measured quantity of the cfDNA of the determined cellular origin at a later time point as compared to an earlier time point is indicative that the subject has suffered or suffers tissue damage from the exposure.

In another aspect, the present invention also relates to a method of treating a subject who has suffered tissue damage from exposure to a toxic agent. In some embodiments, the method comprises administering a treatment for the tissue damage to the subject, in which the subject is determined to have suffered from tissue damage by a method comprising: (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cfDNA is determined when the methylation pattern in the one or more portions is the same as a known cell-type specific methylation patterns; (c) measuring the quantity of the cfDNA of the determined cellular origin, and (d) comparing the measured quantity of the cfDNA of the determined cellular origin with a normal quantity of cfDNA of the determined cellular origin. An increase in the measured quantity of the cfDNA of the determined cellular origin over the normal quantity of cfDNA of the determined cellular origin is indicative that the subject has suffered tissue damage. In other embodiments, the method comprises administering a treatment for the tissue damage to the subject, in which the subject is determined to have suffered from tissue damage by a method comprising, at two or more time points: (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cfDNA is determined when the methylation pattern in the one or more portions is the same as a known cell-type specific methylation patterns; and (c) measuring the quantity of the cfDNA of the determined cellular origin. An increase in the measured quantity of the cfDNA of the determined cellular origin at a later time point as compared to an earlier time point is indicative that the subject has suffered tissue damage.

In yet another aspect, the present invention further relates to a method of treating tissue damage in a subject. In some embodiments, the method comprising administering a treatment for the tissue damage to the subject and monitoring the tissue damage, in which the monitoring comprises: (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cfDNA is determined when the methylation pattern in the one or more portions is the same as a known cell-type specific methylation patterns; (c) measuring the quantity of the cfDNA of the determined cellular origin, and (d) comparing the measured quantity of the cfDNA of the determined cellular origin with a normal quantity of cfDNA of the determined cellular origin. A decrease in the measured quantity of the cfDNA of the determined cellular origin as compared to the normal quantity of cfDNA of the determined cellular origin is indicative that the treatment is effective, and an increase or no change in the measured quantity of the cfDNA of the determined cellular origin over the normal quantity of cfDNA of the determined cellular origin is indicative that the treatment is not effective. In other embodiments, the method comprises administering a treatment for the tissue damage to the subject and monitoring the tissue damage, in which the monitoring comprises, at two or more time points: (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cfDNA is determined when the methylation pattern in the one or more portions is the same as a known cell-type specific methylation patterns; and (c) measuring the quantity of the cfDNA of the determined cellular origin. A decrease in the measured quantity of the cfDNA of the determined cellular origin at later time point as compared to an earlier time point is indicative that the treatment is effective, and an increase or no change in the measured quantity of the cfDNA of the determined cellular origin at a later time point as compared to an earlier time point is indicative that the treatment is not effective.

In some embodiments, the tissue damage is caused by exposure to a toxic agent. In certain embodiments, toxic agent comprises radiation.

The radiation may be for therapeutic purposes, accidental or environmental. In some embodiments, the radiation comprises a radioactive substance. The radioactive substance may be ingested by the subject, inhaled by the subject, or absorbed through body surface contamination by the subject.

In other embodiments, the toxic agent comprises a microorganism. The microorganism may comprise a pathogen, such as a bacterium or virus.

In some embodiments, the toxic agent is from a synthetic chemical source or from a biological source.

In some embodiments, the toxic agent comprises a pharmaceutical therapy.

In some embodiments, the toxic agent comprises a chemical or biological or radioactive substance used a weapon.

In a further aspect, the present invention relates to method of treating a subject in need thereof. In some embodiments, the method comprises administering a treatment to the subject and monitoring whether the treatment causes tissue damage in the subject, in which the monitoring comprises: (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cfDNA is determined when the methylation pattern in the one or more portions is the same as a known cell-type specific methylation patterns; (c) measuring the quantity of the cfDNA of the determined cellular origin, and (d) comparing the measured quantity of the cfDNA of the determined cellular origin with a normal quantity of cfDNA of the determined cellular origin. An increase in the measured quantity of the cfDNA of the determined cellular origin over the normal quantity of cfDNA of the determined cellular origin is indicative that the treatment is causing tissue damage. In other embodiments, the method comprises administering a treatment to the subject and monitoring whether the treatment causes tissue damage in the subject, in which the monitoring comprises, at two or more time points: (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cfDNA is determined when the methylation pattern in the one or more portions is the same as a known cell-type specific methylation patterns; and (c) measuring the quantity of the cfDNA of the determined cellular origin, in which an increase in the measured quantity of the cfDNA of the determined cellular origin at a later time point as compared to an earlier time point is indicative that the treatment is causing tissue damage.

In some embodiments, the methods further comprise adjusting the treatment administered to the subject when the treatment is indicated to be not effective or causing tissue damage.

In some embodiments, the normal quantity of cfDNA comprises a quantity of cfDNA for the determined cellular origin that is generated in a population of individuals who were not exposed to the toxic agent, or who were not administered the treatment.

In yet other aspects, the present invention relates to a method of treating a subject having a tumor. In some embodiments, the method comprises (A) monitoring a response to a first treatment, an adverse reaction to the first treatment, or a combination thereof, in which the monitoring comprises: (i) determining whether there is an adverse reaction to the first treatment, comprising (a) sequencing cfDNA) in a biospecimen from the subject: (b) determining cellular origin of the cfDNA by identifying the methylation patterns in one or more portions of the sequence of the cfDNA that contains methylation sites, in which the cellular origin of the cfDNA is determined when the methylation pattern in the one or more portions is the same as a known cell-type specific methylation patterns; (c) measuring the quantity of the cfDNA of the determined cellular origin, and (d) comparing the measured quantity of the cfDNA of the determined cellular origin with a normal quantity of cfDNA of the determined cellular origin, in which an increase in the measured quantity of the cfDNA of the determined cellular origin over the normal quantity of cfDNA of the determined cellular origin is indicative of an adverse reaction; (ii) determining whether there is a response to the first treatment, comprising: (a) sequencing circulating tumor DNA (cfDNA) in a biospecimen from the subject, determining clonal heterogeneity of cells of the tumor by genotyping the cfDNA, in which the presence of more than one clone of the tumor cells or the presence of a tumor cell clone that has not been previously identified in the subject is indicative of an ineffective response to the first treatment; and (B) either administering the same treatment as the first treatment when it is determined that there is no adverse reaction, that there is not an ineffective response, or a combination thereof; or administering an adjusted treatment when it is determined that there is an adverse reaction, that there is an ineffective response, or a combination thereof. In other embodiments, the method comprises (A) monitoring a response to a first treatment, an adverse reaction to the first treatment, or a combination thereof, in which the monitoring comprises, at two or more time points, (i) determining whether there is an adverse reaction to the first treatment, comprising (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cfDNA is determined when the methylation pattern in the one or more portions is the same as a known cell-type specific methylation patterns; and (c) measuring the quantity of the cfDNA of the determined cellular origin, in which an increase in the measured quantity of the cfDNA of the determined cellular origin at a later time point as compared to an earlier time point is indicative of an adverse reaction; and (ii) determining whether there is a response to the first treatment, comprising (a) sequencing circulating tumor (cfDNA) in a biospecimen from the subject, (b) determining clonal heterogeneity of cells of the tumor by genotyping the cfDNA, in which the presence of more than one clone of the tumor cells or the presence of a tumor cell clone in a subsequent time point that has not been identified at a previous time point is indicative of an ineffective response to the first treatment; and (B) either administering the same treatment as the first treatment when it is determined that there is no adverse reaction, that there is not an ineffective response, or a combination thereof; or administering an adjusted treatment when it is determined that there is an adverse reaction, that there is an ineffective response, or a combination thereof.

In some embodiments, the normal quantity of cfDNA comprises a quantity of cfDNA for the determined cellular origin that is generated in a population of individuals who do not have a tumor. In other embodiments, the normal quantity of cfDNA comprises a quantity of cfDNA for the determined cellular origin that is generated in a population of individuals who did not receive the first treatment.

In some embodiments, the biospecimen comprises a biological fluid. In certain embodiments, the biological fluid is selected from blood, serum, plasma, cerebrospinal fluid, saliva, urine, and sputum. In preferred embodiments, the biological fluid comprises blood, serum, or plasma.

In some embodiments, the methylation pattern comprises a segment of nucleotide sequence containing at least 3 CpG dinucleotides.

In some embodiments, the known methylation patterns are set forth in Table 2.

### BRIEF DESCRIPTION OF THE DRAWING FIGURES

FIG. 1 illustrates an example of the use of predicting treatment response and therapy-related toxicities from combined genetic and epigenetic analyses of cfDNA. Predicting treatment response and therapy-related toxicities from combined genetic and epigenetic analyses of cfDNA. The minimally invasive nature of liquid biopsies allows for serial sampling to monitor changes over time, especially under selective pressures from ongoing therapy. Circulating tumor DNA (CtDNA) can be used to track clonal heterogeneity over time to assess treatment response and detect treatment-resistant clones. Normal cell-specific cfDNA methylation patterns can be used in combination with cfDNA to assess the impact of treatment to the surrounding tumor microenvironment and to monitor for therapy-related toxicities in somatic cell-types. [ctDNA=circulating tumor DNA; cme-DNA=circulating methylated cell-free DNA].

FIG. 2 shows the overall analysis of cell-free methylated DNA in blood to identify origins of radiation-induced cellular damage, as described in the Example. Serial serum samples were collected from human breast cancer patients treated with radiation. In parallel, paired serum and tissue samples were collected from mice receiving radiation at 3Gy or 8Gy doses compared to sham control. Methylome profiling of liquid biopsy samples was performed using a bisulfite-based capture-sequencing methodology optimized for cfDNA inputs. Differential cell type-specific methylation blocks were identified from reference WGBS data compiled from healthy cell-types and tissues in human and mouse. Methylation atlases were generated emphasizing cell-types composing target organs-at-risk from radiation, including the lungs, heart, and liver. Deconvolution analysis of cfDNA using fragment-level CpG methylation patterns at these identified cell-type specific blocks was used to decode the origins of radiation-induced cellular injury.

FIG. 3 shows sensitivity and specificity of identified mouse cell-type specific differentially methylated blocks, as described in the Example. In Panels A-D, the top images are a heatmap of all cell type-specific methylation blocks selected for each target cell-type. All blocks contain 3+CpG sites and have a margin of beta difference greater than or equal to 0.4 separating the target cell-type from all others included in the reference maps. All identified methylation blocks for lung endothelial (n=1,546), hepatocyte (n=616), and cardiomyocyte (n=2,917) mouse cell-types were hypomethylated. In contrast, all identified immune cell-specific blocks (n=148) were hypermethylated relative to other solid organ cell-types in mouse. In Panels A-D, the right images show in-silico mix-in validation of fragment-level probabilistic deconvolution model. Target cell-type read-pairs were in-silico mixed into a background of lymphocyte or buffy coat read-pairs at various known percentages (0, 0.5, 1, 2, 5, 10, 15%) with 10 replicates per proportion. The deconvolution model was validated on these in-silico mixed samples of known cell-type proportions at the blocks selected. The average predicted % target is graphed relative to the known % mixed to assess sensitivity and specificity of the identified cell type-specific blocks and deconvolution model. Data presented as mean±SD; n=3 replicates per proportion. Reference WGBS samples with less than 3 replicates were split into “0.8 train” to select methylation blocks and “0.2 test” to generate in-silico mixed samples. When available, in-silico mixed samples of the same cell-type derived from different aged mice were tested. In addition, bulk tissue of the respective cell-type was tested as well.

FIG. 4 shows sensitivity and specificity of identified human cell-type specific differentially methylated blocks, as described in the Example. In Panels A-F, the top images are heatmaps of all cell type-specific methylation blocks selected for each target cell-type. All blocks contain 3+CpG sites and have a margin of beta difference greater than or equal to 0.4 separating the target cell-type from all others included in the reference maps. In Panels A-F, the bottom images show in-silico mix-in validation of fragment-level probabilistic deconvolution model. Target cell-type read-pairs were in-silico mixed into a background of lymphocyte or buffy coat read-pairs at various known percentages (0, 0.5, 1, 2, 5, 10, 15%). The deconvolution model was validated on these in-silico mixed samples of known cell-type proportions at the blocks selected. The average predicted % target is graphed relative to the known % mixed to assess sensitivity and specificity of the identified cell type-specific blocks and deconvolution model. Data is presented as mean±standard deviation: n=3 replicates per proportion.

FIG. 5 shows characterization of human and mouse cell-type specific reference methylation data, as described in the Example. Panel A shows a tree dendrogram depicting relationship between human reference Whole Genome Bisulfite Sequencing (WGBS) datasets included in the analysis. Methylation status at the top 30,000 variable blocks was used as input data for the unsupervised hierarchical clustering. Samples from cell-types with greater than n=3 replicates were merged. Panel B shows UMAP projection of human WGBS reference datasets, colored by tissue and cell-type. Panel C shows UMAP projection of mouse WGBS reference datasets. [Acronyms: HUVEV=hurman umbilical vein endothelial cell, PAEC=pulmonary artery endothelial cell, CAEC=coronary artery endothelial cell, PMEC=pulmonary microvascular endothelial cell, CMEC=cardiac microvascular endothelial cell, CPEC=joint cardio-pulmonary endothelial cell, LSEC liver sinusoidal endothelial cell, NK=natural killer cell, MK=megakaryocyte.]

FIG. 6 shows characterization of mouse cell-type specific reference methylation data, as described in the Example. Panel A shows a tree dendrogram depicting relationship between mouse reference WGBS datasets included in the analysis. Methylation status at the top 30,000 variable blocks was used as input data for the unsupervised hierarchical clustering. Panel B shows heatmaps of differentially methylated cell type-specific blocks identified from reference WGBS data compiled from healthy cell-types and tissues in mouse. Each cell in the plot marks the average methylation of one genomic region (row) at each of the 9 mouse tissues and cell-types (columns). Up to 100 blocks with the highest methylation score are shown per cell type. Differential blocks identified from cell-types comprising the target organs-at-risk from radiation (lungs, heart, and liver) were selected for generation of a radiation-specific methylation atlas, separating these solid organ cell-types from all other immune cell-types.

FIG. 7 shows identification and biological validation of cell-type specific DNA methylation blocks in human and mouse, as described in the Example. Panels A and B show heatmaps of differentially methylated cell type-specific blocks identified from reference WGBS data compiled from healthy cell-types and tissues in human (Panel A) and mouse (Panel B). Each cell in the plot marks the methylation score of one genomic region (rows) at each of the 20 cell types in human and 9 in mouse (columns). Up to 100 blocks with the highest methylation score are shown per cell type. The methylation score represents the number of fully unmethylated read-pairs/total coverage or fully methylated read-pairs/total coverage for hypo- and hyper-methylated blocks, respectively. Panel C shows heatmap of distance scores between gene-set pathways identified from GeneSetCluster. Genes adjacent to human cell type-specific methylation blocks were identified using HOMER and pathway analysis was performed using both Ingenuity Pathway Analysis (IPA) and GREAT. Significantly enriched gene-set pathways (p<0.05) from differentially methylated blocks identified in immune, cardiomyocyte, hepatocyte, and lung epithelial cell-types were analyzed using GeneSetCluster. Cluster analysis was performed to determine the distance between all identified gene-set pathways based on the degree of overlapping genes from each individual gene-set compared to all others. Over-representation analysis was implemented in the WebgestaltR (ORAperGeneSet) plugin to interpret and functionally label identified gene-set clusters. [Acronyms: HUVEV=human umbilical vein endothelial cell, CPEC=cardio-pulmonary endothelial cell, LSEC=liver sinusoidal endothelial cell, NK=natural killer cell.]

FIG. 8 shows biological function of mouse cell-type specific methylation blocks, as described in the Example. Heatmap of distance scores between gene-set pathways identified from GeneSetCluster. Genes adjacent to cell type-specific methylation blocks were identified using HOMER and pathway analysis was performed using both Ingenuity Pathway Analysis (IPA) and GREAT. Significantly enriched gene-set pathways (p<0.05) from differentially methylated blocks identified in immune, cardiomyocyte, hepatocyte, and lung endothelial cell-types were analyzed using GeneSetCluster. Cluster analysis was performed to determine the distance between all identified gene-set pathways based on the degree of overlapping genes from each individual gene-set compared to all others. Over-representation analysis was implemented in the WebgestaltR (ORAperGeneSet) plugin to interpret and functionally label identified gene-set clusters.

FIG. 9 shows cell type-specific DNA methylation is mostly hypomethylated and enriched at intragenic regions and developmental transcription factor (TF) binding motifs, as described in the Example. Panel A shows a schematic diagram depicting location of human cell-type specific hypo- and hyper-methylated blocks. Genomic annotations of cell type-specific methylation blocks were determined by analysis using HOMER. Panels B and C show distribution of human (Panel B) and mouse (Panel C) cell-type specific methylation blocks relative to genomic regions used in the hybridization capture probes. Captured blocks with less than 5% variance across cell types represent blocks without cell type specificity and were used as background. Panel D shows top 5 TF binding sites enriched among identified cell-type specific hypo- and hypermethylated blocks in human (top) and mouse (bottom), using HOMER motif analysis. The same captured blocks with less than 5% variance amongst cell-types were used as background.

FIG. 10 shows methylation profiling of human endothelial cell-types reveals tissue-specific differences that correspond with changes in RNA expression levels and biological functions, as described in the Example. Panel A shows pathways supporting the biological significance of endothelial-specific methylation blocks (all p<0.05). Panel B shows significant functions of genes adjacent to endothelial-specific methylation blocks. Asterisked genes have nearby hypermethylated regulatory blocks. Non-asterisked genes have nearby hypomethylated regulatory blocks. Panel C shows gene expression at genes adjacent to tissue-specific endothelial-specific methylation blocks. Expression data was generated from paired RNA-sequencing of the same cardiopulmonary endothelial cells (CPEC) and liver sinusoidal endothelial cells (LSEC) used to generate methylation reference data. Pan-endothelial genes upregulated in both populations (ALL) are identified as common endothelial-specific methylation blocks to both LSEC and CPEC populations. Panel D shows top 5 transcription factor binding sites enriched among identified endothelial-specific hypomethylated blocks, using HOMER de novo and known motif analysis. The background for HOMER analysis was composed of the other 3,574 identified cell-type specific hypomethylated blocks in all cell-types besides endothelial. Panel E shows an example of the NOS3 locus specifically unmethylated in endothelial cells. This endothelial-specific, differentially methylated block (DMB) is 157 bp long (7 CpGs), and is located within the NOS3 gene, an endothelial-specific gene (upregulated in paired RNA-sequencing data as well as in vascular endothelial cells, GTEx inset). The alignment from the UCSC genome browser (top) provides the genomic locus organization and is aligned with the average methylation across cardiomyocyte, lung epithelial, liver sinusoidal endothelial (LSEC), cardiopulmonary endothelial (CPEC), hepatocyte, and immune (PBMC) samples (n=3/cell-type group). Results from RNA-sequencing generated from paired cell-types are depicted as well as peak intensity from H3K27ac and H3K4me3 published ChIP-seq data generated in endothelial cells [Acronyms: HUVEV=human umbilical vein endothelial cell, CPEC=cardio-pulmonary endothelial cell, LSEC=liver sinusoidal endothelial cell.]

FIG. 11 shows development of radiation-specific methylation atlas focusing on cell-types from target organs-at-risk (OAR), as described in the Example. Panel A shows representative three-dimensional conformal radiation therapy (3D-CRT) treatment planning for right-sided (i and ii) and left-sided (iii and iv) breast cancer patients, respectively. Computed tomography simulation coronal and sagittal images depicting anatomic position of target volume in relation to nearby organs. The map represents different radiation dose levels or isodose lines (95% of prescription dose, 90% isodose line, 80% isodose line, 70% isodose line, 50% isodose line). Panel B shows heatmaps of differentially methylated cell type-specific blocks identified from all reference WGBS data compiled from healthy human cell-types and tissues. Each cell in the plot marks the average methylation of one genomic region (rows) at each of the 20 human cell-types (columns). Up to 100 blocks with the highest methylation score are shown per cell type. Differential blocks identified from cell-types comprising the target organs-at-risk from radiation (lungs, heart, and liver) were selected for generation of a radiation-specific methylation atlas, separating these solid organ cell-types from all other immune cell-types. [Acronyms: HUVEV=human umbilical vein endothelial cell, PAEC=pulmonary artery endothelial cell, CAEC=coronary artery endothelial cell, PMEC=pulmonary microvascular endothelial cell, CMEC=cardiac microvascular endothelial cell, CPEC=joint cardio-pulmonary endothelial cell, LSEC=liver sinusoidal endothelial cell, NK=natural killer cell, MK=megakaryocyte.]

FIG. 12 shows that dose-dependent radiation damage in mouse tissues correlates with origins of methylated cfDNA in the circulation, as described in the Example. Panel A shows representative hematoxylin and eosin (H&E) staining of mouse lung, heart, and liver tissues treated with 3Gy and 8Gy radiation compared to sham control. Scale bar, 200 μm. Panel B shows quantitative polymerase chain reaction (qPCR) analysis of CDKN1A (p21) marker of apoptosis in mouse tissues treated with 3Gy and 8Gy radiation compared to sham control. The expression of each sample was normalized with expression of house-keeping genes ACTB (actin) and is shown relative to the expression in the sham control. Data presented as mean±SD (n=3). Kruskal-Wallis test was used for comparisons amongst groups; lung tissue p=0.004, heart tissue p=0.025, liver tissue p=0.004. Panels C-F show lung endothelial, cardiomyocyte and hepatocyte methylated cfDNA in the circulation of mice treated with 3Gy and 8Cy radiation compared to sham control expressed in Genome Equivalents (Geq). CfDNA was extracted from 18 mice (n=6 in each group) with cfDNA from 2 mice pooled in each methylome preparation. Mean±SD, n 3 independent methylome preparations. Kruskal-Wallis test was used for comparisons amongst groups. ns, P≥0.05; *, P<0.05 lung endothelial p=0.01, cardiomyocyte p=0.01, hepatocyte p=0.13.

FIG. 13 shows apoptotic damage from radiation in mouse tissues, as described in the Example. qPCR analysis of markers of apoptosis (Trp53, Gadd45a, Aifm3, and Bad) in mouse lung, heart, and liver tissues treated with 3Gy and 8Gy radiation compared to sham control. The expression of each sample was normalized with expression of house-keeping genes ACTB (actin). Data presented as mean±SD (n=3).

FIG. 14 shows radiation-induced effects on immune and solid organ cfDNA, as described in the Example. Panels A-C show the radiation-induced effects in human, and Panels D and E show the radiation-induced effects in mouse. Panel A shows predicted human immune-derived cfDNA in Geq. Human Geq are calculated by multiplying the relative fraction of cell-type specific cfDNA×initial concentration cfDNA ng/mL×the weight of the haploid human genome. Immune cfDNA was assessed at n=222 methylation blocks found to separate immune cell types from solid organ cell-types. (g1=Bcell, CD4Tcell, CD8Tcell, NK, MK, erythroblast, monocyte, macrophage, neutrophil; g2=breast basal/luminal epi, lung epi, hepatocyte, kidney podocyte, pancreas islet, colon epi, cardiomyocyte, LSEC, CPEC, HUVEC, neuron, and skeletal muscle). Panel B shows predicted human solid organ-derived cfDNA in Geq where % solid organ is defined as 100-% immune using these same n=222 methylation blocks. Panel C shows fold change in human immune versus solid organ Geq at EOT and recovery relative to baseline. Data presented as mean±SD; n=15. For Panels A and B, Friedman test was performed for comparisons amongst groups. ns, P>0.05; P<0.05; immune p=0.07, solid organ p=0.008. Panel D shows predicted mouse immune-derived cfDNA in Geq. Mouse Geq are calculated by multiplying the relative fraction of cell-type specific cfDNA×initial concentration cfDNA ng/mL×the weight of the haploid mouse genome. Immune cfDNA was assessed at n=148 methylation blocks found to separate immune cell types from solid organ cell-types. (g1=Bcell, CD4Tcell, CD8Tcell, neutrophil; g2=mammary epi, cardiomyocyte, hepatocyte, lung endothelial, cerebellum, hypothalamus, colon, intestine, kidney). Panel E shows predicted mouse solid organ-derived cfDNA in Geq. For Panels D and E, mean±SD; n=3 independent methylome preparations. Kruskal-Wallis test was used for comparisons amongst groups. ns, P>0.05; *, P<0.05; immune p=0.20, solid organ p=0.01.

FIG. 15 shows radiation-induced hepatocyte and liver endothelial cfDNAs in patient with right-versus left-sided breast cancer, as described in the Example. Panels A and B show hepatocyte cfDNA (in Geq/mL) in serum samples collected at different times. Fragment-level deconvolution using hepatocyte specific methylation blocks (n=200). Wilcoxon matched pairs signed rank test was used for comparison amongst groups and results were considered significant when *P<0.05; ns, P≥0.05; right-sided p≤0.02, left-sided p=0.81 Panel C shows fold change in hepatocyte cfDNA after treatment (EOT) and at recovery relative to baseline. Mean±SD; n=8 right-sided, n=7 left-sided. Panels D and E show LSEC cfDNA (in Geq/mL) in the same serum samples. Fragment-level deconvolution used LSEC specific methylation blocks (n=89). Wilcoxon matched pairs signed rank test was performed between groups and results were considered significant when *P<0.05; ns, P≥0.05: right-sided p=0.02, left-sided p=0.93. Panel F shows fold change in LSEC cfDNA Geq at EOT and recovery relative to baseline levels. Mean±SD; n=8 right-sided, n=7 left-sided.

FIG. 16 shows that radiation-induced cardiopulmonary cfDNAs in patients correlates with the radiation dose and indicates sustained injury to cardiomyocytes, as described in the Example. Panel A shows lung epithelial cfDNA (in Geq/mL) in serum samples collected at different times. Fragment-level deconvolution used lung epithelial specific methylation blocks (n=69). Panel B shows correlation of lung epithelial cfDNA with dosimetry data. EOT/Baseline represents the fraction of lung epithelial cfDNA post-radiation at end-of-treatment (EOT) relative to baseline levels. The volume of the lung receiving 20 Gy dose is represented by Lung V20(%) and the mean dose to the total body represented by total body mean (Cy). Panel C shows fold change in lung epithelial cfDNA at EOT and recovery relative to baseline. Panel D shows CPEC cfDNA (in Geq/mL). Fragment-level deconvolution used CPEC-specific methylation blocks (n=132). Panel E shows correlation of CPEC cfDNA with dosimetry data. The volume of the lung receiving 5 Gy dose is represented by Lung V5(%). Panel F shows fold change in CPEC cfDNA at EOT and recovery relative to baseline levels. Panel C shows cardiomyocyte cfDNA (in Geq/mL). Fragment-level deconvolution used cardiomyocyte-specific methylation blocks (n=375). Panel H shows correlation of cardiomyocyte cfDNA with the maximal heart dose (Gy). Panel I shows fold change in cardiomyocyte cfDNA at EOT and recovery relative to baseline. For Panels A, D, and G, Friedman test was performed comparing paired results at baseline, EOT, and recovery timepoints. The results were considered significant when *P<0.05; ns, P≥0.05; lung epithelial p=0.98, cardiopulmonary endothelial p=0.02, cardiomyocyte p=0.03. For Panels B, E, and H, Pearson correlation r was calculated, and linear correlation was considered significant when *P<0.05. For Panels C, F, and I. Wilcoxon matched-pairs signed rank test was performed between groups and results were considered significant when P<0.05. Data is presented as mean±SD; n=15.

### DETAILED DESCRIPTION OF THE INVENTION

The practice of the present invention can employ, unless otherwise indicated, conventional techniques of genetics, molecular biology, computational biology, genomics, epigenomics, mass spectrometry, and bioinformatics, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Whenever embodiments are described with the language “comprising.” otherwise analogous embodiments described in terms of “consisting of” and/or “consisting essentially of” are included.

Units, prefixes, and symbols are denoted in their Systéme International d'Unités (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value (i.e., intermediate) encompassed by the range, including integers and fractions. For example, a stated range of 5-10 is also a disclosure of 5, 6, 7, 8, 9, and 10 individually, and of 5.2, 7.5, 8.7, and so forth.

Unless otherwise indicated, the terms “at least” or “about” preceding a series of elements is to be understood to refer to every element in the series. The term “about” preceding a numerical value includes=10% of the recited value. For example, a concentration of about 1 mg/mL includes 0.9 mg/mL to 1.1 mg/mL. Likewise, a concentration range of about 1% to 10% (w/v) includes 0.9% (w/v) to 11% (w/v).

As used herein, the terms “cell-free DNA” or “cfDNA” or “circulating cell-free DNA” refers to DNA that is circulating in the peripheral blood of a subject. The DNA molecules in cfDNA may have a median size that is no greater than 1 kb (for example, about 50 bp to 500 bp, or about 80 bp to 400 bp, or about 100 bp to 1 kb), although fragments having a median size outside of this range may be present. This term is intended to encompass free DNA molecules that are circulating in the bloodstream as well as DNA molecules that are present in extra-cellular vesicles (such as exosomes) that are circulating in the bloodstream.

“Methylation site” refers to a CpG dinucleotide.

“Methylation pattern” refers to the pattern generated by the presence of methylated CpGs or non-methylated CpGs in a segment of DNA. For example, in a segment of DNA containing three CpGs, one methylation pattern is all three CpGs being methylated; a different methylation pattern is all three CpGs not being methylated; another methylation pattern is only the first CpG being methylated; yet another methylation pattern is only the second CpG being methylated; yet a different methylation pattern is the first and second CpG being methylated, etc.

“Methylation status” refers to whether a CpG dinucleotide is methylated or not methylated.

As used herein, “hypermethylated” refers to the presence of methylated CpGs. For example, a hypermethylated genomic region means that each CpG in the genomic region is methylated.

As used herein, “hypomethylated” refers to the presence of CpGs that are not methylated. For example, a hypomethylated genomic region means that each CpG in the genomic region is not methylated.

The term “sequencing” as used herein refers to a method by which the identity of at least 10 consecutive nucleotides for example, the identity of at least 20, at least 50, at least 100 or at least 200 or more consecutive nucleotides) of a polynucleotide is obtained.

The term “next-generation sequencing” as used herein refers to the parallelized sequencing-by-synthesis or sequencing-by-ligation platforms currently employed by Illumina, Life Technologies, and Roche, etc. Next-generation sequencing methods may also include nanopore sequencing methods such as that commercialized by Oxford Nanopore Technologies, electronic-detection based methods such as Ion Torrent technology commercialized by Life Technologies, or single-molecule fluorescence-based methods such as that commercialized by Pacific Biosciences.

A “subject” or “individual” or “patient” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

An “effective amount” of an active agent is an amount sufficient to carry out a specifically stated purpose.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. In certain embodiments, a subject is successfully “treated” for a disease or disorder if the patient shows total, partial, or transient alleviation or elimination of at least one symptom or measurable physical parameter associated with the disease or disorder.

Methods Using cfDNA to Determine Tissue Damage

The present invention relates to methods that utilize circulating cfDNA to determine tissue damage. The majority of cfDNA fragments peak around 167 bp, corresponding to the length of DNA wrapped around a nucleosome (147 bp) plus a linker fragment (20 bp). This nucleosomal footprint in cfDNA reflects degradation by nucleases as a by-product of cell death (Heitzer et al., 2020).

DNA methylation typically involves covalent addition of a methyl group to the 5-carbon of cytosine (5 mc) with the human and mouse genomes contain 28 and 13 million CpG sites respectively (Greenberg and Bourc'his, 2019; Michalak et al., 2019). Stable, cell-type specific patterns of DNA methylation are conserved during DNA replication and thus provide the predominant mechanism for inherited cellular memory during cell growth (Kim & Costello, 2017; Dor & Cedar, 2018). DNA methylation changes associated with disease and physiological aging occur at locations throughout the epigenome that are distinct from regions critical to cell-type identity, making methylated cfDNA a robust cell-type specific readout across diverse patient populations (Michalak et al 2019; Dor & Cedar, 2018).

While recent studies have demonstrated the feasibility of Tissue-Of-Origin (TOO) analysis using cfDNA methylation, such studies traditionally averaged the methylation status across a population of fragments present at single CpG sites (Barefoot, et al., 2021; Barefoot et al., 2020). The present invention involves sequencing portions of cfDNA to identify patterns of differential methylation, and using these patterns of differential methylation to determine the cellular origin of the cfDNA.

The use of patterns of differential methylation to determine the cellular origin of cfDNA can be applied to methods of determining if a subject has suffered tissue damage from exposure to a toxic agent. In some embodiments, the methods comprise (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation pattern; (c) measuring the quantity of the cfDNA of the determined cellular origin, and (d) comparing the measured quantity of the cfDNA of the determined cellular origin with a normal quantity of cfDNA of the determined cellular origin. An increase in the measured quantity of the cfDNA of the determined cellular origin over the normal quantity of cfDNA of the determined cellular origin is indicative that the subject has suffered or suffers tissue damage from the exposure.

In some embodiments, the methods of determining if a subject has suffered tissue damage from exposure to a toxic agent comprise, at two or more time points, (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation pattern; and (c) measuring the quantity of the cfDNA of the determined cellular origin. An increase in the measured quantity of the cfDNA of the determined cellular origin at a later time point as compared to an earlier time point is indicative that the subject has suffered or suffers tissue damage from the exposure.

The use of patterns of differential methylation to determine the cellular origin of cfDNA can also be applied to methods of treating a subject who has suffered tissue damage from exposure to a toxic agent. In some embodiments, these methods comprise administering a treatment for the tissue damage to the subject, in which the subject was indicated as suffering tissue damage by a method comprising (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation pattern; (c) measuring the quantity of the cfDNA of the determined cellular origin, and (d) comparing the measured quantity of the cfDNA of the determined cellular origin with a normal quantity of cfDNA of the determined cellular origin. An increase in the measured quantity of the cfDNA of the determined cellular origin over the normal quantity of cfDNA of the determined cellular origin is indicative that the subject has suffered tissue damage.

In some embodiments, the methods of treating a subject who has suffered tissue damage from exposure to a toxic agent comprise administering a treatment for the tissue damage to the subject, in which the subject was indicated as suffering tissue damage by a method comprising, at two or more time points, (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation pattern; and (c) measuring the quantity of the cfDNA of the determined cellular origin. An increase in the measured quantity of the cfDNA of the determined cellular origin at a later time point as compared to an earlier time point is indicative that the subject has suffered tissue damage.

In other embodiments, the methods are for treating tissue damage in a subject. The methods comprise administering a treatment for tissue damage to the subject and monitoring the efficacy of the treatment. The monitoring comprises (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation pattern; (c) measuring the quantity of the cfDNA of the determined cellular origin, and (d) comparing the measured quantity of the cfDNA of the determined cellular origin with a normal quantity of cfDNA of the determined cellular origin. A decrease in the measured quantity of the cfDNA of the determined cellular origin as compared to the normal quantity of cfDNA of the determined cellular origin is indicative that the treatment is effective. An increase or no change in the measured quantity of the cfDNA of the determined cellular origin over the normal quantity of cfDNA of the determined cellular origin is indicative that the treatment is not effective.

In some embodiments, the methods for treating tissue damage comprise administering a treatment for tissue damage to the subject and monitoring the efficacy of the treatment. The monitoring comprises, at two or more time points, (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation pattern; and (c) measuring the quantity of the cfDNA of the determined cellular origin. A decrease in the measured quantity of the cfDNA of the determined cellular origin at a later time point as compared to an earlier time point is indicative that the treatment is effective. An increase or no change in the measured quantity of the cfDNA of the determined cellular origin at a later time point as compared to an earlier time point is indicative that the treatment is not effective.

In some embodiments, the methods may further comprise administering an adjusted treatment when the first treatment is determined to be not effective. In some embodiments, the tissue damage is caused by exposure to a toxic agent.

In some embodiments, the toxic agent comprises radiation. The radiation may be for therapeutic purposes, accidental, or environmental.

In some embodiments, the toxic agent is a radiation therapy. In certain embodiments, the radiation therapy comprises an extremal beam radiation therapy. Examples of extremal beam radiation therapy include, but are not limited to, conventional external beam radiation therapy, stereotactic radiation therapy, three-dimensional conformal radiation therapy, intensity-modulated radiation therapy, volumetric modulated arc therapy, temporally feathered radiation therapy, particle therapy, and auger therapy.

In certain embodiments, the radiation therapy comprises a brachytherapy, in which the radiation is in a sealed source. The brachytherapy may be an interstitial brachytherapy, in which the radiation source is placed directly in the target tissue of the affected site; or the brachytherapy may be a contact brachytherapy, in which the radiation source is placed in a space next to the target tissue, such as a body cavity (intracavitary brachytherapy), a body lumen (intraluminal brachytherapy), or externally (surface brachytherapy).

In certain embodiments, the radiation therapy comprises systemic radioisotope therapy, which delivers the radiation to a targeted site using, for instance, chemical properties of the isotope or attachment of the isotope to another molecule or antibody that guides the isotope to the targeted site.

In some embodiments, the toxic agent is accidental radiation, for example, work-related exposure to radiation.

In some embodiments, the toxic agent is environmental radiation. Environmental radiation include exposure to radiation resulting from, as non-limiting examples, high-attitude flights and space travel.

In some embodiments, the toxic agent comprises a radioactive substance ingested by the subject, inhaled by the subject, or absorbed through body surface contamination by the subject.

In some embodiments, the toxic agent comprises a microorganism. In certain embodiments, the toxic agent comprises a pathogen such as a bacterium or virus. Particular examples of pathogens include, but are not limited to, species of the following genus: Bacillus, Brucella, Clostridium, Corynebacterium, Enterococcus, Escherichia, Klebsiella, Leptospira, Listeria, Mycobacterium, Mycoplasma, Neisseria, Pseudomonas, Staphylococcus, Treponema, Vibrio, and Yersinia.

In some embodiments, the toxic agent comprises a toxin from a synthetic chemical source or from a biological source.

In some embodiments, the toxic agent comprises a pharmaceutical therapy, such as a chemical used for therapeutic purposes.

In some embodiments, the toxic agent comprises a chemical or biological or radioactive substance used as a weapon, for example, in a terrorist attack or in a war.

In yet other embodiments, the methods of treating a subject comprise administering a treatment to the subject and monitoring whether the treatment causes tissue damage in the subject. The monitoring comprises (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation pattern; (c) measuring the quantity of the cfDNA of the determined cellular origin, and (d) comparing the measured quantity of the cfDNA of the determined cellular origin with a normal quantity of cfDNA of the determined cellular origin. An increase in the measured quantity of the cfDNA of the determined cellular origin over the normal quantity of cfDNA of the determined cellular origin is indicative that the treatment is causing tissue damage.

In other embodiments, methods of treating a subject comprise administering a treatment to the subject and monitoring whether the treatment causes tissue damage in the subject. The monitoring comprises, at two or more time points, (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation pattern; and (c) measuring the quantity of the cfDNA of the determined cellular origin. An increase in the measured quantity of the cfDNA of the determined cellular origin at later time point as compared to an earlier time point is indicative that the treatment is causing tissue damage.

In some embodiments, the methods may further comprise administering an adjusted treatment when the first treatment is determined to cause tissue damage.

In some embodiments, the normal quantity of cfDNA comprises a quantity of cfDNA for the determined cellular origin that is generated in a population of individuals who were not exposed to the toxic agent. In other embodiments, the normal quantity of cfDNA comprises a quantity of cfDNA for the determined cellular origin that is generated in a population of individuals who were not administered the treatment.

Another aspect of the present invention is a method of determining organ-, tissue-, or cell-type damage induced by a substance administered to the subject. The method comprises (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation pattern; (c) measuring the quantity of the cfDNA of the determined cellular origin, and (d) comparing the measured quantity of the cfDNA of the determined cellular origin with a normal quantity of cfDNA of the determined cellular origin. An increase in the measured quantity of the cfDNA of the determined cellular origin over the normal quantity of cfDNA of the determined cellular origin is indicative that an organ or tissue of the cell type, or the cell-type itself, has suffered damage. In some embodiments, the substance administered to the subject may be a pharmaceutical, such as an investigational newt drug.

Yet another aspect of the present invention is a method of determining organ-, tissue-, or cell-type damage induced by a substance administered to the subject. The method comprises, at two or more time points, (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation pattern; and (c) measuring the quantity of the cfDNA of the determined cellular origin. An increase in the measured quantity of the cfDNA of the determined cellular origin at a later time point as compared to an earlier time point is indicative that an organ or tissue of the cell type, or the cell-type itself, has suffered damage. In some embodiments, the substance administered to the subject may be a pharmaceutical, such as an investigational new drug.

A further aspect of the present invention is a method of determining the organ-, tissue-, or cell-target of a substance administered to a subject. The method comprises (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation pattern; (c) measuring the quantity of the cfDNA of the determined cellular origin, and (d) comparing the measured quantity of the cfDNA of the determined cellular origin with a normal quantity of cfDNA of the determined cellular origin. An increase in the measured quantity of the cfDNA of the determined cellular origin over the normal quantity of cfDNA of the determined cellular origin is indicative that an organ or tissue of the cell type, or the cell-type itself, is a target of the substance. In embodiments, the substance administered to the subject may be a pharmaceutical, such as an investigational new drug.

Yet, a further aspect of the present invention is a method of determining the organ-, tissue-, or cell-target of a substance administered to a subject. The method comprises, at two or more time points, (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation pattern; and (c) measuring the quantity of the cfDNA of the determined cellular origin. An increase in the measured quantity of the cfDNA of the determined cellular origin at a later time point as compared to an earlier time point is indicative that an organ or tissue of the cell type, or the cell-type itself, is a target of the substance. In embodiments, the substance administered to the subject may be a pharmaceutical, such as an investigational new drug.

In some embodiments, the normal quantity of cfDNA comprises a quantity of cfDNA for the determined cellular origin that is generated in a population of individuals who were not exposed to the toxic agent. In other embodiments, the normal quantity of cfDNA comprises a quantity of cfDNA for the determined cellular origin that is generated in a population of individuals who were not administered the treatment.

In some embodiments, the normal quantity of cfDNA of the determined cellular origin is a quantity of cfDNA for the determined cellular origin that is expected for the determined cellular origin.

In some embodiments, the two or more time points may all be after treatment or exposure to the toxic agent. In some embodiments, at least one of the two or more time points may be before treatment or exposure to the toxic agent.

The time points may be, for instance, one or more days apart, for example, every day, every two days, every three days, every four days, every five days, every six days, every week every two weeks, every three weeks, every four weeks, every month, every two months, every three months, every four months, every five months, every six months, every seven months, every eight months, every nine months, every ten months, every 11 months, every year, or any time therebetween.

The increase in the measured quantity of the cfDNA of the determined cellular origin over the normal quantity of cfDNA of the determined cellular origin, or over a previously measured quantity of cfDNA of the determined cellular origin, may be, for example, a percent increase of about 0.1% to 100%, such as about 0.1%, 0.5%, 1%, 2%, 3%, 4%, 5%, 6% 7%, 8%, 9%, 10%, 20%, 30%, 40%, 50%, 60%, 70%, 80%, 90%, or 100%; or may be a fold increase of at least about 2-fold, such as about 2-fold, or 3-fold, or 4-fold, or 5-fold, or 6-fold, or 7-fold, or 8-fold, or 9-fold, or 10-fold. In some embodiments, the increase may be any increase that is determined to be statistically significant (e.g., p≤005, p≤0.01, etc.) as calculated by statistical methods known in the art.

In some embodiments, the subject has cancer.

The biospecimen may be a biological fluid obtained from the subject, including, but not limited to, whole blood, plasma, serum, urine, or any other fluid sample produced by the subject such as saliva, cerebrospinal fluid, urine, or sputum. In certain embodiments, the biospecimen is whole blood, plasma, or serum.

Methods for quantifying the cfDNA are known in the art and include, but are not limited to, PCR; fluorescence-based quantification methods (e.g., Qubit); chromatography techniques such as gas chromatography, supercritical fluid chromatography, and liquid chromatography, such as partition chromatography, adsorption chromatography, ion exchange chromatography, size exclusion chromatography, thin-layer chromatography, and affinity chromatography: electrophoresis techniques, such as capillary electrophoresis, capillary zone electrophoresis, capillary isoelectric focusing, capillary electrochromatography, micellar electrokinetic capillary chromatography, isotachophoresis, transient isotachophoresis, and capillary gel electrophoresis; comparative genomic hybridization; microarrays; and bead arrays.

### Methods Combining Epigenetic and Genetic Analyses

The use of patterns of differential methylation to determine the cellular origin of cfDNA can be combined with a genetic analysis of the cfDNA. Such a combination can be applied to method of treatment that involves monitoring treatment response and therapy-related adverse events. Combining changes to mutant cfDNA with altered proportions of cell-type specific cfDNA can reflect intervention-based changes. The half-life of cfDNA is between 15 minutes and 2 hours. The rapid clearance allows for serial analysis of disease evolution over time, especially under selective pressures from ongoing therapy. The methods of the invention allow for serial sampling to include a baseline comparison from which therapy-related relative changes may be assessed, taking into account patient specific co-morbidities at an individualized level.

Combining genetic and epigenetic analyses of cell-free DNA has many unique advantages when applied to precision therapeutics in cancer. Liquid biopsies have been shown to accurately characterize tumor genotypes and allow for molecular subtype classification to provide a comprehensive view of intratumor heterogeneity. High sampling frequency allows for modeling of evolutionary dynamics of tumor progression. Also, molecular changes identified after initiation of therapy can provide insight into therapy response as well as track tumor subclones that may lead to emergence of therapy resistance. The systemic view provided by serial liquid biopsies is ideal to monitor widespread changes that may better inform clinical decision making in the face of uncertainty. For example, in the case of surgical removal of the tumor or therapeutic success, liquid biopsies can be used to monitor for minimal residual disease and recurrence. While cfDNA can be used to track molecular changes in the circulation, there is a benefit to monitoring the cancer-related changes to the host microenvironment in tandem requiring a combined genetic and epigenetic analysis. Cell-specific cfDNA methylation patterns of normal cells can be used in combination with ctDNA to assess the impact of treatment also on the surrounding tumor microenvironment. This is particularly useful to surveil for metastatic disease in distant tissue-types from the primary tumor as well as to monitor for therapy-related toxicities in somatic cell types. Further, liquid biopsies can help delineate factors that underlie clinical outcomes, providing a basis for recommending different treatments based on anticipated benefit to the patient. Liquid biopsies can identify predictive biomarkers to guide selection of treatment, recognize off-target effects and develop individualized treatment plans for patients. These applications provide a more complete picture of therapeutic response as well as tissue-specific cellular toxicity to better inform clinical care and management throughout the treatment process.

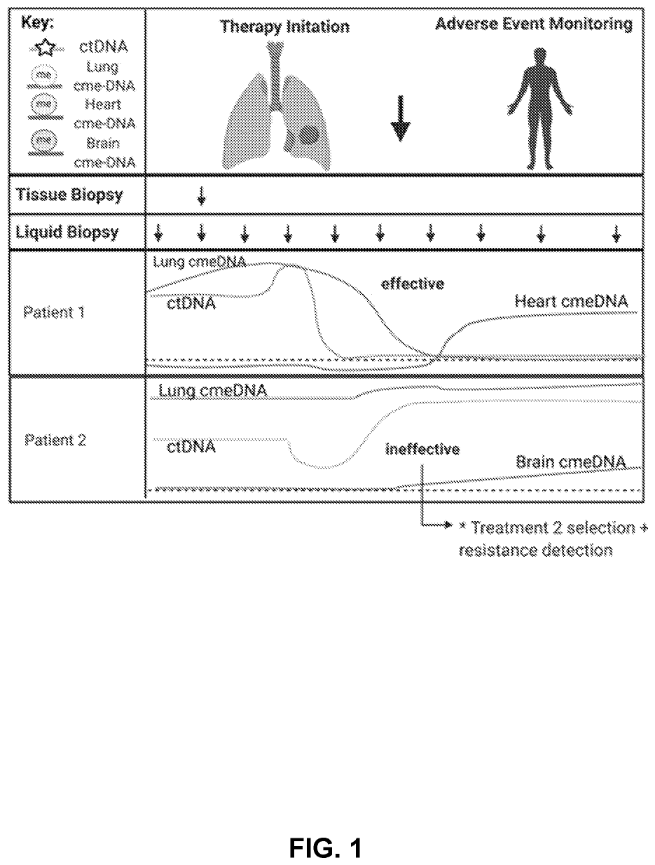

The minimally invasive nature of liquid biopsies allows for serial sampling to monitor changes over time, especially under selective pressures from ongoing therapy. ctDNA can be used to track clonal heterogeneity over time to assess treatment response and detect treatment-resistant clones. Normal cell-specific cfDNA methylation patterns can be used in combination with ctDNA to assess the impact of treatment to the surrounding tumor microenvironment and to monitor for therapy-related toxicities in somatic cell-types (FIG. 1).

The use of patterns of differential methylation to determine the cellular origin of cfDNA in combination with genetic analysis can be applied to methods of treating a subject having a tumor. In some embodiments, the methods comprise (a) monitoring the response to a first treatment, an adverse reaction to the first treatment, or a combination thereof, in which the monitoring comprises, at two or more time points, performing a genetic and epigenetic analysis of cfDNA, ctDNA, or a combination thereof, and optionally comparing to normal cfDNA, cfDNA, or a combination thereof, to determine whether to change the first treatment; and (b) administering an adjusted treatment or continuing the first treatment in accordance with the genetic and epigenetic analysis.

In other embodiments, the methods comprise (A) monitoring a response to a first treatment, an adverse reaction to the first treatment, or a combination thereof, in which the monitoring comprises: (i) determining whether there is an adverse reaction to the first treatment, which comprises (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation patterns; (c) measuring the quantity of the cfDNA of the determined cellular origin; and (d) comparing the measured quantity of the cfDNA of the determined cellular origin with a normal quantity of cfDNA of the determined cellular origin, in which an increase in the measured quantity of the cfDNA of the determined cellular origin over the normal quantity of cfDNA of the determined cellular origin is indicative of an adverse reaction; and (ii) determining whether there is a response to the first treatment, which comprises: (a) sequencing cfDNA in a biospecimen from the subject, and (b) determining clonal heterogeneity of cells of the tumor by genotyping the cfDNA, in which the presence of more than one clone of the tumor cells or the presence of a tumor cell clone that has not been previously identified in the subject is indicative of an ineffective response to the first treatment; and (B) either administering the same treatment as the first treatment when it is determined that there is no adverse reaction, that there is not an ineffective response, or a combination thereof; or administering an adjusted treatment when it is determined that there is an adverse reaction, that there is an ineffective response, or a combination thereof.

In some embodiments, the normal quantity of cfDNA comprises a quantity of cfDNA for the determined cellular origin that is generated in a population of individuals who did not receive the first treatment. In other embodiments, the normal quantity of cfDNA comprises a quantity of cfDNA for the determined cellular origin that is generated in a population of individuals who do not have the tumor.

In some embodiments, the normal quantity of cfDNA of the determined cellular origin is a quantity of cfDNA for the determined cellular origin that is expected for the determined cellular origin.

In yet other embodiments, the methods comprise (A) monitoring a response to a first treatment, an adverse reaction to the first treatment, or a combination thereof, in which the monitoring comprises, at two or more time points, (i) determining whether there is an adverse reaction to the first treatment, which comprises (a) sequencing cfDNA in a biospecimen from the subject; (b) determining cellular origin of the cfDNA by identifying the methylation patterns in one or more portions of the sequence of the cfDNA that contains methylation sites, in which the cellular origin of the cell-free DNA is determined when the methylation pattern in the one or more portions is the same as a known cell-type specific methylation patterns; and (c) measuring the quantity of the cfDNA of the determined cellular origin, wherein an increase in the measured quantity of the cfDNA of the determined cellular origin measured at a later time point as compared to an earlier time point is indicative of an adverse reaction; and (ii) determining whether there is a response to the first treatment, which comprises (a) sequencing cfDNA in a biospecimen from the subject; and (b) determining clonal heterogeneity of cells of the tumor by genotyping the cfDNA, wherein the presence of more than one clone of the tumor cells or the presence of a tumor cell clone in a subsequent time point that has not been identified at a previous time point is indicative of an ineffective response to the first treatment; and (B) either administering the same treatment as the first treatment when it is determined that there is no adverse reaction, that there is not an ineffective response, or a combination thereof; or administering an adjusted treatment when it is determined that there is an adverse reaction, that there is an ineffective response, or a combination thereof.

In some embodiments, the subject has a tumor associated with a cancer. Examples of cancer include, but are not limited to, colorectal cancer, brain cancer, ovarian cancer, prostate cancer, pancreatic cancer, breast cancer, renal cancer, nasopharyngeal carcinoma, hepatocellular carcinoma, melanoma, skin cancer, oral cancer, head and neck cancer, esophageal cancer, gastric cancer, cervical cancer, bladder cancer, lymphoma, chronic or acute leukemia (such as B, T, and myeloid derived), sarcoma, lung cancer and multidrug resistant cancer. Other examples are disease that require drug treatment with chemical compounds (small molecules) or proteins such as insulin or antibodies. Such disease can be metabolic disease such as diabetes mellitus or infections such as bacterial or viral infections such as hepatitis or cardiovascular disease including but not limited to hypertension, coronary artery disease, cerebral vascular disease or peripheral vascular disease.

In some embodiments, cfDNA is used to compare damage to cells from the first treatment with undamaged normal cells from the same tissue.

In some embodiments, methylation patterns are assessed in the cfDNA. In certain embodiments, the methylation patterns of cfDNA from damaged cells and healthy cells are compared.

In some embodiments, the analysis includes comparing damaged cells to healthy cells, to see where the damage originated.

In some embodiments, the treatment comprises a chemotherapy, radiotherapy, targeted therapy, immunotherapy, or a combination thereof.

In some embodiments, the two or more time points may all be after the first treatment. In some embodiments, at least one of the two or more time points may be before the first treatment.

The time points may be, for instance, one or more days apart, for example, every day, every two days, every three days, every four days, every five days, every six days, every week every two weeks, every three weeks, every four weeks, every month, every two months, every three months, every four months, every five months, every six months, every seven months, every eight months, every nine months, every ten months, every 11 months, every year, or any time therebetween.

The increase in the measured quantity of the cfDNA of the determined cellular origin over the normal quantity of cfDNA of the determined cellular origin, or over a previously measured quantity of cfDNA of the determined cellular origin, may be, for example, a percent increase of about 0.1% to 100%, such as about 0.1%, 0.5%, 1%, 2%, 3%, 4%, 5%, 6%, 7%, 8%, 9%, 10%, 20%, 30%, 40%, 50%, 60%, 70%, 80%, 90%, or 100%; or may be a fold increase of at least about 2-fold, such as about 2-fold, or 3-fold, or 4-fold, or 5-fold, or 6-fold, or 7-fold, or 8-fold, or 9-fold, or 10-fold. In some embodiments, the increase may be any increase that is determined to be statistically significant (e.g., p≤0.05, p≤0.01, etc.) as calculated by statistical methods known in the art.

The biospecimen may be a biological fluid obtained from the subject, including, but not limited to, whole blood, plasma, serum, urine, or any other fluid sample produced by the subject such as saliva, cerebrospinal fluid, urine, or sputum. In certain embodiments, the biospecimen is whole blood, plasma, or serum.

Methods for quantifying the cfDNA are known in the art and include, but are not limited to, PCR; fluorescence-based quantification methods (e.g., Qubit); chromatography techniques such as gas chromatography, supercritical fluid chromatography, and liquid chromatography, such as partition chromatography, adsorption chromatography ion exchange chromatography, size exclusion chromatography, thin-layer chromatography, and affinity chromatography; electrophoresis techniques, such as capillary electrophoresis, capillary zone electrophoresis, capillary isoelectric focusing, capillary electrochromatography, micellar electrokinetic capillary chromatography, isotachophoresis, transient isotachophoresis, and capillary gel electrophoresis; comparative genomic hybridization; microarrays; and bead arrays.

Another aspect of the invention relates to methods of detecting and/or quantitating changes in methylated DNA in the circulation of patients undergoing treatment.

A further aspect of the invention relates to probes designed for any tissue and/or cell type in a tissue to detect changes in the abundance of tissue-specific DNA fragments in the circulation.

Analysis of cfDNA

The present invention involves analysis of cfDNA to determine the cellular origin of cfDNA. Determination of the cellular origin of cfDNA comprises identifying methylation patterns in the sequence of the cfDNA and comparing the methylation patterns in the sequence of the cfDNA to know % n methylation patterns associated with different cell types.

Table 1 provides examples of cellular origins associated with different types of tissue.

TABLE 1
Cellular origins, and the different types of tissue
with which they can be associated.

| Cellular Origins | Tissue |
|---|---|
| Mature B-Cell | Blood, Bone Marrow |
| Naïve B-Cell | Blood, Bone Marrow |
| Biliary Epithelial Cell | Liver |
| Breast Basal Cell | Breast |
| Breast Luminal Cell | Breast |
| Bulk Endothelial Cell | Blood Vessels |
| Bulk Epithelial Cell | Any Epithelia |
| Bulk Immune Cell | Immune Organ |
| Cardiomyocyte | Heart |
| Cardiopulmonary Endothelial Cell | Heart, Lung |
| Colon Epithelial Cell | Colon |
| Dermal Epithelial Cell | Skin |
| Granulocyte | Blood, Bone Marrow |
| Hepatocyte | Liver |
| Keratinocyte | Skin |
| Kidney Epithelial Cell | Kidney |
| Liver Endothelial Cell | Liver |
| Liver Stromal Cell | Liver |
| Liver Resident Immune Cell | Liver |
| Lung Epithelial Cell | Lung |
| Megakaryocyte | Bone Marrow |
| Monocytes and Macrophage | Blood |
| Neuron | Neural |
| Natural Killer Cell | Blood |
| Pancreatic Cell | Pancreas |
| Prostate Epithelial Cell | Prostate |
| Skeletal Muscular Cell | Skeletal Muscle |
| Mature T-Cell | Blood |

CfDNA can be obtained by centrifuging the biological fluid, such as whole blood, to remove all cells, and then isolating the DNA from the remaining plasma or serum. Such methods are well known (see, e.g., Lo et al, 1998). Circulating cfDNA and cfDNA can be double-stranded or single-stranded DNA.

Different DNA methylation detection technologies may be used in the present invention. Examples include, but are not limited to, a restriction enzyme digestion approach, which involves cleaving DNA at enzyme-specific CpG sites; an affinity-enrichment method, for instance, methylated DNA immunoprecipitation sequencing (MeDIP-seq) or methyl-CpG-binding domain sequencing (MBD-seq); bisulfite conversion methods such as whole genome bisulfite sequencing (WGBS), reduced representation bisulfite sequencing (RRBS), methylated CpG tandem amplification and sequencing (MCTA-seq), and methylation arrays; enzymatic approaches, such as enzymatic methyl-sequencing (EM-seq) or ten-eleven translocation (TET)-assisted pyridine borane sequencing (TAPS); and other methods that do not require treatment of DNA, for instance, by nanopore-sequencing from Oxford Nanopore Technologies (ONT) and single molecule real-time (SMRT) sequencing from Pacific Biosciences (PacBio).

Comparison of the methylation pattern in sequence of the cfDNA with known methylation patterns may comprise identifying the presence of a methylation pattern in the sequence of the cfDNA, or a portion thereof, that are attributed to specific cell types. In some embodiments, the presence of a methylation pattern was performed by hybridization capture sequencing of cfDNA. In other embodiments, the presence of a methylation pattern was performed using bisulfite amplicon sequencing.

The methylation pattern may comprise a segment of nucleotide sequence containing at least 1 CpG dinucleotide, or at least about 2 CpG dinucleotides, or at least about 3 CpG dinucleotides. In some embodiments, the methylation pattern may comprise a segment of nucleotide sequence containing at least about 4 CpG dinucleotides, or at least about 5 CpG dinucleotides, or at least about 6 CpG dinucleotides, or at least about 7 CpG dinucleotides, or at least about 8 CpG dinucleotides, or at least about 9 CpG dinucleotides, or at least about 10 CpG dinucleotides.

Table 2 provides methylation status at CpG dinucleotides in genomic regions that indicative of different cell types. The presence of a same methylation pattern between the sequence of the cfDNA and the genomic regions set forth in Table 2 indicates the cell-type from which the cfDNA originates. Table 2 provides contiguous methylation status across multiple adjacent CpG sites (patterns) within genomic region.

TABLE 2
Methylation status in genomic regions that are indicative of cell type.

| | | | | Methylation |
|---|---|---|---|---|
| Cell Type | Chromosome | Start* | End* | Status |
| Mature B | chr11 | 68139032 | 68139146 | Hypomethylated |
| Mature B | chr17 | 80829337 | 80829647 | Hypomethylated |
| Mature B | chr6 | 167506945 | 167507168 | Hypomethylated |
| Mature B | chr19 | 1648937 | 1649129 | Hypomethylated |
| Mature B | chr3 | 9694444 | 9695149 | Hypomethylated |
| Mature B | chr18 | 77116085 | 77116618 | Hypomethylated |
| Mature B | chr9 | 135763441 | 135764023 | Hypomethylated |
| Mature B | chr12 | 121686411 | 121686789 | Hypomethylated |
| Mature B | chr6 | 16306332 | 16306681 | Hypomethylated |
| Mature B | chr14 | 96179945 | 96180308 | Hypomethylated |
| Mature B | chr6 | 16306086 | 16306267 | Hypomethylated |
| Mature B | chr16 | 28944140 | 28944468 | Hypomethylated |
| Mature B | chr17 | 73316011 | 73316779 | Hypomethylated |
| Mature B | chr2 | 112917111 | 112917496 | Hypomethylated |
| Mature B | chr7 | 637567 | 637692 | Hypomethylated |
| Mature B | chr17 | 79233329 | 79233604 | Hypomethylated |
| Mature B | chr2 | 240291097 | 240291331 | Hypomethylated |
| Mature B | chr17 | 3493609 | 3493935 | Hypomethylated |
| Mature B | chr11 | 2415602 | 2415708 | Hypomethylated |
| Mature B | chr13 | 111329306 | 111329450 | Hypomethylated |
| Mature B | chr6 | 167507176 | 167507296 | Hypomethylated |
| Mature B | chr10 | 1704918 | 1705006 | Hypomethylated |
| Mature B | chr14 | 104158553 | 104158779 | Hypomethylated |
| Mature B | chr17 | 80873275 | 80873776 | Hypomethylated |
| Mature B | chr16 | 89180214 | 89180662 | Hypomethylated |
| Mature B | chr11 | 64567087 | 64567240 | Hypomethylated |
| Mature B | chr19 | 2324328 | 2324440 | Hypomethylated |
| Mature B | chr22 | 50196818 | 50196977 | Hypomethylated |
| Mature B | chr16 | 88103031 | 88103137 | Hypomethylated |
| Mature B | chr19 | 1621010 | 1621318 | Hypomethylated |
| Mature B | chr1 | 11395635 | 11395863 | Hypomethylated |
| Mature B | chr2 | 464998 | 465071 | Hypomethylated |
| Mature B | chr16 | 1495324 | 1495725 | Hypomethylated |
| Mature B | chr9 | 101754799 | 101755349 | Hypomethylated |
| Mature B | chr5 | 177544974 | 177545232 | Hypomethylated |
| Mature B | chr6 | 159639094 | 159639294 | Hypomethylated |
| Mature B | chr8 | 23083219 | 23083470 | Hypomethylated |
| Mature B | chr14 | 104852775 | 104853229 | Hypomethylated |
| Mature B | chr7 | 2140017 | 2140179 | Hypomethylated |
| Mature B | chr19 | 56156225 | 56156453 | Hypomethylated |
| Mature B | chr10 | 121201654 | 121201754 | Hypomethylated |
| Mature B | chr20 | 57033426 | 57033735 | Hypomethylated |
| Mature B | chr2 | 61199859 | 61200489 | Hypomethylated |
| Mature B | chr15 | 75146404 | 75146784 | Hypomethylated |
| Mature B | chr16 | 85289643 | 85290003 | Hypomethylated |
| Mature B | chr16 | 88765052 | 88765399 | Hypomethylated |
| Mature B | chr7 | 2140231 | 2140348 | Hypomethylated |
| Mature B | chr16 | 773800 | 773999 | Hypomethylated |
| Mature B | chr10 | 13330436 | 13330842 | Hypomethylated |
| Mature B | chr15 | 74714681 | 74715017 | Hypomethylated |
| Naïve B | chr11 | 68139032 | 68139146 | Hypomethylated |
| Naïve B | chr17 | 3493609 | 3493935 | Hypomethylated |
| Naïve B | chr19 | 1648937 | 1649129 | Hypomethylated |
| Naïve B | chr17 | 80829337 | 80829647 | Hypomethylated |
| Naïve B | chr22 | 20760823 | 20761115 | Hypomethylated |
| Naïve B | chr17 | 80873275 | 80873776 | Hypomethylated |
| Naïve B | chr2 | 240291097 | 240291331 | Hypomethylated |
| Naïve B | chr10 | 6262461 | 6262727 | Hypomethylated |
| Naïve B | chr11 | 64567087 | 64567240 | Hypomethylated |
| Naïve B | chr14 | 104158553 | 104158779 | Hypomethylated |
| Naïve B | chr19 | 2324328 | 2324440 | Hypomethylated |
| Naïve B | chr16 | 773800 | 773999 | Hypomethylated |
| Naïve B | chr8 | 16617187 | 16617280 | Hypomethylated |
| Naïve B | chr16 | 88103031 | 88103137 | Hypomethylated |
| Naïve B | chr5 | 177544974 | 177545232 | Hypomethylated |
| Naïve B | chr14 | 96179945 | 96180308 | Hypomethylated |
| Naïve B | chr4 | 185876236 | 185876334 | Hypomethylated |
| Naïve B | chr9 | 135763441 | 135764023 | Hypomethylated |
| Naïve B | chr11 | 2415602 | 2415708 | Hypomethylated |
| Naïve B | chr10 | 121201654 | 121201754 | Hypomethylated |
| Naïve B | chr7 | 2140017 | 2140179 | Hypomethylated |
| Naïve B | chr16 | 2516952 | 2517020 | Hypomethylated |
| Naïve B | chr16 | 75107515 | 75107841 | Hypomethylated |
| Biliary Epithelial | chr10 | 7744476 | 7744775 | Hypomethylated |
| Biliary Epithelial | chr19 | 35534546 | 35535085 | Hypomethylated |
| Biliary Epithelial | chr22 | 39685694 | 39685809 | Hypomethylated |
| Biliary Epithelial | chr16 | 87824936 | 87825139 | Hypomethylated |
| Biliary Epithelial | chr17 | 48626642 | 48627290 | Hypomethylated |
| Biliary Epithelial | chr1 | 19600102 | 19600251 | Hypomethylated |
| Biliary Epithelial | chr3 | 128141404 | 128141587 | Hypomethylated |
| Biliary Epithelial | chr5 | 53223677 | 53224181 | Hypomethylated |
| Biliary Epithelial | chr9 | 137331241 | 137331537 | Hypomethylated |
| Biliary Epithelial | chr11 | 47471307 | 47471401 | Hypomethylated |
| Biliary Epithelial | chr17 | 79042980 | 79043169 | Hypomethylated |
| Biliary Epithelial | chr22 | 37419795 | 37420227 | Hypomethylated |
| Biliary Epithelial | chr2 | 74731371 | 74731414 | Hypomethylated |
| Biliary Epithelial | chr5 | 170876495 | 170876741 | Hypomethylated |
| Biliary Epithelial | chr10 | 135340893 | 135341026 | Hypomethylated |
| Biliary Epithelial | chr20 | 56287198 | 56287355 | Hypomethylated |
| Biliary Epithelial | chr1 | 9324077 | 9324214 | Hypomethylated |
| Biliary Epithelial | chr2 | 241827906 | 241828206 | Hypomethylated |
| Biliary Epithelial | chr14 | 101944626 | 101944805 | Hypomethylated |
| Biliary Epithelial | chr21 | 46893085 | 46893254 | Hypomethylated |
| Biliary Epithelial | chr2 | 241949229 | 241949839 | Hypermethylated |
| Biliary Epithelial | chr17 | 42084940 | 42085300 | Hypermethylated |
| Biliary Epithelial | chr19 | 50095791 | 50096912 | Hypermethylated |
| Biliary Epithelial | chr10 | 11726866 | 11727466 | Hypermethylated |
| Biliary Epithelial | chr3 | 13114628 | 13114763 | Hypermethylated |
| Biliary Epithelial | chr10 | 102882978 | 102883606 | Hypermethylated |
| Biliary Epithelial | chr11 | 2160875 | 2161446 | Hypermethylated |
| Biliary Epithelial | chr19 | 3434917 | 3435465 | Hypermethylated |
| Biliary Epithelial | chr8 | 11565610 | 11565963 | Hypermethylated |
| Biliary Epithelial | chr8 | 11566699 | 11566916 | Hypermethylated |
| Biliary Epithelial | chr11 | 10328197 | 10328509 | Hypermethylated |
| Biliary Epithelial | chr8 | 11565188 | 11565531 | Hypermethylated |
| Biliary Epithelial | chr14 | 38067833 | 38067990 | Hypermethylated |
| Biliary Epithelial | chr16 | 88716968 | 88717851 | Hypermethylated |
| Biliary Epithelial | chr22 | 37464939 | 37465280 | Hypermethylated |
| Biliary Epithelial | chr7 | 44185404 | 44185968 | Hypermethylated |
| Biliary Epithelial | chr16 | 127503 | 127698 | Hypermethylated |
| Biliary Epithelial | chr3 | 197281447 | 197283152 | Hypermethylated |
| Biliary Epithelial | chr18 | 55107463 | 55107887 | Hypermethylated |
| Biliary Epithelial | chr15 | 53087579 | 53087929 | Hypermethylated |
| Biliary Epithelial | chr11 | 2161456 | 2162090 | Hypermethylated |
| Biliary Epithelial | chr14 | 38067054 | 38067566 | Hypermethylated |
| Biliary Epithelial | chr7 | 155597344 | 155597625 | Hypermethylated |
| Biliary Epithelial | chr2 | 128181286 | 128181430 | Hypermethylated |
| Biliary Epithelial | chr3 | 55515070 | 55515736 | Hypermethylated |
| Biliary Epithelial | chr6 | 152128957 | 152129855 | Hypermethylated |
| Biliary Epithelial | chr5 | 176829535 | 176830185 | Hypermethylated |
| Biliary Epithelial | chr2 | 128180566 | 128181267 | Hypermethylated |
| Biliary Epithelial | chr11 | 2164937 | 2165828 | Hypermethylated |
| Biliary Epithelial | chr8 | 27182852 | 27183553 | Hypermethylated |
| Breast Basal | chr2 | 44497562 | 44497807 | Hypomethylated |
| Breast Basal | chr17 | 4453891 | 4454402 | Hypomethylated |
| Breast Basal | chr11 | 57558973 | 57559168 | Hypomethylated |
| Breast Basal | chr11 | 392133 | 392695 | Hypomethylated |
| Breast Basal | chr22 | 47023757 | 47023904 | Hypomethylated |
| Breast Basal | chr2 | 240040057 | 240040254 | Hypomethylated |
| Breast Basal | chr1 | 3321982 | 3322140 | Hypomethylated |
| Breast Basal | chr18 | 77635864 | 77636069 | Hypomethylated |
| Breast Basal | chr22 | 40417354 | 40417567 | Hypomethylated |
| Breast Basal | chr5 | 137803144 | 137803521 | Hypomethylated |
| Breast Basal | chr11 | 391681 | 392015 | Hypomethylated |
| Breast Basal | chr16 | 27781217 | 27781639 | Hypomethylated |
| Breast Basal | chr17 | 2278727 | 2279131 | Hypomethylated |
| Breast Basal | chr17 | 70053558 | 70053834 | Hypomethylated |
| Breast Basal | chr10 | 123738139 | 123738614 | Hypomethylated |
| Breast Basal | chr16 | 81533203 | 81533549 | Hypomethylated |
| Breast Basal | chr18 | 10588980 | 10589063 | Hypomethylated |
| Breast Basal | chr18 | 10589150 | 10589279 | Hypomethylated |
| Breast Basal | chr19 | 17437804 | 17438236 | Hypomethylated |
| Breast Basal | chr19 | 46388006 | 46388262 | Hypomethylated |
| Breast Basal | chr20 | 62045021 | 62045251 | Hypomethylated |
| Breast Basal | chr1 | 2832006 | 2832144 | Hypomethylated |
| Breast Basal | chr16 | 88166706 | 88166848 | Hypomethylated |
| Breast Basal | chr2 | 233198590 | 233198791 | Hypomethylated |
| Breast Basal | chr7 | 4804631 | 4805136 | Hypomethylated |
| Breast Basal | chr8 | 142235391 | 142235926 | Hypomethylated |
| Breast Basal | chr11 | 772689 | 773090 | Hypomethylated |
| Breast Basal | chr19 | 8554999 | 8555062 | Hypomethylated |
| Breast Basal | chr19 | 46387679 | 46387919 | Hypomethylated |
| Breast Basal | chr22 | 29659845 | 29660202 | Hypomethylated |
| Breast Basal | chr14 | 102093931 | 102094226 | Hypomethylated |
| Breast Basal | chr17 | 5983894 | 5984066 | Hypomethylated |
| Breast Basal | chr22 | 47022433 | 47022659 | Hypomethylated |
| Breast Basal | chr7 | 2054500 | 2055180 | Hypomethylated |
| Breast Basal | chr11 | 2170373 | 2170444 | Hypomethylated |
| Breast Basal | chr5 | 1876857 | 1877139 | Hypermethylated |
| Breast Basal | chr10 | 8089332 | 8089925 | Hypermethylated |
| Breast Basal | chr16 | 56703476 | 56703914 | Hypermethylated |
| Breast Basal | chr9 | 135463966 | 135464285 | Hypermethylated |
| Breast Basal | chr10 | 129534178 | 129534481 | Hypermethylated |
| Breast Basal | chr13 | 37004787 | 37005108 | Hypermethylated |
| Breast Basal | chr14 | 95233855 | 95234127 | Hypermethylated |
| Breast Basal | chr6 | 10381523 | 10382075 | Hypermethylated |
| Breast Basal | chr9 | 129372570 | 129372906 | Hypermethylated |
| Breast Basal | chr12 | 49390678 | 49391209 | Hypermethylated |
| Breast Basal | chr1 | 22668552 | 22668874 | Hypermethylated |
| Breast Basal | chr3 | 137489017 | 137489723 | Hypermethylated |
| Breast Basal | chr5 | 1874836 | 1875551 | Hypermethylated |
| Breast Basal | chr12 | 54090151 | 54090388 | Hypermethylated |
| Breast Basal | chr9 | 129372913 | 129373070 | Hypermethylated |
| Breast Luminal | chr11 | 65582724 | 65582909 | Hypomethylated |
| Breast Luminal | chr1 | 3321982 | 3322140 | Hypomethylated |
| Breast Luminal | chr1 | 2832006 | 2832144 | Hypomethylated |
| Breast Luminal | chr5 | 148958721 | 148958920 | Hypomethylated |
| Breast Luminal | chr17 | 2278727 | 2279131 | Hypomethylated |
| Breast Luminal | chr5 | 176764116 | 176764365 | Hypomethylated |
| Breast Luminal | chr12 | 115150184 | 115150549 | Hypomethylated |
| Breast Luminal | chr16 | 90068365 | 90068490 | Hypomethylated |
| Breast Luminal | chr17 | 47467293 | 47467337 | Hypomethylated |
| Breast Luminal | chr18 | 10589150 | 10589279 | Hypomethylated |
| Breast Luminal | chr19 | 48491917 | 48492032 | Hypomethylated |
| Breast Luminal | chr6 | 169641078 | 169641392 | Hypomethylated |
| Breast Luminal | chr2 | 121036351 | 121037152 | Hypomethylated |
| Breast Luminal | chr16 | 30852669 | 30852999 | Hypomethylated |
| Breast Luminal | chr18 | 10588980 | 10589063 | Hypomethylated |
| Breast Luminal | chr19 | 7685239 | 7685435 | Hypomethylated |
| Breast Luminal | chr4 | 757194 | 757416 | Hypomethylated |
| Breast Luminal | chr1 | 24191684 | 24192034 | Hypomethylated |
| Breast Luminal | chr6 | 168797654 | 168797892 | Hypomethylated |
| Breast Luminal | chr9 | 139698878 | 139699275 | Hypomethylated |
| Breast Luminal | chr1 | 9565292 | 9565531 | Hypomethylated |
| Breast Luminal | chr13 | 103454025 | 103454177 | Hypomethylated |
| Breast Luminal | chr3 | 133175047 | 133175550 | Hypomethylated |
| Breast Luminal | chr1 | 3017607 | 3017703 | Hypomethylated |
| Breast Luminal | chr1 | 3474045 | 3474246 | Hypomethylated |
| Breast Luminal | chr5 | 172982435 | 172982535 | Hypomethylated |
| Breast Luminal | chr14 | 105181998 | 105182486 | Hypomethylated |
| Breast Luminal | chr14 | 105269038 | 105269404 | Hypomethylated |
| Breast Luminal | chr3 | 137489017 | 137489723 | Hypermethylated |
| Breast Luminal | chr10 | 22542284 | 22542463 | Hypermethylated |
| Breast Luminal | chr17 | 42287754 | 42288090 | Hypermethylated |
| Breast Luminal | chr5 | 138729659 | 138729816 | Hypermethylated |
| Breast Luminal | chr5 | 174158786 | 174158926 | Hypermethylated |
| Breast Luminal | chr6 | 10393138 | 10393779 | Hypermethylated |
| Breast Luminal | chr9 | 129386162 | 129386326 | Hypermethylated |
| Breast Luminal | chr5 | 174158548 | 174158782 | Hypermethylated |
| Breast Luminal | chr9 | 129372460 | 129372565 | Hypermethylated |
| Breast Luminal | chr10 | 8085311 | 8085801 | Hypermethylated |
| Breast Luminal | chr5 | 1876857 | 1877139 | Hypermethylated |
| Breast Luminal | chr5 | 1879621 | 1879706 | Hypermethylated |
| Breast Luminal | chr9 | 129373594 | 129373647 | Hypermethylated |
| Breast Luminal | chr5 | 1875800 | 1875940 | Hypermethylated |
| Breast Luminal | chr5 | 1874836 | 1875551 | Hypermethylated |
| Breast Luminal | chr9 | 129388506 | 129388993 | Hypermethylated |
| Breast Luminal | chr5 | 1877986 | 1878242 | Hypermethylated |
| Breast Luminal | chr6 | 10381523 | 10382075 | Hypermethylated |
| Breast Luminal | chr9 | 129388068 | 129388495 | Hypermethylated |
| Breast Luminal | chr9 | 129372570 | 129372906 | Hypermethylated |
| Breast Luminal | chr9 | 129372913 | 129373070 | Hypermethylated |
| Bulk Endothelial | chr9 | 139406515 | 139406839 | Hypomethylated |
| Bulk Endothelial | chr6 | 1635704 | 1635851 | Hypomethylated |
| Bulk Endothelial | chr14 | 69931518 | 69931952 | Hypomethylated |
| Bulk Endothelial | chr17 | 80803660 | 80804189 | Hypomethylated |
| Bulk Endothelial | chr7 | 4746712 | 4746898 | Hypomethylated |
| Bulk Endothelial | chr12 | 52291132 | 52291323 | Hypomethylated |
| Bulk Endothelial | chr7 | 150690506 | 150691038 | Hypomethylated |
| Bulk Endothelial | chr6 | 157877066 | 157877221 | Hypomethylated |
| Bulk Endothelial | chr16 | 2220378 | 2221058 | Hypomethylated |
| Bulk Endothelial | chr7 | 736404 | 736961 | Hypomethylated |
| Bulk Endothelial | chr6 | 46889597 | 46889761 | Hypomethylated |
| Bulk Endothelial | chr17 | 1975093 | 1975641 | Hypomethylated |
| Bulk Endothelial | chr12 | 121717850 | 121717948 | Hypomethylated |
| Bulk Endothelial | chr13 | 29329007 | 29329215 | Hypomethylated |
| Bulk Endothelial | chr19 | 11707038 | 11707247 | Hypomethylated |
| Bulk Endothelial | chr6 | 167028939 | 167029195 | Hypomethylated |
| Bulk Endothelial | chr7 | 65617005 | 65617363 | Hypomethylated |
| Bulk Endothelial | chr11 | 86662732 | 86663161 | Hypomethylated |
| Bulk Endothelial | chr14 | 105796697 | 105796899 | Hypomethylated |
| Bulk Endothelial | chr1 | 3038085 | 3038257 | Hypomethylated |
| Bulk Endothelial | chr5 | 141059753 | 141060199 | Hypomethylated |
| Bulk Endothelial | chr10 | 13726446 | 13726682 | Hypomethylated |
| Bulk Endothelial | chr11 | 76290451 | 76290615 | Hypomethylated |
| Bulk Endothelial | chr16 | 1562314 | 1562661 | Hypomethylated |
| Bulk Endothelial | chr2 | 89128482 | 89128683 | Hypomethylated |
| Bulk Endothelial | chr11 | 126301685 | 126301953 | Hypomethylated |
| Bulk Endothelial | chr7 | 131314332 | 131314441 | Hypomethylated |
| Bulk Endothelial | chr9 | 138900552 | 138900629 | Hypomethylated |
| Bulk Endothelial | chr6 | 132270337 | 132270745 | Hypomethylated |
| Bulk Endothelial | chr17 | 73506140 | 73506303 | Hypomethylated |
| Bulk Endothelial | chr17 | 79170799 | 79170897 | Hypomethylated |
| Bulk Endothelial | chr4 | 1227142 | 1227401 | Hypomethylated |
| Bulk Endothelial | chr4 | 151504816 | 151505028 | Hypomethylated |
| Bulk Endothelial | chr2 | 128430937 | 128431443 | Hypomethylated |
| Bulk Endothelial | chr5 | 38466902 | 38467249 | Hypomethylated |
| Bulk Endothelial | chr9 | 35909661 | 35910091 | Hypomethylated |
| Bulk Endothelial | chr10 | 504484 | 504785 | Hypomethylated |
| Bulk Endothelial | chr7 | 2646484 | 2646629 | Hypomethylated |
| Bulk Endothelial | chr19 | 18233987 | 18234213 | Hypomethylated |
| Bulk Endothelial | chr22 | 47188803 | 47188952 | Hypomethylated |
| Bulk Endothelial | chr4 | 1227729 | 1227950 | Hypomethylated |
| Bulk Endothelial | chr17 | 15394429 | 15394554 | Hypomethylated |
| Bulk Endothelial | chr7 | 131217702 | 131217878 | Hypomethylated |
| Bulk Endothelial | chr7 | 142984797 | 142984913 | Hypomethylated |
| Bulk Endothelial | chr17 | 73509751 | 73509964 | Hypomethylated |
| Bulk Endothelial | chr9 | 137763917 | 137764041 | Hypomethylated |
| Bulk Endothelial | chr11 | 72295847 | 72296047 | Hypermethylated |
| Bulk Endothelial | chr11 | 72295460 | 72295843 | Hypermethylated |
| Bulk Endothelial | chr19 | 8398826 | 8399120 | Hypermethylated |
| Bulk Endothelial | chr8 | 10588820 | 10589153 | Hypermethylated |
| Bulk Epithelial | chr17 | 37862101 | 37862814 | Hypomethylated |
| Bulk Epithelial | chr11 | 2397153 | 2397487 | Hypomethylated |
| Bulk Epithelial | chr11 | 27490421 | 27491031 | Hypomethylated |
| Bulk Epithelial | chr7 | 985538 | 985720 | Hypomethylated |
| Bulk Epithelial | chr10 | 45406802 | 45407004 | Hypomethylated |
| Bulk Epithelial | chr6 | 168789503 | 168789732 | Hypomethylated |
| Bulk Epithelial | chr16 | 1349427 | 1349853 | Hypomethylated |
| Bulk Epithelial | chr21 | 46840048 | 46840156 | Hypomethylated |
| Bulk Epithelial | chr1 | 1099754 | 1100006 | Hypomethylated |
| Bulk Epithelial | chr10 | 101841184 | 101841419 | Hypomethylated |
| Bulk Epithelial | chr19 | 1907759 | 1907994 | Hypomethylated |
| Bulk Epithelial | chr2 | 97171293 | 97171450 | Hypomethylated |
| Bulk Epithelial | chr9 | 97803917 | 97804138 | Hypomethylated |
| Bulk Epithelial | chr11 | 128558170 | 128558419 | Hypomethylated |
| Bulk Epithelial | chr17 | 8190993 | 8191301 | Hypomethylated |
| Bulk Epithelial | chr7 | 27163145 | 27163499 | Hypomethylated |
| Bulk Epithelial | chr17 | 79949983 | 79950241 | Hypomethylated |
| Bulk Epithelial | chr16 | 27237906 | 27238021 | Hypomethylated |
| Bulk Epithelial | chr16 | 85424260 | 85424547 | Hypomethylated |
| Bulk Epithelial | chr21 | 46678570 | 46678673 | Hypomethylated |
| Bulk Epithelial | chr1 | 1216970 | 1217402 | Hypomethylated |
| Bulk Epithelial | chr11 | 65414458 | 65414631 | Hypomethylated |
| Bulk Epithelial | chr14 | 100032685 | 100032895 | Hypomethylated |
| Bulk Epithelial | chr1 | 2782910 | 2783117 | Hypomethylated |
| Bulk Epithelial | chr17 | 48179459 | 48179672 | Hypomethylated |
| Bulk Epithelial | chr5 | 1183107 | 1183283 | Hypomethylated |
| Bulk Epithelial | chr10 | 134079332 | 134079426 | Hypomethylated |
| Bulk Epithelial | chr11 | 34622158 | 34622496 | Hypomethylated |
| Bulk Epithelial | chr14 | 100621848 | 100622287 | Hypomethylated |
| Bulk Epithelial | chr16 | 27375732 | 27375974 | Hypomethylated |
| Bulk Epithelial | chr8 | 29177514 | 29177650 | Hypomethylated |
| Bulk Epithelial | chr9 | 130504037 | 130504267 | Hypomethylated |
| Bulk Epithelial | chr13 | 20805487 | 20805590 | Hypermethylated |
| Bulk Epithelial | chr15 | 101991828 | 101991977 | Hypermethylated |
| Bulk Epithelial | chr16 | 1584118 | 1584218 | Hypermethylated |
| Bulk Epithelial | chr16 | 86962660 | 86962748 | Hypermethylated |
| Bulk Epithelial | chr4 | 4765147 | 4765382 | Hypermethylated |
| Bulk Epithelial | chr19 | 11516988 | 11517209 | Hypermethylated |
| Bulk Epithelial | chr5 | 132161281 | 132161485 | Hypermethylated |
| Bulk Epithelial | chr15 | 96866665 | 96866787 | Hypermethylated |
| Bulk Epithelial | chr11 | 124750355 | 124750431 | Hypermethylated |
| Bulk Epithelial | chr15 | 96887036 | 96887138 | Hypermethylated |
| Bulk Epithelial | chr5 | 132161628 | 132161741 | Hypermethylated |
| Bulk Epithelial | chr7 | 155598820 | 155598969 | Hypermethylated |
| Bulk Epithelial | chr15 | 96885028 | 96885331 | Hypermethylated |
| Bulk Epithelial | chr8 | 8204381 | 8204920 | Hypermethylated |
| Bulk Epithelial | chr19 | 3671800 | 3672121 | Hypermethylated |
| Bulk Epithelial | chr9 | 134148465 | 134149034 | Hypermethylated |
| Bulk Epithelial | chr7 | 155599119 | 155599231 | Hypermethylated |
| Bulk Epithelial | chr12 | 124941781 | 124942249 | Hypermethylated |
| Bulk Immune | chr3 | 67706044 | 67706706 | Hypomethylated |
| Bulk Immune | chr19 | 3179171 | 3180240 | Hypomethylated |
| Bulk Immune | chr12 | 51717273 | 51718156 | Hypomethylated |
| Bulk Immune | chr9 | 27093573 | 27093899 | Hypomethylated |
| Bulk Immune | chr9 | 36458395 | 36458863 | Hypomethylated |
| Bulk Immune | chr1 | 25291486 | 25291893 | Hypomethylated |
| Bulk Immune | chr12 | 47700770 | 47701310 | Hypomethylated |
| Bulk Immune | chr17 | 76361115 | 76361254 | Hypomethylated |
| Bulk Immune | chr11 | 111093580 | 111094004 | Hypomethylated |
| Bulk Immune | chr10 | 135202560 | 135202871 | Hypomethylated |
| Bulk Immune | chr6 | 25041762 | 25042379 | Hypomethylated |
| Bulk Immune | chr16 | 29675766 | 29676065 | Hypomethylated |
| Bulk Immune | chr2 | 43397998 | 43398155 | Hypomethylated |
| Bulk Immune | chr7 | 149566913 | 149567085 | Hypomethylated |
| Bulk Immune | chr2 | 133403571 | 133403807 | Hypomethylated |
| Bulk Immune | chr8 | 145808729 | 145808892 | Hypomethylated |
| Bulk Immune | chr11 | 2321770 | 2322051 | Hypomethylated |
| Bulk Immune | chr6 | 168107235 | 168107375 | Hypomethylated |
| Bulk Immune | chr11 | 63974540 | 63974842 | Hypomethylated |
| Bulk Immune | chr19 | 5139390 | 5139647 | Hypomethylated |
| Bulk Immune | chr3 | 196367455 | 196367896 | Hypomethylated |
| Bulk Immune | chr16 | 28996021 | 28996366 | Hypomethylated |
| Bulk Immune | chr19 | 2446619 | 2446783 | Hypomethylated |
| Bulk Immune | chr9 | 123657071 | 123657231 | Hypermethylated |
| Bulk Immune | chr2 | 233251922 | 233252099 | Hypermethylated |
| Bulk Immune | chr5 | 176827023 | 176827233 | Hypermethylated |
| Bulk Immune | chr15 | 77320552 | 77320923 | Hypermethylated |
| Bulk Immune | chr2 | 54087173 | 54087424 | Hypermethylated |
| Bulk Immune | chr8 | 120685466 | 120685687 | Hypermethylated |
| Bulk Immune | chr10 | 26855881 | 26856468 | Hypermethylated |
| Bulk Immune | chr7 | 27154911 | 27155334 | Hypermethylated |
| Bulk Immune | chr11 | 63687764 | 63688071 | Hypermethylated |
| Bulk Immune | chr1 | 25257622 | 25257987 | Hypermethylated |
| Bulk Immune | chr15 | 66999862 | 66999969 | Hypermethylated |
| Bulk Immune | chr19 | 2540684 | 2541138 | Hypermethylated |
| Bulk Immune | chr5 | 148961435 | 148961719 | Hypermethylated |
| Bulk Immune | chr10 | 101282185 | 101282353 | Hypermethylated |
| Bulk Immune | chr1 | 92946803 | 92947227 | Hypermethylated |
| Bulk Immune | chr7 | 27152969 | 27153160 | Hypermethylated |
| Bulk Immune | chr10 | 101281123 | 101281332 | Hypermethylated |
| Bulk Immune | chr16 | 67682047 | 67682428 | Hypermethylated |
| Bulk Immune | chr19 | 1070772 | 1071122 | Hypermethylated |
| Bulk Immune | chr7 | 27153188 | 27153848 | Hypermethylated |
| Bulk Immune | chr12 | 107974824 | 107975402 | Hypermethylated |
| Bulk Immune | chr19 | 49841801 | 49842135 | Hypermethylated |
| Bulk Immune | chr19 | 6475589 | 6476186 | Hypermethylated |
| Bulk Immune | chr11 | 47376572 | 47377213 | Hypermethylated |
| Bulk Immune | chr1 | 45082704 | 45083125 | Hypermethylated |
| Bulk Immune | chr19 | 49842322 | 49843076 | Hypermethylated |
| Bulk Immune | chr19 | 10444874 | 10445594 | Hypermethylated |
| Cardiomyocyte | chr5 | 150028928 | 150029306 | Hypomethylated |
| Cardiomyocyte | chr22 | 26138136 | 26138600 | Hypomethylated |
| Cardiomyocyte | chr8 | 124664836 | 124665047 | Hypomethylated |
| Cardiomyocyte | chr10 | 29186554 | 29186755 | Hypomethylated |
| Cardiomyocyte | chr1 | 16341849 | 16342452 | Hypomethylated |
| Cardiomyocyte | chr13 | 113384765 | 113384930 | Hypomethylated |
| Cardiomyocyte | chr2 | 236877092 | 236877616 | Hypomethylated |
| Cardiomyocyte | chr10 | 855857 | 856183 | Hypomethylated |
| Cardiomyocyte | chr12 | 3364736 | 3365604 | Hypomethylated |
| Cardiomyocyte | chr20 | 55981966 | 55982252 | Hypomethylated |
| Cardiomyocyte | chr5 | 80529795 | 80530214 | Hypomethylated |
| Cardiomyocyte | chr19 | 1419166 | 1419762 | Hypomethylated |
| Cardiomyocyte | chr8 | 41517980 | 41518301 | Hypomethylated |
| Cardiomyocyte | chr2 | 160031535 | 160031871 | Hypomethylated |
| Cardiomyocyte | chr18 | 19780872 | 19781199 | Hypomethylated |
| Cardiomyocyte | chr1 | 45106147 | 45106400 | Hypomethylated |
| Cardiomyocyte | chr12 | 106631875 | 106632366 | Hypomethylated |
| Cardiomyocyte | chr18 | 19780465 | 19780821 | Hypomethylated |
| Cardiomyocyte | chr6 | 17032349 | 17032642 | Hypomethylated |
| Cardiomyocyte | chr11 | 78534066 | 78534252 | Hypomethylated |
| Cardiomyocyte | chr13 | 114108292 | 114108653 | Hypomethylated |
| Cardiomyocyte | chr2 | 74645622 | 74645750 | Hypomethylated |
| Cardiomyocyte | chr3 | 11606624 | 11606964 | Hypomethylated |
| Cardiomyocyte | chr8 | 141559360 | 141559711 | Hypomethylated |
| Cardiomyocyte | chr9 | 134164500 | 134164670 | Hypomethylated |
| Cardiomyocyte | chr13 | 114137827 | 114138226 | Hypomethylated |
| Cardiomyocyte | chr16 | 46781221 | 46781714 | Hypomethylated |
| Cardiomyocyte | chr20 | 60858231 | 60858540 | Hypomethylated |
| Cardiomyocyte | chr7 | 44159762 | 44160044 | Hypomethylated |
| Cardiomyocyte | chr2 | 128430937 | 128431443 | Hypomethylated |
| Cardiomyocyte | chr2 | 240879235 | 240879556 | Hypomethylated |
| Cardiomyocyte | chr3 | 18390643 | 18390959 | Hypomethylated |
| Cardiomyocyte | chr3 | 122675153 | 122675540 | Hypomethylated |
| Cardiomyocyte | chr8 | 28923946 | 28924153 | Hypomethylated |
| Cardiomyocyte | chr20 | 60861460 | 60861874 | Hypomethylated |
| Cardiomyocyte | chr7 | 43917672 | 43917891 | Hypomethylated |
| Cardiomyocyte | chr13 | 114106471 | 114106734 | Hypomethylated |
| Cardiomyocyte | chr4 | 186578455 | 186578679 | Hypomethylated |
| Cardiomyocyte | chr9 | 135929763 | 135929928 | Hypomethylated |
| Cardiomyocyte | chr15 | 90784663 | 90784846 | Hypomethylated |
| Cardiomyocyte | chr22 | 26149485 | 26150074 | Hypomethylated |
| Cardiomyocyte | chr6 | 504028 | 504466 | Hypomethylated |
| Cardiomyocyte | chr10 | 81059280 | 81059434 | Hypomethylated |
| Cardiomyocyte | chr1 | 1478629 | 1478779 | Hypomethylated |
| Cardiomyocyte | chr2 | 241533173 | 241533990 | Hypomethylated |
| Cardiomyocyte | chr6 | 6746438 | 6746730 | Hypomethylated |
| Cardiomyocyte | chr6 | 158464164 | 158464475 | Hypomethylated |
| Cardiomyocyte | chr7 | 820811 | 821315 | Hypomethylated |
| Cardiomyocyte | chr7 | 4824534 | 4824952 | Hypomethylated |
| Cardiomyocyte | chr3 | 192125874 | 192126438 | Hypermethylated |
| Cardiopulmonary | chr11 | 128698175 | 128698361 | Hypomethylated |
| Endothelial | | | | |
| Cardiopulmonary | chr7 | 150690506 | 150691038 | Hypomethylated |
| Endothelial | | | | |
| Cardiopulmonary | chr16 | 2220378 | 2221058 | Hypomethylated |
| Endothelial | | | | |
| Cardiopulmonary | chr10 | 466647 | 467242 | Hypomethylated |
| Endothelial | | | | |
| Cardiopulmonary | chr6 | 167028939 | 167029195 | Hypomethylated |
| Endothelial | | | | |
| Cardiopulmonary | chr7 | 5549534 | 5549731 | Hypomethylated |
| Endothelial | | | | |
| Cardiopulmonary | chr8 | 96572161 | 96572434 | Hypomethylated |
| Endothelial | | | | |
| Cardiopulmonary | chr9 | 139406515 | 139406839 | Hypomethylated |
| Endothelial | | | | |
| Cardiopulmonary | chr11 | 70266256 | 70266421 | Hypomethylated |
| Endothelial | | | | |
| Cardiopulmonary | chr12 | 19565936 | 19566177 | Hypomethylated |
| Endothelial | | | | |
| Cardiopulmonary | chr17 | 80803660 | 80804189 | Hypomethylated |
| Endothelial | | | | |
| Cardiopulmonary | chr6 | 1635704 | 1635851 | Hypomethylated |
| Endothelial | | | | |
| Cardiopulmonary | chr5 | 141059753 | 141060199 | Hypomethylated |
| Endothelial | | | | |
| Cardiopulmonary | chr9 | 139406957 | 139407311 | Hypomethylated |
| Endothelial | | | | |
| Cardiopulmonary | chr7 | 142984797 | 142984913 | Hypomethylated |
| Endothelial | | | | |
| Cardiopulmonary | chr11 | 134231502 | 134231622 | Hypomethylated |
| Endothelial | | | | |
| Cardiopulmonary | chr14 | 105796697 | 105796899 | Hypomethylated |
| Endothelial | | | | |
| Cardiopulmonary | chr6 | 1616682 | 1617275 | Hypomethylated |
| Endothelial | | | | |
| Cardiopulmonary | chr17 | 73506140 | 73506303 | Hypomethylated |
| Endothelial | | | | |
| Cardiopulmonary | chr15 | 83781607 | 83781804 | Hypomethylated |
| Endothelial | | | | |
| Cardiopulmonary | chr2 | 235905958 | 235906259 | Hypomethylated |
| Endothelial | | | | |
| Cardiopulmonary | chr14 | 77351761 | 77352081 | Hypomethylated |
| Endothelial | | | | |
| Cardiopulmonary | chr15 | 74637558 | 74637731 | Hypomethylated |
| Endothelial | | | | |
| Cardiopulmonary | chr9 | 139549426 | 139549603 | Hypomethylated |
| Endothelial | | | | |
| Cardiopulmonary | chr14 | 69931518 | 69931952 | Hypomethylated |
| Endothelial | | | | |
| Cardiopulmonary | chr4 | 5753985 | 5754218 | Hypomethylated |
| Endothelial | | | | |
| Cardiopulmonary | chr9 | 35909661 | 35910091 | Hypomethylated |
| Endothelial | | | | |
| Cardiopulmonary | chr16 | 8943021 | 8943199 | Hypomethylated |
| Endothelial | | | | |
| Cardiopulmonary | chr6 | 1624186 | 1624283 | Hypomethylated |
| Endothelial | | | | |
| Cardiopulmonary | chr8 | 102464327 | 102464407 | Hypomethylated |
| Endothelial | | | | |
| Cardiopulmonary | chr9 | 138900552 | 138900629 | Hypomethylated |
| Endothelial | | | | |
| Cardiopulmonary | chr12 | 121717850 | 121717948 | Hypomethylated |
| Endothelial | | | | |
| Cardiopulmonary | chr17 | 79170799 | 79170897 | Hypomethylated |
| Endothelial | | | | |
| Cardiopulmonary | chr1 | 3038085 | 3038257 | Hypomethylated |
| Endothelial | | | | |
| Cardiopulmonary | chr13 | 29329007 | 29329215 | Hypomethylated |
| Endothelial | | | | |
| Cardiopulmonary | chr19 | 474408 | 474475 | Hypomethylated |
| Endothelial | | | | |
| Cardiopulmonary | chr19 | 3765019 | 3766508 | Hypomethylated |
| Endothelial | | | | |
| Cardiopulmonary | chr2 | 128430937 | 128431443 | Hypomethylated |
| Endothelial | | | | |
| Cardiopulmonary | chr7 | 131217702 | 131217878 | Hypomethylated |
| Endothelial | | | | |
| Cardiopulmonary | chr10 | 30317521 | 30317689 | Hypomethylated |
| Endothelial | | | | |
| Cardiopulmonary | chr19 | 17374974 | 17375446 | Hypomethylated |
| Endothelial | | | | |
| Cardiopulmonary | chr2 | 237074693 | 237074856 | Hypermethylated |
| Endothelial | | | | |
| Cardiopulmonary | chr13 | 79182267 | 79182623 | Hypermethylated |
| Endothelial | | | | |
| Cardiopulmonary | chr8 | 10588820 | 10589153 | Hypermethylated |
| Endothelial | | | | |
| Cardiopulmonary | chr19 | 8398826 | 8399120 | Hypermethylated |
| Endothelial | | | | |
| Cardiopulmonary | chr16 | 58535446 | 58535596 | Hypermethylated |
| Endothelial | | | | |
| Colon Epithelial | chr2 | 97427531 | 97428080 | Hypomethylated |
| Colon Epithelial | chr13 | 114189623 | 114190065 | Hypomethylated |
| Colon Epithelial | chr17 | 80535398 | 80535834 | Hypomethylated |
| Colon Epithelial | chr7 | 150068607 | 150068986 | Hypomethylated |
| Colon Epithelial | chr13 | 30707459 | 30707773 | Hypomethylated |
| Colon Epithelial | chr6 | 38141763 | 38142021 | Hypomethylated |
| Colon Epithelial | chr19 | 10823619 | 10823914 | Hypomethylated |
| Colon Epithelial | chr2 | 106959820 | 106960122 | Hypomethylated |
| Colon Epithelial | chr20 | 55959154 | 55959798 | Hypomethylated |
| Colon Epithelial | chr17 | 76991224 | 76991699 | Hypomethylated |
| Colon Epithelial | chr1 | 1062975 | 1063187 | Hypomethylated |
| Colon Epithelial | chr12 | 132423665 | 132423879 | Hypomethylated |
| Colon Epithelial | chr14 | 104547801 | 104548104 | Hypomethylated |
| Colon Epithelial | chr9 | 140683345 | 140683528 | Hypomethylated |
| Colon Epithelial | chr11 | 66631299 | 66631470 | Hypomethylated |
| Colon Epithelial | chr16 | 2141861 | 2142285 | Hypomethylated |
| Colon Epithelial | chr17 | 5993587 | 5993793 | Hypomethylated |
| Colon Epithelial | chr17 | 77073929 | 77075062 | Hypomethylated |
| Colon Epithelial | chr17 | 79224246 | 79224909 | Hypomethylated |
| Colon Epithelial | chr9 | 130675536 | 130676110 | Hypomethylated |
| Colon Epithelial | chr8 | 142984528 | 142984693 | Hypomethylated |
| Colon Epithelial | chr17 | 63289695 | 63289933 | Hypomethylated |
| Colon Epithelial | chr19 | 2305151 | 2305259 | Hypomethylated |
| Colon Epithelial | chr1 | 6421300 | 6421678 | Hypomethylated |
| Colon Epithelial | chr5 | 664202 | 664415 | Hypomethylated |
| Colon Epithelial | chr7 | 834375 | 834637 | Hypomethylated |
| Colon Epithelial | chr7 | 157372332 | 157372448 | Hypomethylated |
| Colon Epithelial | chr11 | 1444733 | 1445067 | Hypomethylated |
| Colon Epithelial | chr11 | 68175988 | 68176320 | Hypomethylated |
| Colon Epithelial | chr16 | 87778775 | 87779046 | Hypomethylated |
| Colon Epithelial | chr14 | 93155127 | 93155269 | Hypomethylated |
| Colon Epithelial | chr19 | 2278406 | 2278613 | Hypomethylated |
| Colon Epithelial | chr1 | 1063255 | 1063374 | Hypomethylated |
| Colon Epithelial | chr6 | 35109390 | 35109799 | Hypomethylated |
| Colon Epithelial | chr17 | 79223904 | 79224188 | Hypomethylated |
| Colon Epithelial | chr7 | 959725 | 960076 | Hypomethylated |
| Colon Epithelial | chr9 | 132371085 | 132371258 | Hypomethylated |
| Colon Epithelial | chr9 | 139419864 | 139420019 | Hypomethylated |
| Colon Epithelial | chr15 | 40641488 | 40642094 | Hypomethylated |
| Colon Epithelial | chr1 | 1061483 | 1061760 | Hypomethylated |
| Colon Epithelial | chr19 | 2278728 | 2278941 | Hypomethylated |
| Colon Epithelial | chr8 | 145721522 | 145722011 | Hypomethylated |
| Colon Epithelial | chr11 | 1258312 | 1258485 | Hypomethylated |
| Colon Epithelial | chr19 | 3966588 | 3966908 | Hypomethylated |
| Colon Epithelial | chr10 | 11206756 | 11207474 | Hypermethylated |
| Colon Epithelial | chr14 | 38679781 | 38680291 | Hypermethylated |
| Colon Epithelial | chr7 | 156798471 | 156798811 | Hypermethylated |
| Colon Epithelial | chr7 | 156797298 | 156797842 | Hypermethylated |
| Colon Epithelial | chr17 | 70215747 | 70216403 | Hypermethylated |
| Colon Epithelial | chr7 | 156797845 | 156798469 | Hypermethylated |
| Dermal Endothelial | chr6 | 167028939 | 167029195 | Hypomethylated |
| Dermal Endothelial | chr17 | 80803660 | 80804189 | Hypomethylated |
| Dermal Endothelial | chr10 | 121169671 | 121170069 | Hypomethylated |
| Dermal Endothelial | chr8 | 140748972 | 140749334 | Hypomethylated |
| Dermal Endothelial | chr17 | 700763 | 701051 | Hypomethylated |
| Dermal Endothelial | chr16 | 2220378 | 2221058 | Hypomethylated |
| Dermal Endothelial | chr11 | 128698175 | 128698361 | Hypomethylated |
| Dermal Endothelial | chr11 | 134231502 | 134231622 | Hypomethylated |
| Dermal Endothelial | chr19 | 17374974 | 17375446 | Hypomethylated |
| Dermal Endothelial | chr4 | 38690557 | 38691119 | Hypomethylated |
| Dermal Endothelial | chr19 | 11707038 | 11707247 | Hypomethylated |
| Dermal Endothelial | chr7 | 150690506 | 150691038 | Hypomethylated |
| Dermal Endothelial | chr22 | 19508947 | 19509559 | Hypomethylated |
| Dermal Endothelial | chr1 | 4237464 | 4237671 | Hypomethylated |
| Dermal Endothelial | chr3 | 3079964 | 3080198 | Hypomethylated |
| Dermal Endothelial | chr17 | 5993587 | 5993793 | Hypomethylated |
| Dermal Endothelial | chr17 | 79170799 | 79170897 | Hypomethylated |
| Dermal Endothelial | chr19 | 18233987 | 18234213 | Hypomethylated |
| Dermal Endothelial | chr9 | 139406515 | 139406839 | Hypomethylated |
| Dermal Endothelial | chr2 | 109891992 | 109892086 | Hypomethylated |
| Dermal Endothelial | chr9 | 75094281 | 75094544 | Hypomethylated |
| Dermal Endothelial | chr19 | 17000794 | 17001198 | Hypomethylated |
| Dermal Endothelial | chr1 | 54110431 | 54110775 | Hypomethylated |
| Dermal Endothelial | chr2 | 43270898 | 43271814 | Hypomethylated |
| Dermal Endothelial | chr12 | 121717850 | 121717948 | Hypomethylated |
| Dermal Endothelial | chr19 | 19304585 | 19304917 | Hypomethylated |
| Dermal Endothelial | chr22 | 27026220 | 27026502 | Hypomethylated |
| Dermal Endothelial | chr1 | 7550010 | 7550091 | Hypomethylated |
| Dermal Endothelial | chr7 | 2753426 | 2753730 | Hypomethylated |
| Dermal Endothelial | chr7 | 158415936 | 158416191 | Hypomethylated |
| Dermal Endothelial | chr16 | 81731208 | 81731484 | Hypomethylated |
| Dermal Endothelial | chr5 | 179806847 | 179806982 | Hypomethylated |
| Dermal Endothelial | chr10 | 3613740 | 3613899 | Hypomethylated |
| Dermal Endothelial | chr13 | 114926731 | 114926860 | Hypomethylated |
| Dermal Endothelial | chr16 | 4815857 | 4815987 | Hypomethylated |
| Dermal Endothelial | chr17 | 79170598 | 79170789 | Hypomethylated |
| Dermal Endothelial | chr7 | 2003132 | 2003772 | Hypomethylated |
| Dermal Endothelial | chr11 | 72295460 | 72295843 | Hypermethylated |
| Dermal Endothelial | chr3 | 128209966 | 128210732 | Hypermethylated |
| Dermal Endothelial | chr11 | 128555051 | 128555481 | Hypermethylated |
| Dermal Endothelial | chr8 | 10590177 | 10590330 | Hypermethylated |
| Dermal Endothelial | chr3 | 129062831 | 129063119 | Hypermethylated |
| Dermal Endothelial | chr7 | 5468088 | 5469462 | Hypermethylated |
| Dermal Endothelial | chr2 | 177022967 | 177023205 | Hypermethylated |
| Dermal Endothelial | chr11 | 72300978 | 72301585 | Hypermethylated |
| Dermal Endothelial | chr6 | 5998982 | 5999270 | Hypermethylated |
| Dermal Endothelial | chr2 | 177022693 | 177022963 | Hypermethylated |
| Dermal Endothelial | chr2 | 177021874 | 177022246 | Hypermethylated |
| Dermal Endothelial | chr15 | 41218119 | 41218738 | Hypermethylated |
| Dermal Endothelial | chr9 | 124888894 | 124889382 | Hypermethylated |
| Granulocyte | chr14 | 102676909 | 102677377 | Hypomethylated |
| Granulocyte | chr16 | 88906304 | 88906775 | Hypomethylated |
| Granulocyte | chr19 | 1423555 | 1423830 | Hypomethylated |
| Granulocyte | chr19 | 1423535 | 1423830 | Hypomethylated |
| Granulocyte | chr13 | 114263370 | 114263522 | Hypomethylated |
| Granulocyte | chr16 | 88907037 | 88907250 | Hypomethylated |
| Granulocyte | chr17 | 4081291 | 4081574 | Hypomethylated |
| Granulocyte | chr9 | 139812189 | 139812430 | Hypomethylated |
| Granulocyte | chr17 | 694980 | 695078 | Hypomethylated |
| Granulocyte | chr4 | 89446367 | 89446620 | Hypomethylated |
| Granulocyte | chr16 | 3639093 | 3639262 | Hypomethylated |
| Granulocyte | chr9 | 129184164 | 129184294 | Hypomethylated |
| Granulocyte | chr3 | 42265452 | 42265625 | Hypomethylated |
| Granulocyte | chr10 | 119794366 | 119794600 | Hypomethylated |
| Granulocyte | chr12 | 133248726 | 133249006 | Hypomethylated |
| Granulocyte | chr16 | 8943315 | 8943550 | Hypomethylated |
| Granulocyte | chr17 | 79239868 | 79240158 | Hypomethylated |
| Granulocyte | chr1 | 1695444 | 1695532 | Hypomethylated |
| Granulocyte | chr8 | 142180100 | 142180164 | Hypomethylated |
| Granulocyte | chr2 | 209223877 | 209224762 | Hypomethylated |
| Granulocyte | chr14 | 23586796 | 23587093 | Hypomethylated |
| Granulocyte | chr16 | 85561155 | 85561229 | Hypomethylated |
| Granulocyte | chr17 | 33425704 | 33427253 | Hypomethylated |
| Granulocyte | chr17 | 78748036 | 78748234 | Hypomethylated |
| Granulocyte | chr12 | 124908476 | 124908602 | Hypomethylated |
| Granulocyte | chr15 | 101093772 | 101093974 | Hypomethylated |
| Granulocyte | chr17 | 79244235 | 79244356 | Hypomethylated |
| Granulocyte | chr2 | 120516628 | 120516725 | Hypomethylated |
| Granulocyte | chr5 | 177956161 | 177956259 | Hypomethylated |
| Granulocyte | chr10 | 73498445 | 73498902 | Hypomethylated |
| Granulocyte | chr20 | 1785056 | 1785521 | Hypomethylated |
| Granulocyte | chr9 | 136919731 | 136919892 | Hypomethylated |
| Granulocyte | chr13 | 114262862 | 114263522 | Hypomethylated |
| Granulocyte | chr20 | 62522393 | 62522519 | Hypomethylated |
| Granulocyte | chr8 | 131000173 | 131000872 | Hypomethylated |
| Hepatocyte | chr19 | 2790708 | 2791240 | Hypomethylated |
| Hepatocyte | chr2 | 118674801 | 118675049 | Hypomethylated |
| Hepatocyte | chr12 | 133249223 | 133249419 | Hypomethylated |
| Hepatocyte | chr2 | 128176259 | 128176803 | Hypomethylated |
| Hepatocyte | chr19 | 16627211 | 16627478 | Hypomethylated |
| Hepatocyte | chr16 | 27226453 | 27227400 | Hypomethylated |
| Hepatocyte | chr19 | 59022243 | 59023070 | Hypomethylated |
| Hepatocyte | chr2 | 119980528 | 119980922 | Hypomethylated |
| Hepatocyte | chr17 | 41019749 | 41020862 | Hypomethylated |
| Hepatocyte | chr2 | 44065003 | 44065200 | Hypomethylated |
| Hepatocyte | chr4 | 155507816 | 155508049 | Hypomethylated |
| Hepatocyte | chr11 | 47267183 | 47267393 | Hypomethylated |
| Hepatocyte | chr22 | 50644494 | 50644958 | Hypomethylated |
| Hepatocyte | chr14 | 70263750 | 70263921 | Hypomethylated |
| Hepatocyte | chr14 | 103573762 | 103574056 | Hypomethylated |
| Hepatocyte | chr17 | 48540012 | 48540441 | Hypomethylated |
| Hepatocyte | chr19 | 11347217 | 11347465 | Hypomethylated |
| Hepatocyte | chr20 | 60753603 | 60754165 | Hypomethylated |
| Hepatocyte | chr22 | 38212483 | 38213122 | Hypomethylated |
| Hepatocyte | chr9 | 130551728 | 130551831 | Hypomethylated |
| Hepatocyte | chr16 | 1991285 | 1991497 | Hypomethylated |
| Hepatocyte | chr17 | 80197816 | 80197957 | Hypomethylated |
| Hepatocyte | chr15 | 64996479 | 64997466 | Hypomethylated |
| Hepatocyte | chr16 | 12354976 | 12355424 | Hypomethylated |
| Hepatocyte | chr16 | 31473751 | 31474229 | Hypomethylated |
| Hepatocyte | chr17 | 80052709 | 80053033 | Hypomethylated |
| Hepatocyte | chr4 | 185724474 | 185724838 | Hypomethylated |
| Hepatocyte | chr12 | 57625174 | 57625730 | Hypomethylated |
| Hepatocyte | chr2 | 44065230 | 44065928 | Hypomethylated |
| Hepatocyte | chr2 | 127818028 | 127818420 | Hypomethylated |
| Hepatocyte | chr7 | 1912065 | 1912694 | Hypomethylated |
| Hepatocyte | chr7 | 158673837 | 158674009 | Hypomethylated |
| Hepatocyte | chr20 | 62365874 | 62366578 | Hypomethylated |
| Hepatocyte | chr1 | 11106576 | 11107083 | Hypomethylated |
| Hepatocyte | chr2 | 44066294 | 44066867 | Hypomethylated |
| Hepatocyte | chr11 | 679700 | 680254 | Hypomethylated |
| Hepatocyte | chr17 | 27493494 | 27493786 | Hypomethylated |
| Hepatocyte | chr22 | 50644200 | 50644478 | Hypomethylated |
| Hepatocyte | chr12 | 3194286 | 3194554 | Hypomethylated |
| Hepatocyte | chr16 | 72981972 | 72982166 | Hypomethylated |
| Hepatocyte | chr19 | 3659410 | 3659740 | Hypomethylated |
| Hepatocyte | chr20 | 43108645 | 43109079 | Hypomethylated |
| Hepatocyte | chr14 | 95028058 | 95028332 | Hypomethylated |
| Hepatocyte | chr9 | 139840215 | 139840491 | Hypomethylated |
| Hepatocyte | chr12 | 109639260 | 109639506 | Hypomethylated |
| Hepatocyte | chr17 | 17463355 | 17463878 | Hypomethylated |
| Hepatocyte | chr22 | 18324579 | 18324626 | Hypomethylated |
| Hepatocyte | chr3 | 126060006 | 126060381 | Hypomethylated |
| Hepatocyte | chr4 | 6755163 | 6755373 | Hypomethylated |
| Hepatocyte | chr12 | 7280736 | 7281344 | Hypomethylated |
| Keratinocyte | chr7 | 4802065 | 4802254 | Hypomethylated |
| Keratinocyte | chr8 | 143463390 | 143463895 | Hypomethylated |
| Keratinocyte | chr2 | 98349354 | 98349712 | Hypomethylated |
| Keratinocyte | chr11 | 65306835 | 65307074 | Hypomethylated |
| Keratinocyte | chr11 | 391681 | 392015 | Hypomethylated |
| Keratinocyte | chr11 | 392133 | 392695 | Hypomethylated |
| Keratinocyte | chr1 | 3321982 | 3322140 | Hypomethylated |
| Keratinocyte | chr5 | 178565867 | 178566128 | Hypomethylated |
| Keratinocyte | chr17 | 61558935 | 61559387 | Hypomethylated |
| Keratinocyte | chr16 | 3129975 | 3130351 | Hypomethylated |
| Keratinocyte | chr17 | 55037093 | 55037359 | Hypomethylated |
| Keratinocyte | chr6 | 36936452 | 36936717 | Hypomethylated |
| Keratinocyte | chr16 | 1017921 | 1018739 | Hypomethylated |
| Keratinocyte | chr20 | 62705520 | 62705726 | Hypomethylated |
| Keratinocyte | chr1 | 3322151 | 3322267 | Hypomethylated |
| Keratinocyte | chr5 | 465181 | 465615 | Hypomethylated |
| Keratinocyte | chr17 | 79244962 | 79245204 | Hypomethylated |
| Keratinocyte | chr22 | 47022433 | 47022659 | Hypomethylated |
| Keratinocyte | chr2 | 97423178 | 97423608 | Hypomethylated |
| Keratinocyte | chr8 | 143869435 | 143869513 | Hypomethylated |
| Keratinocyte | chr16 | 2334301 | 2334759 | Hypomethylated |
| Keratinocyte | chr20 | 18295551 | 18295861 | Hypomethylated |
| Keratinocyte | chr1 | 2309948 | 2310095 | Hypomethylated |
| Keratinocyte | chr9 | 94572657 | 94572869 | Hypomethylated |
| Keratinocyte | chr12 | 3190418 | 3190860 | Hypomethylated |
| Keratinocyte | chr12 | 98986220 | 98986393 | Hypomethylated |
| Keratinocyte | chr6 | 167506945 | 167507168 | Hypomethylated |
| Keratinocyte | chr20 | 18167947 | 18168360 | Hypomethylated |
| Keratinocyte | chr11 | 460362 | 460681 | Hypomethylated |
| Keratinocyte | chr17 | 76027640 | 76027890 | Hypomethylated |
| Keratinocyte | chr7 | 99227275 | 99227516 | Hypomethylated |
| Keratinocyte | chr11 | 65582724 | 65582909 | Hypomethylated |
| Keratinocyte | chr7 | 5648142 | 5648380 | Hypomethylated |
| Keratinocyte | chr8 | 102076454 | 102076803 | Hypomethylated |
| Keratinocyte | chr8 | 142235391 | 142235926 | Hypomethylated |
| Keratinocyte | chr10 | 13771352 | 13771658 | Hypomethylated |
| Keratinocyte | chr11 | 131707430 | 131707776 | Hypomethylated |
| Keratinocyte | chr9 | 79631906 | 79632226 | Hypermethylated |
| Keratinocyte | chr9 | 129386162 | 129386326 | Hypermethylated |
| Keratinocyte | chr10 | 8094621 | 8094861 | Hypermethylated |
| Keratinocyte | chr10 | 119292088 | 119292379 | Hypermethylated |
| Keratinocyte | chr12 | 54384508 | 54385130 | Hypermethylated |
| Keratinocyte | chr10 | 119292418 | 119292676 | Hypermethylated |
| Keratinocyte | chr12 | 54359358 | 54359894 | Hypermethylated |
| Keratinocyte | chr12 | 54338915 | 54339051 | Hypermethylated |
| Keratinocyte | chr10 | 119294251 | 119294603 | Hypermethylated |
| Keratinocyte | chr9 | 129372913 | 129373070 | Hypermethylated |
| Keratinocyte | chr9 | 129388068 | 129388495 | Hypermethylated |
| Keratinocyte | chr9 | 129388506 | 129388993 | Hypermethylated |
| Keratinocyte | chr12 | 54338568 | 54338887 | Hypermethylated |
| Kidney Epithelial | chr10 | 64041172 | 64041512 | Hypomethylated |
| Kidney Epithelial | chr4 | 1625248 | 1625493 | Hypomethylated |
| Kidney Epithelial | chr4 | 8642130 | 8642286 | Hypomethylated |
| Kidney Epithelial | chr4 | 42363614 | 42363736 | Hypomethylated |
| Kidney Epithelial | chr12 | 4554786 | 4554972 | Hypomethylated |
| Kidney Epithelial | chr17 | 74908539 | 74908715 | Hypomethylated |
| Kidney Epithelial | chr10 | 44163409 | 44163531 | Hypomethylated |
| Kidney Epithelial | chr22 | 39685694 | 39685809 | Hypomethylated |
| Kidney Epithelial | chr10 | 134945578 | 134945672 | Hypomethylated |
| Kidney Epithelial | chr9 | 91321534 | 91321820 | Hypomethylated |
| Kidney Epithelial | chr11 | 132582078 | 132582512 | Hypomethylated |
| Kidney Epithelial | chr11 | 132912309 | 132912375 | Hypomethylated |
| Kidney Epithelial | chr7 | 157427089 | 157427365 | Hypomethylated |
| Kidney Epithelial | chr12 | 53517541 | 53517743 | Hypomethylated |
| Kidney Epithelial | chr19 | 1961122 | 1961657 | Hypomethylated |
| Kidney Epithelial | chr5 | 176225786 | 176225897 | Hypomethylated |
| Kidney Epithelial | chr11 | 132031389 | 132031554 | Hypomethylated |
| Kidney Epithelial | chr4 | 188097977 | 188098247 | Hypomethylated |
| Kidney Epithelial | chr22 | 23607590 | 23607758 | Hypomethylated |
| Kidney Epithelial | chr12 | 132661484 | 132661640 | Hypomethylated |
| Kidney Epithelial | chr16 | 895422 | 895536 | Hypomethylated |
| Kidney Epithelial | chr10 | 504484 | 504785 | Hypomethylated |
| Kidney Epithelial | chr12 | 125242868 | 125242959 | Hypomethylated |
| Kidney Epithelial | chr17 | 75695335 | 75695465 | Hypomethylated |
| Kidney Epithelial | chr18 | 7231222 | 7232053 | Hypomethylated |
| Kidney Epithelial | chr2 | 34902628 | 34903058 | Hypomethylated |
| Kidney Epithelial | chr11 | 116484298 | 116484379 | Hypomethylated |
| Kidney Epithelial | chr18 | 76151279 | 76151406 | Hypomethylated |
| Kidney Epithelial | chr1 | 3680249 | 3680473 | Hypomethylated |
| Kidney Epithelial | chr4 | 8642296 | 8642353 | Hypomethylated |
| Kidney Epithelial | chr14 | 104834370 | 104834458 | Hypomethylated |
| Kidney Epithelial | chr4 | 619473 | 619638 | Hypomethylated |
| Kidney Epithelial | chr10 | 135120066 | 135120641 | Hypomethylated |
| Kidney Epithelial | chr4 | 1641960 | 1642062 | Hypomethylated |
| Kidney Epithelial | chr8 | 122961356 | 122961731 | Hypomethylated |
| Kidney Epithelial | chr19 | 34178390 | 34178526 | Hypomethylated |
| Kidney Epithelial | chr1 | 4193830 | 4193883 | Hypomethylated |
| Kidney Epithelial | chr4 | 100574404 | 100574537 | Hypomethylated |
| Kidney Epithelial | chr7 | 157258798 | 157258884 | Hypomethylated |
| Kidney Epithelial | chr16 | 3142948 | 3143251 | Hypomethylated |
| Kidney Epithelial | chr17 | 80192099 | 80192259 | Hypomethylated |
| Kidney Epithelial | chr19 | 10823619 | 10823914 | Hypomethylated |
| Kidney Epithelial | chr5 | 72677195 | 72677378 | Hypermethylated |
| Kidney Epithelial | chr1 | 47911646 | 47911941 | Hypermethylated |
| Kidney Epithelial | chr5 | 72597296 | 72597969 | Hypermethylated |
| Kidney Epithelial | chr5 | 72677395 | 72677689 | Hypermethylated |
| Kidney Epithelial | chr10 | 102586191 | 102586498 | Hypermethylated |
| Kidney Epithelial | chr10 | 102588392 | 102589472 | Hypermethylated |
| Kidney Epithelial | chr10 | 102586514 | 102588293 | Hypermethylated |
| Liver Endothelial | chr11 | 128698175 | 128698361 | Hypomethylated |
| Liver Endothelial | chr10 | 121169671 | 121170069 | Hypomethylated |
| Liver Endothelial | chr17 | 80803660 | 80804189 | Hypomethylated |
| Liver Endothelial | chr19 | 11707038 | 11707247 | Hypomethylated |
| Liver Endothelial | chr13 | 29329007 | 29329215 | Hypomethylated |
| Liver Endothelial | chr19 | 18233987 | 18234213 | Hypomethylated |
| Liver Endothelial | chr1 | 3038085 | 3038257 | Hypomethylated |
| Liver Endothelial | chr7 | 150690506 | 150691038 | Hypomethylated |
| Liver Endothelial | chr19 | 4983547 | 4983872 | Hypomethylated |
| Liver Endothelial | chr1 | 4237464 | 4237671 | Hypomethylated |
| Liver Endothelial | chr16 | 1562314 | 1562661 | Hypomethylated |
| Liver Endothelial | chr6 | 167028939 | 167029195 | Hypomethylated |
| Liver Endothelial | chr14 | 69931518 | 69931952 | Hypomethylated |
| Liver Endothelial | chr13 | 29328750 | 29328980 | Hypomethylated |
| Liver Endothelial | chr17 | 79170799 | 79170897 | Hypomethylated |
| Liver Endothelial | chr7 | 142984797 | 142984913 | Hypomethylated |
| Liver Endothelial | chr9 | 35909661 | 35910091 | Hypomethylated |
| Liver Endothelial | chr2 | 109891992 | 109892086 | Hypomethylated |
| Liver Endothelial | chr6 | 1635704 | 1635851 | Hypomethylated |
| Liver Endothelial | chr11 | 70266256 | 70266421 | Hypomethylated |
| Liver Endothelial | chr12 | 52291132 | 52291323 | Hypomethylated |
| Liver Endothelial | chr16 | 2220378 | 2221058 | Hypomethylated |
| Liver Endothelial | chr16 | 8943021 | 8943199 | Hypomethylated |
| Liver Endothelial | chr12 | 117113249 | 117113487 | Hypomethylated |
| Liver Endothelial | chr1 | 101705632 | 101705773 | Hypomethylated |
| Liver Endothelial | chr7 | 131217702 | 131217878 | Hypomethylated |
| Liver Endothelial | chr9 | 138900552 | 138900629 | Hypomethylated |
| Liver Endothelial | chr17 | 1975093 | 1975641 | Hypomethylated |
| Liver Endothelial | chr6 | 159549081 | 159549217 | Hypomethylated |
| Liver Endothelial | chr9 | 139406515 | 139406839 | Hypomethylated |
| Liver Endothelial | chr12 | 121717850 | 121717948 | Hypomethylated |
| Liver Endothelial | chr15 | 83781607 | 83781804 | Hypomethylated |
| Liver Endothelial | chr11 | 72300978 | 72301585 | Hypermethylated |
| Liver Endothelial | chr19 | 8398826 | 8399120 | Hypermethylated |
| Liver Stromal | chr6 | 125583698 | 125584141 | Hypomethylated |
| Liver Stromal | chr19 | 10927122 | 10928380 | Hypomethylated |
| Liver Stromal | chr8 | 96572161 | 96572434 | Hypomethylated |
| Liver Stromal | chr1 | 115826123 | 115826716 | Hypomethylated |
| Liver Stromal | chr2 | 238187070 | 238187250 | Hypomethylated |
| Liver Stromal | chr2 | 10501114 | 10501274 | Hypomethylated |
| Liver Stromal | chr15 | 67457630 | 67458134 | Hypomethylated |
| Liver Stromal | chr16 | 88121144 | 88121410 | Hypomethylated |
| Liver Stromal | chr6 | 22568625 | 22569458 | Hypomethylated |
| Liver Stromal | chr16 | 87260962 | 87261334 | Hypomethylated |
| Liver Stromal | chr6 | 109274387 | 109274515 | Hypomethylated |
| Liver Stromal | chr2 | 168614623 | 168615088 | Hypomethylated |
| Liver Stromal | chr2 | 239860016 | 239860285 | Hypomethylated |
| Liver Stromal | chr9 | 139256433 | 139256703 | Hypomethylated |
| Liver Stromal | chr17 | 48263099 | 48264061 | Hypomethylated |
| Liver Stromal | chr1 | 879443 | 879810 | Hypomethylated |
| Liver Stromal | chr7 | 1162971 | 1163186 | Hypomethylated |
| Liver Stromal | chr12 | 124514696 | 124514989 | Hypomethylated |
| Liver Stromal | chr16 | 87261416 | 87261576 | Hypomethylated |
| Liver Stromal | chr19 | 11276278 | 11277072 | Hypomethylated |
| Liver Stromal | chr20 | 48600915 | 48601197 | Hypomethylated |
| Liver Stromal | chr3 | 141162003 | 141163508 | Hypomethylated |
| Liver Stromal | chr7 | 616047 | 616267 | Hypomethylated |
| Liver Stromal | chr7 | 1953582 | 1954011 | Hypomethylated |
| Liver Stromal | chr12 | 9478964 | 9479222 | Hypomethylated |
| Liver Stromal | chr6 | 2579510 | 2579743 | Hypomethylated |
| Liver Stromal | chr10 | 131813008 | 131813140 | Hypomethylated |
| Liver Stromal | chr8 | 97166708 | 97167180 | Hypermethylated |
| Liver Stromal | chr4 | 174415351 | 174415831 | Hypermethylated |
| Liver Stromal | chr5 | 92907765 | 92907931 | Hypermethylated |
| Liver Stromal | chr19 | 48833395 | 48833967 | Hypermethylated |
| Liver Stromal | chr5 | 92908300 | 92908694 | Hypermethylated |
| Liver Stromal | chr7 | 35295220 | 35295353 | Hypermethylated |
| Liver Stromal | chr7 | 35297778 | 35298218 | Hypermethylated |
| Liver Stromal | chr15 | 37402482 | 37402724 | Hypermethylated |
| Liver Stromal | chr15 | 76634052 | 76634571 | Hypermethylated |
| Liver Stromal | chr15 | 76634581 | 76634822 | Hypermethylated |
| Liver Stromal | chr16 | 86530027 | 86530682 | Hypermethylated |
| Liver Stromal | chr7 | 35295997 | 35296479 | Hypermethylated |
| Liver Stromal | chr16 | 86537916 | 86538268 | Hypermethylated |
| Liver Stromal | chr16 | 86538276 | 86538344 | Hypermethylated |
| Liver Stromal | chr5 | 92907932 | 92908202 | Hypermethylated |
| Liver Stromal | chr5 | 122434379 | 122434629 | Hypermethylated |
| Liver Stromal | chr8 | 72917066 | 72917696 | Hypermethylated |
| Liver Stromal | chr16 | 86535785 | 86536240 | Hypermethylated |
| Liver Stromal | chr5 | 122435167 | 122435525 | Hypermethylated |
| Liver Stromal | chr16 | 86528332 | 86529004 | Hypermethylated |
| Liver Stromal | chr16 | 86529012 | 86529284 | Hypermethylated |
| Liver Stromal | chr16 | 86540831 | 86541510 | Hypermethylated |
| Liver Stromal | chr16 | 86529518 | 86529935 | Hypermethylated |
| Liver Resident | chr1 | 167485764 | 167486300 | Hypomethylated |
| Immune | | | | |
| Liver Resident | chr13 | 100004173 | 100004739 | Hypomethylated |
| Immune | | | | |
| Liver Resident | chr10 | 62671028 | 62672100 | Hypomethylated |
| Immune | | | | |
| Liver Resident | chr5 | 1172977 | 1173240 | Hypomethylated |
| Immune | | | | |
| Liver Resident | chr16 | 89408213 | 89408323 | Hypomethylated |
| Immune | | | | |
| Liver Resident | chr12 | 122711771 | 122712065 | Hypomethylated |
| Immune | | | | |
| Liver Resident | chr5 | 125798690 | 125800185 | Hypomethylated |
| Immune | | | | |
| Liver Resident | chr19 | 10226740 | 10226846 | Hypomethylated |
| Immune | | | | |
| Liver Resident | chr6 | 159457246 | 159457551 | Hypomethylated |
| Immune | | | | |
| Liver Resident | chr11 | 69240780 | 69240893 | Hypomethylated |
| Immune | | | | |
| Liver Resident | chr14 | 61799217 | 61801202 | Hypomethylated |
| Immune | | | | |
| Liver Resident | chr2 | 232396314 | 232396622 | Hypomethylated |
| Immune | | | | |
| Liver Resident | chr13 | 24825770 | 24826000 | Hypomethylated |
| Immune | | | | |
| Liver Resident | chr3 | 134634509 | 134634904 | Hypomethylated |
| Immune | | | | |
| Liver Resident | chr22 | 28501414 | 28501559 | Hypomethylated |
| Immune | | | | |
| Liver Resident | chr8 | 55788511 | 55789245 | Hypomethylated |
| Immune | | | | |
| Liver Resident | chr8 | 129089049 | 129089294 | Hypomethylated |
| Immune | | | | |
| Liver Resident | chr10 | 8373235 | 8373451 | Hypomethylated |
| Immune | | | | |
| Liver Resident | chr11 | 67254108 | 67254405 | Hypomethylated |
| Immune | | | | |
| Liver Resident | chr1 | 228004834 | 228005102 | Hypomethylated |
| Immune | | | | |
| Liver Resident | chr13 | 53507709 | 53507874 | Hypomethylated |
| Immune | | | | |
| Liver Resident | chr3 | 15492693 | 15493298 | Hypomethylated |
| Immune | | | | |
| Liver Resident | chr11 | 119897856 | 119898003 | Hypomethylated |
| Immune | | | | |
| Liver Resident | chr19 | 14231243 | 14232111 | Hypomethylated |
| Immune | | | | |
| Liver Resident | chr8 | 588952 | 589567 | Hypomethylated |
| Immune | | | | |
| Liver Resident | chr12 | 42627503 | 42629139 | Hypomethylated |
| Immune | | | | |
| Liver Resident | chr1 | 182925302 | 182926777 | Hypomethylated |
| Immune | | | | |
| Liver Resident | chr17 | 4079397 | 4079653 | Hypomethylated |
| Immune | | | | |
| Liver Resident | chr20 | 57412376 | 57413281 | Hypomethylated |
| Immune | | | | |
| Liver Resident | chr2 | 11775213 | 11775828 | Hypomethylated |
| Immune | | | | |
| Liver Resident | chr5 | 1793815 | 1794340 | Hypomethylated |
| Immune | | | | |
| Liver Resident | chr6 | 2414426 | 2415245 | Hypomethylated |
| Immune | | | | |
| Liver Resident | chr12 | 9106413 | 9107244 | Hypomethylated |
| Immune | | | | |
| Liver Resident | chr3 | 45984585 | 45986499 | Hypomethylated |
| Immune | | | | |
| Liver Resident | chr3 | 60621448 | 60622527 | Hypomethylated |
| Immune | | | | |
| Liver Resident | chr12 | 133413238 | 133413408 | Hypomethylated |
| Immune | | | | |
| Liver Resident | chr2 | 96933283 | 96933947 | Hypomethylated |
| Immune | | | | |
| Liver Resident | chr5 | 133452036 | 133452371 | Hypomethylated |
| Immune | | | | |
| Liver Resident | chr6 | 158985919 | 158986382 | Hypomethylated |
| Immune | | | | |
| Liver Resident | chr9 | 101754799 | 101755349 | Hypomethylated |
| Immune | | | | |
| Liver Resident | chr10 | 63746813 | 63747406 | Hypomethylated |
| Immune | | | | |
| Liver Resident | chr12 | 122712075 | 122712154 | Hypomethylated |
| Immune | | | | |
| Liver Resident | chr1 | 167486587 | 167487296 | Hypomethylated |
| Immune | | | | |
| Liver Resident | chr7 | 155024838 | 155025349 | Hypomethylated |
| Immune | | | | |
| Liver Resident | chr8 | 10210347 | 10210483 | Hypomethylated |
| Immune | | | | |
| Liver Resident | chr11 | 61672040 | 61672078 | Hypermethylated |
| Immune | | | | |
| Liver Resident | chr2 | 173293597 | 173294219 | Hypermethylated |
| Immune | | | | |
| Liver Resident | chr11 | 64009996 | 64010074 | Hypermethylated |
| Immune | | | | |
| Liver Resident | chr9 | 19050493 | 19050678 | Hypermethylated |
| Immune | | | | |
| Liver Resident | chr10 | 85955136 | 85955226 | Hypermethylated |
| Immune | | | | |
| Lung Epithelial | chr2 | 234394496 | 234394646 | Hypomethylated |
| Lung Epithelial | chr6 | 7204780 | 7204927 | Hypomethylated |
| Lung Epithelial | chr6 | 163731158 | 163731308 | Hypomethylated |
| Lung Epithelial | chr11 | 113216805 | 113217041 | Hypomethylated |
| Lung Epithelial | chr13 | 114304531 | 114304702 | Hypomethylated |
| Lung Epithelial | chr22 | 46929575 | 46929762 | Hypomethylated |
| Lung Epithelial | chr20 | 56296443 | 56296576 | Hypomethylated |
| Lung Epithelial | chr2 | 235237949 | 235238118 | Hypomethylated |
| Lung Epithelial | chr11 | 66454486 | 66454682 | Hypomethylated |
| Lung Epithelial | chr12 | 66983978 | 66984318 | Hypomethylated |
| Lung Epithelial | chr20 | 56296470 | 56296576 | Hypomethylated |
| Lung Epithelial | chr16 | 85517269 | 85517471 | Hypomethylated |
| Lung Epithelial | chr17 | 79953059 | 79953137 | Hypomethylated |
| Lung Epithelial | chr1 | 2266262 | 2266414 | Hypomethylated |
| Lung Epithelial | chr12 | 26261448 | 26262385 | Hypomethylated |
| Lung Epithelial | chr14 | 104048108 | 104048201 | Hypomethylated |
| Lung Epithelial | chr13 | 111935187 | 111935468 | Hypomethylated |
| Lung Epithelial | chr7 | 2770561 | 2770802 | Hypomethylated |
| Lung Epithelial | chr9 | 136728413 | 136728520 | Hypomethylated |
| Lung Epithelial | chr10 | 1257678 | 1257979 | Hypomethylated |
| Lung Epithelial | chr10 | 112889204 | 112889350 | Hypomethylated |
| Lung Epithelial | chr17 | 80247859 | 80248026 | Hypomethylated |
| Lung Epithelial | chr11 | 111171223 | 111172653 | Hypomethylated |
| Lung Epithelial | chr6 | 46889597 | 46889761 | Hypomethylated |
| Lung Epithelial | chr8 | 142157078 | 142157174 | Hypomethylated |
| Lung Epithelial | chr17 | 9088165 | 9088467 | Hypomethylated |
| Lung Epithelial | chr2 | 234393573 | 234394479 | Hypomethylated |
| Lung Epithelial | chr16 | 677895 | 678174 | Hypomethylated |
| Lung Epithelial | chr17 | 71548667 | 71548755 | Hypomethylated |
| Lung Epithelial | chr1 | 2059898 | 2060168 | Hypomethylated |
| Lung Epithelial | chr20 | 56296660 | 56296827 | Hypomethylated |
| Lung Epithelial | chr2 | 234394372 | 234394479 | Hypomethylated |
| Lung Epithelial | chr16 | 12354976 | 12355424 | Hypomethylated |
| Lung Epithelial | chr7 | 33725751 | 33726087 | Hypomethylated |
| Lung Epithelial | chr10 | 13771352 | 13771658 | Hypomethylated |
| Lung Epithelial | chr22 | 46838804 | 46839245 | Hypomethylated |
| Lung Epithelial | chr12 | 94093563 | 94093982 | Hypomethylated |
| Lung Epithelial | chr6 | 155568960 | 155569279 | Hypomethylated |
| Lung Epithelial | chr13 | 114115128 | 114115433 | Hypomethylated |
| Lung Epithelial | chr4 | 1977200 | 1977236 | Hypomethylated |
| Lung Epithelial | chr4 | 8248193 | 8248391 | Hypomethylated |
| Lung Epithelial | chr6 | 46743464 | 46744227 | Hypomethylated |
| Lung Epithelial | chr13 | 98869837 | 98870145 | Hypomethylated |
| Lung Epithelial | chr16 | 3763054 | 3763348 | Hypomethylated |
| Lung Epithelial | chr19 | 8554999 | 8555062 | Hypomethylated |
| Lung Epithelial | chr7 | 5262422 | 5262575 | Hypomethylated |
| Lung Epithelial | chr17 | 41024622 | 41025029 | Hypomethylated |
| Lung Epithelial | chr21 | 43546441 | 43546655 | Hypomethylated |
| Lung Epithelial | chr4 | 8436008 | 8436223 | Hypomethylated |
| Lung Epithelial | chr22 | 19754439 | 19754839 | Hypermethylated |
| Megakaryocyte | chr16 | 1814980 | 1815151 | Hypomethylated |
| Megakaryocyte | chr16 | 3188474 | 3188511 | Hypomethylated |
| Megakaryocyte | chr1 | 7911113 | 7911210 | Hypomethylated |
| Megakaryocyte | chr10 | 467813 | 468083 | Hypomethylated |
| Megakaryocyte | chr19 | 1872715 | 1873024 | Hypomethylated |
| Megakaryocyte | chr9 | 71681914 | 71682266 | Hypomethylated |
| Megakaryocyte | chr14 | 103566265 | 103566681 | Hypomethylated |
| Megakaryocyte | chr16 | 88568536 | 88568989 | Hypomethylated |
| Megakaryocyte | chr17 | 61498395 | 61498815 | Hypomethylated |
| Megakaryocyte | chr8 | 145674184 | 145674298 | Hypomethylated |
| Megakaryocyte | chr12 | 8941393 | 8941494 | Hypomethylated |
| Megakaryocyte | chr5 | 481723 | 481794 | Hypomethylated |
| Megakaryocyte | chr2 | 239023319 | 239023500 | Hypomethylated |
| Megakaryocyte | chr5 | 37208967 | 37209202 | Hypomethylated |
| Megakaryocyte | chr11 | 68522449 | 68522793 | Hypomethylated |
| Megakaryocyte | chr19 | 56113869 | 56114292 | Hypomethylated |
| Megakaryocyte | chr8 | 142180962 | 142181506 | Hypomethylated |
| Megakaryocyte | chr14 | 92956120 | 92956450 | Hypomethylated |
| Megakaryocyte | chr14 | 105167146 | 105167290 | Hypomethylated |
| Megakaryocyte | chr19 | 16629936 | 16630066 | Hypomethylated |
| Megakaryocyte | chr19 | 14066489 | 14067377 | Hypomethylated |
| Megakaryocyte | chr5 | 37209212 | 37209381 | Hypomethylated |
| Megakaryocyte | chr7 | 1785741 | 1786129 | Hypomethylated |
| Megakaryocyte | chr7 | 137230659 | 137230997 | Hypomethylated |
| Megakaryocyte | chr8 | 10273760 | 10274076 | Hypomethylated |
| Megakaryocyte | chr7 | 631951 | 632605 | Hypomethylated |
| Megakaryocyte | chr7 | 632621 | 632817 | Hypomethylated |
| Megakaryocyte | chr11 | 63977307 | 63977540 | Hypomethylated |
| Megakaryocyte | chr1 | 1871823 | 1871974 | Hypomethylated |
| Megakaryocyte | chr19 | 5593710 | 5594329 | Hypomethylated |
| Megakaryocyte | chr22 | 39909921 | 39910015 | Hypomethylated |
| Megakaryocyte | chr11 | 68898452 | 68898667 | Hypomethylated |
| Megakaryocyte | chr19 | 12799867 | 12800212 | Hypomethylated |
| Megakaryocyte | chr19 | 49254245 | 49254406 | Hypomethylated |
| Megakaryocyte | chr7 | 149129744 | 149130209 | Hypomethylated |
| Megakaryocyte | chr15 | 74714681 | 74715017 | Hypomethylated |
| Megakaryocyte | chr19 | 50376109 | 50376389 | Hypomethylated |
| Megakaryocyte | chr8 | 145814448 | 145814812 | Hypomethylated |
| Megakaryocyte | chr14 | 102963357 | 102963929 | Hypomethylated |
| Megakaryocyte | chr16 | 88557690 | 88558056 | Hypomethylated |
| Megakaryocyte | chr2 | 179640101 | 179641010 | Hypomethylated |
| Megakaryocyte | chr3 | 194968078 | 194968601 | Hypomethylated |
| Megakaryocyte | chr5 | 1924041 | 1924100 | Hypomethylated |
| Megakaryocyte | chr5 | 53223677 | 53224181 | Hypomethylated |
| Megakaryocyte | chr8 | 141312889 | 141313313 | Hypomethylated |
| Megakaryocyte | chr16 | 1545317 | 1545676 | Hypomethylated |
| Megakaryocyte | chr3 | 194835984 | 194836405 | Hypomethylated |
| Megakaryocyte | chr8 | 53323520 | 53323719 | Hypomethylated |
| Megakaryocyte | chr16 | 85551464 | 85551806 | Hypomethylated |
| Megakaryocyte | chr16 | 86755593 | 86756024 | Hypomethylated |
| Monocytes and | chr3 | 196351807 | 196352171 | Hypomethylated |
| Macrophage | | | | |
| Monocytes and | chr12 | 6659484 | 6659682 | Hypomethylated |
| Macrophage | | | | |
| Monocytes and | chr13 | 32888845 | 32889052 | Hypomethylated |
| Macrophage | | | | |
| Monocytes and | chr3 | 195897812 | 195898123 | Hypomethylated |
| Macrophage | | | | |
| Monocytes and | chr19 | 8568502 | 8568649 | Hypomethylated |
| Macrophage | | | | |
| Monocytes and | chr16 | 85577692 | 85577882 | Hypomethylated |
| Macrophage | | | | |
| Monocytes and | chr12 | 132469818 | 132470033 | Hypomethylated |
| Macrophage | | | | |
| Monocytes and | chr17 | 80581522 | 80581992 | Hypomethylated |
| Macrophage | | | | |
| Monocytes and | chr3 | 128370161 | 128370565 | Hypomethylated |
| Macrophage | | | | |
| Monocytes and | chr4 | 3531428 | 3531642 | Hypomethylated |
| Macrophage | | | | |
| Monocytes and | chr9 | 95799785 | 95800025 | Hypomethylated |
| Macrophage | | | | |
| Monocytes and | chr18 | 74824341 | 74824414 | Hypomethylated |
| Macrophage | | | | |
| Monocytes and | chr12 | 56731566 | 56731735 | Hypomethylated |
| Macrophage | | | | |
| Monocytes and | chr16 | 56892384 | 56892556 | Hypomethylated |
| Macrophage | | | | |
| Monocytes and | chr16 | 85873312 | 85873440 | Hypomethylated |
| Macrophage | | | | |
| Monocytes and | chr2 | 134866101 | 134866288 | Hypomethylated |
| Macrophage | | | | |
| Monocytes and | chr8 | 602054 | 602159 | Hypomethylated |
| Macrophage | | | | |
| Monocytes and | chr5 | 79422700 | 79423348 | Hypomethylated |
| Macrophage | | | | |
| Monocytes and | chr16 | 3597075 | 3597441 | Hypomethylated |
| Macrophage | | | | |
| Monocytes and | chr6 | 26385502 | 26385842 | Hypomethylated |
| Macrophage | | | | |
| Monocytes and | chr14 | 69091384 | 69091490 | Hypomethylated |
| Macrophage | | | | |
| Monocytes and | chr2 | 240225004 | 240225176 | Hypomethylated |
| Macrophage | | | | |
| Monocytes and | chr21 | 45773615 | 45773734 | Hypomethylated |
| Macrophage | | | | |
| Monocytes and | chr11 | 47350123 | 47350267 | Hypomethylated |
| Macrophage | | | | |
| Monocytes and | chr19 | 2073026 | 2073176 | Hypomethylated |
| Macrophage | | | | |
| Monocytes and | chr22 | 37309307 | 37309488 | Hypomethylated |
| Macrophage | | | | |
| Monocytes and | chr2 | 28697740 | 28697866 | Hypomethylated |
| Macrophage | | | | |
| Neuron | chr6 | 163558238 | 163558675 | Hypomethylated |
| Neuron | chr1 | 2006859 | 2007311 | Hypomethylated |
| Neuron | chr16 | 81731208 | 81731484 | Hypomethylated |
| Neuron | chr9 | 138775499 | 138775772 | Hypomethylated |
| Neuron | chr17 | 30815173 | 30815470 | Hypomethylated |
| Neuron | chr8 | 143463390 | 143463895 | Hypomethylated |
| Neuron | chr1 | 14113094 | 14113343 | Hypomethylated |
| Neuron | chr1 | 110082688 | 110083518 | Hypomethylated |
| Neuron | chr17 | 76675816 | 76676403 | Hypomethylated |
| Neuron | chr18 | 42324837 | 42325188 | Hypomethylated |
| Neuron | chr3 | 47932918 | 47934038 | Hypomethylated |
| Neuron | chr11 | 117232011 | 117232455 | Hypomethylated |
| Neuron | chr17 | 76818594 | 76819359 | Hypomethylated |
| Neuron | chr10 | 131694516 | 131694773 | Hypomethylated |
| Neuron | chr7 | 636539 | 636950 | Hypomethylated |
| Neuron | chr11 | 6425899 | 6426501 | Hypomethylated |
| Neuron | chr1 | 2236562 | 2236748 | Hypomethylated |
| Neuron | chr1 | 6363384 | 6363883 | Hypomethylated |
| Neuron | chr1 | 111145683 | 111147040 | Hypomethylated |
| Neuron | chr2 | 1828347 | 1828664 | Hypomethylated |
| Neuron | chr9 | 138627849 | 138628084 | Hypomethylated |
| Neuron | chr10 | 3283414 | 3283766 | Hypomethylated |
| Neuron | chr1 | 2238334 | 2238847 | Hypomethylated |
| Neuron | chr19 | 4770747 | 4770983 | Hypomethylated |
| Neuron | chr7 | 150816696 | 150817291 | Hypomethylated |
| Neuron | chr17 | 77083111 | 77083194 | Hypomethylated |
| Neuron | chr2 | 43019573 | 43020395 | Hypermethylated |
| Neuron | chr3 | 49941216 | 49941579 | Hypermethylated |
| Neuron | chr19 | 1467071 | 1467140 | Hypermethylated |
| Neuron | chr6 | 166267918 | 166268069 | Hypermethylated |
| Neuron | chr11 | 31841762 | 31842091 | Hypermethylated |
| Neuron | chr17 | 8924080 | 8924226 | Hypermethylated |
| Neuron | chr21 | 34755391 | 34755801 | Hypermethylated |
| Neuron | chr19 | 38886319 | 38886635 | Hypermethylated |
| Neuron | chr19 | 46142860 | 46142980 | Hypermethylated |
| Neuron | chr9 | 38672385 | 38672729 | Hypermethylated |
| Neuron | chr10 | 103534498 | 103534585 | Hypermethylated |
| Neuron | chr11 | 64066758 | 64067406 | Hypermethylated |
| Neuron | chr12 | 130529868 | 130530228 | Hypermethylated |
| Neuron | chr19 | 1455008 | 1456059 | Hypermethylated |
| Neuron | chr17 | 72352876 | 72353282 | Hypermethylated |
| Neuron | chr19 | 46379859 | 46380198 | Hypermethylated |
| Neuron | chr4 | 8200669 | 8201295 | Hypermethylated |
| Neuron | chr12 | 49366026 | 49366367 | Hypermethylated |
| Neuron | chr7 | 140346986 | 140347127 | Hypermethylated |
| Neuron | chr7 | 131514718 | 131515081 | Hypermethylated |
| Neuron | chr11 | 62693619 | 62693987 | Hypermethylated |
| Neuron | chr8 | 145697840 | 145698117 | Hypermethylated |
| Neuron | chr2 | 97151733 | 97152183 | Hypermethylated |
| Neuron | chr14 | 70038530 | 70038749 | Hypermethylated |
| Natural Killer | chr6 | 159457324 | 159457551 | Hypomethylated |
| Natural Killer | chr6 | 10733158 | 10733571 | Hypomethylated |
| Natural Killer | chr17 | 1104805 | 1105072 | Hypomethylated |
| Natural Killer | chr5 | 10377133 | 10377320 | Hypomethylated |
| Natural Killer | chr2 | 426239 | 426502 | Hypomethylated |
| Natural Killer | chr6 | 158985966 | 158986382 | Hypomethylated |
| Natural Killer | chr16 | 2892965 | 2893055 | Hypomethylated |
| Natural Killer | chr10 | 72358669 | 72358804 | Hypomethylated |
| Natural Killer | chr10 | 72362893 | 72363273 | Hypomethylated |
| Natural Killer | chr19 | 4657680 | 4658152 | Hypomethylated |
| Natural Killer | chr13 | 114874439 | 114874735 | Hypomethylated |
| Natural Killer | chr3 | 39322103 | 39322776 | Hypomethylated |
| Natural Killer | chr21 | 47845235 | 47846073 | Hypomethylated |
| Natural Killer | chr22 | 46685653 | 46686131 | Hypomethylated |
| Natural Killer | chr1 | 11395635 | 11395863 | Hypomethylated |
| Natural Killer | chr11 | 120827449 | 120827536 | Hypomethylated |
| Natural Killer | chr19 | 10226728 | 10226846 | Hypomethylated |
| Natural Killer | chr13 | 77565285 | 77565577 | Hypomethylated |
| Natural Killer | chr10 | 72357918 | 72358611 | Hypomethylated |
| Natural Killer | chr16 | 89336192 | 89336628 | Hypomethylated |
| Natural Killer | chr21 | 47830003 | 47830198 | Hypomethylated |
| Natural Killer | chr16 | 84553497 | 84553694 | Hypomethylated |
| Pancreatic | chr7 | 97841826 | 97842223 | Hypomethylated |
| Pancreatic | chr17 | 80395185 | 80395451 | Hypomethylated |
| Pancreatic | chr7 | 97843854 | 97844802 | Hypomethylated |
| Pancreatic | chr16 | 3706036 | 3706908 | Hypomethylated |
| Pancreatic | chr1 | 22303212 | 22303543 | Hypomethylated |
| Pancreatic | chr9 | 139394558 | 139395025 | Hypomethylated |
| Pancreatic | chr19 | 56658298 | 56658723 | Hypomethylated |
| Pancreatic | chr6 | 35762792 | 35763566 | Hypomethylated |
| Pancreatic | chr11 | 794340 | 794623 | Hypomethylated |
| Pancreatic | chr4 | 186742139 | 186742364 | Hypomethylated |
| Pancreatic | chr16 | 75252683 | 75252951 | Hypomethylated |
| Pancreatic | chr17 | 78337515 | 78338076 | Hypomethylated |
| Pancreatic | chr9 | 135929763 | 135929928 | Hypomethylated |
| Pancreatic | chr9 | 135944497 | 135946085 | Hypomethylated |
| Pancreatic | chr8 | 145685382 | 145685798 | Hypomethylated |
| Pancreatic | chr16 | 630101 | 630351 | Hypomethylated |
| Pancreatic | chr17 | 77923890 | 77924179 | Hypomethylated |
| Pancreatic | chr8 | 103942869 | 103943309 | Hypomethylated |
| Pancreatic | chr17 | 705656 | 706287 | Hypomethylated |
| Pancreatic | chr19 | 39691205 | 39691581 | Hypomethylated |
| Pancreatic | chr19 | 49253497 | 49253771 | Hypomethylated |
| Pancreatic | chr9 | 139393948 | 139394113 | Hypomethylated |
| Pancreatic | chr16 | 75255120 | 75255591 | Hypomethylated |
| Pancreatic | chr10 | 130546138 | 130546266 | Hypomethylated |
| Pancreatic | chr16 | 88473427 | 88473645 | Hypomethylated |
| Pancreatic | chr5 | 1702312 | 1702471 | Hypomethylated |
| Pancreatic | chr5 | 10757768 | 10758645 | Hypomethylated |
| Pancreatic | chr7 | 97846514 | 97847025 | Hypomethylated |
| Pancreatic | chr11 | 1321822 | 1322132 | Hypomethylated |
| Pancreatic | chr12 | 110353054 | 110353218 | Hypomethylated |
| Pancreatic | chr12 | 126676605 | 126676791 | Hypomethylated |
| Pancreatic | chr17 | 37328203 | 37328335 | Hypomethylated |
| Pancreatic | chr19 | 49254245 | 49254406 | Hypomethylated |
| Pancreatic | chr7 | 97857387 | 97857615 | Hypomethylated |
| Pancreatic | chr1 | 22854310 | 22854475 | Hypomethylated |
| Pancreatic | chr7 | 150034206 | 150034367 | Hypomethylated |
| Pancreatic | chr1 | 24463665 | 24463848 | Hypomethylated |
| Pancreatic | chr6 | 35764704 | 35765094 | Hypomethylated |
| Pancreatic | chr20 | 17633665 | 17633859 | Hypomethylated |
| Pancreatic | chr4 | 1372943 | 1373417 | Hypomethylated |
| Pancreatic | chr7 | 156797845 | 156798469 | Hypermethylated |
| Pancreatic | chr13 | 28496765 | 28497184 | Hypermethylated |
| Pancreatic | chr13 | 28497826 | 28498280 | Hypermethylated |
| Pancreatic | chr11 | 119227020 | 119227708 | Hypermethylated |
| Pancreatic | chr14 | 105714520 | 105715440 | Hypermethylated |
| Pancreatic | chr7 | 157476754 | 157477033 | Hypermethylated |
| Pancreatic | chr15 | 53089968 | 53090394 | Hypermethylated |
| Pancreatic | chr19 | 47960704 | 47960978 | Hypermethylated |
| Pancreatic | chr12 | 65218052 | 65218948 | Hypermethylated |
| Pancreatic | chr13 | 28491265 | 28491526 | Hypermethylated |
| Prostate Epithelial | chr20 | 18295551 | 18295861 | Hypomethylated |
| Prostate Epithelial | chr7 | 2054500 | 2055180 | Hypomethylated |
| Prostate Epithelial | chr17 | 40823869 | 40824215 | Hypomethylated |
| Prostate Epithelial | chr17 | 9088165 | 9088467 | Hypomethylated |
| Prostate Epithelial | chr22 | 40415244 | 40415401 | Hypomethylated |
| Prostate Epithelial | chr1 | 27220656 | 27221119 | Hypomethylated |
| Prostate Epithelial | chr7 | 5549534 | 5549731 | Hypomethylated |
| Prostate Epithelial | chr16 | 81533203 | 81533549 | Hypomethylated |
| Prostate Epithelial | chr11 | 70022863 | 70023099 | Hypomethylated |
| Prostate Epithelial | chr7 | 1491787 | 1491952 | Hypomethylated |
| Prostate Epithelial | chr8 | 143386225 | 143386548 | Hypomethylated |
| Prostate Epithelial | chr8 | 142235391 | 142235926 | Hypomethylated |
| Prostate Epithelial | chr19 | 8554999 | 8555062 | Hypomethylated |
| Prostate Epithelial | chr10 | 116080333 | 116080495 | Hypomethylated |
| Prostate Epithelial | chr19 | 14636919 | 14637391 | Hypomethylated |
| Prostate Epithelial | chr9 | 140708598 | 140709066 | Hypomethylated |
| Prostate Epithelial | chr16 | 23158676 | 23159171 | Hypomethylated |
| Prostate Epithelial | chr19 | 30298144 | 30298342 | Hypomethylated |
| Prostate Epithelial | chr6 | 6894045 | 6894183 | Hypomethylated |
| Prostate Epithelial | chr18 | 77231461 | 77231655 | Hypomethylated |
| Prostate Epithelial | chr20 | 60953594 | 60953740 | Hypomethylated |
| Prostate Epithelial | chr1 | 17307773 | 17308093 | Hypomethylated |
| Prostate Epithelial | chr1 | 19208516 | 19208778 | Hypomethylated |
| Prostate Epithelial | chr2 | 174147832 | 174148599 | Hypomethylated |
| Prostate Epithelial | chr8 | 8748227 | 8749041 | Hypomethylated |
| Prostate Epithelial | chr11 | 129802069 | 129803188 | Hypomethylated |
| Prostate Epithelial | chr3 | 124859057 | 124859348 | Hypomethylated |
| Prostate Epithelial | chr3 | 197121225 | 197121420 | Hypomethylated |
| Prostate Epithelial | chr19 | 48796188 | 48796381 | Hypomethylated |
| Prostate Epithelial | chr4 | 1225408 | 1225653 | Hypomethylated |
| Prostate Epithelial | chr19 | 1425504 | 1425707 | Hypomethylated |
| Prostate Epithelial | chr1 | 3615514 | 3615746 | Hypomethylated |
| Prostate Epithelial | chr10 | 5567123 | 5567503 | Hypomethylated |
| Prostate Epithelial | chr10 | 81915434 | 81915647 | Hypomethylated |
| Prostate Epithelial | chr11 | 120007968 | 120008155 | Hypomethylated |
| Prostate Epithelial | chr20 | 18167947 | 18168360 | Hypomethylated |
| Prostate Epithelial | chr7 | 551344 | 551759 | Hypomethylated |
| Prostate Epithelial | chr1 | 38493014 | 38493557 | Hypomethylated |
| Prostate Epithelial | chr6 | 33740395 | 33740572 | Hypomethylated |
| Prostate Epithelial | chr7 | 26480584 | 26480922 | Hypomethylated |
| Prostate Epithelial | chr10 | 133796508 | 133796631 | Hypomethylated |
| Prostate Epithelial | chr22 | 43463194 | 43463331 | Hypomethylated |
| Prostate Epithelial | chr22 | 47023029 | 47023196 | Hypomethylated |
| Prostate Epithelial | chr1 | 2359816 | 2359937 | Hypomethylated |
| Prostate Epithelial | chr21 | 46232702 | 46232873 | Hypomethylated |
| Prostate Epithelial | chr22 | 43164347 | 43164751 | Hypomethylated |
| Prostate Epithelial | chr4 | 1317409 | 1317679 | Hypomethylated |
| Prostate Epithelial | chr10 | 30317521 | 30317689 | Hypomethylated |
| Prostate Epithelial | chr16 | 73099735 | 73099893 | Hypermethylated |
| Prostate Epithelial | chr6 | 10420567 | 10421060 | Hypermethylated |
| Skeletal Muscular | chr7 | 148497704 | 148497985 | Hypomethylated |
| Skeletal Muscular | chr18 | 46289370 | 46289545 | Hypomethylated |
| Skeletal Muscular | chr10 | 99330013 | 99330204 | Hypomethylated |
| Skeletal Muscular | chr11 | 129613860 | 129614016 | Hypomethylated |
| Skeletal Muscular | chr1 | 9324223 | 9324364 | Hypomethylated |
| Skeletal Muscular | chr18 | 55922618 | 55922960 | Hypomethylated |
| Skeletal Muscular | chr3 | 14582440 | 14582904 | Hypomethylated |
| Skeletal Muscular | chr17 | 78724929 | 78725376 | Hypomethylated |
| Skeletal Muscular | chr17 | 3769455 | 3770035 | Hypomethylated |
| Skeletal Muscular | chr10 | 23353063 | 23354028 | Hypomethylated |
| Skeletal Muscular | chr18 | 55923895 | 55924181 | Hypomethylated |
| Skeletal Muscular | chr1 | 1490850 | 1491199 | Hypomethylated |
| Skeletal Muscular | chr2 | 201342380 | 201342659 | Hypomethylated |
| Skeletal Muscular | chr7 | 611233 | 612221 | Hypomethylated |
| Skeletal Muscular | chr3 | 52869808 | 52871468 | Hypomethylated |
| Skeletal Muscular | chr17 | 65040730 | 65041120 | Hypomethylated |
| Skeletal Muscular | chr7 | 1351324 | 1351753 | Hypomethylated |
| Skeletal Muscular | chr17 | 76672617 | 76673183 | Hypomethylated |
| Skeletal Muscular | chr1 | 54856305 | 54856608 | Hypomethylated |
| Skeletal Muscular | chr4 | 25097386 | 25097706 | Hypomethylated |
| Skeletal Muscular | chr4 | 100572972 | 100573578 | Hypomethylated |
| Skeletal Muscular | chr7 | 613015 | 613422 | Hypomethylated |
| Skeletal Muscular | chr14 | 20903959 | 20904321 | Hypomethylated |
| Skeletal Muscular | chr17 | 79898313 | 79898988 | Hypomethylated |
| Skeletal Muscular | chr5 | 138730990 | 138731670 | Hypomethylated |
| Skeletal Muscular | chr17 | 11461316 | 11461624 | Hypomethylated |
| Skeletal Muscular | chr7 | 1351335 | 1351753 | Hypomethylated |
| Skeletal Muscular | chr7 | 56149083 | 56150012 | Hypomethylated |
| Skeletal Muscular | chr14 | 104636600 | 104636829 | Hypomethylated |
| Skeletal Muscular | chr7 | 612876 | 613698 | Hypomethylated |
| Skeletal Muscular | chr10 | 134894695 | 134894939 | Hypomethylated |
| Skeletal Muscular | chr17 | 11461335 | 11461638 | Hypomethylated |
| Skeletal Muscular | chr4 | 126572121 | 126572399 | Hypomethylated |
| Skeletal Muscular | chr16 | 89258456 | 89259792 | Hypermethylated |
| Mature T | chr13 | 24825869 | 24826000 | Hypomethylated |
| Mature T | chr22 | 37544974 | 37545667 | Hypomethylated |
| Mature T | chr1 | 17054071 | 17054128 | Hypomethylated |
| Mature T | chr10 | 13330036 | 13330429 | Hypomethylated |
| Mature T | chr10 | 1156420 | 1156511 | Hypomethylated |
| Mature T | chr2 | 85667088 | 85667542 | Hypomethylated |
| Mature T | chr13 | 111841565 | 111842079 | Hypomethylated |
| Mature T | chr14 | 99715693 | 99716236 | Hypomethylated |
| Mature T | chr7 | 625073 | 625320 | Hypomethylated |
| Mature T | chr11 | 60775001 | 60775404 | Hypermethylated |
| Mature T | chr3 | 42113514 | 42114020 | Hypermethylated |

*The start and end points of the genomic region is with reference to the Homo sapiens full genome as provided by University of California Santa Cruz, version hg19 (Genome Reference Consortium GRCh37, February 2009).

Analysis of ctDNA

Aspects of the present invention involve analysis of cfDNA to determine clonal heterogeneity of tumor cells. The determination of the heterogeneity of cells of the tumor cells comprises genotyping the cfDNA in order to obtain a genotype profile of the cfDNA. The genotype profile of the cfDNA can be compared with the genotype profile of cfDNA previously obtained from the subject and is well established in the genotyping of cancers for signature mutations or for previously unknown mutations. These mutations may be a point mutation, methylation changes, tumor-specific rearrangements (e.g., inversions, translocations, insertions and deletions), or cancer-derived viral sequences.

Examples of methods that can be used in genotyping include, but are not limited to, sequencing such as whole-genome sequencing or whole-exome sequencing; PCR; the Sanger-based cfDNA detection method (Newman et al., 2014); BEAMing (beads, emulsion, amplification, and magnetics) developed by Diehl et al (2008); and cancer personalized profiling by deep sequencing (CAPP-seq) (Newman et al., 2014).

### EXAMPLES

A study was conducted to establish sequencing-based, cell-type specific DNA methylation reference maps of human and mouse tissues to enable the assignment of DNA released from dying cells into the circulation back to its cellular origin. The study showed that cell-free, methylated DNA in blood samples revealed tissue-specific, cellular damage from radiation treatment.

### Methods

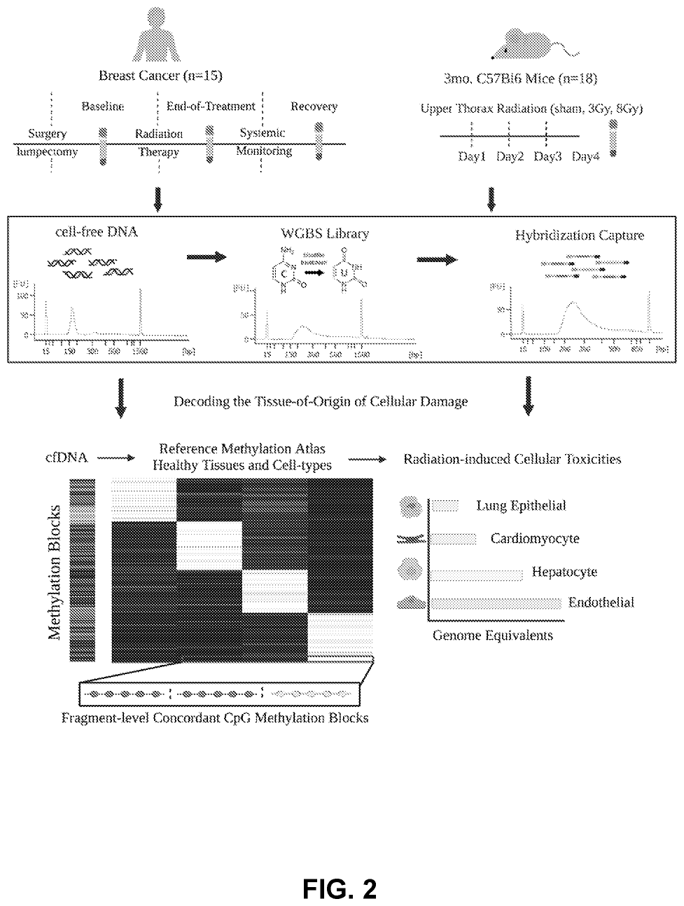

Human serum sample collection. Breast cancer patients undergoing adjuvant radiation-therapy participated in the study. For serum isolation, peripheral blood (˜8-12 ml) was collected and allowed to clot at room temperature for 30 minutes before centrifugation at 1500×g for 20 min at 4° C. to separate the serum fraction. The serum was aliquoted in 0.5 mL fractions and stored at −80° C. until use. Serial serum samples were collected from 15 breast cancer patients at Baseline (before radiation treatment), End-of-Treatment (EOT; 30 minutes after the last radiation treatment), and Recovery (one month after cessation of radiation treatment), thus allowing for a within-patient internal control and baseline. A schematic of the time series for sample collection can be found in FIG. 2. Patients received either three-dimensional conformal RT (3D-CRT) or a combination of proton beam therapy (PB) and 3D-CRT. Patient characteristics and treatment details including radiation dosimetry are summarized in Table 3 and in Barefoot et al., 2022, Supplemental Table 8.

Mouse serum and tissue collection. C57B16 mice (n=18) were irradiated to the upper thorax at varying dose (sham control, 3Gy, 8Cy) for 3 consecutive treatments. Serum and tissues were collected 24 hours after the last radiation dose. For serum isolation, blood was collected via cardiac puncture (˜1 mL) and allowed to clot at room temperature for 30 minutes before centrifugation at 1500×g for 20 min at 4° C. to separate the serum fraction. Heart, lung, and liver tissues were harvested and sectioned to be both flash frozen and formalin fixed for subsequent analysis.

Cell isolation. Reference methylomes were generated for mouse immune cell-types and human endothelial cell-types to augment publicly available datasets. Peripheral blood and bone marrow were isolated and spleens from healthy C57B16 mice were dissociated to single cells and FACS sorted using cell-type specific antibodies. Buffy coat (n=4), bone marrow (n=3), CD19+ B cell (n=1) CD4 T cell (n=1), CD8 T cell (n=1) and Gr1+Neutrophil (n=1) methylomes were generated using the following antibodies: FITC anti-mouse CD45, Alexa Fluor 647 anti-mouse CD3, Brilliant Violet 711 anti-mouse CD4, Brilliant Violet 421 anti-mouse CD8a, PE anti-mouse CD19, PE/Cy7 anti-mouse Ly-6G/Ly-6C (Gr-1) (all BioLegend 1:20). Cryopreserved passage 1 human liver sinusoidal endothelial cells (LSEC) were purchased. Purity was determined by immunofluorescence with antibodies specific to vWF/Factor VIII and CD31 (PECAM). Cryopreserved passage 2 human coronary artery, cardiac microvascular, pulmonary artery, and pulmonary microvascular endothelial cells were isolated from single donor healthy human tissues purchased. All endothelial cell populations were CD31 positive and Dil-Ac-LDL uptake positive. Paired RNA-seq data was generated from the same cell-populations used for DNA methylome profiling to validate the identity of purchased cell populations through analysis of cell-type expression markers.

RNA isolation, RNA-sequencing, and RT-qPCR analysis. RNA was isolated from tissues or sorted cells using the RNeasy Kit following homogenization step using the MagNA Lyser according to the manufacturer's protocol and quantified by Qubit RNA BR assay. Total RNA samples were validated using an Agilent RNA 6000 nano assay on the 2100 Bioanalyzer TapeStation. The resulting RNA Integrity number (RIN) of samples selected for downstream qPCR or RNAseq analysis was at least 7. Reverse transcription was done using iScript cDNA Synthesis Kit according to the manufacturer's protocol. Real-time quantitative RT-PCR was performed with iQ SYBR Green Supermix. Primers used for RT-qPCR were purchased from Integrated DNA Technologies. Fold change was calculated as a percentage normalized to housekeeping gene human actin (ACTB) using the delta-Ct method. All RT-qPCR assays were done in triplicate. RNA-sequencing libraries were prepared using TruSeq Total RNA library Prep Kit at Novogene Corporation Inc., and 150 bp paired-end sequencing was performed on an Illumina Hiseq 4000 with a depth of 50 million paired reads per sample. A reference index was generated using GTF annotation from GENCODEv28. Raw FASTQ files were aligned to GRCh38 or GRCm38 with HISAT2. Derived counts per million and P-value were used to create a rank ordered list, which was then used for subsequent analysis and confirmation of the identity of isolated cell-types for methylome analysis. Expression levels at know % n cell type markers from single cell expression databases were used to validate the identity of isolated cell-type populations for methylome analysis (Khan et al., 2018).

Isolation of circulating c/DNA. Circulating cfDNA was extracted from 3-4 mL human serum and 0.5 mL mouse serum, using the Q1Aamp Circulating Nucleic Acid kit according to the manufacturer's instructions. CfDNA was quantified via Qubit fluorometer using both the dsDNA High Sensitivity Assay Kit. As a quality control, fragment size distribution of isolated cfDNA was verified based on analysis using a 2100 Bioanalyzer TapeStation. Additional purification using Beckman Coulter beads was implemented to remove high-molecular weight DNA reflective of cell-lysis and leukocyte contamination as previously described (Maggi et al., 2018). Size distribution of cfDNA fragments were re-verified using 2100 Bioanalyzer TapeStation analysis following purification.

Isolation and fragmentation of genomic DNA. Genomic DNA from tissues was extracted with DNeasy Blood and Tissue Kit following the manufacturer's instructions and quantified via Qubit fluorometer dsDNA BR Assay Kit. Genomic DNA was fragmented via sonification using a Covaris E220 instrument to the recommended 150-200 base pairs before library preparation. Lambda phage DNA was also fragmented and included as a spike-in to all DNA samples at 0.5% w/w, serving as an internal unmethylated control. Bisulfite conversion efficiency was calculated through assessing the number of unconverted C's on unmethylated lambda phage DNA. The SeqCap Epi capture pool contains probes to capture the lambda genomic region from base 4500 to 6500. The conversion rate was calculated as follows: conversion rate=1−(sum(C_count)/sum(CT_count)) across the lambda genomic region captured.

Bisulfite capture-sequencing library preparation. Bisulfite capture-sequencing libraries were generated from either cfDNA or reference DNA inputs according to the same protocol. As a first step, WGBS libraries were generated using the Zymo Research Pico Methyl-Seq Library Prep Kit (D5455) with the following modifications. Bisulfite-conversion was carried out using the Zymo EZ DNA Methylation Gold kit instead of the EZ DNA Methylation-Lightning Kit. For mouse samples, cfDNA from two mice in the same group was pooled as input to library preparation. An additional 2 PCR cycles were added to the recommended cycle number based on total input cfDNA amounts. WGBS libraries were eluted in 15 μL 10 mM Tris-HC buffer, pH 8. Library quality control was performed with an Agilent 2100 Bioanalyzer and quantity determined via KAPA Library Quantification Kit.

Cell-free WGBS libraries were pooled to meet the required 1 μg DNA input necessary for targeted enrichment. However, no more than four WGBS libraries were pooled in a single hybridization reaction and the 1 μg input DNA was divided evenly between the libraries to be multiplexed. Hybridization capture was carried out according to the SeqCap Epi Enrichment System protocol using SeqCap Epi CpGiant probe pools for human samples and SeqCap Epi Developer probes for mouse samples with xGen Universal Blocker-TS Mix as the blocking reagent. Washing and recovering of the captured library, as well as PCR amplification and final purification, were carried out as recommended by the manufacturer. The capture library products were assessed by Agilent Bioanalyzer DNA 1000 assays. Bisulfite capture-sequencing libraries with inclusion of 15-20% spike-in PhiX Control v3 library were clustered on an Illumina Novaseq 6000 S4 flow cell followed by 150-bp paired-end sequencing.

Bisulfite sequencing data alignment and preprocessing. Paired-end FASTQ files were trimmed using Trim Galore (https://github.com/FelixKrueger/TrimGalore) with parameters “-paid-q 20-clip_R1 10-clip_R2 10-three_prime_clip_R1 10-three_prime_clip_R2 10” (https://github.com/FelixKrueger/Bismark). Trimmed paired-end FASTQ reads were mapped to the human genome (GRCh37/hg build) using Bismark (V 0.22.3) with parameters “-non-directional”, then converte to BAM files using Santools (V. 1.12). BAM files were sorted and indexed using Santools (V1.12). Reads were stripped from non-CpG nucleotides and converted to BETA and PAT files using webstools (V 0.1.0), a tool suite for working with WGBS data while preserving read-specific intrinsic dependencies (https://github.com/nloyfer/wgbs_tools) (Loyfer et al., 2022; Loyfer & Kaplan).

Reference DNA methylation data from healthy tissues and cells. Controlled access to reference WGBS data from normal human tissues and cell-types was requested from public consortia participating in the International Human Epigenome Consortium (IHEC) and upon approval downloaded from the European Genome-Phenome Archive (EGA), Japanese Genotype-phenotype Archive (JGA), and database of Genotypes an d Phenotypes (dbGAP) data repositories (Table 4; see also Barefoot et al., 2022, Supplemental Table 1). Reference mouse WGBS data from normal tissues and cell-types was downloaded from select GEO and SRA datasets (Table 5). Downloaded FASTQs were processed and realigned in a similar manner as the locally generated bisulfite-sequencing libraries described above. However, parameters were adjusted to account for each respective WGBS library type at both trimming and alignment steps as previously described in the Bismark User Guide (http://felixkrueger.github.io/Bismark/Docs/). WBGS libraries were deduplicated using deduplicate_bismark (V 0.22.3). Special consideration of bisulfite conversion efficiency was given to samples prepared by the μWGBS protocol and reads with a bisulfite conversion rate below 90% or with fewer than three cytosines outside a CpG context were removed.

Segmentation and clustering analysis. The genome was segmented into blocks of homogenous methylation as previously described in Loyfer et al. 2022 using wgbstools (with parameters segment-max_bp 5000) (Loyfer et al., 2022; Loyfer & Kaplan). In brief, a multi-channel Dynamic Programming segmentation algorithm was used to divide the genome into continuous genomic regions (blocks) showing homogenous methylation levels across multiple CpGs, for each sample. The segmentation algorithm was applied to 278 human reference WGBS methylomes and retained 351,395 blocks covered by the hybridization capture panel used in the analysis of cfDNA in human serum (captures 80 Mb, ˜20% of CpGs). Likewise, segmentation of 103 mouse WGBS datasets from healthy cell types and tissues identified 1,344,889 blocks covered by the mouse hybridization capture panel (captures 210 Mb, ˜75% of CpGs). The hierarchical relationship between reference tissue and cell type WGBS datasets was visualized through creation of a tree dendrogram. The top 30,000 most variant methylation blocks containing at least three CpG sites and coverage across 90% of samples were selected. The average methylation for each block and sample was computed using wgbstools (-beta_to_table). Trees were assembled using the unweighted pair-group method with arithmetic mean (UPGMA), using scipy (V 1.7.1) and L1 distance, and then visualized in R with the ggtree package (V 2.41). The similarity between samples was assessed by the degree of variation in distance between samples of the same cell-type (average 23,056) compared to samples between different cell-types (average 273,018). Dimensional reduction was also performed on the selected blocks using the UMAP package (V 0.2.8.2.0). Default UMAP parameters were used (15 neighbors, 2 components, Euclidean metric, and a minimum distance of 0.1).

Identification of cell-type specific methylation blocks. The original 278 human WGBS samples were reduced to a final set of 104 samples to identify differentially methylated cell-type specific blocks. Samples from bulk tissues and those that did not have sufficient coverage (missing values in >50% of methylation blocks) were excluded. Outlier replicates, or those clustering with fibroblasts or stromal cell types were excluded, due to possible contamination. Only immune cell methylomes that were reprocessed from raw sequencing data to PAT files were used to identify DMBs. The final 104 human reference samples were organized into groupings of 20 cell-types (see Table 4 and Barefoot et al., 2022, Supplemental Table 1). Similarly, the starting 103 mouse WGBS samples were reduced to a final set of 44 samples that were organized into a final grouping of 9 cell-types and tissues (see Table 5 and Barefoot et al., 2022, Supplemental Table 2). Tissue and cell-type specific methylation blocks were identified from the final reduced reference WGBS data using custom scripts. A one-vs-all comparison was performed to identify differentially methylated blocks unique for each group. This was done separately for human and mouse. First, blocks covering a minimum of three CpG sites, with length less than 2 Kb and at least 10 observations, were identified. Then, be average methylation per block/sample was calculated, as the ratio of methylated CpG observations across all sequenced reads from that block. Differential blocks were sorted by the margin of separation, termed “delta beta”, defined as the minimal difference between the average methylation in any sample from the target group versus all other samples. Blocks with a delta-beta≥0.4 in human and ≥0.35 in mouse were then selected. This resulted in a variable number of cell-type specific blocks available for each tissue and cell-type. Each DNA fragment was characterized as U (mostly unmethylated), M (mostly methylated) or X (mixed) based on the fraction of methylated CpG sites as previously described (Loyfer et al., 2022). Thresholds of ≤33% methylated CpGs for U reads and ≥66% methylated CpGs for M were used. A methylation score was calculated for each identified cell-type specific block based on the proportion of U/X/m reads among all reads. The U proportion was used to define hypomethylated blocks and the M proportion was used to define hyper methylated blocks. Selected human and mouse blocks for cell-types of interest can be found in Barefoot et al., 2022, Supplemental Tables 3 and 4. Heatmaps were generated using the pretty heatmap function in the RStudio Package for the R Bioconductor.

Likelihood-based probabilistic model for fragment-level deconvolution. The cell type origins of cfDNA were determined using a probabilistic fragment-level deconvolution algorithm. Using this model, the likelihood of each cfDNA molecule was calculated using a 4th order Markov Model, considering the joint methylation status of up to 5 adjacent CpG sites. Within individual tissue and cell-type specific blocks, this model is used to predict whether each molecule is classified as belonging to the tissue of interest or alternatively is classified as background. The posterior probability of each cfDNA molecule is calculated based on the log-likelihood that the origins of the specific read-pair came from the target cell-type times the prior knowledge of the probability that any read should originate from the target cell-type. The model was trained on reference bisulfite-sequencing data from normal cells and tissues to learn the distribution of each marker in the target tissue/cell-type of interest compared to background. Then the model was applied to test cfDNA methylomes for binary classification of the origins of each cfDNA molecule. The proportion of molecules assigned to the tissue of interest across all cell-type specific blocks was then summed and used to determine the relative abundance of cfDNA derived from that tissue origins in each respective sample. The resulting proportions were adjusted to have a sum of 1 through imposing a normalization constraint. Relative tissue-of-origin percentages were converted to genome equivalents and reported as an absolute measure (Geq/mL) considering the initial cfDNA concentrations [i.e., fraction cell-type specific cfDNA×initial concentration cfDNA ng/mL×3.3×10-12 grams per human haploid genome equivalent (or ×3.0×10-12 grams per mouse haploid genome equivalent)].

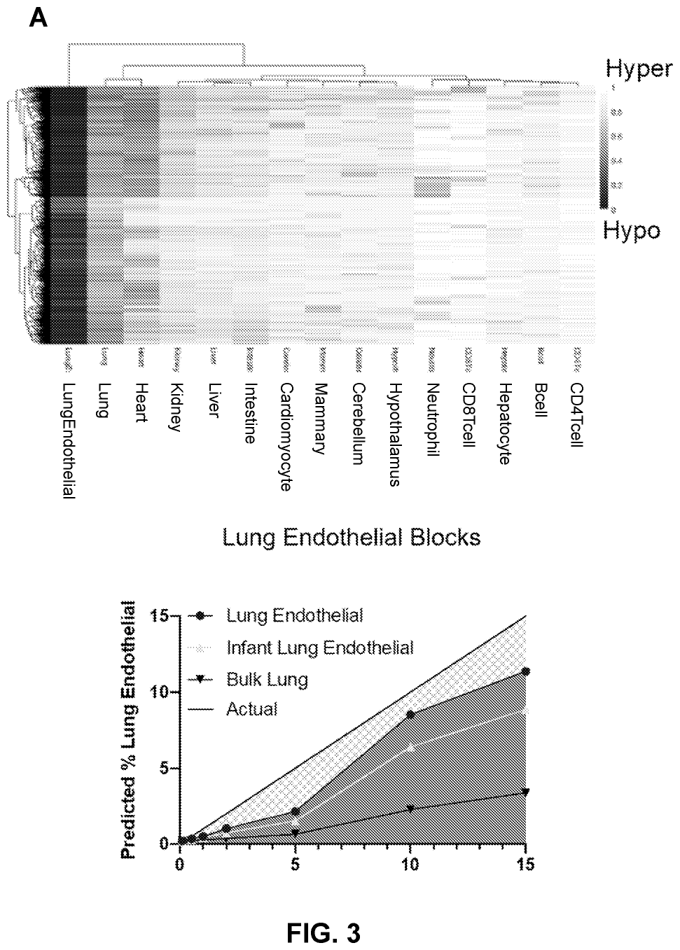

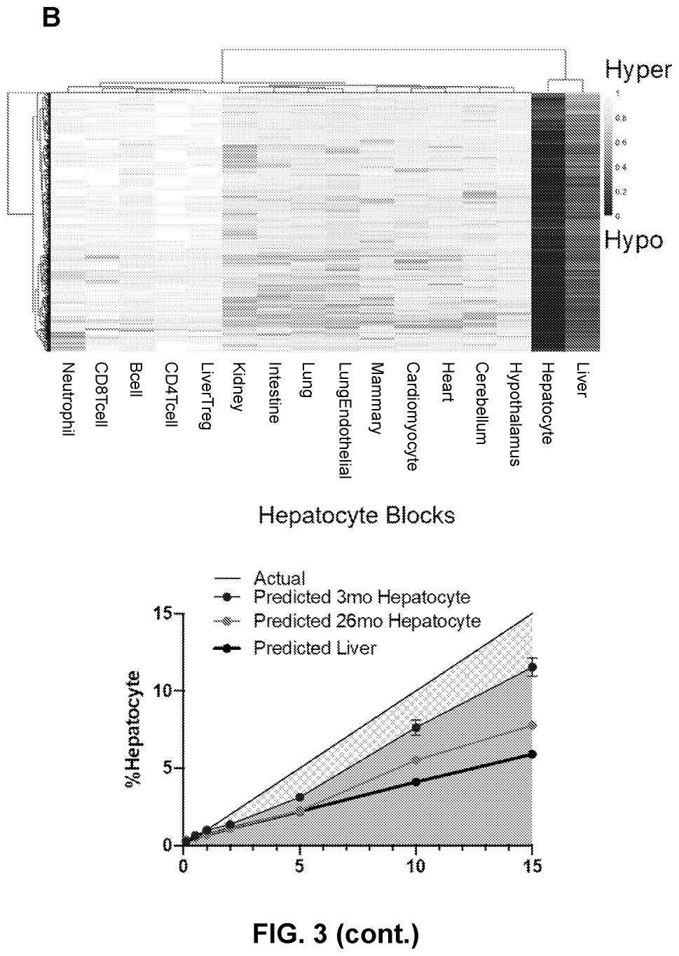

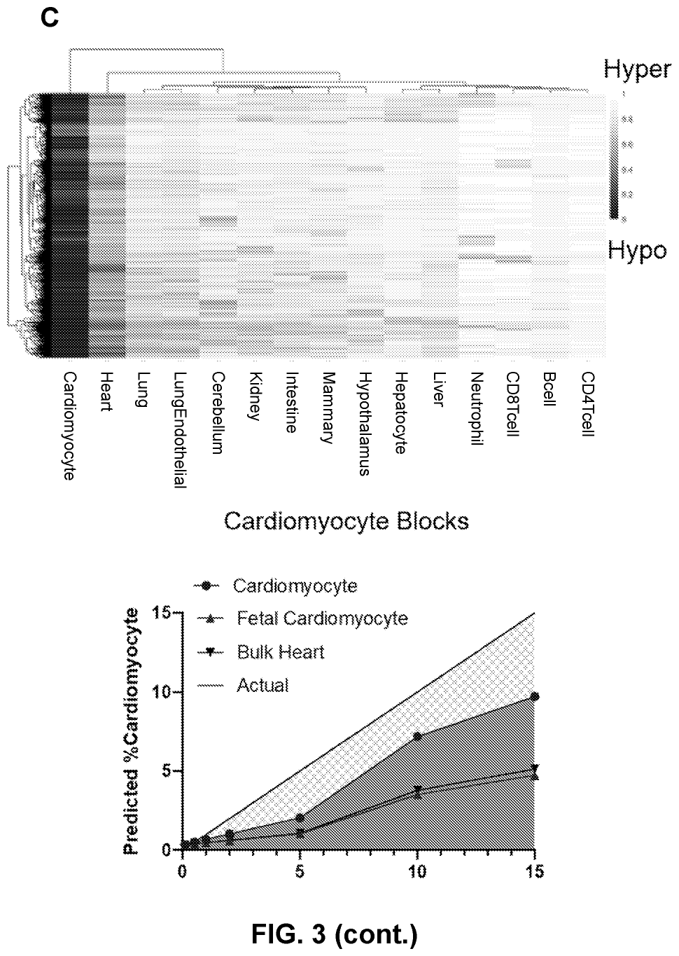

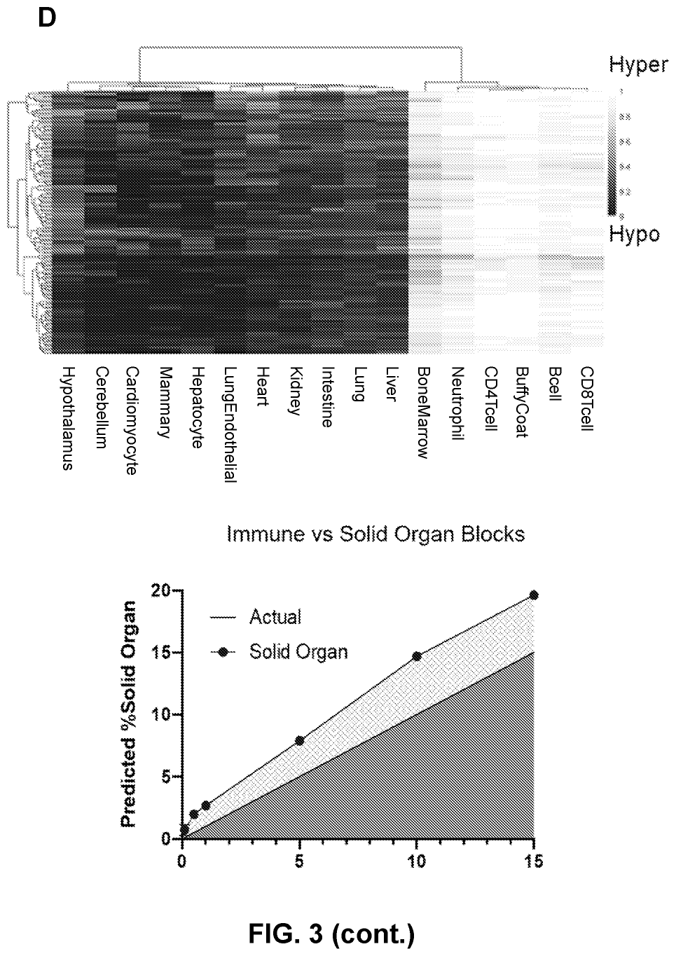

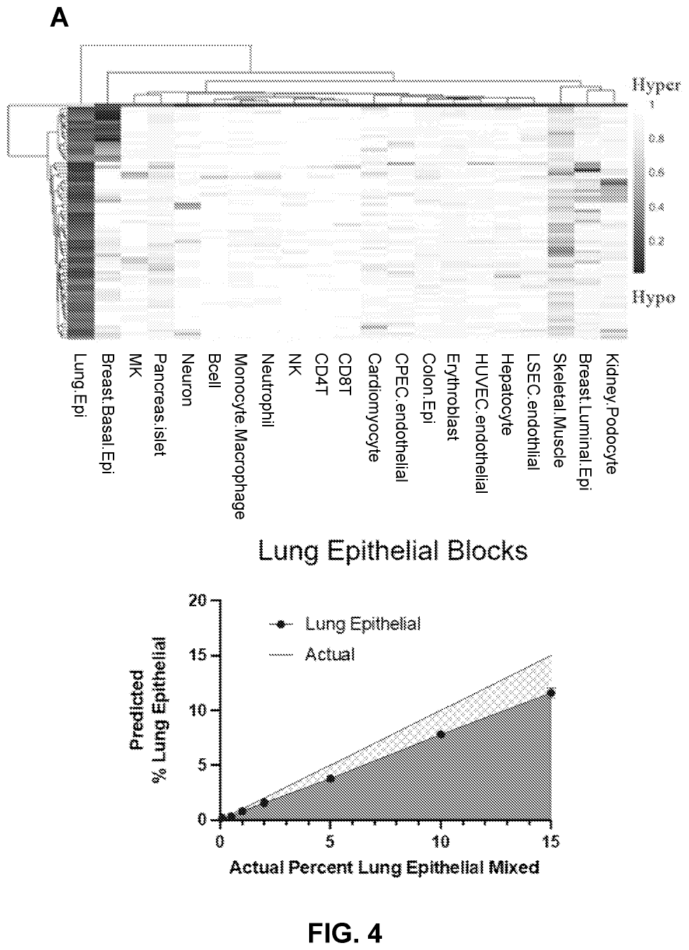

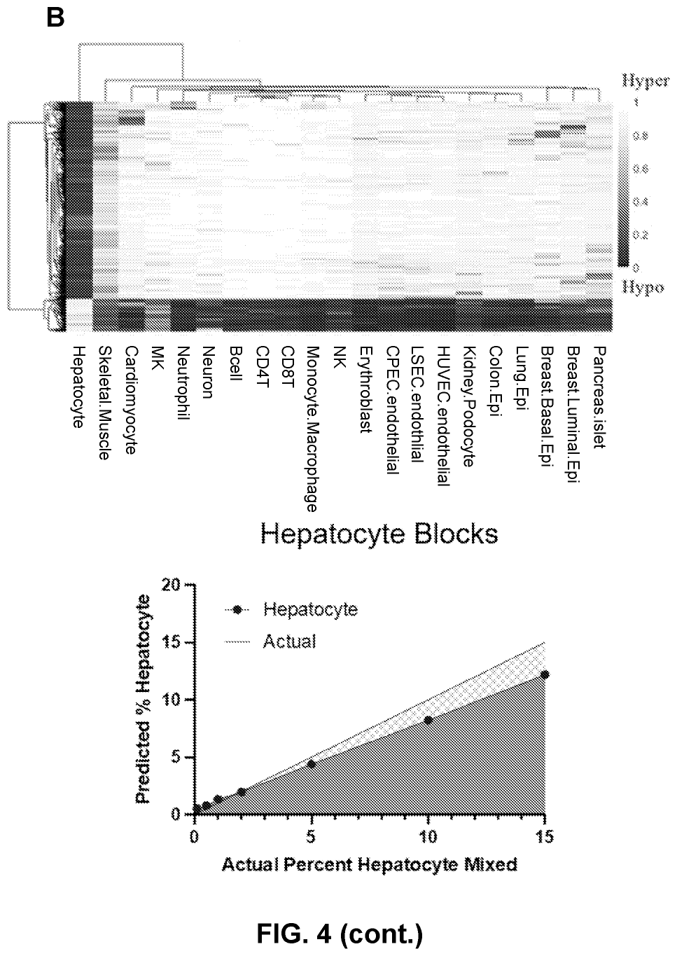

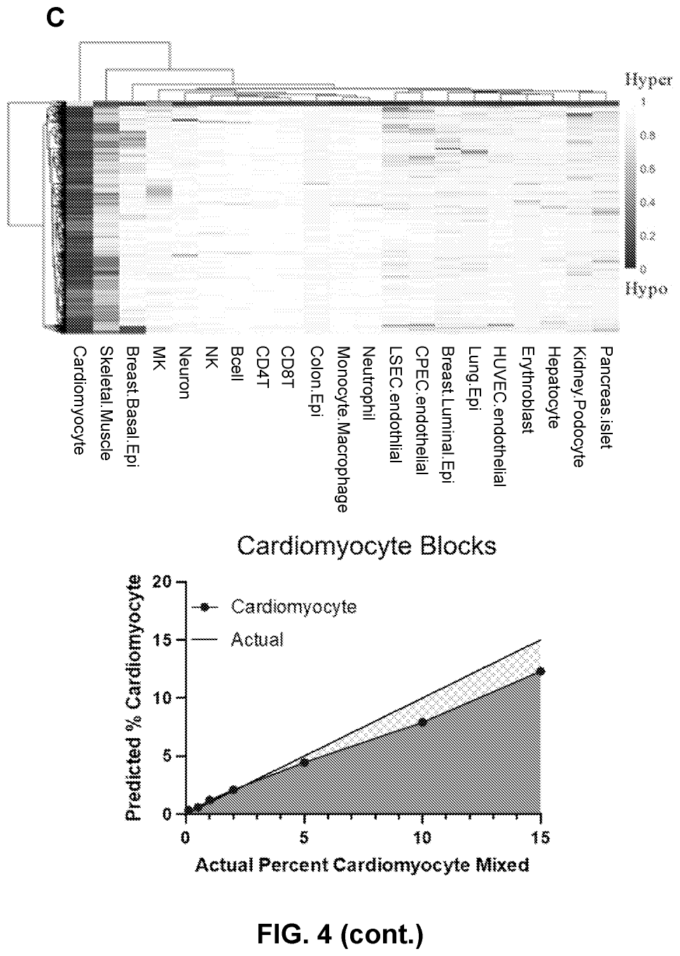

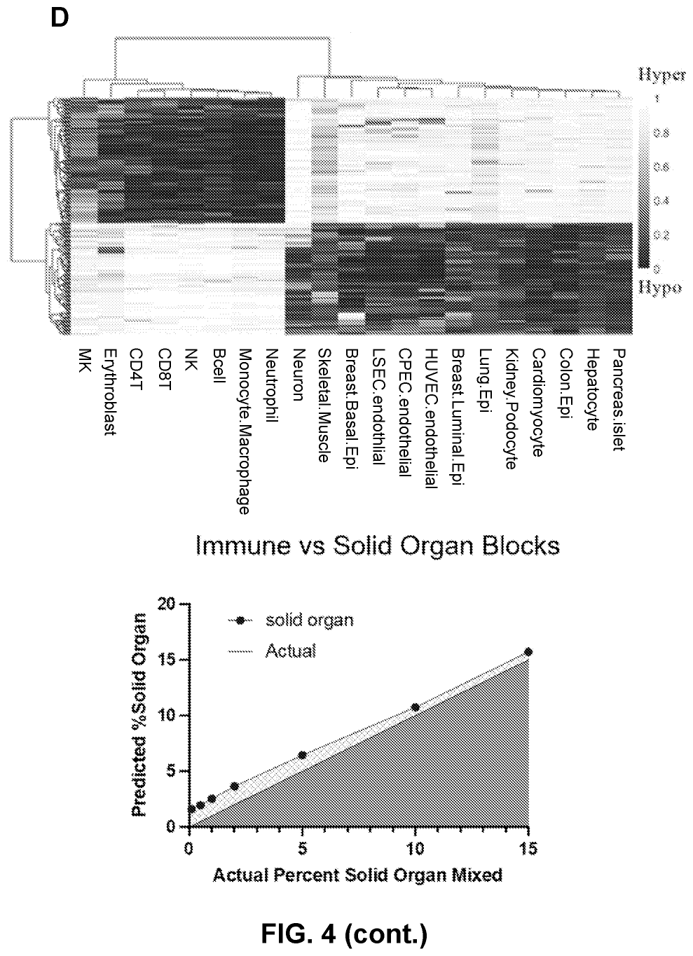

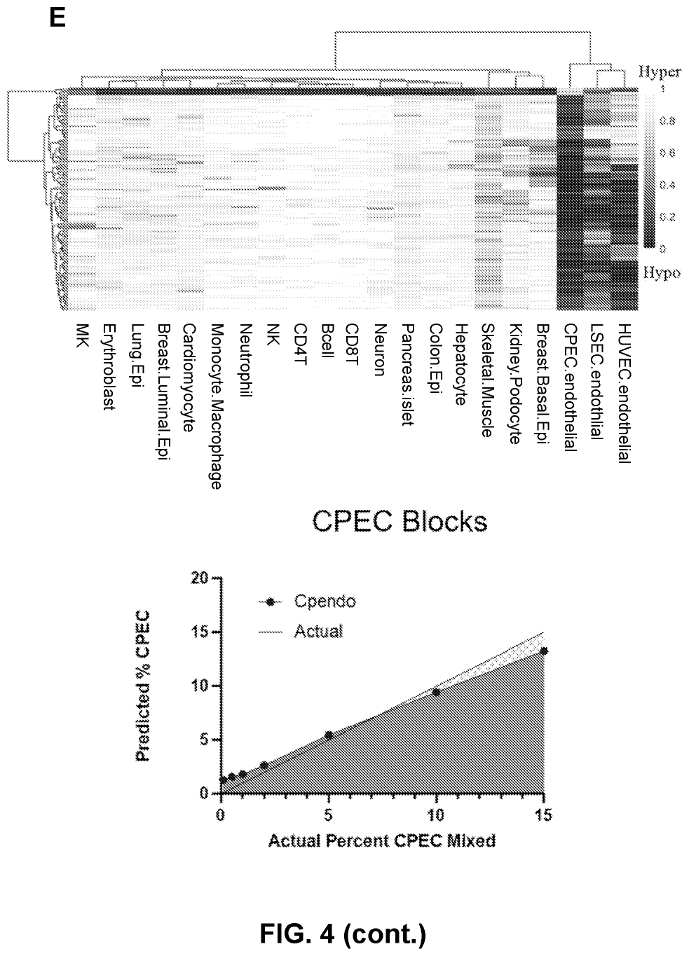

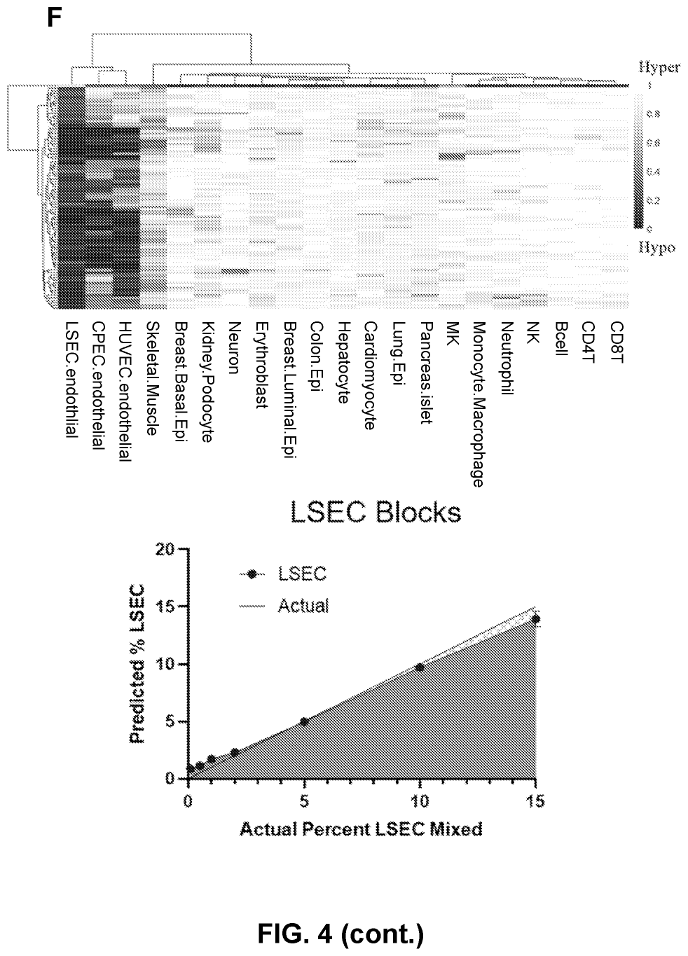

In-silico simulations WGBS deconvolution. In silico mix-in simulations were performed to validate the fragment-level deconvolution algorithm at identified cell-type specific blocks included in the radiation-specific methylation atlas (FIGS. 3 and 4). Reference data with greater than three replicates per cell-type was split into independent training and testing sets, leaving at least one replicate out for testing. Since the mouse cardiomyocyte reference WGBS data had less than three replicates, fragments were merged across replicates for this cell-type and split into training (80%) and testing (20%) sets. For each cell-type profiled, known proportions of target fragments were mixed into a background of leukocyte fragments across identified cell-type specific methylation blocks (leukocyte fragments obtained from n=4 buffy coat samples in mouse and n=10 buffy coat samples in human). Ten replicates for each admixture ratio assessed (0.001, 0.005, 0.01, 0.02, 0.05, 0.1, 0.15) were performed, and the average predicted proportion and standard deviation across all replicates was presented. Model accuracy was assessed through correct classification of the actual percent target mixed and relative degree of incremental change with increasing amount of target reads admixed was used to assess accuracy in estimating proportional changes across groups (mouse) and timepoints from serial samples (human). The cell-type specific blocks included in the radiation-specific methylation atlas were constructed using training set fragments only. Merging, splitting, and mixing of reads were preformed using wgbstools (Loyfer & Kaplan).

Longitudinal analysis of serial serum samples. Longitudinal analysis was performed on serial serum samples collected from breast cancer patients. Changing cell-type proportions of cfDNA at the end of treatment (EOT) and at Recovery were evaluated relative to baseline levels before the start of therapy (Baseline). Fold change (FC) from baseline was used to represent the percent cell-type cfDNA at EOT and Recovery relative to Baseline within the same individual. An exploratory correlation analysis was performed to evaluate linear relationship of changing cell-type proportions from EOT relative to Baseline, using Pearson's Correlation Coefficient.

Functional annotation and pathway analysis. Identified cell-type specific methylation blocks were provided as input for analysis in HOMER (http://homer.ucsd.edu/homer/). Each block was associated with its closest nearby gene and provided a genomic annotation. By default, TSS (transcription start site) was defined from −1kb to +100 bp, TTS (transcription termination site) was defined from −100 bp to +1kb, and CpG islands were defined as a genomic segment with GC content≥50%, genomic length>200 bp and the ratio of observed/expected CpG number>0.6. Prediction of known and de-novo transcription factor binding motifs were also assessed by HOMER. The top 5 motifs based on p value were selected from each analysis. Pathway analysis of identified tissue and cell-type specific methylation blocks was performed using Ingenuity Pathway Analysis (IPA) and Genomic Regions Enrichment of Annotations Tool (GREAT) (McLean et al., 2010). GeneSetCluster was used to cluster identified gene-set pathways based on shared genes (Ewing et al, 2020). Then WebgestaltR (ORAperGeneSet) plugin was used to interpret and functionally label identified gene-set clusters by reducing all identified significant gene-set pathways to the topmost representative one. Integration of methylome and transcriptome data generated from tissue-specific endothelial cells was performed using an expanded set of cell-type specific blocks (-bg.quant 0.2) compared to the more restricted set of blocks used for deconvolution analysis in the circulation (-bg.quant 0.1) The extended endothelial-specific methylation blocks can be found in Barefoot et al., 2022, Supplemental Table 10.

Cluster analysis and visualization techniques. The hierarchical relationship between reference tissue and cell-type WGBS datasets was visualized through creation of a tree dendrogram. The top 30,000 most variant methylation blocks containing at least three CpG sites and coverage across 90% of samples were selected. The average methylation for each block and sample was computed using wgbstools (-beta_to_table). Trees were assembled using the unweighted pair-group method with arithmetic mean (UPGMA) and visualized in R with the ggtree package. Dimensional reduction was also performed on the selected blocks using the UMAP algorithm. Default UMAP parameters were used (15 neighbors, 2 components, Euclidean metric, and a minimum distance of 0.1). Heatmaps were generated using the pretty heatmap function in the RStudio Package for the R bioconductor (RStudioTeam, 2015). Statistical analyses for group comparisons and correlations were performed using Prism and R. Sequencing reads were visualized using the Integrative Genomics Viewer (IGV) using the bisulfite CG mode for alignment coloring (Robinson et al, 2011). The BEDTools suite and AWK programming were used to overlay the sequencing data across samples to compare across sample groups and replicates. Python was used to operate WGBS tools and also to create visualization plots.

### Results

DNA methylation is highly cell-type specific and reflects cell lineage specification. Access to reference human and mouse WGBS datasets was obtained from publicly available databases and identified cell-type specific differential DNA methylation patterns, preferentially from primary cells isolated from healthy human and mouse tissues. Additionally, cell-type specific methylomes were generated for purified mouse immune cell-types (CD19+ B cell, Gr1+ Neutrophil, CD4+ T cell, and CD8+ T cell) and human tissue-specific endothelial cell-types (coronary artery, pulmonary artery, cardiac microvascular, pulmonary microvascular, and liver sinusoidal endothelial). Due to limited cell-type specific data available for mouse, reference data from mouse bulk tissues were included if none was available from purified cell-types within those tissues. This resulted in curation of methylation data from 10 different cell-types and 18 tissues for mouse and over 30 distinct cell-types for human (Tables 4 and 5; see also Barefoot et al., 2022, Supplemental Table 10).

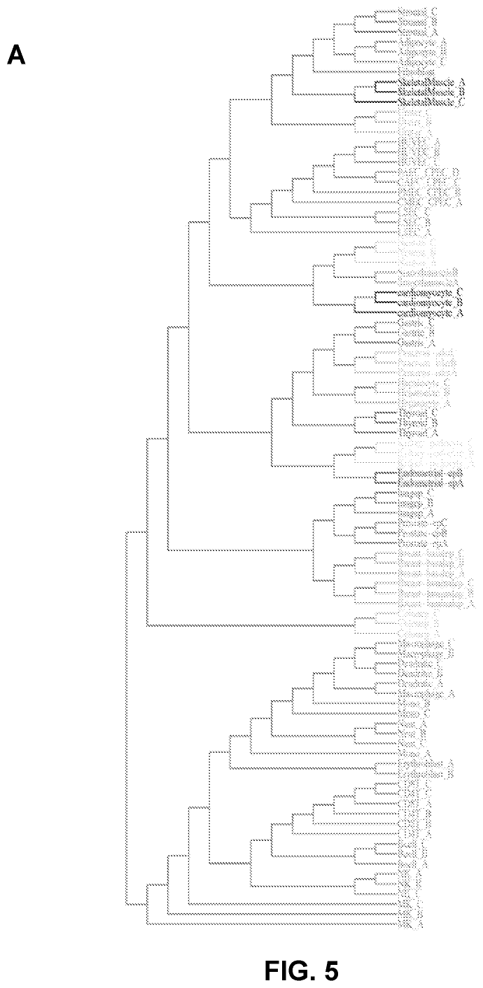

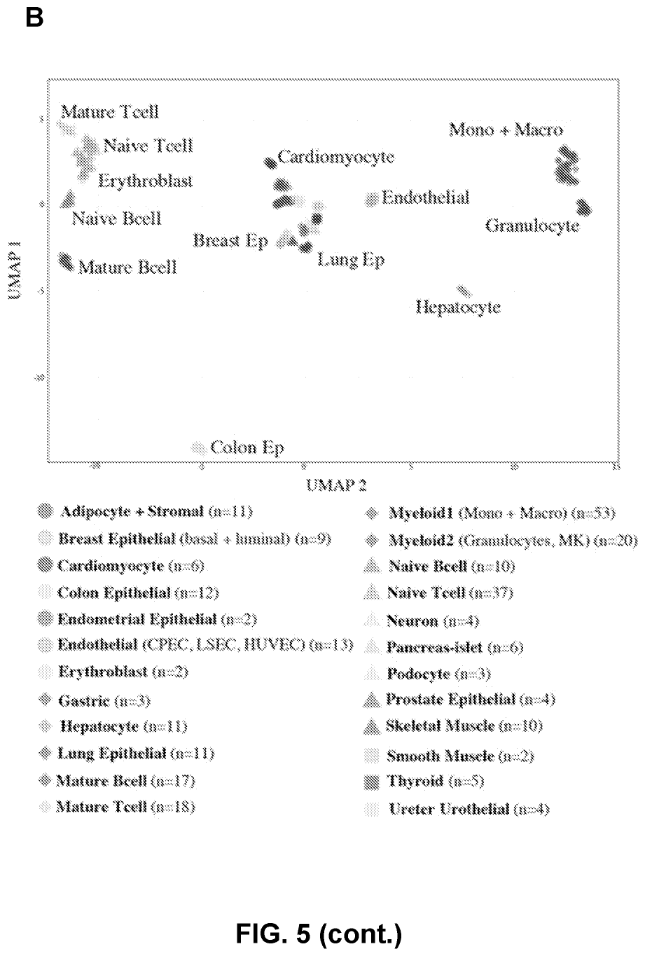

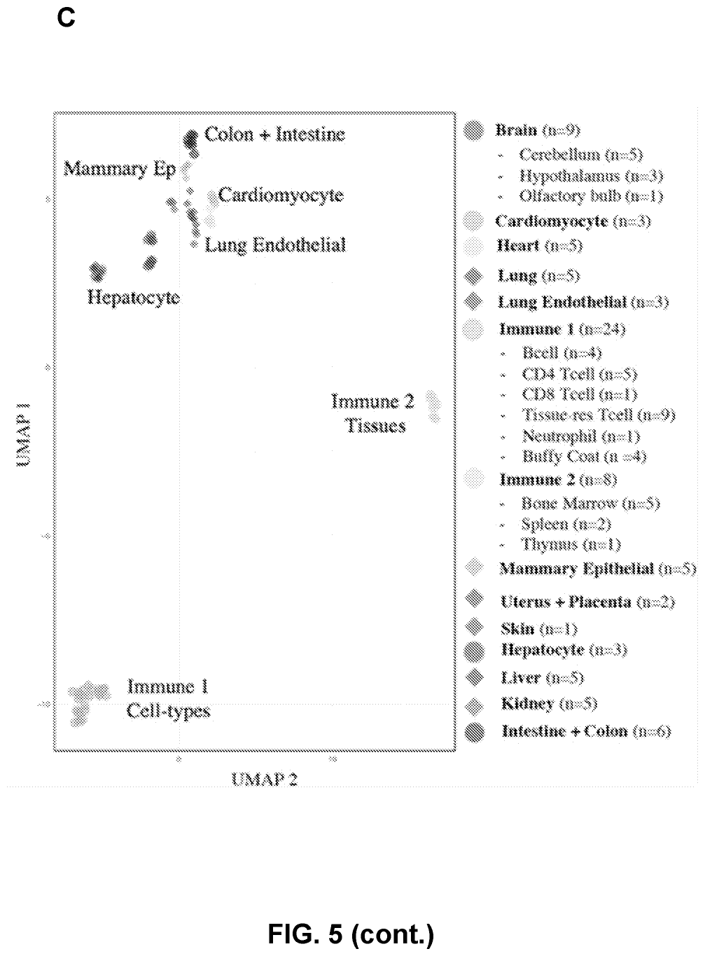

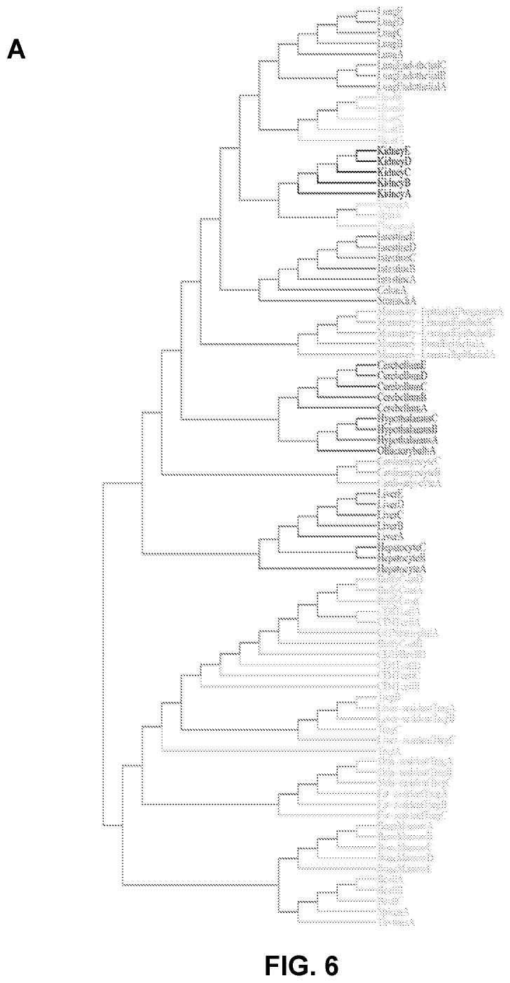

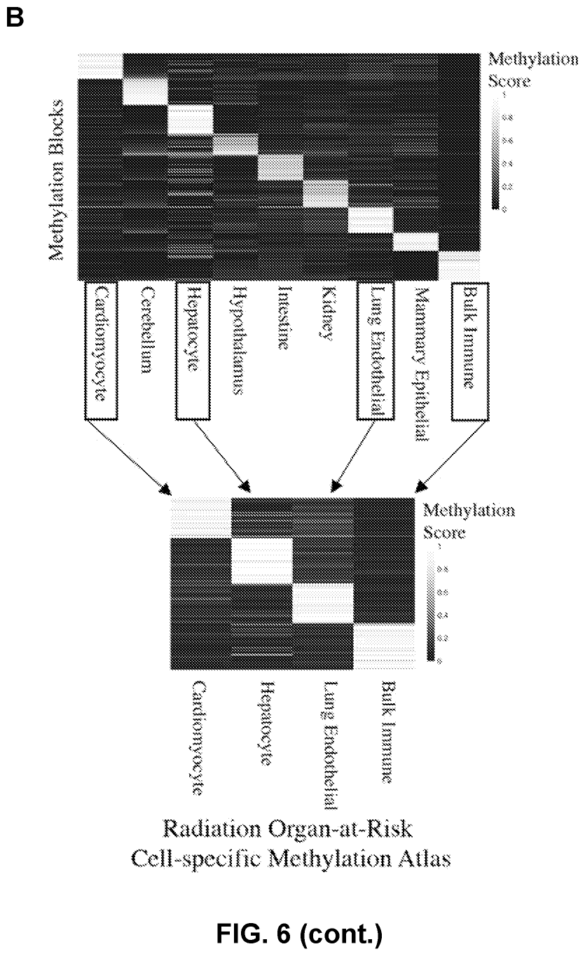

To better understand the epigenomic landscape of these healthy human and mouse cell-types in tissues, the methylomes were characterized by first segmenting the data into homogenously methylated blocks where DNA methylation status at adjacent CpG sites is highly co-regulated due to the processivity of methylation enzymes (Loyfer et al., 2022). Exploring the epigenetic variation amongst cell-types at the block-level increased robustness of down-stream analysis, proving more resistant to noise introduced as a by-product of the bisulfite sequencing. The segmentation was applied to 275 publicly available human WGBS datasets from purified cell-types to identify 351,395 blocks that are contained in the probes used for hybridization capture sequencing to enrich for cfDNA in human serum (Table 4). Segmentation of 83 WGBS datasets from normal cell-types and tissues in mouse identified 1,344,889 #blocks that are contained in the mouse hybridization capture probes (Table 5). On average, each block was greater than 300 bp with 4-8 CpG sites per block. Unsupervised hierarchical clustering analysis of the top 30,000 most variable methylation blocks in human and mouse, respectively shows the relationship between samples as a dendrogram and UMAP projection (FIGS. 5 and 6). The tightly correlated relationship between methylomes of the same cell-type observed from the cluster analysis reinforces the concept that methylation status is conserved at regions critical to cell-type identify. The within cell-type variation is noticeably reduced compared to the between cell-type variation. This stability allows methylated DNA to serve as a robust biomarker in the face of patient heterogeneity, capable of being generalized across diverse patient populations. For the most part, cell-types composing distinct lineages remain closely related, including immune, epithelial, muscle, neuron, endothelial, and stromal cell-types. Examples are tissue-specific endothelial and tissue-resident immune cells that cluster with endothelial or immune cells respectively, independent of the germ layer origin of their tissues of residence. Also, some cell types cluster separately from their bulk tissue counterparts. For instance, cardiomyocytes cluster separately from heart tissue in the mouse dendrogram, indicating heterogenous composition and distinct embryonic origins of different cell-types that contribute to organs (FIG. 6, Panel A). Surprisingly, a large epigenetic distance between immune cells of hematopoietic origins and solid organ cells from other lineages was observed (FIG. 5, Panels A and B). This is important for the tissue-of-origin analysis of cfDNA in the circulation, to distinguish solid organ from the hematopoietic origins of the DNA. Quite unexpectedly, a large number of epigenetic signatures capable of distinguishing amongst immune cells was also found, with cell-types of lymphoid and myeloid lineages forming distinct clusters. Within the immune cell cohort, increased separation of terminally differentiated cells compared to precursors was observed, with naïve B and T cells clustering separately from their more mature central and effector memory counterparts (FIG. 5, Panel B). Collectively, these findings support that DNA methylation is highly cell-type specific and reflects cell lineage specification.

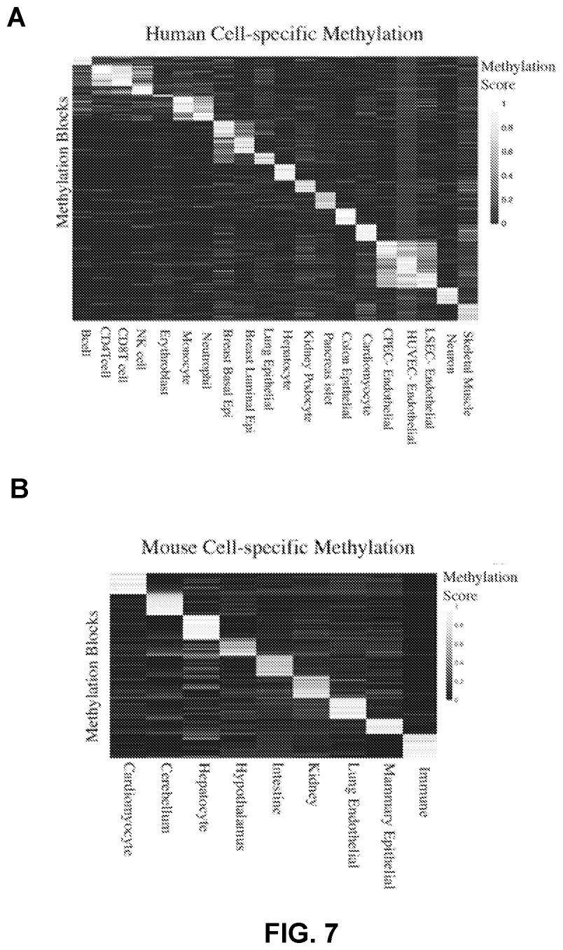

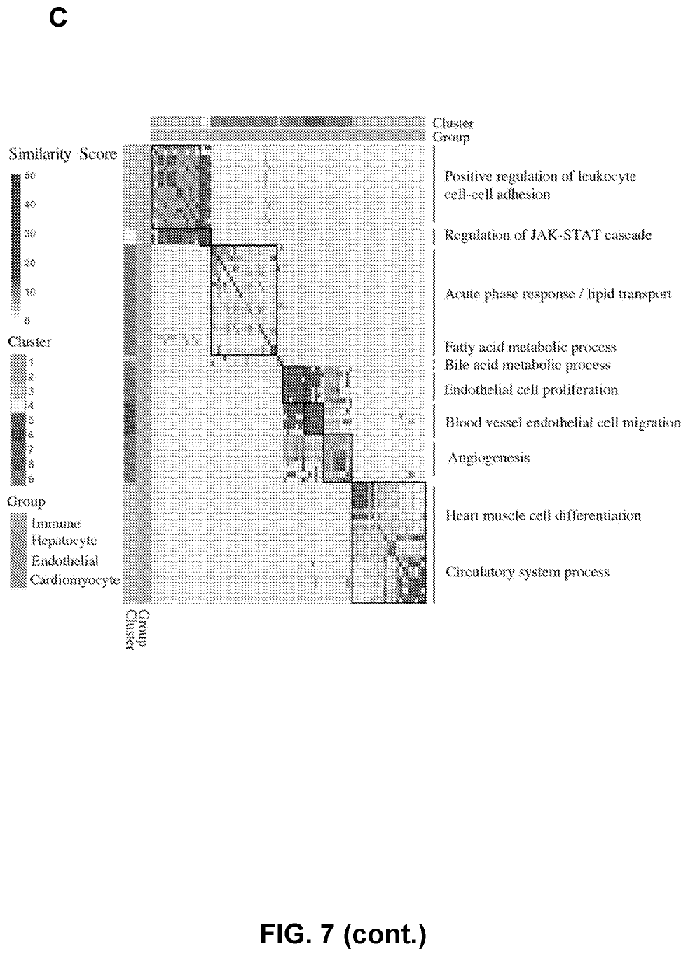

Differential DNA methylation distinguishes amongst cell-types in healthy human and mouse tissues. Based on the above unsupervised clustering analysis, the inclusion/exclusion criteria were further refined to select a final set of reference methylomes used to identify differentially methylated cell-type specific blocks. Low coverage WGBS samples were excluded from bulk tissues. Also, samples that did not cluster with other replicates were excluded from the same cell-type and instead clustered with fibroblast and other stromal cell-types. This resulted in a reduction of the starting 278 human WGBS samples to a final set of 104 samples that were organized into a grouping of 20 cell-types. Similarly, the starting 103 mouse WGBS samples were reduced to a final set of 44 samples that were organized into a final grouping of 9 cell-types and tissues. Subsets of some related cell-types were considered together to form the final groups (i.e., monocytes grouped together with macrophages and colon grouped together with small intestine). This final combination of groups was found to best represent the cell-specific epigenetic variation as a whole without overlap, using this publicly available data. Cell-type specific differentially methylated blocks (DMBs) that contained a minimum of 3 CpG sites were identified. The co-methylation status of neighboring CpG sites in these blocks were able to distinguish amongst all cell-types included in the final groups. 4,502 human and 7,344 mouse DMBs (see Barefoot et al., 2022, Supplemental Tables 3 and 4) with a lower margin of separation for mouse (0.35) versus human (0.40) due to more limited data were identified. A complete summary of human and mouse cell-type specific methylation blocks identified is in Tables 6 and 7. A variable number of blocks was required to achieve the same specificity for each cell-type based on the depth of coverage, purity, and degree of separation from other tissues and cell-types included in the atlas. Similar to others, we found enhanced separation of reference datasets using methylomes of purified cell-types as opposed to more heterogenous mixtures from bulk tissues (Moss et al., 2018). This is evident from the low number of DMBs identified from mouse bulk tissues as compared to mouse purified cell-types (average of 310 DMBs/tissue versus 1,488 DMBs/cell-type). Although over 85% of cell-type specific DMBs are hypomethylated, the blocks were depicted as a heatmap using a methylation score that is agnostic to the directionality of the methylation status and emphasizes the degree of separation of both hypo- and hyper-methylated blocks in the target group relative to all other groups. The methylation score calculates the number of fully unmethylated or methylated read-pairs divided by total coverage for hypo- and hyper-methylated blocks, respectively. The heatmaps in FIG. 7 depicts up to 100 blocks for each cell-type group with the highest methylation score.

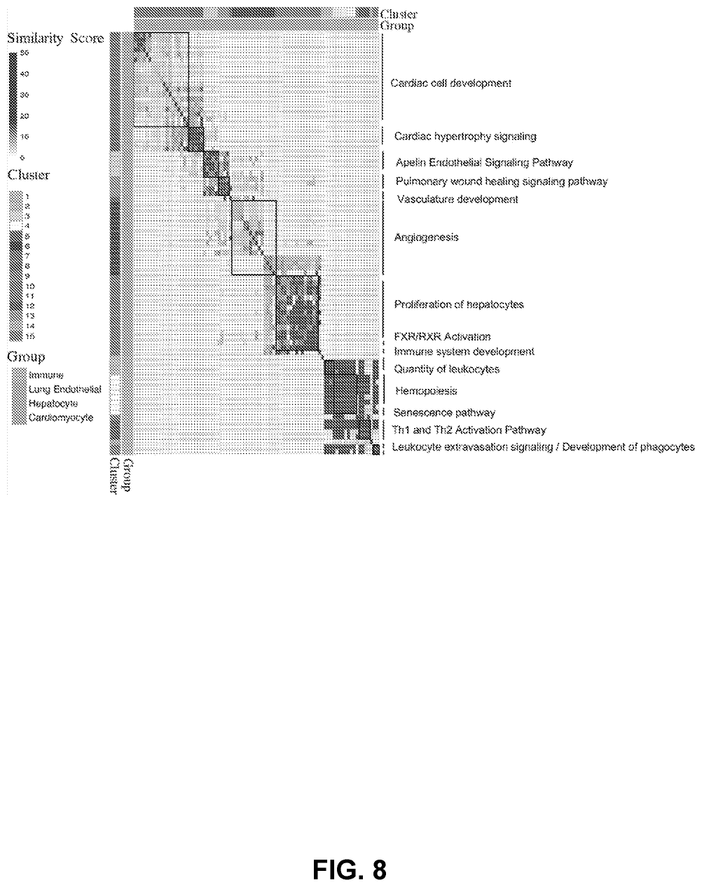

Differential DNA methylation is closely linked to regulation of cell-type specific functions. The role of cell-type specific methylation in shaping cellular identity and function was investigated. Genes adjacent to cell-type specific methylation blocks were identified using HOMER and performed pathway analysis of annotated genes using both Ingenuity Pathway Analysis (IPA) and GREAT. GeneSetCluster was used to group significantly enriched pathways based on shared genes and WebgestaltR functionally labeled each cluster by its top defining biological process (FIG. 7, Panel C; and FIG. 8). Gene-set pathways largely clustered within independent cell-type groups, reinforcing that cell-specific differential methylation occurs adjacent to unique genes integral to cell-type specific functions. Collectively, cell-type specific methylation was preferentially located adjacent to genes with biological functions involving cell development, movement, proliferation, differentiation, and morphology. In addition, transcriptional machinery genes including transcription factors and co-regulators were significantly associated with cell-type specific DNA methylation, specifically those involving assembly of RNA polymerase III complex and pre-mRNA catabolic process (see Table 11). However, despite these commonalities, important biological differences were also observed in the gene sets identified based on specific processes unique to the cell-types profiled. For example, the biological function of genes associated with immune cell-type specific methylation reflects processes of leukocyte cell-cell adhesion, immune response-regulating signaling, and hematopoietic system development (FIG. 7, Panel C). In contrast, fatty acid metabolic process, lipid metabolism, and acute phase response signaling were identified for hepatocytes. These findings suggest that cell-type specific methylation is involved in regulation of these cellular processes. Significantly enriched biological pathways and functions for genes associated with differential methylation in each cell-type examined are provided in Table 11.

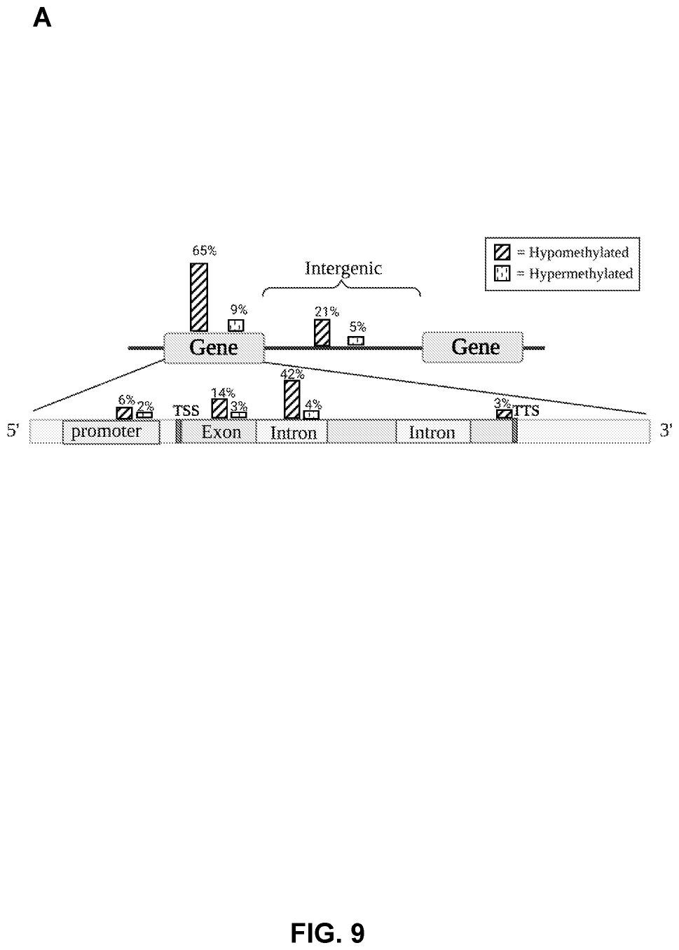

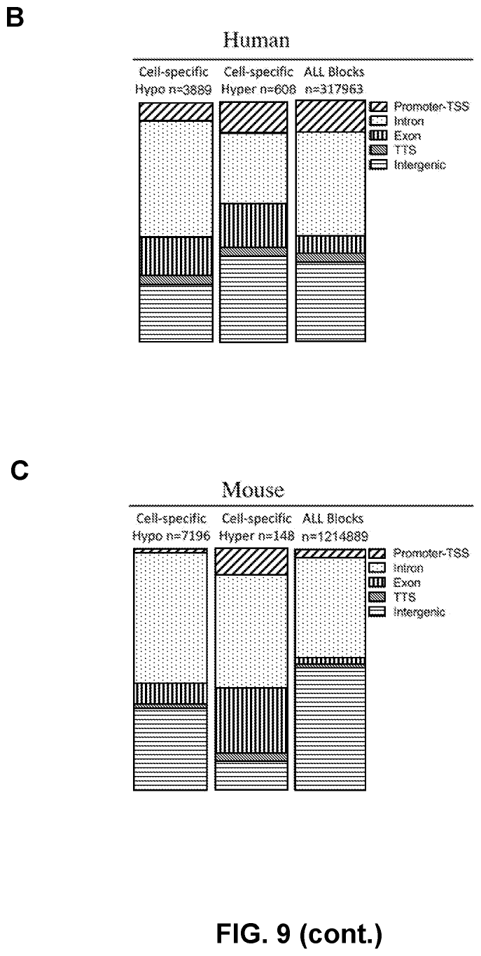

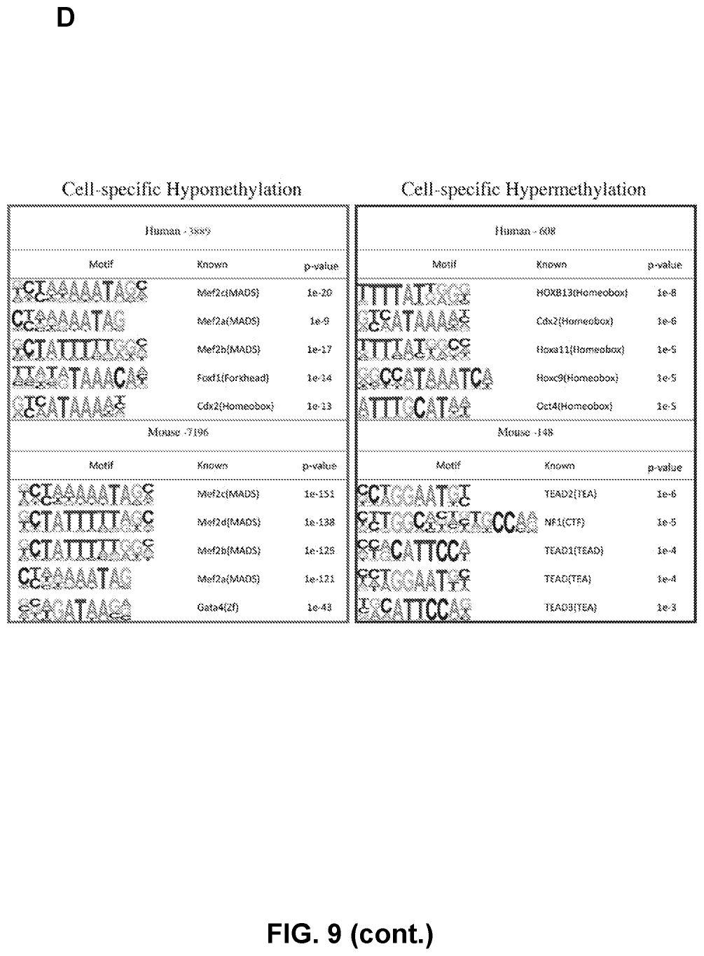

Cell-type specific DNA methylation is majority hypomethylated and enriched at intragenic regions containing developmental TF binding motifs. The majority of identified human and mouse cell-type specific blocks were hypomethylated, consistent with the proposed mechanisms of methylation resetting during embryonic development that leads to highly regulated cell-type specific differences (Greenberg & Bourc'his, 2019; Dor & Cedar, 2018). It was found that, in human samples, 86% of cell-type specific DMBs hypomethylated and only 14% hypermethylated. Strikingly in the mouse samples, 98% of cell-type specific DMBs were hypomethylated and only 2% were hypermethylated. The schematic in FIG. 9, Panel A depicts the location of identified human cell-type specific hypo- and hyper-methylated blocks. Interestingly, regardless of directionality the majority of cell-type specific blocks were located within intragenic regions. To see if this distribution was enriched, the genomic loci of cell-type specific blocks were compared to blocks that did not vary amongst cell-types (FIG. 9, Panels B and C; Table 8). It was found that for both human and mouse, there was a significant enrichment of cell-type specific blocks within intragenic regions relative to other captured regions (p<0.05). Furthermore, the intragenic distribution of cell-type specific blocks showed a significant increase of locations within exons and decrease in promoter-TSS segments (p<0.05). There was also a significant relationship between directionality and intragenic distribution, with a larger proportion of cell-type specific blocks being hypermethylated in exons and hypomethylated in introns (p<0.05). The similar distribution of cell-type specific methylation blocks in human and mouse suggests a conserved biological function of these genomic regions across species.

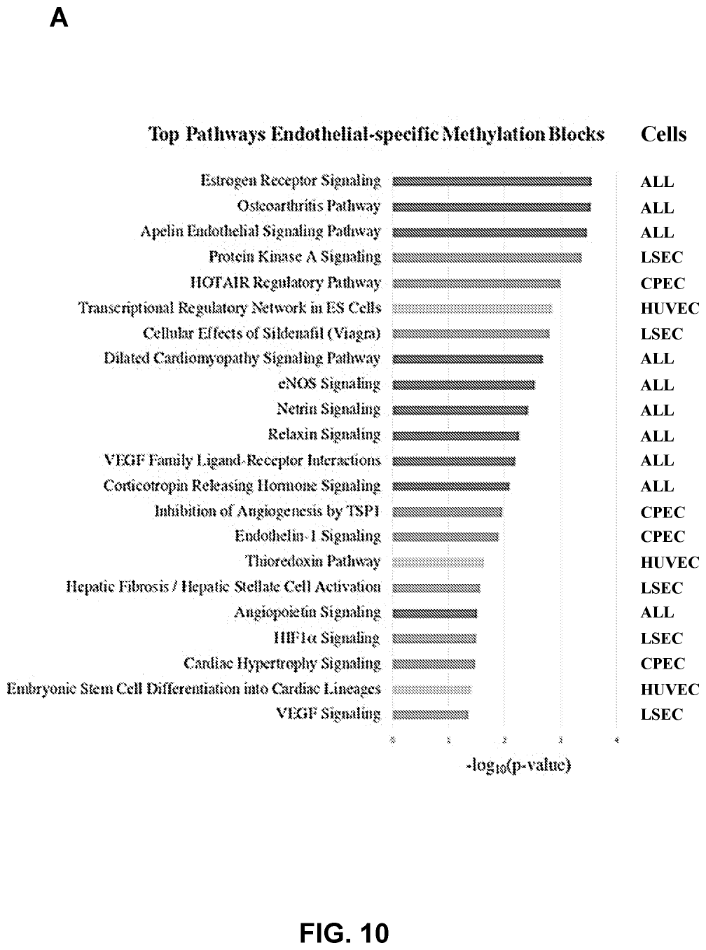

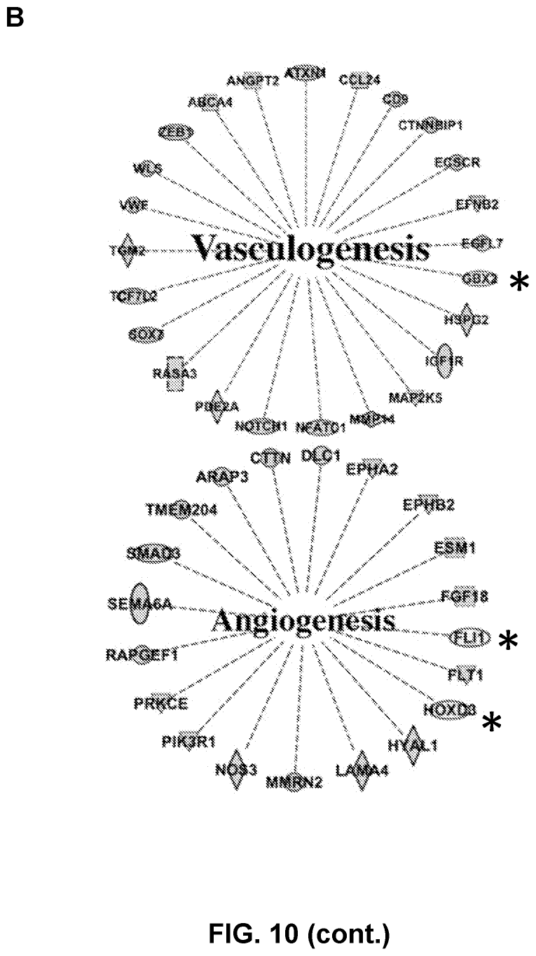

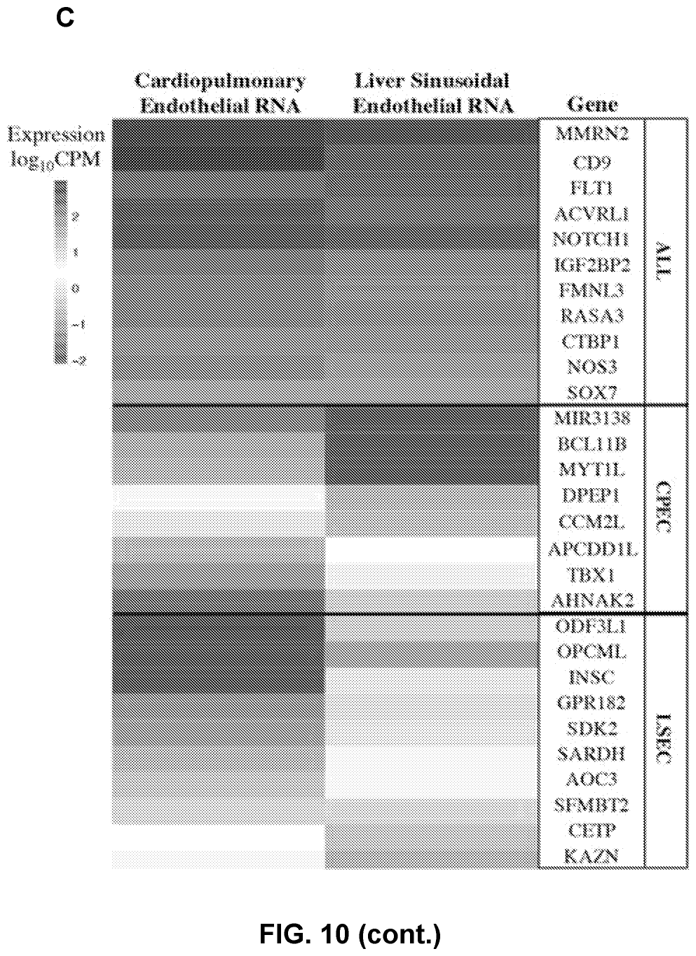

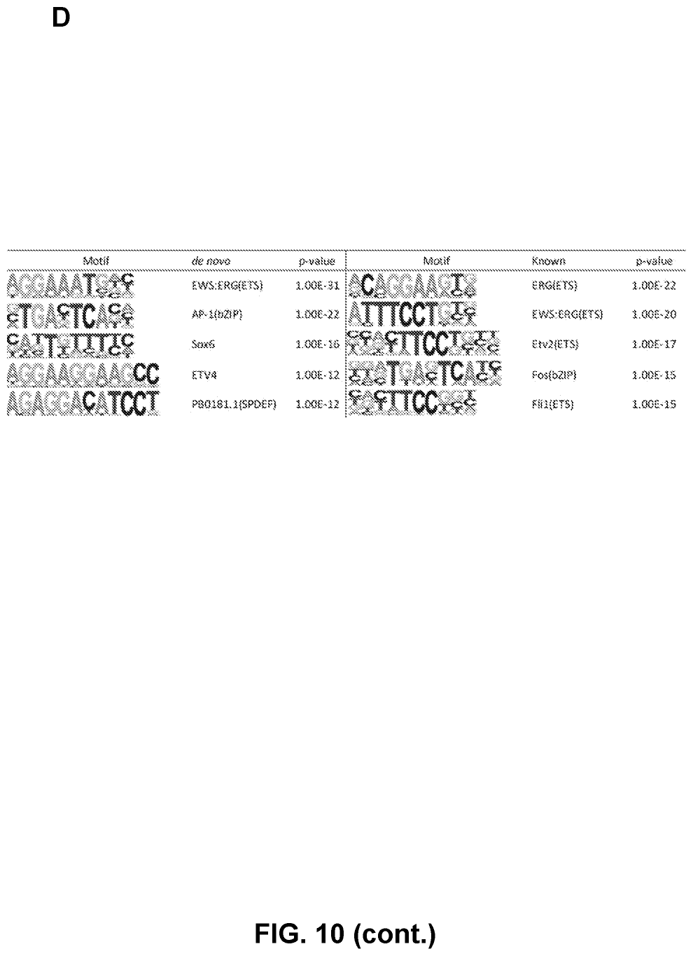

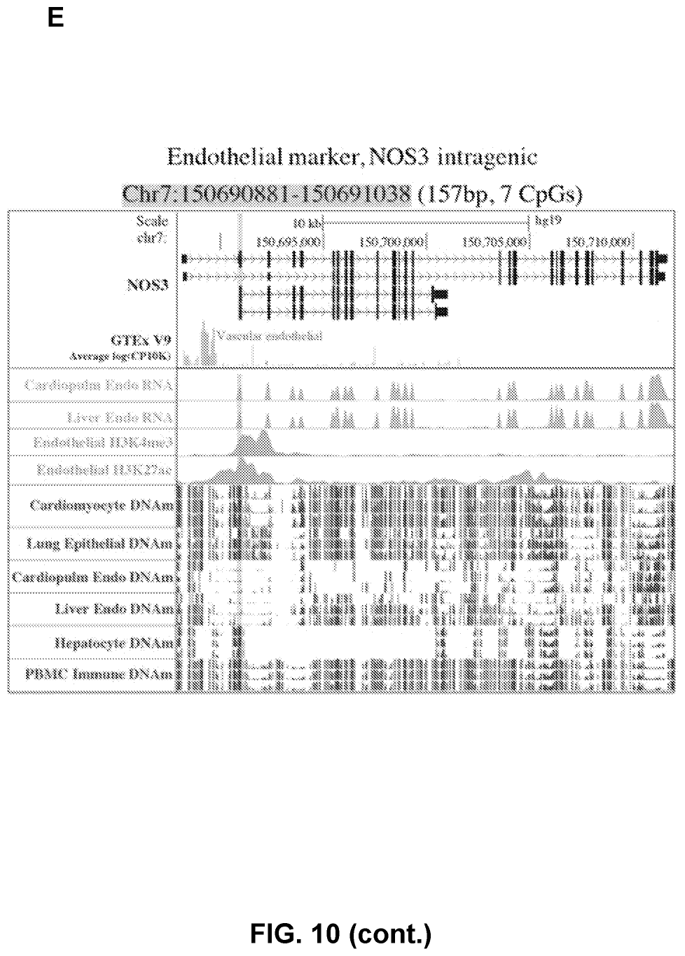

To further explore what common purpose these identified regions may have in human and mouse development, motif analysis was performed using HOMER to see if there were commonly enriched transcription factor binding sites (TFBS). MADS motifs bound by MEF2 transcription factors were significantly enriched in both human and mouse cell-type specific hypomethylated blocks (FIG. 9, Panel D, left). The MEF2 transcription factors are established developmental regulators with roles in the differentiation of many cell-types from distinct lineages. In comparison, Homeobox motifs bound by several different HOX TFs were enriched in the human cell-type specific hypermethylated blocks (FIG. 9, Panel D, right). Specifically, HOXB13 was the top TF associated with binding at sites within the human hypermethylated DMBs. Recently, HOXB13 has been found to control cell state through binding to super-enhancer regions, suggesting a novel regulatory function for cell-type specific hypermethylation. In addition to the common TFBS enriched by all cell-type specific blocks, endothelial-specific TFs were found to be enriched in the endothelial-cell hypomethylated blocks, including EWS, ERG, Fli1, ETV2/4, and SOX6 (see FIG. 10, Panel D). As a whole, this data reveals unknown functions of these cell-type specific blocks that represent cell-specific biology.

Methylation profiling of tissue-specific endothelial cell-types reveals epigenetic heterogeneity associated with differential gene expression. Radiation-induced endothelial damage is a major complicating factor of radiotherapy that is thought to be a leading cause for development of late-onset cardiovascular disease (Tapio, 2016; Wagner & Dimmeler, 2019). The microvasculature is particularly sensitive to radiation, with dysfunction of these cells potentially contributing to damage in a variety of tissues (Wijerathne et al., 2021; Park et al., 2012). Thus, tissue-specific endothelial methylomes and paired transcriptomes were generated in order to profile damage from distinct populations of microvascular and large vessel endothelial cell-types including coronary artery, pulmonary artery, cardiac microvascular, pulmonary microvascular, and liver sinusoidal endothelial. Also made use were publicly available umbilical vein endothelial methylomes from the Blueprint Epigenome Consortium to complement our data (Table 4; see also Barefoot et al., 2022, Supplemental Table 1). Previous studies support modeling the heart and lung as an integrated system in the development of radiation damage since the heart and lungs are linked by the cardiopulmonary circulation (Barazzuol et al., 2020). Therefore, cardiac and pulmonary endothelial cell-types were merged together to generate a joint cardiopulmonary endothelial signal and identified the specific methylation blocks for cardiopulmonary (CPEC, n=132), liver sinusoidal endothelial (LSEC, n=89), and umbilical vein endothelial (HUVEC, n=116) cell-types. Pathway analysis of genes associated with these methylation blocks confirmed endothelial cell identity, revealing genes involved in regulation of vasculogenesis, angiogenesis, and vascular development (FIG. 10, Panel B). In addition, unique pathways were identified capturing the tissue-specific epigenetic diversity of these different endothelial cell populations. For example, Hepatic Fibrosis Signaling was found to be LSEC-specific, Cardiac Hypertrophy Signaling identified as CPEC-specific, and Thioredoxin Pathway activity was specific to HUVEC (FIG. 10, Panel A). The identity of starting material used to generate these human endothelial methylomes was validated through paired RNA-sequencing analysis. Integrative analysis of DNA methylation and paired RNA expression allowed for better understanding of the relationship between cell-type specific DNA methylation and corresponding changes in gene expression. Methylation status at several identified blocks was found to correspond with RNA expression of known endothelial-specific genes, confirming the identity of the LSEC and CPEC populations isolated (FIG. 10, Panel C and E; Barefoot et al., 2022, Supplemental Table 10). For example, hypomethylation was associated with increased expression at several pan-endothelial genes, including NOTCH1, ACVRL1, FLT1, MMRN2, NOS3 and SOX7. Likewise, hypomethylation at CPEC- and LSEC-specific genes led to differential expression when comparing the two populations, reflecting tissue-specific differences. CPEC- and LSEC-specific expression of selected genes have been reported in previous studies examining vascular heterogeneity at the transcriptome level (Feng et al., 2019; Sabbagh et al., 2018; Nolan et al., 2013; Cleuren et al., 2019). However, linking these expression patterns with cell-type specific methylation is a novel feature. While the majority of endothelial-specific methylation blocks were hypomethylated, select hypermethylated blocks were identified as well, including CCM2L in CPEC that corresponded with decreased gene expression compared with LSEC. As a relatively abundant cell-type in the circulation, the ability to non-invasively detect distinct damage to different types of endothelial cell populations could prove useful to monitor tissue-specific damages.

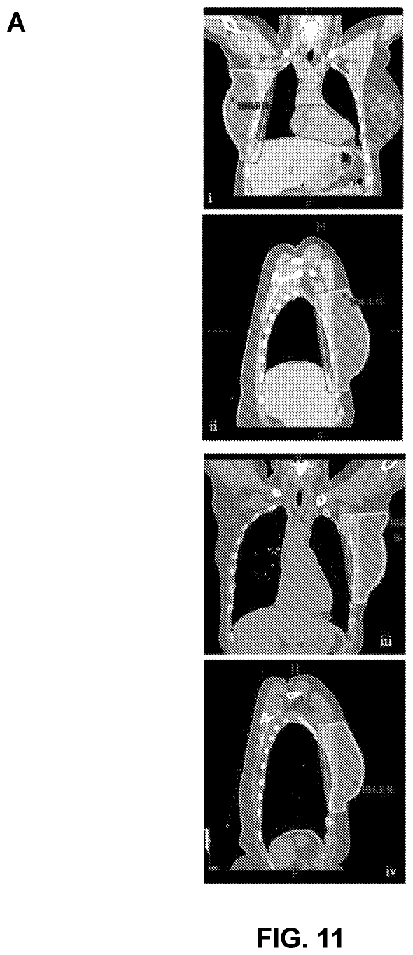

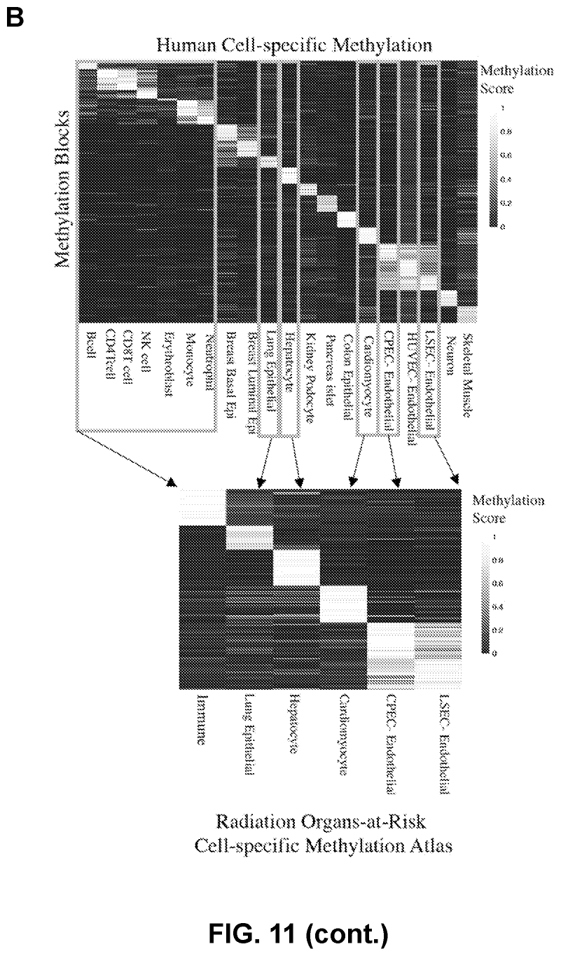

Development of a radiation-specific methylation atlas focusing on ceil-types from target organs-at-risk (OAR). After ensuring specificity of identified cell-type specific methylation blocks by comparison to all other cell-types with available WGBS data, the assessment of cfDNA origins in the circulation was limited to select cell-types originating from target organs-at-risk for radiation damage. Restriction to a focused radiation-specific methylation atlas helped to maintain sensitivity of radiation-induced damage to cell-types of interest based on prior knowledge of organs targeted and damaged due to existing clinical correlates. Representative treatment planning for breast cancer patients receiving adjuvant radiation provides an estimate organ volume impacted and radiation dose level for target organs-at-risk from radiation damage, including the heart and lungs (FIG. 11, Panel A). In addition to organs that are in close proximity with the target treatment area, the liver is another organ that may receive a substantial dose from radiation, especially in right-sided breast cancer patients. Differential blocks identified from cell-types comprising these target organs-at-risk from radiation (lungs, heart, and liver) were selected for generation of a radiation-specific methylation atlas, separating these solid organ cell-types of interest from all other immune cell-types (FIG. 11, Panel B; FIG. 6, Panel B). The human and mouse blocks specific to these cell-types can be found in Barefoot et al., 2022, Supplemental Tables 3 and 4. Due to the large degree of separation of the epigenetic signature of hematopoietic cells from other solid organ cell lineages, all hematopoietic cell-types were merged into one joint “immune” super-group. This approach also accounts for the majority hematopoietic origins of cfDNA at baseline and helps reveal signals coming from solid organ cell-types of interest. Focus was on these same target organs in both human and mouse, resulting in a final curation of six groups for human (immune, lung epithelial, cardiopulmonary endothelial, cardiomyocyte, hepatocyte, and liver sinusoidal endothelial) and four groups for mouse (immune, lung endothelial, cardiomyocyte, hepatocyte) based on the reference cell type data available.

Cell-free Methylated DNA in blood identifies origins of radiation-induced cellular damage in tissues. Serial serum samples were collected from breast cancer patients undergoing standard radiation therapy. In addition, paired serum and tissue samples were collected from mice receiving radiation. Unbiased methylome-wide hybridization capture sequencing of DNA from human or mouse serum samples was performed. Deconvolution analysis was used to trace the origins of cfDNA fragments allowing for minimally invasive monitoring of radiation-induced cellular toxicities from blood samples (FIG. 2). In comparison to previous studies using single CpG sites, the sequencing-based approach allows for fragment-level cfDNA analysis using CpG methylation patterns (Scott et al., 2020; Li et al, 2018). For this, the co-methylation status was modeled of adjacent CpG sites on the same molecule implemented by a novel probabilistic deconvolution method. The model was applied using cell-type specific blocks from the human and mouse radiation-specific methylation atlases described above. The prediction accuracy of the fragment-level deconvolution was validated through in silico mix-in simulations for each tissue and cell-type of interest (FIGS. 3 and 4).

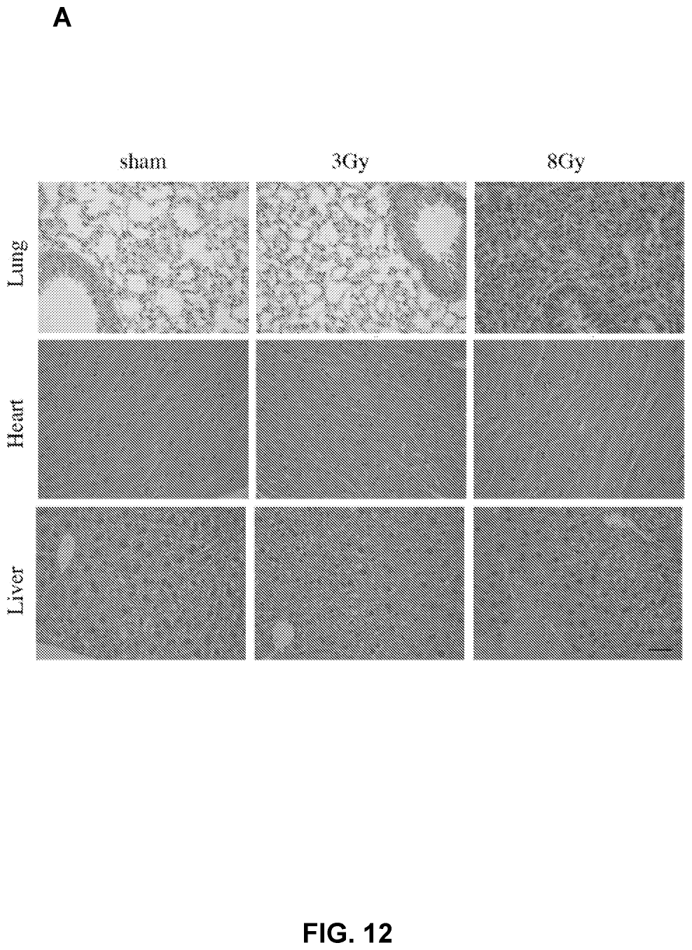

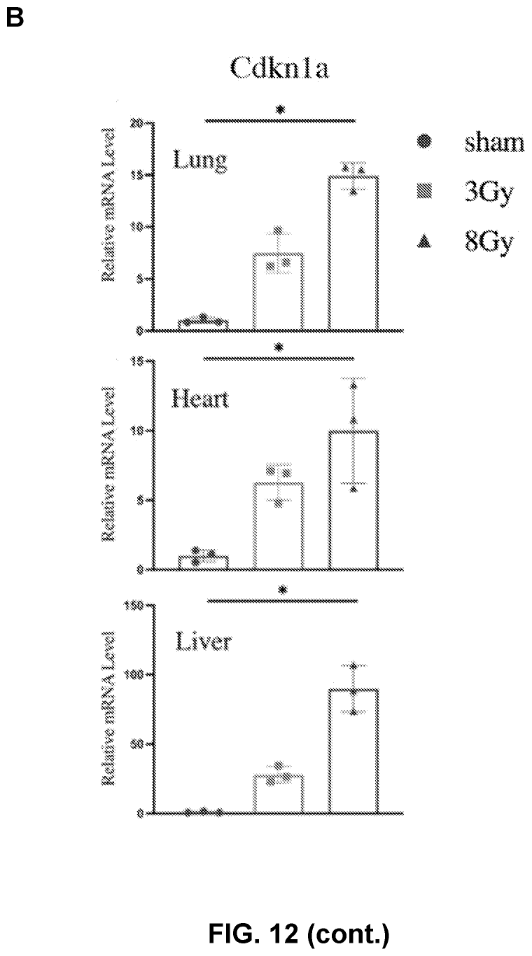

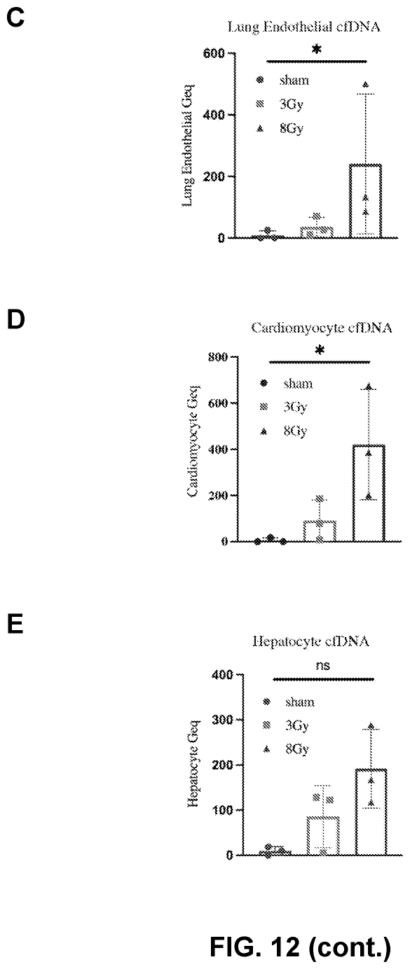

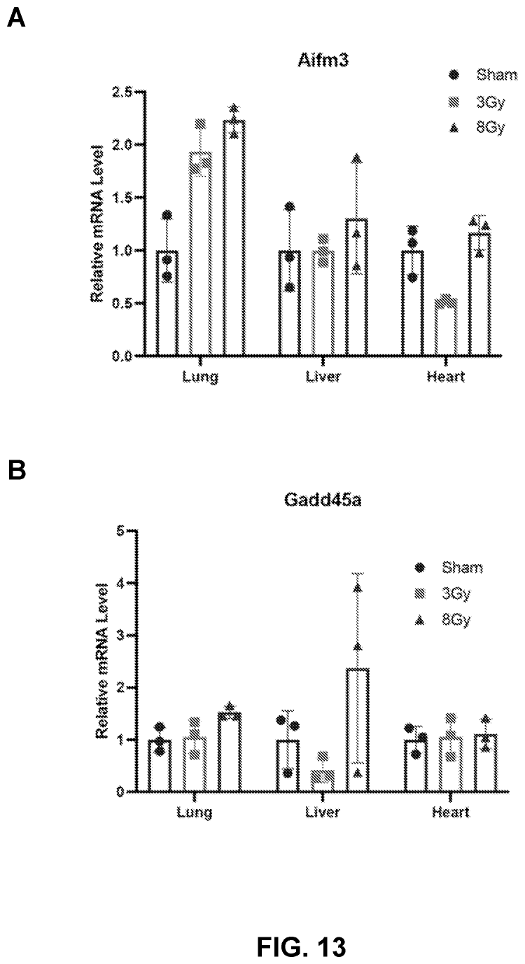

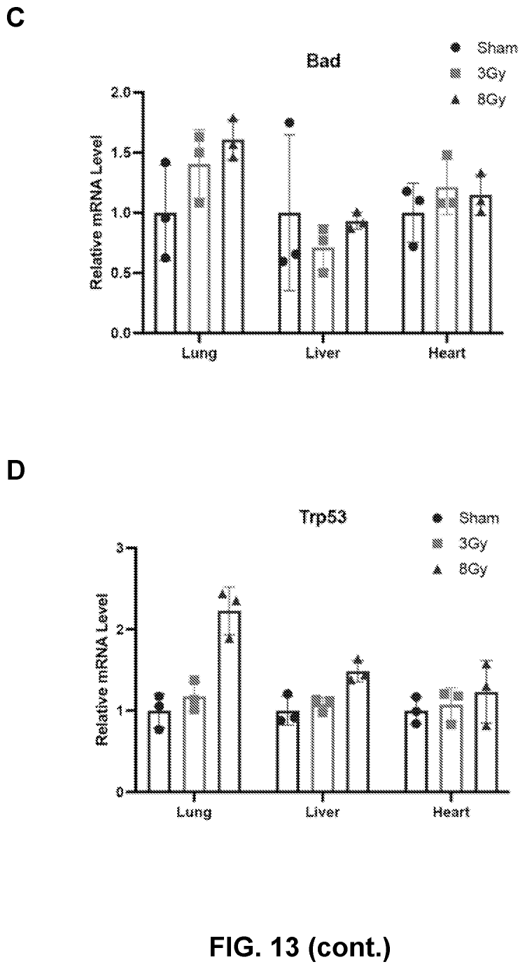

Dose-dependent indicators of radiation damage in mice. To explore the relationship between radiation-induced damage in tissues to changing proportions of cfDNA origins in the circulation, mice were used to model exposure from different radiation doses. Mice received upper thorax radiation at 3Gy or 8Gy doses relative to sham control, forming three groups for comparison (FIG. 2). Tissues and serum were harvested 24 hours after the last fraction of treatment and tissues in line with the path of the radiation-beam (heart, lung, and liver) were targeted for subsequent analyses. Through histological analysis, dysregulated tissue architecture corresponding to higher dose radiation was observed (FIG. 12, Panel A). These changes were most apparent in tissue sections of the lungs showing noticeable alveolar collapse with increased radiation dose. Liver tissues showed increased fibrosis with increased radiation doses and only minor changes were apparent in cardiac tissues matching with its higher resilience to radiation. Tissue effects were also assessed through qPCR analysis of established indicators of radiation effects, including expression of CDKN1A (p21), that exhibited a dose-dependent increase in expression in response to radiation in all tissues (FIG. 12, Panel B; FIG. 13) (Hyduke et al., 2013).

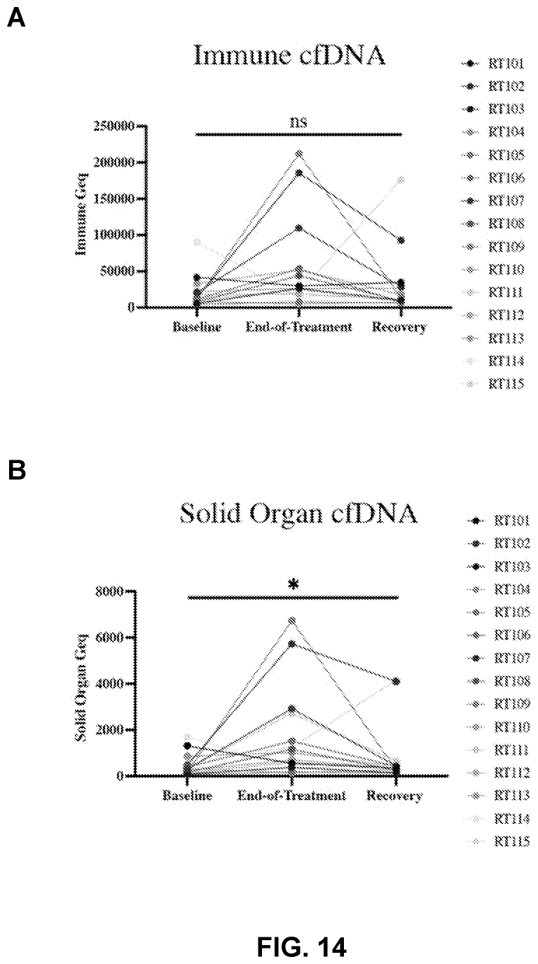

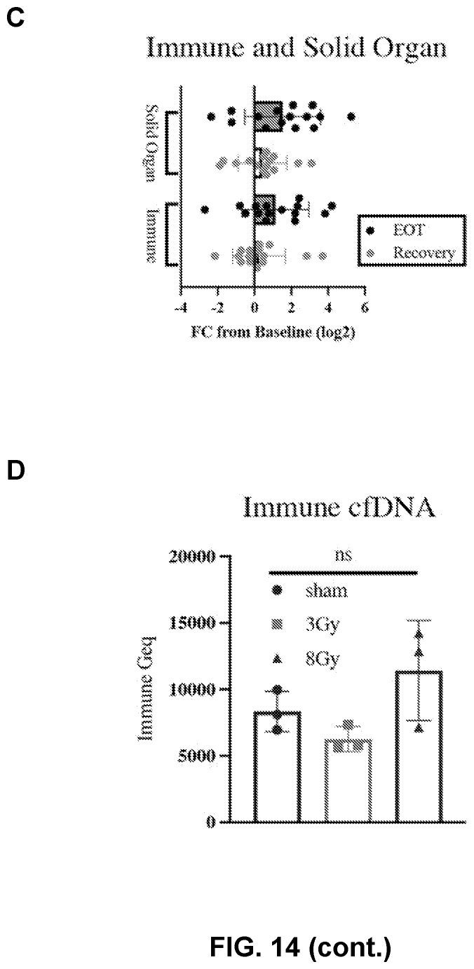

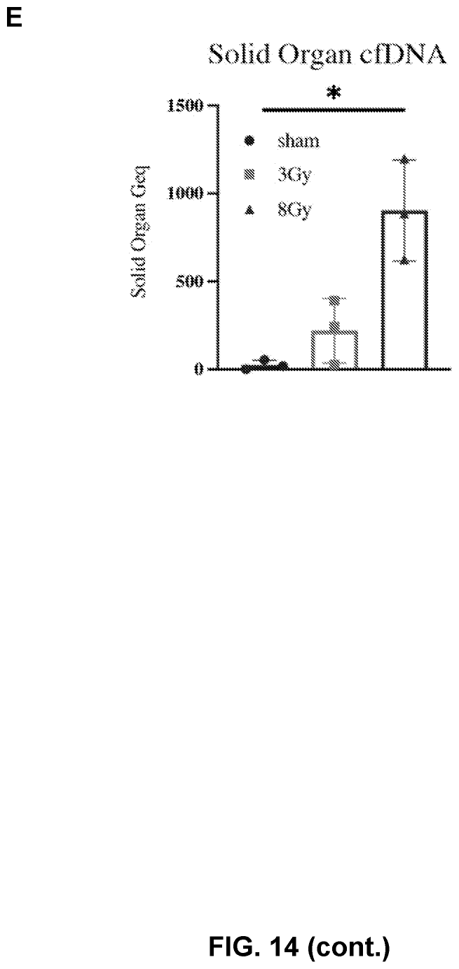

To assess indicators of heart lung, and liver damage in serum samples, data from capture sequencing of methylated cfDNA was analyzed (FIG. 2). For the analysis, the above-described mouse cardiomyocyte (n=2,917), lung endothelial (n=1,546), hepatocyte (n=616) and immune (n=148) cell-type specific methylation blocks derived from the radiation atlas for target organs-at-risk was used. Combining signals from 3Gy and 8Gy treated mice, a significant increase was found in percent lung endothelial, cardiomyocyte and hepatocyte cfDNA in the radiation-treated group relative to sham control that correlated with apoptotic cell death in the corresponding tissues. In addition, a significant dose-dependent increase was observed in percent lung endothelial, cardiomyocyte and combined solid organ cfDNA across all three treatment groups that correlated with radiation-induced cell death in the corresponding tissues (p<0.05, Kruskal-Wallis Test) (FIG. 12, Panels C and D; FIG. 14, Panel E). However, there was no dose-dependent increase in hepatocyte or immune cfDNA (FIG. 12, Panel E; FIG. 14, Panel D). As proof of principle, this supports that methylated DNA in blood can indicate the source of radiation-induced cellular damage in tissues.

Radiation treatment of/patients with breast cancer. To evaluate whether changes in cfDNA patterns could indicate damages to tissues in patients after radiation, serum samples were collected from breast cancer patients at three timepoints during their standard-of-care radiation therapy after surgery (FIG. 2). A baseline sample was taken for each patient before onset of radiation-therapy and after a total of 20-30 treatments a second End-Of-Treatment (EOT) sample was taken 30 minutes after the last treatment. Finally, a recovery sample was taken one month after completion of radiation-therapy. Demographic information and clinical characteristics of patients enrolled in this study are in Table 3 and in Barefoot et al., 2022, Supplemental Table 8. For analysis of cfDNA focus was on cell-types composing heart, lung, and liver tissues.

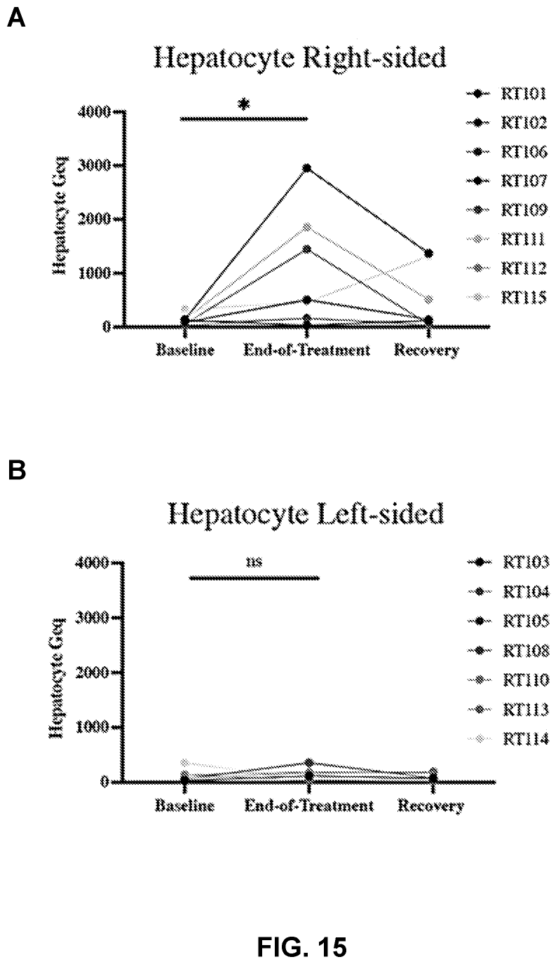

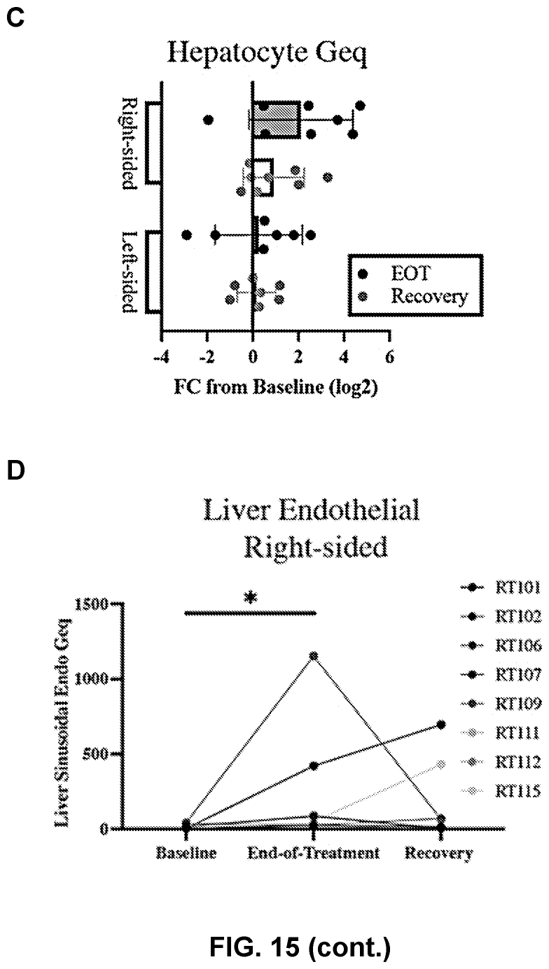

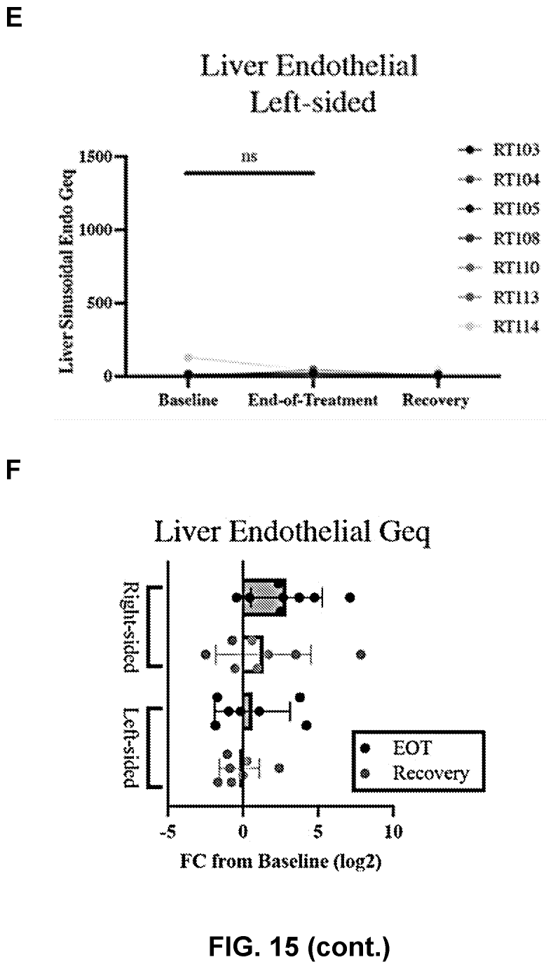

Radiation-induced liver damage. While liver damage is not a common radiation-induced toxicity experienced by breast cancer patients, a substantial dose may still be administered to the liver, especially with right-sided tumors (FIG. 11, Panel A). The top hepatocyte (n=200) and liver sinusoidal endothelial (n=89) methylation blocks were used to assess the sequence data for the presence of liver-derived cfDNA. Surprisingly, in patients receiving radiation treatment of right-sided breast cancer, an increase in hepatocyte plus liver sinusoidal endothelial methylated DNA in the circulation indicated significant radiation-induced cellular damage in the liver (p<0.05, Wilcoxon matched-pairs signed rank test) (FIG. 15, Panels A-F). Elevated levels of either hepatocyte and/or liver sinusoidal endothelial cfDNA were detected in seven of the eight breast cancer patients with right-sided tumors. In contrast, there was not significant increase in hepatocyte or liver sinusoidal endothelial cfDNA in patients with left-sided breast cancer.

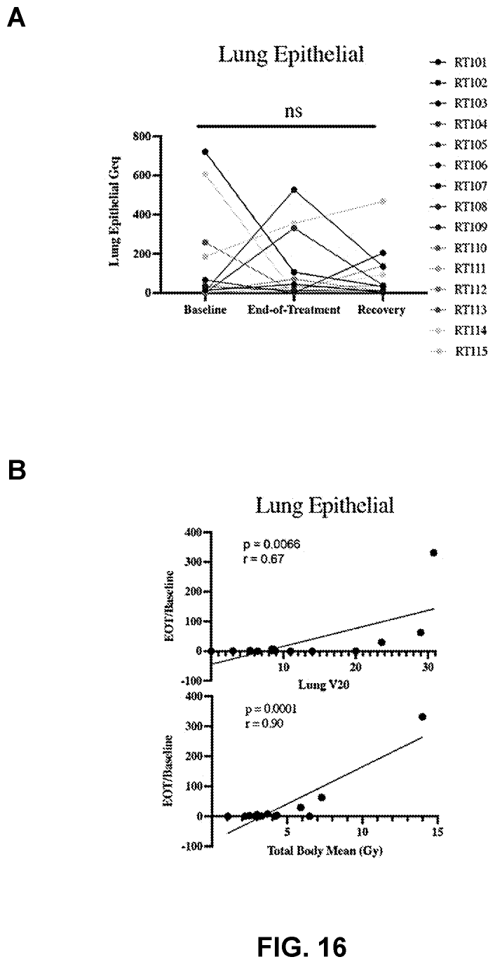

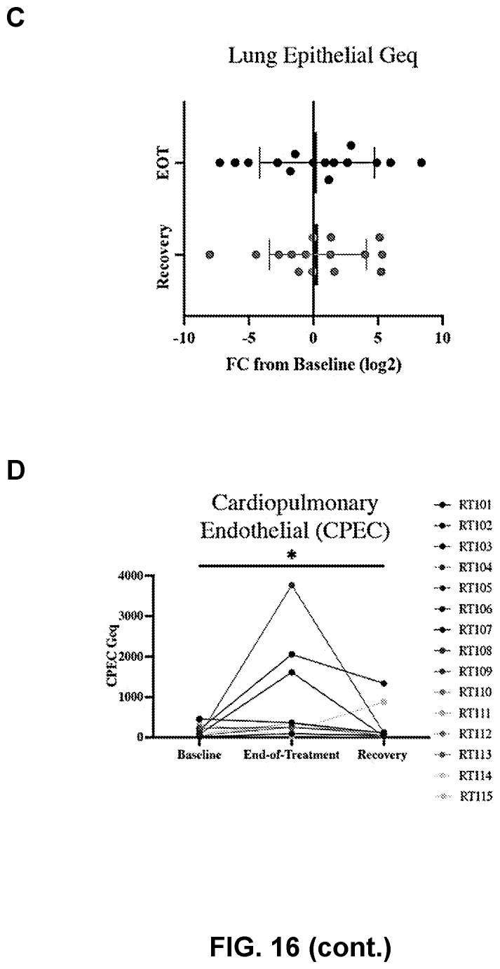

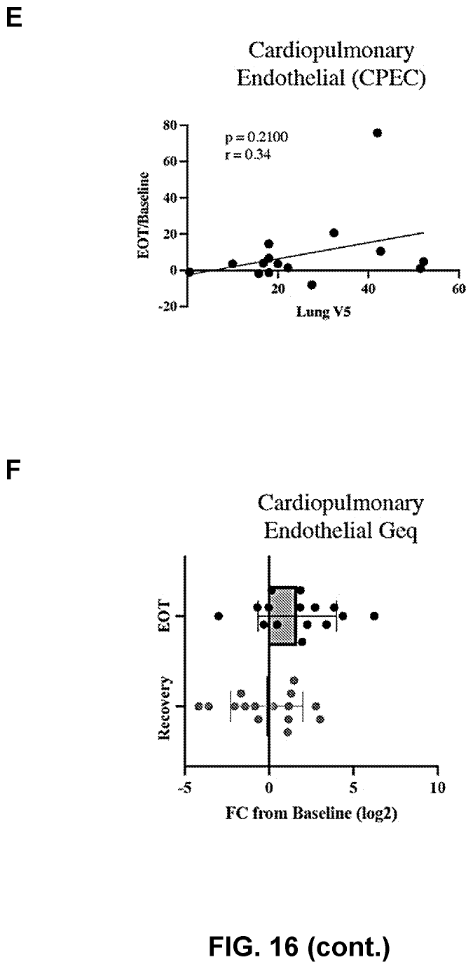

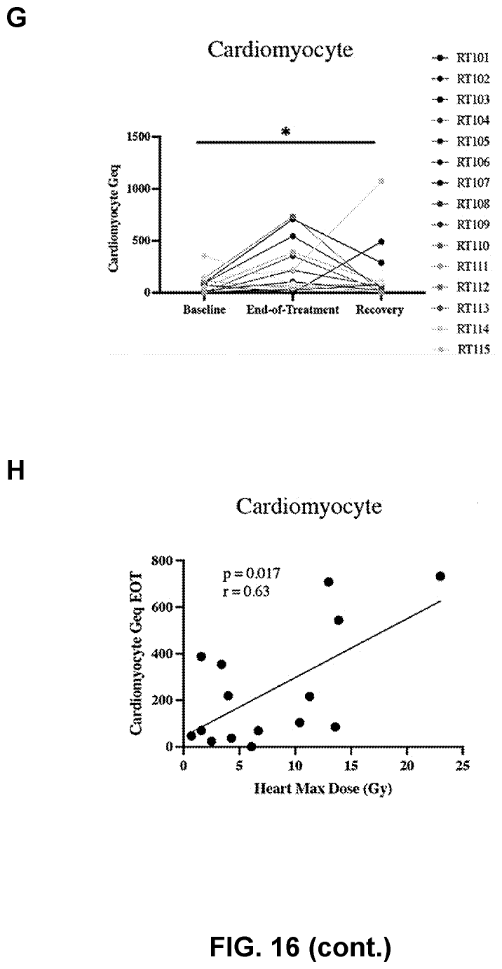

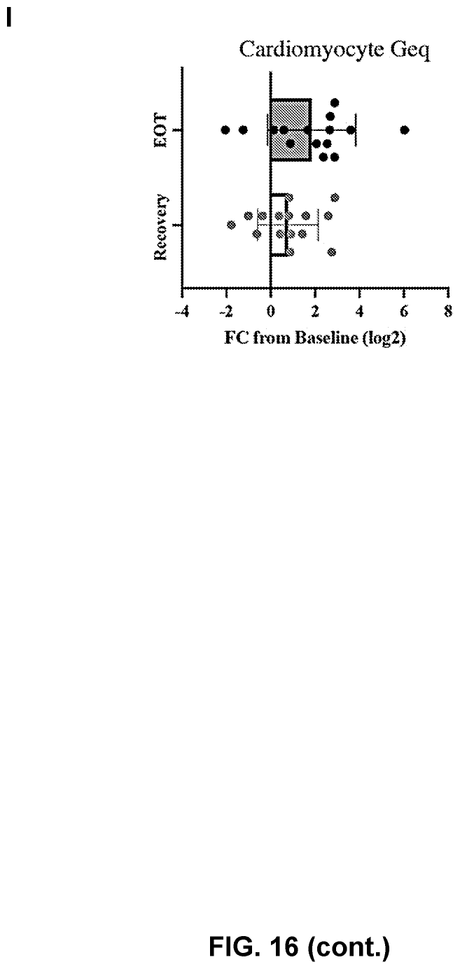

Radiation-induced heart and lung damage. Due to close proximity with the target treatment area, the heart and lungs are common organs-at-risk for breast cancer patients undergoing radiotherapy. To assess radiation-induced lung damage, cfDNAs from serum were examined for the presence of lung epithelial methylated DNA blocks (n=69). Interestingly, no significant increase in lung epithelial cfDNA across all patients was observed (p≥0.05, Friedman Test) (FIG. 16, Panel A). However, a few patients showed increased lung epithelial cfDNA indicating lung damage that correlated with increasing dose and volume of the lungs targeted (FIG. 16, Panel B). Specifically, longitudinal changes in lung epithelial cfDNA after radiation were found to correlate with the volume of the ipsilateral lung receiving 20Gy dose (Lung V20) (Pearson's r=0.67, p<0.05) and the total body mean dose (Pearson's r=0.90, p<0.05). In addition to lung injury, cardiovascular disease is one of the most serious complications from radiation exposure that is associated with increasing morbidity and mortality (White & Joiner, 2006; Brownlee et al., 2018). Through deconvolution using cardiopulmonary endothelial (CPEC, n=132) and cardiomyocyte-specific (n=375) DNA methylation blocks, increased CPEC and cardiomyocyte cfDNA was found in the serum samples indicating significant cardiovascular cell damage across all breast cancer patients (p<0.05, Friedman Test) (FIG. 16, Panels D and G). Surprisingly, cardiomyocyte-specific methylated DNA in the circulation correlated with the maximum radiation dose to the heart (Pearson's r=−0.63, p<0.05), but not the mean dose to the heart (Pearson's r=−0.09, p≥0.05) (FIG. 16, Panel H). This suggests that cardiomyocyte susceptibility to radiation-induced damage requires a sufficiently high dose, reinforcing the resilience of this cell-type to radiation damage compared to corresponding epithelial and endothelial cell-types from the heart and lungs.

Distinct endothelial and epithelial damages from radiation. Distinct epithelial and endothelial cell-type responses to radiation across the different tissues profiled were observed. Different responses to radiation were observed when comparing hepatocyte to lung epithelial damages (FIG. 15, Panels A-C versus FIG. 16, Panels A-C), demonstrating the ability of methylated DNA to distinguish between tissue-specific epithelial cell-types from serum samples. Likewise, analysis for tissue-specific endothelial populations reveals differences in cardiopulmonary microvascular and liver sinusoidal endothelial responses to radiation (FIG. 15, Panels D-F vs FIG. 16, Panels D-F). In general, there was greater magnitude of damage to the endothelium compared to the epithelium in different organs. The endothelium forms a layer of cells lining blood as well as lymphatic vessels. As a result, turnover from this cell-type likely may contribute to the high amplitude of signal detected from serum (Moss et al., 2018). This could, however, also be a result of the different sensitivities of endothelial versus epithelial cell-types to radiation-induced damage. There was a five-fold higher signal from CPEC cfDNA compared to lung epithelial cfDNA. Likewise, there was a two-fold increase in LSEC cfDNA compared to hepatocyte in right-sided cases. Also, in comparison to epithelial- and endothelial-derived cfDNA, sustained injury and delayed recovery is indicated by elevated cardiomyocyte cfDNA (FIG. 16, Panels C, F, and I). This may reflect important differences in cell turnover rates leading to differential processes of regeneration and repair in these cell-types. Notably, one month after completion of radiation therapy, epithelial damage signatures detected from cfDNA had returned to baseline levels although increased turnover of endothelial cells and cardiomyocytes indicate lingering tissue remodeling. Taken as a whole, these findings demonstrate applicability of this approach to uncover distinct cellular damages in different tissues during the course of treatment with a minimally invasive approach.

Comparison of results in humans and mice. Comparing the cfDNA origins after radiation, similar radiation-related changes were observed in both human and mouse serum samples. In both human and mouse, there was a significant increase in lung endothelial and cardiomyocyte cfDNAs after radiation. Likewise, there was an overall increase in cfDNA derived from any solid-organ tissue post-radiation in both breast cancer patients and mice receiving radiation (FIG. 14). The total concentration of cfDNA was elevated in some breast cancer patients at EOT as well, suggesting an overall increase in cfDNA shortly after radiation treatment (Table 9). Changes in mouse cfDNA concentration with increasing radiation dose were not significant (Table 13) as similarly reported in previous studies.78,79

This study demonstrated the ability of tissue-of-origin analysis of cell-free methylated DNA to monitor systemic responses to radiotherapy. The assignment of DNA fragments extracted from serum samples from patients undergoing treatment as well as from experimental animals to specific cell types required in-depth analysis of tissue- and cell-type methylation patterns. It was surprising that there was a significant association of the cell-type specific DNA methylation blocks with cell-type specific gene expression, transcription factor binding motifs and signaling pathway regulation. This study resulted in the development of a methylation atlas containing cell-type specific methylation patterns from target organs-at-risk from radiation damage, including the heart, lungs, and liver. It was found that methylated DNA in blood samples is an indicator of radiation damage that may be useful to predict patients who are more likely to develop severe adverse effects.

TABLE 3
Characteristics of breast cancer patients enrolled in the study.
Demographics

| Age | Range | Average | STDEV |
|---|---|---|---|
| | 38-76 | 60 | 13.6 |
| Race | AA | BA | Other |
| | 26.67% | 66.67% | 6.67% |

Tumor Characteristics

| | | | | |
|---|---|---|---|---|
| Stage | stage 0 | stage 1 | stage 2 | Stage 3 |
| | 25.00% | 26.70% | 33.00% | 20.00% |
| Histology | DC1S | IDC | ILC | |
| | 20.00% | 53.33% | 26.67% | |
| Laterality of breast cancer | Right | Left | | |
| | 53.33% | 46.67% | | |
| Hormone Receptors | ER+/PR+/HER2+ | ER+/PR+/HER2− | Triple Negative | |
| | 6.67% | 86.67% | 6.67% | |

Cancer Treatment

| | | | |
|---|---|---|---|
| Radiotherapy | 3D-CRT | PBT | SBRT |
| | 100.00% | 6.67% | 6.67% |
| Prior Radiation | Yes | No | |
| | 20.00% | 80.00% | |
| LR Node Irradiation | Yes | No | |
| | 26.67% | 73.00% | |
| Mean Lung Gy | Range | Mean | STDEV |
| | 0.6-16 | 6.8 | 4.71 |
| Meen Heart Gy | Range | Mean | STDEV |
| | 0.1-1.65 | 0.64 | 0.42 |
| Total Body Mean Gy | Range | Mean | STDEV |
| | 1.05-14 | 4.6 | 3.2 |
| Fx | Range | Mean | STDEV |
| | 5.0-30 | 22 | 7.2 |
| TO dose (cGY) | Range | Mean | STDEV |
| | 3000-6000 | 4988 | 876.2 |
| Adjuvant hormone therapy | Yes | No | |
| | 40.00% | 60.00% | |
| Adjuvant Chemotherapy | Yes | No | |
| | 26.67% | 73.00% | |

Medical History/Comorbidities

| | | |
|---|---|---|
| Diabetes | Yes | No |
| | 26.67% | 73.33% |

TABLE 4
Human reference methylation data from healthy tissues and cell-types.

| Consortium/ | | | | |
|---|---|---|---|---|
| Source | Tissue/Cell-type | Samples | DatasetID | Included in Atlas |
| Blueprint | Macrophage | 6 | EGAD00001002732; | X |
| | | | EGAD00001000923; | |
| | | | EGAD00001001192; | |
| | | | EGAD00001002501 | |
| Blueprint | Bcell | 6 | EGAD00001002732; | X |
| | | | EGAD00001000710; | |
| | | | EGAD00001001304 | |
| Blueprint | Monocyte | 6 | EGAD00001002732; | X |
| | | | EGAD00001000673; | |
| | | | EGAD00001000941; | |
| | | | EGAD00001001206 | |
| Blueprint | Megakaryocyte | 3 | EGAD00001002732; | X |
| | | | EGAD00001000932; | |
| | | | EGAD00001002311 | |
| Blueprint | CD4Tcell | 6 | EGAD00001002732; | X |
| | | | EGAD00001001157; | |
| | | | EGAD00001001516 | |
| Blueprint | CD8Tcell | 6 | EGAD00001002732; | X |
| | | | EGAD00001000921; | |
| | | | EGAD00001001189 | |
| Blueprint | NKcell | 6 | EGAD00001002732; | X |
| | | | EGAD00001001128; | |
| | | | EGAD00001002403 | |
| Blueprint | Eosinophil | 6 | EGAD00001002732; | |
| | | | EGAD00001001507; | |
| | | | EGAD00001002309 | |
| Blueprint | Erythroblast | 2 | EGAD00001000909; | X |
| | | | EGAD00001001133; | |
| | | | EGAD00001002423 | |
| Blueprint | Neutrophil | 6 | EGAD00001002732; | X |
| | | | EGAD00001000673; | |
| | | | EGAD00001000935; | |
| | | | EGAD00001001201; | |
| | | | EGAD00001002508 | |
| Blueprint | Endothelial cell | 2 | EGAD00001002294 | X |
| | of umbilical vein | | | |
| | (large vessel | | | |
| | endothelial) | | | |
| KNIH | Adipocyte | 2 | EGAD00001002755; | |
| | | | EGAD00001002756 | |
| KNIH | Podocyte | 4 | EGAD00001002758; | X |
| | | | EGAD00001002759; | |
| | | | EGAD00001003469; | |
| | | | EGAD00001003470 | |
| AMED-CREST/ | Hepatocyte | 10 | JGAD000026; | X |
| DEEP | | | EGAD00001002527 | |
| CEEHRC/KNIH | Skeletal muscle | 21 | EGAD00001001289; | X |
| | | | EGAD00001003871; | |
| | | | EGAD00001003872; | |
| | | | EGAD00001003873; | |
| | | | EGAD00001003875; | |
| | | | EGAD00001003876; | |
| | | | EGAD00001003877 | |
| ENCODE | Smooth muscle | 1 | ENCSR076OHG | |
| NIH Roadmap/ | Breast luminal | 5 | EGAD00001005060; | X |
| CEEHRC | epithelium | | EGAD00001005335; | |
| | | | EGAD00001006220 | |
| NIH Roadmap/ | Breast basal | 4 | EGAD00001005060; | |
| CEEHRC | epithelium | | EGAD00001005335; | |
| KNIH | Pancreatic islet | 5 | EGAD00001002750; | X |
| | cell | | EGAD00001002751; | |
| | | | EGAD00001002752; | |
| | | | EGAD00001002753; | |
| | | | EGAD00001002754 | |
| KNIH | Neuron | 2 | EGAD00001003474; | X |
| | | | EGAD00001003477 | |
| CEEHRC | Thyroid | 8 | EGAD00001001228 | |
| Stueve et al. 2017 | Lung epithelium | 12 | PRJNA375086 | X |
| (PMID:28854564) | | | | |
| AMED-CREST | Endometrial | 2 | JGAD000073 | |
| | epithelium | | | |
| ENCODE | Esophagus | 2 | ENCSR515MHO; | |
| | squamous | | ENCSR853BXB | |
| | epithelium | | | |
| AMED-CREST | Colon epithelial | 12 | JGAD000078 | X |
| | cells | | | |
| Gilsbach et al. 2018 | Cardiomyocyte | 5 | PRJNA353755 | X |
| (PMID:29374152) | | | | |
| Pidsley et al. 2017 | Prostate | 4 | PRJNA342657 | |
| (PMID:27717381) | epitheliim | | | |
| CUHK CNARG | Ureter Urothelial | 4 | Data generated by | |
| | | | “Chen THT et al in | |
| | | | Clin Biochem. | |
| | | | 2017;50:496-501”. | |
| CUHK CNARG | Bladder Epithelial | 1 | Data generated by | |
| | | | “Chen THT et al in | |
| | | | Clin Biochem. | |
| | | | 2017;50:496-501”. | |
| NIH Roadmap/ | Gastric | 2 | ENCSR999RWT; | |
| ENCODE | epithelium/ | | ENCSR669BAL | |
| | stomach | | | |
| Jamil et al. 2020 | Liver sinusoidal | 6 | PRJNA596240 | X |
| (PMID:33096636)/ | endothelial | | | |
| in-house | | | | |
| in-house | Cardiopulmonary | 6 | | X |
| | endothelial | | | |

TABLE 5
Mouse reference methylation data from healthy tissues and cell-types.

| Consortium/Source | Tissue/Cell-type | Samples | DatasetID | Included in Atlas |
|---|---|---|---|---|
| Duncan et al. 2018 | Bcell | 4 | PRJNA391196 | X |
| (PMID:29326230)/ | | | | |
| in-house | | | | |
| Delacher et al. 2017 | CD4Tcell | 5 | PRJEB14591 | X |
| (PMID:28783152)/ | | | | |
| in-house | | | | |
| in-house | CD8Tcell | 1 | | X |
| in-house | Neutrophil | 1 | | X |
| in-house | Buffy coat | 4 | | X |
| ENCODE/Hon et | Bone marrow | 5 | ENCBS218HTH; | X |
| al 2013 | | | ENCBS430NOM; | |
| (PMID:23995138) | | | GSM1051150; | |
| | | | ENCFF802SFU; | |
| | | | ENCFF703DEV; | |
| | | | ENCFF306ZPW; | |
| | | | ENCFF340YVI | |
| Delacher et al. 2017 | Tissue-resident | 9 | PRJEB14591 | |
| (PMID:28783152) | Treg | | | |
| Hon et al 2013 | Spleen | 1 | GSM1051163 | |
| (PMID:23995138) | | | | |
| Hon et al 2013 | Thy mus | 1 | GSM1051165 | |
| (PMID:23995138) | | | | |
| Gilsbach et al. 2014 | Cardiomyocyte | 3 | PRJNA229470 | X |
| (PMID:25335909) | | | | |
| ENCODE/Hon et | Heart | 8 | ENCSR633CON; | |
| al 2013 | | | ENCSR835OJU; | |
| (PMID:23995138) | | | ENCSR258MDR; | |
| | | | ENCSR397YEG; | |
| | | | ENCSR641SDF; | |
| | | | ENCSR265OMO; | |
| | | | ENCSR149GUT; | |
| | | | ENCSR050EXR; | |
| | | | GSM1051154 | |
| Gravina et al. 2016 | Hepatocyte | 3 | PRJNA310298 | X |
| | | (13) | | |
| ENCODE/Hon et | Liver | 8 | ENCSR550CYA; | |
| al 2013 | | | ENCSR660CKG; | |
| (PMID:23995138) | | | ENCSR788XSZ; | |
| | | | ENCSR334GBD; | |
| | | | ENCSR129SBE; | |
| | | | ENCSR324NAF; | |
| | | | ENCSR033PGF; | |
| | | | GSM1051157 | |
| Sabbagh et al. 2018 | Endothelial | 8 | GSE111839 | |
| (PMID:30188322) | | | | |
| Schlereth et al. 2017 | Lung endothelial | 3 | PRJNA344551 | X |
| (PMID:29749927) | | | | |
| ENCODE/Hon et | Lung | 5 | ENCSR191UKH; | |
| al 2013 | | | ENCSR409HKJ; | |
| (PMID:23995138) | | | ENCSR535YCH; | |
| | | | ENCSR027ICI; | |
| | | | GSM1051158 | |
| ENCODE/Hon et | Cerebellum | 5 | GSM1051151; | X |
| al 2013 | | | ENCBS358CVB; | |
| (PMID:23995138) | | | ENCBS588RGY | |
| Lagger et al. 2017 | Hypothalamus | 3 | PRJNA329552 | X |
| (PMID:28498846) | | | | |
| Hon et al 2013 | Olfactory Bulb | 1 | GSM1051159 | |
| (PMID:23995138) | | | | |
| ENCODE/Hon et | Intestine | 5 | ENCSR089FFK; | X |
| al 2013 | | | ENCSR217TMK; | |
| (PMID:23995138) | | | ENCSR353IFP; | |
| | | | ENCSR842QTB; | |
| | | | GSM1051155 | |
| Hon et al 2013 | Colon | 1 | GSM1051152 | |
| (PMID:23995138) | | | | |
| ENCODE/Hon et | Stomach | 5 | ENCSR953WFU; | |
| al 2013 | | | ENCSR013VIR; | |
| (PMID:23995138) | | | ENCSR28600J; | |
| | | | ENCSR545WRA; | |
| | | | GSM1051164 | |
| ENCODE/Hon et | Kidney | 5 | ENCSR425NDU; | X |
| al 2013 | | | ENCSR128HOP; | |
| (PMID:23995138) | | | ENCSR841TRV; | |
| | | | ENCSR906HLA; | |
| | | | GSM1051156 | |
| Hon et al 2013 | Pancreas | 1 | GSM1051160 | |
| (PMID:23995138) | | | | |
| Gravina et al. 2016 | Fibroblast | 2 | PRJNA310298 | |
| dos Santos et al. | Mammary | 5 | GSE67386 | X |
| 2015 | epithelial | | | |
| (PMID:25959817) | | | | |
| Hon et al 2013 | Placenta | 1 | GSM1051161 | |
| (PMID:23995138) | | | | |
| Hon et al 2013 | Uterus | 1 | GSM1051166 | |
| (PMID:23995138) | | | | |
| Hon et al 2013 | Skin | 1 | GSM1051162 | |
| (PMID:23995138) | | | | |

TABLE 6
Summary of identified human cell-type specific
methylation blocks (AMF > |0.4|, minimum 3CpG sites).

| Cell-type | #Hypomethylated | #Hypermethylated |
|---|---|---|
| Bcell | 53 | 0 |
| Cardiopulmonary | 128 | 4 |
| Endothelial (CPEC) | | |
| Breast Basal Epithelial | 705 | 155 |
| Breast Luminal Epithelial | 287 | 43 |
| Cardiomyocyte | 649 | 15 |
| CD4Tcell | 52 | 2 |
| CD8Tcell | 73 | 2 |
| Colon Epithelial | 519 | 58 |
| Large Vessel Endothelial | 104 | 12 |
| (LVEC) | | |
| Erythroblast | 12 | 1 |
| Hepatocyte | 482 | 79 |
| Kidney Podocyte | 60 | 13 |
| Liver Sinusoidal | 88 | 1 |
| Endothelial (LSEC) | | |
| Lung Epithelial | 68 | 1 |
| Monocyte/Macrophage | 97 | 0 |
| Neuron | 106 | 197 |
| Neutrophil | 51 | 0 |
| Nkcell | 52 | 0 |
| Skeletal Muscle | 211 | 3 |
| Pancreas Islet | 92 | 22 |
| Bulk Immune | 117 | 105 |

TABLE 7
Summary of identified mouse cell-type specific
methylation blocks (AMF > |0.4|, minimum 3CpG sites).

| Cell-type | #Hypomethylated | #Hypermethylated |
|---|---|---|
| Bulk Immune | 0 | 148 |
| Cardiomyocyte | 2917 | 0 |
| Hepatocyte | 616 | 0 |
| Lung Endothelial | 1546 | 0 |
| Cerebellum | 1229 | 0 |
| Mammary Epithelial | 874 | 0 |
| Intestine | 6 | 0 |
| Hypothalamus | 4 | 0 |
| Kidney | 4 | 0 |

TABLE 8
Genomic annotation of identified
human and mouse cell-type specific
hypomethylated and hypermethylated
blocks relative to all captured blocks.

| Human_Hypomethylated | |
|---|---|
| blocks (n = 3889) | |
| intergenic | 915 |
| intragenic | 2942 |
| promoter-TSS | 274 |
| TTS | 142 |
| exon | 631 |
| intron | 1895 |
| Human_Hypomethylated | |
| blocks (n = 608) | |
| intergenic | 204 |
| Intragenic | 404 |
| promoter-TSS | 80 |
| TTS | 24 |
| exon | 117 |
| intron | 183 |
| Human Background | |
| Captured Blocks | |
| (n = 317963) | |
| intergenic | 98202 |
| intragenic | 219761 |
| promoter | 39216 |
| TTS | 9943 |
| exon | 39260 |
| intron | 131342 |
| Mouse_Hypomethylated | |
| blocks (n = 7964) | |
| intergenic | 2700 |
| intragenic | 5264 |
| promoter-TSS | 119 |
| TTS | 136 |
| exon | 716 |
| intron | 4293 |
| Mouse_Hypomethylated | |
| blocks (n = 148) | |
| intergenic | 17 |
| Intragenic | 131 |
| promoter-TSS | 16 |
| TTS | 5 |
| exon | 40 |
| intron | 70 |
| Mouse Background | |
| Captured Blocks | |
| (n = 1214889) | |
| intergenic | 608763 |
| intragenic | 606126 |
| promoter | 42339 |
| TTS | 16699 |
| exon | 55317 |
| intron | 491771 |

TABLE 9
Human cfDNA sample concentrations and predicted precents from deconvolution
analysis at identified cell-type specific blocks for target cell-types

| | | | | | | % Liver | |
|---|---|---|---|---|---|---|---|
| | | | | | | Sinusoidal | |
| | | % Lung | | | % Cardiopulmonary | Endothelial | |
| Sample | ng/mL | Epithelial | % Hepatocyte | % Cardiomyocyte | Endothelial (CPEC) | (LSEC) | % Immune |
| 101.1 | 142.0 | 1.675904524 | 0.287332084 | 0.029971414 | 1.057076778 | 0 | 96.9497152 |
| 101.2 | 100.4 | 0.348632095 | 0.105411607 | 0.078332303 | 1.203961225 | 0.09027696 | 98.1733858 |
| 101.3 | 116.4 | 0.0942617 | 0.336054406 | 0.220777008 | 0.312584367 | 0.03922258 | 98.9970999 |
| 102.1 | 68.4 | 0.159638429 | 0.415568659 | 0.465308433 | 0.374905104 | 0.07881589 | 98.5057635 |
| 102.2 | 372.0 | 0.011095914 | 0.446790142 | 0.628657336 | 1.430265589 | 0.07488822 | 97.4083028 |
| 102.3 | 96.8 | 0.051613574 | 0.47360535 | 0.984144776 | 0 | 0 | 98.4906363 |
| 103.1 | 19.1 | 0.256707102 | 0.563417383 | 0.28425303 | 0.24044444 | 0.18948499 | 98.4656931 |
| 103.2 | 88.0 | 0.16686356 | 0.426237369 | 0.390857292 | 0.347458494 | 0.08661508 | 98.5819682 |
| 103.3 | 33.6 | 0.045541196 | 0.710362406 | 0.217893557 | 0.382335646 | 0.05237159 | 98.5914956 |
| 104.1 | 74.7 | 0.04140701 | 0.358170659 | 0.281567649 | 0.420281174 | 0 | 98.8985735 |
| 104.2 | 88.8 | 0.263052706 | 0.624499172 | 0.259036654 | 1.283134955 | 0.15643783 | 97.4138387 |
| 104.3 | 90.1 | 0.032511234 | 0.674608147 | 0.304792838 | 0.22554669 | 0.04420491 | 98.7183362 |
| 105.1 | 15.6 | 0.057912071 | 0.403966669 | 0.250549456 | 0.583406255 | 0.36333617 | 98.3408294 |
| 105.2 | 26.9 | 0.033082363 | 0.333533186 | 0.460896505 | 1.211293535 | 0.18785466 | 97.7733397 |
| 105.3 | 21.5 | 0.107636608 | 0.293577302 | 0.487267321 | 1.051777845 | 0.15455843 | 97.9051825 |
| 106.1 | 39.7 | 0.069472616 | 0.250285823 | 0.774141116 | 1.90445977 | 0.29539538 | 96.7062453 |
| 106.2 | 182.7 | 0.951953399 | 0.294805455 | 0.981969082 | 0.457496611 | 0.04868031 | 97.2650951 |
| 106.3 | 24.5 | 1.80978571 | 0.465130627 | 0.812485429 | 1.729335411 | 0.93515001 | 94.2481128 |
| 107.1 | 44.3 | 0.497611236 | 1.053494562 | 0.542070916 | 1.447219552 | 0.02240723 | 96.4371965 |
| 107.2 | 632.0 | 0 | 1.542284221 | 0.157889618 | 1.071853544 | 0.21960132 | 97.0083713 |
| 107.3 | 320.0 | 0.211757934 | 1.416242727 | 0.505639938 | 1.376137521 | 0.71798595 | 95.7722359 |
| 108.1 | 29.1 | 0 | 0.688671158 | 0.382439275 | 0.603764532 | 0.16302918 | 98.1620959 |
| 108.2 | 148.8 | 0.735053495 | 0.783024014 | 0.479429223 | 0.568956322 | 0.01666383 | 97.4168731 |
| 108.3 | 32.3 | 0.408248008 | 0.779447652 | 0.605693045 | 0.64876015 | 0.14714937 | 97.4107018 |
| 109.1 | 38.4 | 0 | 0.474231281 | 0.248280445 | 0.426939593 | 0.3631607 | 98.487388 |
| 109.2 | 722.7 | 0.013676029 | 0.65980246 | 0.161370039 | 1.718976989 | 0.52657702 | 96.9195975 |
| 109.3 | 45.9 | 0.272650817 | 0.361996258 | 0.103163038 | 0.803899375 | 0.45873763 | 97.9995529 |
| 110.1 | 30.9 | 0.4742599 | 1.403818565 | 1.191351612 | 0.392211011 | 0.16481115 | 96.3735478 |
| 110.2 | 17.6 | 0.242255832 | 0.794249572 | 0.882501569 | 0.680428153 | 0.08111527 | 97.3194496 |
| 110.3 | 26.3 | 0.372535977 | 0.825663635 | 1.085022006 | 0.99582761 | 0.23200288 | 96.4889479 |
| 111.1 | 30.9 | 0.080724628 | 1.502603313 | 0.55841863 | 0.934586383 | 0.16762099 | 96.7560461 |
| 111.2 | 92.8 | 0.167643817 | 6.60401336 | 1.377649585 | 1.211801446 | 0.31783789 | 90.3210539 |
| 111.3 | 26.7 | 0.091107031 | 6.340253715 | 1.181319466 | 0.091158051 | 0.13543535 | 92.1607264 |
| 112.1 | 59.7 | 0.021991448 | 0.551215254 | 0.005929387 | 0.134739401 | 0 | 99.2861245 |
| 112.2 | 64.8 | 0.04623058 | 0.747756312 | 0.358843279 | 1.814499911 | 0.12394989 | 96.90872 |
| 112.3 | 36.5 | 1.2638928 | 0.632178819 | 0.016952957 | 0.492033467 | 0 | 97.594942 |
| 113.1 | 111.5 | 0.767210315 | 0.405267296 | 0.418209983 | 0.979478906 | 0 | 97.4298335 |
| 113.2 | 174.9 | 0.014953727 | 0.357760609 | 1.38343133 | 0.078292337 | 0.08876883 | 98.0767932 |
| 113.3 | 61.1 | 0 | 0.895662241 | 1.435923237 | 0 | 0 | 97.6684145 |
| 114.1 | 304.0 | 0.65783443 | 0.377453852 | 0.382912335 | 0.275136473 | 0.14017883 | 98.1664841 |
| 114.2 | 46.9 | 0.02814705 | 0.327169124 | 0.60028721 | 1.103292289 | 0.27702703 | 97.6640773 |
| 114.3 | 68.5 | 0.458553005 | 0.969397206 | 0.496765788 | 0.386149925 | 0.19633411 | 97.4928 |
| 115.1 | 47.2 | 1.3088228 | 2.28363898 | 1.013656941 | 0.755232344 | 0.26146117 | 94.3771878 |
| 115.2 | 72.8 | 1.609908701 | 2.064005032 | 0.995458717 | 0.684814019 | 0.23204816 | 94.4137654 |
| 115.3 | 594.7 | 0.259518205 | 0.730465023 | 0.596255318 | 0.488497441 | 0.23923304 | 97.686031 |

TABLE 10
Mouse cfDNA sample concentrations and predicted
precents from deconvolution analysis at
identified cell-type specific blocks for target cell-types.

| | | % | % | % Lung | % |
|---|---|---|---|---|---|
| Sample | ng/ml | cardiomyocyte | hepatocyte | Endothelial | Immune |
| shamA | 25.12 | 0.221790643 | 0.12116778 | 0.310984938 | 99.34605664 |
| shamB | 21.04 | 0 | 0.26485898 | 0.007434279 | 99.72770674 |
| shamC | 29.92 | 0 | 0 | 0 | 100 |
| 3GyA | 23.92 | 2.406149764 | 1.586386567 | 0.918922438 | 95.08854123 |
| 3GyB | 18.32 | 0.14614797 | 0.107333799 | 0.188790206 | 99.55772802 |
| 3GyC | 18.32 | 1.34771749 | 2.179727824 | 0.452849459 | 96.01970523 |
| 8GyA | 47.52 | 2.772568704 | 1.20751631 | 3.597659949 | 92.42225504 |
| 8GyB | 48.16 | 1.35983355 | 1.946827849 | 0.589794119 | 96.10354448 |
| 8GyC | 23.2 | 8.351210369 | 1.467364049 | 1.660177126 | 88.52124846 |

TABLE 11
Enriched biological pathways and functions for genes associated with differential
methylation in each cell-type examined.

| | | |
|---|---|---|
| Biological Pathways | log(p- | |
| and Functions | value) | Molecules |

HUMAN LUNG EPITHELIAL

| | | |
|---|---|---|
| Binding of pulmonary | 4.72 | HS6ST1, SDC1 |
| fibroblasts | | |
| Abnormal | 4.64 | BMPER, FSTL1, TBX1 |
| morphology of larynx | | |
| Abnormality of | 3.43 | ADRA2A, BMPER, CCN4, FSTL1, TBX1 |
| cartilage tissue | | |
| Cough | 3.18 | ADORA2B, ADRA2A, ATP4B |
| Quantity of type II | 3.13 | EPAS1, FSTL1 |
| pneumocytes | | |
| Hyperpolarization of | 3.08 | BMPER, FSTL1 |
| membrane | | |
| Lower respiratory | 3.04 | ADORA2B, ADRA2A, ATP4B, BMPER, FSTL1, |
| tract disorder | | HS6ST1, USP40 |
| Pulmonary | 2.90 | ADORA2B, ADRA2A, EPAS1, RPTOR |
| Hypertension | | |
| Migration of | 2.89 | RPTOR, SIRPA |
| Langerhans cells | | |
| Respiratory failure | 2.86 | ADRA2A, EPAS1, TBX1, WNT7B |
| Abnormal | 2.77 | FSTL1, PRKCZ |
| morphology of | | |
| tracheal ring | | |
| α-Adrenergic | 2.58 | ADRA2A, GNA12, PRKCZ |
| Signaling | | |
| AMPK Signaling | 2.48 | ADRA2A, GNA12, PPM1H, RPTOR |
| Expression of RNA | 2.46 | BHLHE41, EHMT1, EPAS1, GNA12, GRIP1, |
| | | HMG20A, LIMS1, MAD1L1, MCF2L, PRKCZ, |
| | | RREB1, SIRPA, STAU2, TBX1, TCF25, VAV2, |
| | | WNT7B |
| Migration of lung cell | 2.45 | ARHGEF7, MCF2L |
| lines | | |
| Circadian Rhythm | 2.33 | BHLHE41, CSNK1D, PRKCZ, RPTOR |
| Signaling | | |
| Xenobiotic | 1.91 | GRIP1, HS6ST1, PRKCZ |
| Metabolism CAR | | |
| Signaling Pathway | | |
| 3-phosphoinositide | 1.90 | PALD1, PPM1H, SIRPA |
| Degradation | | |
| Xenobiotic | 1.89 | GRIP1, HS6ST1, PRKCZ |
| Metabolism PXR | | |
| Signaling Pathway | | |
| Role of WNT/GSK- | 1.77 | CSNK1D, WNT7B |
| 3β Signaling in the | | |
| Pathogenesis of | | |
| Influenza | | |
| Integrin Signaling | 1.77 | ARHGEF7, LIMS1, TNK2 |
| RHOGDI Signaling | 1.76 | ARHGEF7, GNA12, GRIP1 |
| lung morphogenesis | 1.59 | CELSR1, RDH10, WNT7B |
| lung development | 1.38 | CELSR1, EPAS1, HS6ST1, RDH10, WNT7B |
| Pulmonary Fibrosis | 1.30 | CCN4, GNA12, WNT7B |
| Idiopathic Signaling | | |
| Pathway | | |

HUMAN CARDIOMYOCYTE

| | | |
|---|---|---|
| myofibril assembly | 35.02 | ACTC1, ACTG1, CASQ2, LDB3, LMOD2, |
| | | LMOD3, MEF2A, MYBPC3, MYH6, MYL2, |
| | | MYLK3, MYOM2, MYOZ1, MYOZ3, MYPN, |
| | | TPM1, TTN |
| sarcomere | 31.96 | ACTG1, CASQ2, LDB3, LMOD2, MYBPC3, |
| organization | | MYH6, MYLK3, MYOM2, MYPN, TPM1, TTN |
| actomyosin structure | 30.16 | ACTC1, ACTG1, CASQ2, FRMD5, LDB3, |
| organization | | LMOD2, LMOD3, MEF2A, MYBPC3, MYH6, |
| | | MYL2, MYLK3, MYOM2, MYOZ1, MYOZ3, |
| | | MYPN, PHACTR1, SORBS1, TPM1, TTN |
| cellular component | 28.36 | ACTC1, ACTG1, CASQ2, GPC1, LDB3, LMOD2, |
| assembly involved in | | LMOD3, MEF2A, MYBPC3, MYH6, MYL2, |
| morphogenesis | | MYLK3, MYOM2, MYOZ1, MYOZ3, MYPN, |
| | | TENM4, TPM1, TTN |
| striated muscle cell | 28.22 | ACTC1, ACTG1, CASQ2, FHL2, LDB3, LMOD2, |
| development | | LMOD3, LRRC10, MEF2A, MYBPC3, MYH6, |
| | | MYL2, MYLK3, MYOM2, MYOZ1, MYOZ3, |
| | | MYPN, NPPA, PDLIM5, SORBS2, TBX3, TPM1, |
| | | TTN |
| actin filament-based | 27.40 | ABLIM2, ACTC1, ACTG1, ARHGAP26, BAIAP2, |
| process | | BAIAP2L1, CACNA1C, CAP2, CAPN10, CASQ2, |
| | | COBL, CORO6, DES, DNAJB6, DTNBP1, EVL, |
| | | FGF12, FNBP1L, FRMD5, FRY, GAB1, INF2, |
| | | INPP5K, KCND3, KCNQ1, LDB3, LMOD2, |
| | | LMOD3, MEF2A, MICAL3, MYBPC3, MYH6, |
| | | MYH7, MYL2, MYLK3, MY05A, MYOM2, |
| | | MYOZ1, MYOZ3, MYPN, NEDD4L, NISCH, |
| | | NPHP4, PARVA, PARVB, PDGFA, PDLIM3, |
| | | PDPK1, PHACTR1, PKP2, PPARGC1B, SCN5A, |
| | | SNTA1, SORBS1, SORBS2, SPTBN5, SYNE2, |
| | | TNNC1, TPM1, TTN, WASL, XIRP1 |
| muscle cell | 26.60 | ACTC1, ACTG1, ATP2A2, CASQ2, ENG, FHL2, |
| development | | LDB3, LMOD2, LMOD3, LRRC10, MEF2A, |
| | | MYBPC3, MYH6, MYL2, MYLK3, MYOM2, |
| | | MYOZ1, MYOZ3, MYPN, NPPA, PDLIM5, |
| | | SORBS2, TBX3, TPM1, TRIM54, TTN |
| Morphology of heart | 21.57 | ACACB, ACTC1, ADAM19, ADCY5, ADCY6, |
| | | ADRB1, AGO2, ANK3, ATP2A2, BNIP3, CABIN1, |
| | | CACNA1C, CASP9, CASQ2, CASZ1, CCND1, |
| | | CKMT2, CUX1, DCHS1, DES, DPF3, F3, FBXO32, |
| | | FGF12, FHL2, FITM1, GATA6, HAND1, HCN4, |
| | | HDAC4, HSPB7, IGF1R, JARID2, JPH2, KCNQ1, |
| | | KIDINS220, LDB3, LIMS2, LMOD2, LRRC10, |
| | | MEF2A, mir-208, mir-486, MYBPC3, MYH7, |
| | | MYL2, MYL9, MYLK3, MYOCD, MYPN, |
| | | MYZAP, NEDD4L, NPPA, PDLIM5, PDPK1, |
| | | PKP2, PLEC, PPARGC1B, PRDM16, RARB, |
| | | RPL3L, RPS6KA2, RXRA, SAV1, SCN5A, SGTA, |
| | | SLC25A21, SMARCD3, SMYD1, SNTA1, SPEG, |
| | | SYNE2, TBX5, TNNC1, TNS1, TPM1, TRAF3, |
| | | TRIM63, TTN, UBE4B, UCN, XIRP1, ZMIZ1 |
| cardiac muscle tissue | 19.82 | ACTC1, BMPR1A, ENG, FHL2, FOXC1, GATA6, |
| development | | HAND1, LRRC10, MEF2A, MYBPC3, MYH6, |
| | | MYH7, MYL2, MYLK3, MYOCD, MYPN, |
| | | NDRG4, NPPA, PDLIM5, PKP2, RARB, RXRA, |
| | | SORBS2, TBX3, TBX5, TENM4, TNNC1, TPM1, |
| | | TTN, UBE4B |
| Cardiogenesis | 17.87 | ACTC1, ADAM19, APC, ATP2A2, CACNA1C, |
| | | CASQ2, CASZ1, CCND1, COL5A1, CTBP2, |
| | | CUX1, DCHS1, FGF12, GAB1, GATA6, GYS1, |
| | | HAND1, HDAC4, IFT140, JARID2, JPH2, KCNQ1, |
| | | KLF7, LDB3, LIMS2, LMOD2, LRRC10, mir-208, |
| | | MTHFD1, MYBPC3, MYH7, MYL2, MYLK3, |
| | | MYO18B, MYOCD, MYOM2, MYPN, NPPA, |
| | | PDPK1, PKP2, PLEC, PPARGC1B, PTCH1, |
| | | RPS6KA2, RXRA, SAV1, SCN5A, SMARCD3, |
| | | SMYD1, SPEG, SYNPO2L, TBX5, TENM4, |
| | | TNNC1, TPM1, TRIM63, TTN, VGLL4, XIRP1, |
| | | ZMIZ1 |
| Dilated | 13.70 | ACTC1, ADCY5, ADCY6, ADCY9, ADRB1, |
| Cardiomyopathy | | ATP2A2, CACNA1C, CAMK2B, CASP9, DES, |
| Signaling Pathway | | GAB1, ITPR1, MYBPC3, MYH7, MYH7B, MYL2, |
| | | MYL9, MYO18B, PRKAR1B, SCN5A, TNNC1, |
| | | TPM1, TTN |
| Morphology of | 13.39 | ACTC1, ADCY6, ADRB1, ATP2A2, CABIN1, |
| muscle cells | | CASQ2, COL13A1, CUX1, DES, FBXO32, FHL2, |
| | | GATA6, HSPB7, ILF3, JARID2, JPH2, LDB3, |
| | | LIMS2, LMO7, LMOD2, LRRC10, MEF2A, mir- |
| | | 208, MYBPC3, MYLK3, MYOCD, NPPA, PARVA, |
| | | PDLIM5, PDPK1, PLEC, PSAP, RPS6KA2, SGCA, |
| | | SMARCD3, SNTA1, SPEG, TBX5, TRIM63, TTN, |
| | | UBE4B, UCN, XIRP1 |
| Morphology of | 12.31 | ACTC1, ADCY6, ADRB1, ATP2A2, CABIN1, |
| cardiac muscle | | CASP9, CASQ2, CUX1, DES, FBXO32, FHL2, |
| | | GATA6, HSPB7, JPH2, LIMS2, LRRC10, mir-208, |
| | | MYBPC3, MYL9, MYLK3, MYOCD, NPPA, |
| | | PDPK1, PKP2, PLEC, PPARGC1B, RPS6KA2, |
| | | RXRA, SMARCD3, TBX5, TRIM63, TTN, UBE4B, |
| | | UCN, XIRP1 |
| Morphology of | 10.00 | ACTC1, ADCY6, ADRB1, ATP2A2, CABIN1, |
| cardiomyocytes | | CASQ2, CUX1, DES, FBXO32, FHL2, GATA6, |
| | | HSPB7, JPH2, LIMS2, LRRC10, mir-208, |
| | | MYBPC3, MYLK3, MYOCD, NPPA, PDPK1, |
| | | PLEC, RPS6KA2, TBX5, TRIM63, TTN, UBE4B, |
| | | UCN |
| Calcium Signaling | 7.20 | ACTC1, ATP2A2, CABIN1, CACNA1C, CAMK2B, |
| | | CASQ2, CREBBP, HDAC4, ITPR1, LETM1, |
| | | MEF2A, MYH7, MYH7B, MYL2, MYL9, |
| | | MYO18B, PRKAR1B, TNNC1, TPM1 |
| White Adipose Tissue | 5.52 | ADCY5, ADCY6, ADCY9, CACNA1C, CREBBP, |
| Browning Pathway | | CTBP2, ITPR1, NPPA, NRF1, PRDM16, |
| | | PRKAR1B, RARB, RXRA |
| ILK Signaling | 4.39 | ACTC1, CCND1, CREBBP, FBLIM1, LIMS2, |
| | | MYH7, MYH7B, MYL2, MYL9, MYO18B, |
| | | PARVA, PARVB, PDPK1, PPP2R5C |
| Factors Promoting | 4.33 | APC, CAMK2B, CCND1, CREBBP, LRP5, MYH7, |
| Cardiogenesis in | | MYL2, MYOCD, NPPA, PLCL2, SCN5A, TBX5 |
| Vertebrates | | |
| Role of NFAT in | 3.31 | ADCY5, ADCY6, ADCY9, CABIN1, CACNA1C, |
| Cardiac Hypertrophy | | CAMK2B, HAND1, HDAC4, IGF1R, ITPR1, |
| | | MEF2A, PLCL2, PRKAR1B |
| Assembly of RNA | 2.73 | GTF3A, GTF3C1, GTF3C5 |
| Polymerase III | | |
| Complex | | |
| Cardiac Hypertrophy | 2.69 | ADCY5, ADCY6, ADCY9, ADRB1, CACNA1C, |
| Signaling | | CREBBP, HAND1, IGF1R, MEF2A, MYL2, MYL9, |
| | | PLCL2, PRKAR1B |
| Cardiac β-adrenergic | 2.05 | ADCY5, ADCY6, ADCY9, ADRB1, ATP2A2, |
| Signaling | | CACNA1C, PPP1R7, PPP2R5C, PRKAR1B |
| Thrombin Signaling | 2.02 | ADCY5, ADCY6, ADCY9, CAMK2B, GATA6, |
| | | ITPR1, MYL2, MYL9, PDPK1, PLCL2 |
| Cardiomyocyte | 2.00 | MYH7, MYL2, NPPA |
| Differentiation via | | |
| BMP Receptors | | |
| Apelin | 1.34 | ATP2A2, ITPR1, MYL2, MYL9, PLCL2 |
| Cardiomyocyte | | |
| Signaling Pathway | | |

HUMAN HEPATOCYTE

| | | |
|---|---|---|
| FXR/RXR Activation | 21.80 | A1BG, ABCG5, ABCG8, AHSG, APOB, APOC2, |
| | | APOC4, APOF, CLU, CYP8B1, FASN, FBP1, FGA, |
| | | FOXA1, HPR, HPX, IL1RN, LIPC, ORM1, ORM2, |
| | | PON1, RBP4, RXRA, SLC10A1, SLC27A5, |
| | | SLC51B, VTN |
| LXR/RXR Activation | 19.60 | A1BG, ABCG5, ABCG8, AHSG, APOA5, APOB, |
| | | APOC2, APOC4, APOF, CLU, FASN, FGA, HPR, |
| | | HPX, IL1RN, LBP, NCOR2, ORM1, ORM2, PON1, |
| | | RBP4, RXRA, SCD, UGT1A3, VTN |
| organic anion | 16.63 | ABCG8, ACACB, ACSL1, AGXT, AKR1C4, |
| transport | | APOA5, APOC2, CYB5R2, DRD4, GOT2, MPC1, |
| | | PLA2G12B, PLA2G2C, PLA2G2F, RXRA, |
| | | SERINC2, SERINC3, SLC10A1, SLC13A5, |
| | | SLC16A1, SLC16A11, SLC16A13, SLC16A3, |
| | | SLC17A1, SLC17A3, SLC19A1, SLC22A1, |
| | | SLC22A3, SLC22A9, SLC25A25, SLC25A4, |
| | | SLC25A42, SLC27A5, SLC35A3, SLC38A10, |
| | | SLC38A7, SLC38A8, SLC43A1, SLC51B, |
| | | SLC5A12, SLC6A1, SLC7A10, SLC7A5, SLC7A9, |
| | | THRSP |
| Liver lesion | 12.91 | A1BG, A2M, ABCG5, ABCG8, ACACB, ACSL1, |
| | | ADM, AGBL1, AGTR1, AHSG, AKR1C4, ALDH2, |
| | | ALDH8A1, ALPL, ANGPTL7, ANPEP, AP1M1, |
| | | APOA5, APOB, APOF, ARC, ARHGAP44, |
| | | ARMC5, ARNTL, ATP2B2, BDH1, C1R, C1S, C5, |
| | | CABLES2, CAMK2G, CAMTA2, CAPN5, |
| | | CBFA2T3, CCDC57, CDK9, CDKN1B, CENPP, |
| | | CHRD, CHRNA4, CLASP1, CLU, COL18A1, |
| | | CPN2, CTSB, CUX2, CYP2E1, CYP8B1, DAB2IP, |
| | | DCPS, DHCR24, DNAJB12, DOCK6, DOCK9, |
| | | DUSP1, DUSP3, ELFN1, EPPK1, ERICH1, |
| | | EXOC3L4, F12, F2, FAAP100, FAM20C, FASN, |
| | | FBP1, FGA, FGF1, FOXD2-AS1, GNPNAT1, |
| | | GOT1, GOT2, GPER1, GPRC5C, GRHPR, GYS2, |
| | | H6PD, HAAO, HAGH, HIF1AN, HP, HPN, HPX, |
| | | HR, IGF2R, IGFALS, IHH, IL1RN, INHBE, |
| | | INPP5A, INS-IGF2, INTS6, IRS1, ITGB4, ITIH1, |
| | | LBP, LPAL2, LRP5, MAD1L1, MASP1, MASP2, |
| | | MAT1A, MEGF6, MGMT, MGRN1, MICAL3, |
| | | MINK1, mir-122, MLXIP, MYO1C, MYO7A, |
| | | NAGS, NAT8, NCMAP, NCOR2, NFIC, NINL, |
| | | NLRP6, NPC1L1, NRDE2, NTN1, OPLAH, ORM1, |
| | | OSGIN1, PAH, PC, PCK1, PCSK6, PEMT, PIEZO1, |
| | | PLEC, PMEPA1, PMFBP1, PML, POLE, PRLR, |
| | | PROC, PROZ, PTPRF, RBM33, RBP4, RFC1, |
| | | RIN3, RNF220, RPS6KA2, RXRA, SARM1, SCD, |
| | | SEBOX, SELENOP, SERINC2, SH3BP2, SH3BP4, |
| | | SH3PXD2A, SIGIRR, SLC10A1, SLC16A13, |
| | | SLC22A1, SLC22A9, SLC25A4, SLC25A42, |
| | | SLC25A47, SLC27A5, SLC29A1, SLC43A1, |
| | | SLC51B, SLC6A1, SLC7A10, SLC7A5, SLC7A9, |
| | | SMAD3, SNED1, SNTG1, SORBS2, SOX10, |
| | | SOX11, SPTBN1, SQSTM1, SS18L1, STK24, |
| | | SUN1, TBC1D14, TEDC1, TEX2, TFR2, TIMP2, |
| | | TM6SF2, TMEM33, TMEM82, TNK2, TNS3, |
| | | TOP1MT, TRAF3, TRAPPC4, TRIP10, UBE2V2, |
| | | UGT1A1, UGT1A3, UGT1A4, UROC1, VAV2, |
| | | VPS37B, VPS4A, VTN, WDR62, WNT5A, |
| | | ZC3H10, ZC3H7B, ZFPM1, ZFYVE28, ZMYM4, |
| | | ZNF358, ZNF444 |
| Synthesis of lipid | 12.23 | ABCG5, ABCG8, ACACB, ACSL1, ADM, AGTR1, |
| | | AKR1C4, ALDH8A1, ANPEP, APOA5, APOB, |
| | | APOC2, ARMC5, ARNTL, ATAD3A, C5, |
| | | CAMK2G, CYP2E1, CYP8B1, DGAT1, DGKD, |
| | | DHCR24, ETNPPL, F2, FASN, FGF1, FOXA1, |
| | | GATA4, GPD1, GPER1, GYS2, H6PD, HSD17B1, |
| | | IL1RN, LIPC, NPC1L1, NTN1, PC, PCK1, PEMT, |
| | | PIP5K1C, PLA2G2F, PLPP3, PON1, PRLR, |
| | | PTK2B, RXRA, SCD, SERINC2, SIGIRR, |
| | | SLC22A1, SLC27A5, SLC9A3R2, SMAD3, |
| | | SRD5A1, ST6GALNAC6, THRSP, VTN |
| pre-mRNA catabolic | 11.50 | ZNF259 |
| process | | |
| Quantity of steroid | 11.30 | ABCG5, ABCG8, ACACB, ADM, AGTR1, |
| | | APOA5, APOB, ARNTL, CDKN1B, CLU, CRY2, |
| | | CYP8B1, DGAT1, DHCR24, DUSP1, FASN, |
| | | GATA4, GPER1, H6PD, HIF1AN, HP, HPN, |
| | | HSD17B1, IHH, IL1RN, IRS1, LIPC, LRP5, mir- |
| | | 122, NPC1L1, PEMT, PON1, PRLR, RXRA, SCD, |
| | | SMAD3, SRD5A1, TM6SF2, TRAF3, UGT1A1, |
| | | VAV2 |
| Liver cancer | 10.27 | A1BG, ABCG5, ABCG8, ACACB, ACSL1, |
| | | AGBL1, AHSG, AKR1C4, ALDH2, ALDH8A1, |
| | | ALPL, ANPEP, AP1M1, APOA5, APOB, APOF, |
| | | ARC, ARHGAP44, ARMC5, ARNTL, ATP2B2, |
| | | BDH1, C1R, C1S, C5, CABLES2, CAMK2G, |
| | | CAMTA2, CBFA2T3, CCDC57, CDK9, CDKN1B, |
| | | CENPP, CHRD, CHRNA4, CLASP1, CLU, |
| | | COL18A1, CTSB, CUX2, CYP8B1, DAB2IP, |
| | | DHCR24, DNAJB12, DOCK9, DUSP3, EPPKI, |
| | | ERICH1, F12, F2, FAAP100, FAM20C, FASN, |
| | | FBP1, FGA, FOXD2-AS1, GNPNAT1, GOT1, |
| | | GPER1, GPRC5C, GRHPR, GYS2, H6PD, HAAO, |
| | | HAGH, HP, HPX, HR, IGF2R, IHH, INHBE, |
| | | INPP5A, INS-IGF2, INTS6, IRS1, ITGB4, ITIH1, |
| | | LBP, LPAL2, LRP5, MAD1L1, MASP1, MASP2, |
| | | MEGF6, MGMT, MGRN1, MICAL3, MINK1, mir- |
| | | 122, MLXIP, MYO1C, MYO7A, NAT8, NCMAP, |
| | | NCOR2, NFIC, NLRP6, NRDE2, NTN1, OPLAH, |
| | | ORM1, OSGIN1, PAH, PC, PCK1, PCSK6, |
| | | PIEZO1, PLEC, PMEPA1, PMFBP1, PML, POLE, |
| | | PROZ, PTPRF, RBM33, RBP4, RFC1, RIN3, |
| | | RNF220, RPS6KA2, RXRA, SARM1, SCD, |
| | | SEBOX, SELENOP, SERINC2, SH3BP2, |
| | | SH3PXD2A, SLC10A1, SLC16A13, SLC22A1, |
| | | SLC22A9, SLC25A42, SLC25A47, SLC27A5, |
| | | SLC29A1, SLC6A1, SLC7A10, SLC7A5, SLC7A9, |
| | | SMAD3, SNED1, SNTG1, SOX10, SOX11, |
| | | SPTBN1, SQSTM1, STK24, SUN1, TBC1D14, |
| | | TEDC1, TEX2, TFR2, TIMP2, TM6SF2, TMEM33, |
| | | TMEM82, TNK2, TNS3, TOP1MT, TRAPPC4, |
| | | TRIP10, UBE2V2, UGT1A1, UGT1A3, UGT1A4, |
| | | UROC1, VAV2, VPS37B, VPS4A, VTN, WDR62, |
| | | ZC3H10, ZC3H7B, ZFPM1, ZFYVE28, ZMYM4, |
| | | ZNF358 |
| Concentration of | 9.67 | ABCG5, ABCG8, ACACB, ADM, APOA5, APOB, |
| cholesterol | | ARNTL, CDKN1B, CYP8B1, DGAT1, DHCR24, |
| | | DUSP1, FASN, GPER1, HIF1AN, HP, HPN, |
| | | IL1RN, IRS1, LIPC, LRP5, mir-122, NPC1L1, |
| | | PEMT, PON1, RXRA, SCD, TM6SF2, UGT1A1 |
| Hepatobiliary | 9.33 | A1BG, A2M, ABCG5, ABCG8, ACACB, ACSL1, |
| neoplasm | | AGBL1, AHSG, AKR1C4, ALDH2, ALDH8A1, |
| | | ALPL, ANGPTL7, ANPEP, AP1M1, APOA5, |
| | | APOB, APOF, ARC, ARHGAP44, ARMC5, |
| | | ARNTL, ATP2B2, BDH1, C1R, C1S, C5, |
| | | CABLES2, CAMK2G, CAMTA2, CBFA2T3, |
| | | CCDC57, CDK9, CDKN1B, CENPP, CHRD, |
| | | CHRNA4, CLASP1, CLU, COL18A1, CPN2, |
| | | CTSB, CUX2, CYP8B1, DAB2IP, DCPS, DHCR24, |
| | | DNAJB12, DOCK6, DOCK9, DUSP1, DUSP3, |
| | | ELFN1, EPPK1, ERICH1, EXOC3L4, F12, F2, |
| | | FAAP100, FAM20C, FASN, FBP1, FGA, FOXD2- |
| | | AS1, GNPNAT1, GNRH2, GOT1, GPER1, |
| | | GPRC5C, GRHPR, GYS2, H6PD, HAAO, HAGH, |
| | | HIF1AN, HP, HPN, HPX, HR, IGF2R, IGFALS, |
| | | IHH, INHBE, INPP5A, INS-IGF2, INTS6, IRS1, |
| | | ITGB4, ITIH1, KATNAL1, LBP, LPAL2, LRP5, |
| | | MAD1L1, MASP1, MASP2, MEGF6, MGMT, |
| | | MGRN1, MICAL3, MINK1, mir-122, MLXIP, |
| | | MYO1C, MYO7A, NAGS, NAT8, NCMAP, |
| | | NCOR2, NFIC, NINL, NLRP6, NRDE2, NTN1, |
| | | OPLAH, ORM1, OSGIN1, PAH, PC, PCK1, |
| | | PCSK6, PIEZO1, PLEC, PMEPA1, PMFBP1, PML, |
| | | POLE, PRLR, PROZ, PTPRF, RBM33, RBP4, |
| | | RFC1, RIN3, RNF220, RPS6KA2, RXRA, SARM1, |
| | | SCD, SEBOX, SELENOP, SERINC2, SH3BP2, |
| | | SH3PXD2A, SLC10A1, SLC16A13, SLC22A1, |
| | | SLC22A9, SLC25A42, SLC25A47, SLC27A5, |
| | | SLC29A1, SLC43A1, SLC6A1, SLC7A10, |
| | | SLC7A5, SLC7A9, SMAD3, SNED1, SNTG1, |
| | | SORBS2, SOX10, SOX11, SPTBN1, SQSTM1, |
| | | SS18L1, STK24, SUN1, TBC1D14, TEDC1, TEX2, |
| | | TFR2, TIMP2, TM6SF2, TMEM33, TMEM82, |
| | | TNK2, TNS3, TOP1MT, TRAPPC4, TRIP10, |
| | | UBE2V2, UGT1A1, UGT1A3, UGT1A4, UROC1, |
| | | VAV2, VPS37B, VPS4A, VTN, WDR62, WNT5A, |
| | | ZC3H10, ZC3H7B, ZFAND2A, ZFHX3, ZFPM1, |
| | | ZFYVE28, ZMYM4, ZNF358, ZNF444 |
| Acute Phase Response | 7.09 | A2M, AHSG, C1R, C1S, C5, F2, FGA, HP, HPX, |
| Signaling | | HRG, IL1RN, ITIH3, LBP, ORM1, ORM2, RBP4 |
| Clathrin-mediated | 6.86 | AP1M1, APOB, APOC2, APOC4, APOF, CLU, F2, |
| Endocytosis Signaling | | FGF1, FGF3, ITGB4, ORM1, ORM2, PIP5K1C, |
| | | PON1, RBP4, SH3BP4 |
| LPS/IL-1 Mediated | 5.92 | ABCG5, ABCG8, ACSF2, ACSL1, ALAS1, |
| Inhibition of RXR | | ALDH2, ALDH8A1, APOC2, APOC4, CYP2E1, |
| Function | | IL1RN, LBP, LIPC, MGMT, RXRA, SLC10A1, |
| | | SLC27A5 |
| Morphology of liver | 5.73 | AGL, ARHGAP1, CDKN1B, CYP2E1, DUSP1, |
| | | FGA, FGF1, GATA4, GPER1, HIF1AN, IGF2R, |
| | | IRS1, MAT1A, mir-122, PCK1, PEMT, PROC, |
| | | RXRA, SLC13A5, SLC22A1, SMAD3, SPTBN1, |
| | | WNT5A, ZFPM1 |
| Extrinsic Prothrombin | 5.35 | F12, F2, F7, FGA, PROC |
| Activation Pathway | | |
| Growth of epithelial | 5.06 | ADM, AGTR1, C5, CAMK2G, CBFA2T3, |
| tissue | | CDKN1B, CLU, COL18A1, CTSB, CYBA, |
| | | DAB2IP, EPPK1, F12, F2, FGF1, FGF3, HIF1AN, |
| | | HPN, IHH, IL1RN, ITGB4, NFIC, NLRP6, NTN1, |
| | | PC, PML, PRLR, PROC, RXRA, SEPTIN9, |
| | | SLC7A5, SLC9A3R2, SMAD3, SOX11, SPTBN1, |
| | | TIMP2, TOP1MT, VAV2, WNT5A |
| TR/RXR Activation | 4.99 | APOA5, FASN, FGA, HP, NCOR2, PCK1, RXRA, |
| | | SLC16A3, THRSP |
| Coagulation System | 4.68 | A2M, F12, F2, F7, FGA, PROC |
| liver development | 4.41 | CEBPB, CEBPG, FPGS, GNPNAT1, IGF2R, IHH, |
| | | MPST, ONECUT2, PROC, SEBOX, SMAD3, |
| | | SRD5A1, UGT1A1, UGT1A8 |
| Complement System | 3.47 | C1R, C1S, C5, MASP1, MASP2 |
| Fatty Acid Activation | 2.76 | ACSF2, ACSL1, SLC27A5 |
| Bile Acid | 2.52 | AKR1C4, CYP8B1, SLC27A5 |
| Biosynthesis, Neutral | | |
| Pathway | | |
| Iron homeostasis | 2.07 | ATP6V0D1, HP, HPX, SLC25A37, SMAD3, |
| signaling pathway | | STEAP3, TFR2 |
| Hepatic Cholestasis | 1.84 | ABCG5, ABCG8, CYP8B1, IL1RN, LBP, RXRA, |
| | | SLC10A1, TJP2 |
| Hepatic Fibrosis/ | 1.81 | A2M, AGTR1, COL18A1, CYP2E1, FGF1, LBP, |
| Hepatic Stellate Cell | | SMAD3, TIMP2 |
| Activation | | |

HUMAN ENDOTHELIAL

| | | |
|---|---|---|
| Development of | 13.09 | ABCA4, ANGPT2, APLNR, ARAP3, ATXN1, |
| vasculature | | CCL24, CCN1, CCN2, CD9, CTNNBIP1, CTTN, |
| | | DLC1, ECSCR, EFNB2, EGFL7, EPHA2, EPHB2, |
| | | ESM1, FAT1, FGF18, FLI1, FLT1, FOXC1, GBX2, |
| | | HOXD3, HSPG2, HYAL1, IGF1R, JCAD, LAMA4, |
| | | MAP2K5, MMP14, MMRN2, NFATC1, NOS3, |
| | | NOTCH1, PDE2A, PIK3R1, PLPP3, PRKCE, |
| | | RAPGEF1, RASA3, SEMA6A, SMAD3, SOX7, |
| | | TCF7L2, TGM2, TMEM204, VWF, WLS, ZEB1 |
| Angiogenesis | 13.08 | ABCA4, ANGPT2, APLNR, ARAP3, ATXN1, |
| | | CCL24, CCN1, CCN2, CD9, CTNNBIP1, ECSCR, |
| | | EFNB2, EGFL7, EPHA2, EPHB2, ESM1, FAT1, |
| | | FGF18, FLI1, FLT1, FOXC1, GBX2, HOXD3, |
| | | HSPG2, HYAL1, IGF1R, JCAD, LAMA4, |
| | | MAP2K5, MMP14, NFATC1, NOS3, NOTCH1, |
| | | PDE2A, PIK3R1, PLPP3, PRKCE, RAPGEF1, |
| | | RASA3, SEMA6A, SMAD3, SOX7, TCF7L2, |
| | | TGM2, TMEM204, VWF, WLS, ZEB1 |
| Morphology of | 10.10 | ANGPT2, ANK3, APLNR, ATXN1, CACNA1D, |
| cardiovascular system | | CAPZB, CCN2, CTTN, EFNB2, EPHA2, FGF18, |
| | | FLT1, GBX2, GRK5, HDAC4, HOXD3, HSPG2, |
| | | IGF1R, LAMA4, LDLRAD3, MAP2K5, MIR5093, |
| | | MME, MMP14, MMRN2, MTERF4, NCOR2, |
| | | NFATC1, NOS3, NOTCH1, PARD3, PIK3R1, |
| | | PLPP3, PRDM16, PRKCE, RHEB, RPS6KA2, |
| | | SMAD3, TGM2, VWF, ZEB1 |
| Vasculogenesis | 9.97 | ABCA4, ANGPT2, APLNR, ATXN1, CCL24, |
| | | CCN1, CCN2, CD9, CTNNBIP1, ECSCR, EFNB2, |
| | | EGFL7, EPHA2, EPHB2, FAT1, FLI1, FLT1, |
| | | FOXC1, GBX2, HSPG2, IGF1R, MAP2K5, |
| | | MMP14, NFATC1, NOS3, NOTCH1, PDE2A, |
| | | PLPP3, PRKCE, RASA3, SEMA6A, SMAD3, |
| | | SOX7, TCF7L2, TGM2, VWF, WLS, ZEB1 |
| Cell movement of | 8.20 | ANGPT2, CCL24, CCN1, CCN2, CD9, DLC1, |
| endothelial cells | | ECSCR, EFNB2, EGFL7, FGF18, FLT1, FOXC1, |
| | | HSPG2, MAP2K5, MMP14, NOS3, NOTCH1, |
| | | PDE2A, PLPP3, PRKCE, SMAD3, VWF |
| Transcription of RNA | 7.39 | ABLIM2, AEBP2, ANKRD33, ATXN1, CAMKK2, |
| | | CCN1, CCN2, CCNDBP1, CD9, CRCP, CTBP1, |
| | | CTNNBIP1, FLI1, FOXC1, FOXL1, GBX2, GLI2, |
| | | GRHL2, HDAC4, HOXD3, IBTK, IGF1R, INO80, |
| | | ITGA6, KANK2, MAD1L1, MAMSTR, MAP2K5, |
| | | MME, MTA1, MXD3, MYEF2, MYT1L, NACC2, |
| | | NCOR2, NFATC1, NOTCH1, PDE2A, PIAS4, |
| | | PIK3R1, PRDM16, PRKCE, RAPGEF1, RAX2, |
| | | SMAD3, SOX2, SOX7, TCF7L2, TFCP2L1, |
| | | TRAF7, TRIM44, ZEB1, ZHX2 |
| Transcription of DNA | 6.27 | ABLIM2, AEBP2, ANKRD33, ATXN1, CAMKK2, |
| | | CCN1, CRCP, CTBP1, CTNNBIP1, FLI1, FOXC1, |
| | | FOXL1, GBX2, GLI2, GRHL2, HDAC4, HOXD3, |
| | | IGF1R, INO80, ITGA6, KANK2, MAMSTR, |
| | | MAP2K5, MXD3, MYEF2, MYT1L, NACC2, |
| | | NCOR2, NFATC1, NOTCH1, PDE2A, PIAS4, |
| | | PIK3R1, PRDM16, RAX2, SMAD3, SOX2, SOX7, |
| | | TCF7L2, TFCP2L1, TRAF7, TRIM44, ZEB1, ZHX2 |
| Migration of vascular | 5.49 | ANGPT2, CCN1, CD9, DLC1, ECSCR, EFNB2, |
| endothelial cells | | FLT1, FOXC1, MMP14, PDE2A, PLPP3, SMAD3 |
| Development of | 4.79 | CCN1, CD9, FLT1, MMP14, NOTCH1, RASA3 |
| vascular endothelial | | |
| cells | | |
| Apelin Endothelial | 4.43 | ADCY4, APLNR, GNG7, HDAC4, NOS3, PIK3R1, |
| Signaling Pathway | | PRKCE, SMAD3 |
| IGF-1 Signaling | 4.41 | CCN1, CCN2, GRB10, IGF1R, NEDD4, PIK3R1, |
| | | PRKAR1B |
| Nitric Oxide Signaling | 4.04 | CACNA1D, FLT1, NOS3, PDE2A, PIK3R1, |
| in the Cardiovascular | | PRKAR1B, PRKCE |
| System | | |
| Pulmonary Fibrosis | 3.80 | AEBP2, CCN2, EFNB2, FGF18, GLI2, MMP14, |
| Idiopathic Signaling | | NOTCH1, PIK3R1, RPS6KA2, SMAD3, TCF7L2 |
| Pathway | | |
| Opioid Signaling | 3.72 | ADCY4, CACNA1D, GNG7, GRK5, MAP2K5, |
| Pathway | | NOS3, PRKAR1B, PRKCE, RPS6KA2, TCF7L2 |
| Oxytocin Signaling | 3.71 | CACNA1D, CAMKK2, GNG7, MAP2K5, |
| Pathway | | MYO18A, NFATC1, NOS3, PIK3R1, PRKAR1B, |
| | | PRKCE |
| White Adipose Tissue | 3.68 | ADCY4, ANGPT2, CACNA1D, CAMKK2, CTBP1, |
| Browning Pathway | | PRDM16, PRKAR1B |
| Estrogen Receptor | 3.56 | ADCY4, CACNA1D, CTBP1, GNG7, IGF1R, |
| Signaling | | MMP14, NCOR2, NOS3, NOTCH1, PIK3R1, |
| | | PRKAR1B, PRKCE |
| Transcriptional | 2.88 | CDYL, FOXC1, GBX2, SOX2 |
| Regulatory Network | | |
| in Embryonic Stem | | |
| Cells | | |
| Cellular Effects of | 2.68 | ADCY4, CACNA1D, MYO18A, NOS3, PDE2A, |
| Sildenafil (Viagra) | | PRKAR1B |
| Relaxin Signaling | 2.59 | ADCY4, GNG7, NOS3, PDE2A, PIK3R1, |
| | | PRKAR1B |
| eNOS Signaling | 2.54 | ADCY4, FLT1, NOS3, PIK3R1, PRKAR1B, |
| | | PRKCE |
| Netrin Signaling | 2.42 | ABLIM2, CACNA1D, NFATC1, PRKAR1B |
| VEGF Family Ligand- | 2.19 | FLT1, NOS3, PIK3R1, PRKCE |
| Receptor Interactions | | |
| Endothelin-1 | 1.55 | ADCY4, CASP2, NOS3, PIK3R1, PRKCE |
| Signaling | | |
| HIF1α Signaling | 1.43 | FLT1, MAP2K5, MMP14, PIK3R1, PRKCE |

HUMAN IMMUNE

| | | |
|---|---|---|
| Proliferation of | 10.46 | CARMIL2, CD37, CD6, CD79B, CR2, CUL3, |
| immune cells | | DEF6, EFNB2, ETS1, GFI1, IL15, IL6R, ITGA2B, |
| | | JAK3, LCP1, MAD1L1, MAP4K1, MBL2, |
| | | NCKAP1L, NCOR2, NFATC1, OSM, PLA2G6, |
| | | PTPN6, PTPRCAP, RPS6KA1, RPTOR, RUNX3, |
| | | S100B, S1PR4, SKAP1, SOCS3, STING1 |
| Proliferation of | 10.43 | CARMIL2, CD37, CD6, CD79B, CR2, CUL3, |
| mononuclear | | DEF6, EFNB2, ETS1, GFI1, IL15, IL6R, ITGA2B, |
| leukocytes | | JAK3, LCP1, MAD1L1, MAP4K1, MBL2, |
| | | NCKAP1L, NCOR2, NFATC1, OSM, PLA2G6, |
| | | PTPN6, PTPRCAP, RPTOR, RUNX3, S100B, |
| | | S1PR4, SKAP1, SOCS3, STING1 |
| Differentiation of | 10.31 | CD6, CD79B, DOCK1, FGF6, GFI1, GNA15, |
| progenitor cells | | HOXA3, HOXA5, IL15, IL6R, ITGA2B, JAK3, |
| | | KCNAB2, MAD1L1, MAFK, MAP4K1, OLIG2, |
| | | OSM, PTPN6, RPS6KA1, RPTOR, RXRA, S1PR4, |
| | | SOCS3, TEK |
| Proliferation of | 9.93 | CARMIL2, CD37, CD6, CD79B, CR2, CUL3, |
| lymphocytes | | DEF6, EFNB2, ETS1, GFI1, IL15, IL6R, ITGA2B, |
| | | JAK3, LCP1, MAD1L1, MAP4K1, NCKAP1L, |
| | | NCOR2, NFATC1, OSM, PLA2G6, PTPN6, |
| | | PTPRCAP, RPTOR, RUNX3, S100B, S1PR4, |
| | | SKAP1, SOCS3, STING1 |
| Proliferation of blood | 9.87 | CARMIL2, CD37, CD6, CD79B, CR2, CUL3, |
| cells | | DEF6, EFNB2, ETS1, GFI1, HOXA5, IL15, IL6R, |
| | | ITGA2B, JAK3, LCP1, MAD1L1, MAP4K1, MBL2, |
| | | NCKAP1L, NCOR2, NFATC1, OSM, PLA2G6, |
| | | PTPN6, PTPRCAP, RPS6KA1, RPTOR, RUNX3, |
| | | S100B, S1PR4, SKAP1, SOCS3, STING1 |
| Cell proliferation of T | 9.42 | CARMIL2, CD37, CD6, CUL3, DEF6, EFNB2, |
| lymphocytes | | ETS1, GFI1, IL15, IL6R, ITGA2B, JAK3, LCP1, |
| | | MAD1L1, MAP4K1, NCKAP1L, NCOR2, OSM, |
| | | PTPN6, PTPRCAP, RPTOR, RUNX3, S100B, |
| | | S1PR4, SKAP1, SOCS3, STING1 |
| Quantity of | 9.24 | C1QTNF6, CD6, CD79B, CR2, DEF6, EFNB2, |
| mononuclear | | ETS1, FGF6, GFI1, HOXA3, HVCN1, IL15, IL6R, |
| leukocytes | | JAK3, JARID2, LGMN, LSP1, MBL2, NEDD9, |
| | | NFATC1, NKX2-3, OSM, PTPN6, RASAL3, |
| | | RPTOR, RUNX3, SERPINB6, SIPA1, SOCS3, |
| | | STING1, TEK |
| Quantity of lymphatic | 8.91 | C1QTNF6, CD6, CD79B, CR2, DEF6, EFNB2, |
| system cells | | ETS1, GFI1, HOXA3, HVCN1, IL15, IL6R, JAK3, |
| | | LGMN, LSP1, MBL2, NEDD9, NFATC1, NKX2-3, |
| | | OSM, PTPN6, PTPRCAP, RASAL3, RFTN1, |
| | | RPTOR, RUNX3, SERPINB6, SIPA1, SOCS3, |
| | | STING1, TEK |
| Cell movement of | 8.86 | C1QTNF6, CAMK2D, CD37, CD6, CR2, DEF6, |
| blood cells | | DOCK1, EFNB2, ETS1, EVL, IL15, IL6R, ITGA2B, |
| | | JAK3, LCP1, LGMN, LSP1, MAP4K1, NCKAP1L, |
| | | NEDD9, NFATC1, NKX2-3, OSM, PLA2G6, |
| | | PTPN6, RPTOR, RUNX3, RXRA, S100B, S1PR4, |
| | | SKAP1, SOCS3, STING1, TEK, ZBP1 |
| Differentiation of | 8.73 | CD6, CD79B, DOCK1, GFI1, GNA15, HOXA3, |
| hematopoietic cells | | HOXA5, IL15, IL6R, ITGA2B, JAK3, KCNAB2, |
| | | MAD1L1, MAFK, MAP4K1, OSM, PTPN6, |
| | | RPS6KA1, RXRA, TEK |
| Quantity of | 8.45 | C1QTNF6, CD6, CD79B, CR2, DEF6, EFNB2, |
| lymphocytes | | ETS1, GFI1, HOXA3, HVCN1, IL15, IL6R, JAK3, |
| | | LGMN, LSP1, MBL2, NEDD9, NFATC1, NKX2-3, |
| | | OSM, PTPN6, RASAL3, RPTOR, RUNX3, |
| | | SERPINB6, SIPA1, SOCS3, STING1, TEK |
| Leukocyte migration | 8.38 | C1QTNF6, CAMK2D, CD37, CD6, CR2, DEF6, |
| | | DOCK1, EFNB2, ETS1, EVL, IL15, IL6R, ITGA2B, |
| | | JAK3, LCP1, LGMN, LSP1, MAP4K1, NCKAP1L, |
| | | NEDD9, NFATC1, NKX2-3, OSM, PLA2G6, |
| | | PTPN6, RPTOR, RUNX3, RXRA, S100B, S1PR4, |
| | | SKAP1, SOCS3, STING1, TEK |
| Differentiation of | 8.12 | CD6, CD79B, DOCK1, GFI1, GNA15, HOXA3, |
| hematopoietic | | HOXA5, IL15, IL6R, ITGA2B, KCNAB2, |
| progenitor cells | | MAD1L1, MAFK, MAP4K1, OSM, PTPN6, |
| | | RPS6KA1, RXRA, TEK |
| Development of | 8.03 | AGO2, CD79B, ETS1, GFI1, HOXA5, IL15, IL6R, |
| hematopoietic system | | ITGA2B, JAK3, LGMN, MAFK, OSM, PLA2G6, |
| | | PTPN6, RPS6KA1, RPTOR, RUNX3, SOCS3, TEK |
| Lymphopoiesis | 7.18 | CARMIL2, CD6, CD79B, CR2, DEF6, EFNB2, |
| | | ETS1, GFI1, IL15, IL6R, JAK3, LCP1, LSP1, |
| | | MAD1L1, MAP2K2, NCKAP1L, NFATC1, NKX2- |
| | | 3, OSM, PTGS1, PTPN6, RFTN1, RPTOR, RUNX3, |
| | | SOCS3 |
| Binding of leukocytes | 7.12 | CD37, CD6, CR2, EVL, IL15, IL6R, JAK3, LCP1, |
| | | LSP1, MAP4K1, MBL2, NCKAP1L, NEDD9, OSM, |
| | | PLA2G6, PTPN6, SIPA1, SKAP1 |
| Development of bone | 6.98 | GFI1, HOXA5, IL15, IL6R, ITGA2B, LGMN, |
| marrow | | MAFK, OSM, PLA2G6, PTPN6, RPS6KA1, |
| | | RPTOR, RUNX3, TEK |
| T cell development | 5.90 | CARMIL2, CD6, CD79B, DEF6, EFNB2, ETS1, |
| | | GFI1, IL15, IL6R, JAK3, MAD1L1, NCKAP1L, |
| | | NFATC1, OSM, PTGS1, PTPN6, RFTN1, RPTOR, |
| | | RUNX3, SOCS3 |
| TGF-β Signaling | 3.21 | MAP2K2, MAP4K1, PMEPAI, RUNX3, SMAD6 |
| Role of JAK family | 3.14 | IL6R, OSM, SOCS3 |
| kinases in IL-6-type | | |
| Cytokine Signaling | | |
| Th1 and Th2 | 2.84 | GFI1, IL6R, JAK3, NFATC1, RUNX3, SOCS3 |
| Activation Pathway | | |
| Th1 Pathway | 2.74 | IL6R, JAK3, NFATC1, RUNX3, SOCS3 |
| JAK/STAT Signaling | 2.56 | JAK3, MAP2K2, PTPN6, SOCS3 |
| PI3K Signaling in B | 2.46 | CAMK2D, CD79B, CR2, MAP2K2, NFATC1 |
| Lymphocytes | | |
| CXCR4 Signaling | 2.14 | DOCK1, FNBP1, GNA15, GNG7, MAP2K2 |

MOUSE CARDIOMYOCYTE

| | | |
|---|---|---|
| cardiac cell | 82.47 | Actc1, Actn2, Alpk3, Atg5, Atg7, Bmp10, Cav3, |
| development | | Cdk1, Csrp3, Fhl2, Fhod3, Lrrc10, Map2k4, Mef2a, |
| | | Myh6, Myl2, Mylk3, Myo18b, Mypn, Nexn, Nppb, |
| | | Pdcd4, Pitx2, Popdc2, Prox1, Slc8a1, Speg, Tcap, |
| | | Tgfbr3, Ttn, Vegfa, Xirp1 |
| myofibril assembly | 81.23 | Actc1, Actn2, Casq2, Csrp3, Fhod3, Foxp1, Ldb3, |
| | | Lmod2, Mef2a, Mybpc3, Myh6, Myl2, Mylk3, |
| | | Myom1, Myom2, Myoz1, Myoz2, Myoz3, Mypn, |
| | | Prkar1a, Prox1, Tcap, Tmod1, Tnnt2, Tpm1, Ttn, |
| | | Xirp1 |
| actomyosin structure | 79.42 | Actc1, Actn2, Casq2, Cdc42bpa, Csrp3, Epb41l1, |
| organization | | Epb41l3, Epb41l4a, Epb41l4b, Fhod3, Foxp1, |
| | | Frmd5, Frmd6, Itgb5, Ldb3, Limch1, Lmod2, Mef2a, |
| | | Mybpc3, Myh6, Myl2, Mylk3, Myo18a, Myom1, |
| | | Myom2, Myoz1, Myoz2, Myoz3, Mypn, Pdcd6ip, |
| | | Phactr1, Prkar1a, Prox1, Sorbs1, Tcap, Tmod1, |
| | | Tnnt2, Tpm1, Trpm7, Ttn, Xirp1 |
| striated muscle cell | 77.01 | Actc1, Actn2, Alpk3, Atg5, Atg7, Bmp10, Capzb, |
| development | | Casq2, Cav3, Cdk1, Csrp3, Fhl2, Fhod3, Flnc, |
| | | Foxp1, Homer1, Ldb3, Lef1, Lmod2, Lrrc10, |
| | | Map2k4, Mef2a, Mybpc3, Myh6, Myl2, Mylk3, |
| | | Myo18b, Myom1, Myom2, Myoz1, Myoz2, Myoz3, |
| | | Mypn, Nexn, Nfatc2, Nppb, Pitx2, Popdc2, Prkar1a, |
| | | Prox1, Ptcd2, Slc8a1, Smyd3, Speg, Tcap, Tmod1, |
| | | Tnnt2, Tpm1, Ttn, Vegfa, Wfikkn2, Xirp1 |
| cardiac muscle | 75.80 | Actc1, Ank2, Cacna1c, Cacna1d, Cacna1g, |
| contraction | | Cacna2d1, Camk2d, Casq2, Cav3, Csrp3, Gja5, |
| | | Gpd1l, Kcnd3, Kcne1l, Kcne2, Kcnh2, Kcnj2, Kcnj5, |
| | | Kcnj8, Kcnn2, Kcnq1, Mybpc3, Myh6, Myh7, Myl2, |
| | | Myl3, Pkp2, Ryr2, Scn3b, Scn5a, Slc8a1, Snta1, |
| | | Tcap, Tnnc1, Tnni3, Tnnt2, Tpm1, Ttn |
| cardiocyte | 58.31 | Acadm, Actc1, Actn2, Akap13, Alpk3, Atg5, Atg7, |
| differentiation | | Bmp10, Bmp2, Cacybp, Cav3, Cdk1, Cited2, Csrp3, |
| | | Ctnnb1, Eomes, Fhl2, Fhod3, Foxp1, Gata4, Gata6, |
| | | Hand2, Hes1, Isl1, Lrp6, Lrrc10, Map2k4, Mef2a, |
| | | Myh6, Myl2, Mylk3, Myo18b, Myocd, Mypn, Nexn, |
| | | Nppb, Pax3, Pdcd4, Pitx2, Popdc2, Prok2, Prox1, |
| | | Rarb, Rbpj, Rxrb, Slc8a1, Sox6, Speg, T, Tbx2, |
| | | Tcap, Tenm4, Tgfb2, Tgfbr3, Ttn, Twist1, Vegfa, |
| | | Xirp1 |
| Morphology of heart | 26.40 | ACACB, ACADL, ACADM, ACTC1, ADAMTS9, |
| | | ADCY5, ADCY6, ADGRL2, ADRA1B, ADRA2A, |
| | | ADRB1, AGTR1, AKAP13, ALKBH7, ANGPT1, |
| | | ANK3, ANKRD1, ATE1, ATP2A2, BMP10, |
| | | C10orf71, CABIN1, CACNA1C, CAMK2D, |
| | | CASP7, CASQ2, CAST, CASZ1, CAV3, CCN2, |
| | | CD36, CDH2, CISD2, CKM, CKMT2, CORIN, |
| | | CREB1, CRYAB, CSRP3, CTNNA3, CTSD, |
| | | CXCL12, DAG1, DDIAS, DES, DHRS3, DOCK1, |
| | | DTNBP1, DYRK1A, EDN1, EGLN1, EIF4EBP1, |
| | | ERBB4, ERBIN, ETV6, F3, FABP3, FASN, FAT4, |
| | | FBXO32, FGF9, FHL2, FHOD3, FNIP1, Foxp1, |
| | | FXR1, GATA4, GHR, GJA1, GJC1, GRB2, GRK5, |
| | | HDAC4, HDAC9, HGF, HIF1A, HSPB7, HSPG2, |
| | | IGF1R, IL17RA, IL1B, INSR, JPH2, KCNE2, |
| | | KCNJ11, KCNQ1, KIDINS220, LAMA4, LIF, |
| | | LMOD2, LRRC10, LTBP1, MAPKAPK2, MB, |
| | | MBNL2, MEF2D, mir-1, mir-133, mir-208, MMP15, |
| | | MORF4L1, Morrbid, MRTFB, MYBPC3, MYH6, |
| | | MYL2, MYLK3, MYOCD, MYOM1, MYOZ2, |
| | | MYZAP, NCOA2, NDUFS6, NEXN, NF1, Nppb, |
| | | NR2F2, NT5E, NTF3, NTRK3, PARK7, PARP1, |
| | | PDCD5, PDGFA, PDLIM5, PFKM, PIK3R1, PIM1, |
| | | PITX2, PKP2, Pln, POSTN, PPARGC1A, PRKAA2, |
| | | PRKAR1A, PRKCE, PRKG1, PROX1, PTH1R, |
| | | PTHLH, RAF1, RARB, RBPJ, RCAN2, RGS2, |
| | | RGS6, ROCK1, ROCK2, RPS6KA2, RPS6KB2, |
| | | RRM2B, RXRB, RYR2, SEMA3A, SGCD, SGCG, |
| | | SIRPA, SLC8A1, SMAD3, SMAD7, SMYD1, SP3, |
| | | SPEG, SRSF10, STAB2, TAB1, TEAD1, TERT, |
| | | TGFB2, TGFBR2, TGFBR3, TGM2, TLL1, |
| | | TMEM38A, TMOD1, TNNI3, TNNT2, TNS1, |
| | | Tpm1, TRIM54, TRIM55, TRIM63, TRPC3, TTN, |
| | | UTRN, VAV2, VAV3, VEGFA, XIRP1, ZFPM2 |
| Abnormal | 25.50 | ACACB, ACADL, ACADM, ACTC1, ADAMTS9, |
| morphology of | | ADCY5, ADCY6, ADGRL2, ADRA1B, ADRA2A, |
| cardiovascular system | | ADRB1, ADTRP, AGTR1, AKAP13, ANGPT1, |
| | | ANK3, ANKRD1, ARID4B, ATE1, ATP2A2, |
| | | BCAM, BMP10, BMP2, CABIN1, CACNA1C, |
| | | CAMK2D, CASP7, CASQ2, CAST, CAV3, |
| | | CBS/CBSL, CCN2, CD36, CDH2, CISD2, CKM, |
| | | CKMT2, CORIN, CREB1, CRHR2, CRYAB, |
| | | CSRP3, CTNNA3, CTSD, CXCL12, DAG1, |
| | | DDIAS, DES, DHRS3, DOCK1, DYRK1A, E2F8, |
| | | EDN1, EGLN1, EIF4EBP1, ERBB4, ERBIN, ETV6, |
| | | F3, FABP3, FASN, FAT4, FBLN1, FBN1, FBXO32, |
| | | FGF9, FHL2, FHOD3, FNIP1, Foxp1, FXR1, FZD5, |
| | | GAB1, GATA4, GDNF, GHR, GJA1, GRB2, GRK5, |
| | | GSC, HDAC4, HDAC9, HGF, HIF1A, HSPB7, |
| | | HSPG2, IGF1R, IL17RA, IL1B, INSR, JPH2, |
| | | KCNE2, KCNJ11, KCNQ1, KIDINS220, KLF2, |
| | | LAMA4, LIF, LPL, LRRC10, LTBP1, MAPKAPK2, |
| | | MB, MBNL2, MEF2D, mir-1, mir-122, mir-133, |
| | | mir-208, MMP15, MORF4L1, Morrbid, MRTFB, |
| | | MYBPC3, MYH6, MYL2, MYLK3, MYOCD, |
| | | MYOM1, MYOZ2, MYZAP, NCOA2, NDUFS6, |
| | | NEXN, NF1, Nppb, NR2F2, NT5E, NTF3, NTRK3, |
| | | PARK7, PARP1, PDGFA, PDLIM5, PFKM, |
| | | PIK3R1, PIM1, PITX2, PKP2, Pln, PLPP3, POSTN, |
| | | PPARGC1A, PRKAA2, PRKAR1A, PRKCE, |
| | | PRKG1, PROX1, RAF1, RARB, RBPJ, RCAN2, |
| | | RGS2, ROCK1, ROCK2, RPS6KA2, RPS6KB2, |
| | | RRM2B, RXRB, RYR2, SEMA3A, SETD2, SGCD, |
| | | SGCG, SIRPA, SLC8A1, SMAD3, SMAD7, |
| | | SMYD1, SP3, SPEG, SRSF10, STAB2, TAB1, |
| | | TEAD1, TERT, TGFB2, TGFBR2, TGFBR3, |
| | | TGM2, TLL1, TMEM38A, TNNI3, TNNT2, TNS1, |
| | | Tpm1, TRIM54, TRIM55, TRIM63, TRPC3, TTN, |
| | | UTRN, VAV2, VAV3, VEGFA, XIRP1, ZFPM2 |
| Enlargement of heart | 25.12 | ACTC1, ADCY5, ADCY6, ADRA2A, ADRB1, |
| | | AGTR1, AKAP13, ANGPT1, ANK3, ANKRD1, |
| | | ATP2A2, BMP10, CABIN1, CACNA1C, CAMK2D, |
| | | CASQ2, CAST, CAV3, CCN2, CD36, CDH2, CKM, |
| | | CKMT2, CORIN, CREB1, CRYAB, CSRP3, |
| | | CTNNA3, CTSD, CXCL12, DAG1, DDIAS, DES, |
| | | DYRK1A, EDN1, EGLN1, EIF4EBP1, ERBIN, F3, |
| | | FABP3, FASN, FBXO32, FGF9, FHL2, FHOD3, |
| | | FNIP1, GATA4, GJA1, GRB2, GRK5, HDAC4, |
| | | HDAC9, HGF, HIF1A, IGF1R, IL17RA, IL1B, |
| | | INSR, KCNE2, KCNJ11, KCNQ1, LAMA4, LIF, |
| | | LRRC10, MAPKAPK2, MB, MBNL2, MEF2D, mir- |
| | | 1, mir-133, mir-208, MORF4L1, Morrbid, MYBPC3, |
| | | MYH6, MYL2, MYLK3, MYOCD, MYOM1, |
| | | MYOZ2, MYZAP, NCOA2, NDUFS6, NEXN, NF1, |
| | | NT5E, NTF3, NTRK3, PARK7, PARP1, PDGFA, |
| | | PDLIM5, PFKM, PIK3R1, PIM1, PITX2, Pln, |
| | | POSTN, PPARGC1A, PRKAA2, PRKAR1A, |
| | | PRKCE, PRKG1, PROX1, RAF1, RCAN2, RGS2, |
| | | ROCK1, ROCK2, RYR2, SEMA3A, SGCD, SIRPA, |
| | | SLC8A1, SMAD3, SMYD1, SP3, SPEG, STAB2, |
| | | TERT, TGFBR2, TGFBR3, TNNI3, TNNT2, Tpm1, |
| | | TRIM54, TRIM55, TRIM63, TRPC3, TTN, UTRN, |
| | | VAV2, VAV3, VEGFA, XIRP1 |
| Cardiogenesis | 19.82 | ACADM, ACTC1, ACTN2, ADGRL2, ADRA1B, |
| | | AGTR1, AKAP13, ALPK3, ANGPT1, ARID2, |
| | | ASB2, ATE1, ATP2A2, BICC1, BMP10, BMP2, |
| | | C10orf71, Clorf127, CASP7, CASQ2, CASZ1, |
| | | CAV3, CBS/CBSL, CCDC39, CCN1, CSRP3, |
| | | CTBP2, CXCL12, DHRS3, DLC1, DSP, DTNBP1, |
| | | EDN1, EGLN1, ERBB4, ESRRG, F2RL2, FAT4, |
| | | FGF20, FGF9, FHOD3, FLRT3, Foxp1, FREM2, |
| | | GAB1, GATA4, GJA1, GJC1, GLI2, GSC, HDAC4, |
| | | HDAC9, HEG1, HIF1A, HSPG2, IL1B, INSR, |
| | | JPH2, KCNQ1, LDB3, LIF, LMOD2, LRP6, |
| | | LRRC10, LTBP1, MB, mir-1, mir-133, mir-208, |
| | | MORF4L1, MRTFB, MYBPC3, MYH6, MYL2, |
| | | MYLK3, MYO18B, MYOCD, MYOZ1, MYOZ2, |
| | | NCOA2, NDST1, NF1, NR2F2, NTF3, NTRK3, |
| | | PAX3, PCSK5, PCSK6, PDE2A, PDGFA, PITX2, |
| | | PKP2, Pln, PPARGC1A, PRICKLE1, PRKAR1A, |
| | | PROK2, PROX1, PTCD2, RBPJ, ROBO1, |
| | | RPS6KA2, RXRB, SCN5A, SETD2, SGCD, SGCG, |
| | | SLC8A1, SMAD3, SMAD7, SMYD1, SPEG, |
| | | SYNPO2L, TAB1, TBXT, TCAP, TEAD1, TGFB2, |
| | | TGFBR2, TGFBR3, TLL1, TMOD1, TNNI3, |
| | | TNNT2, TP53BP2, Tpm1, TRIM63, TTN, UTRN, |
| | | VEGFA, VGLL4, WNT11, XIRP1, ZFPM2 |
| Function of cardiac | 14.36 | ADCY6, ADRA1B, ADRB1, ASPH, ATE1, |
| muscle | | ATP1A1, ATP2A2, C10orf71, CACNA2D1, |
| | | CASQ2, CAV3, CKM, CORIN, CRHR2, CSRP3, |
| | | CTNNA3, DES, DSG2, DTNBP1, EDN1, ESRRG, |
| | | GAB1, GATA4, KCNIP2, LAMA4, LIF, LRRC10, |
| | | MB, mir-1, mir-133, mir-208, MYBPC3, MYH6, |
| | | MYLK3, NEXN, PDLIM5, Pln, PPARGC1A, |
| | | PRKG1, PTH1R, RBPJ, RYR2, SGCD, SLC8A1, |
| | | SLN, SMAD7, SOD1, SRL, SRSF10, TMEM38A, |
| | | TNNT2, Tpm1, TRIM54, TRIM55, TRIM63, TTN, |
| | | VAV3, VEGFA, XIRP1 |
| Dilated | 11.60 | ABCC9, ACTC1, ADCY5, ADCY6, ADCY9, |
| Cardiomyopathy | | ADRB1, ATP2A2, BAD, BAG3, CACNA1C, |
| Signaling Pathway | | CACNA2D1, CAMK2D, DES, DSG2, GAB1, |
| | | ITPR1, KDMIA, LAMA4, MYBPC3, MYH6, |
| | | MYH7, MYH7B, MYL2, MYL3, MYO10, |
| | | MYO18A, MYO18B, PDE2A, PRKAR1A, |
| | | PRKAR2A, PRKCE, RBM20, RYR2, SCN5A, |
| | | SGCD, TNNC1, TNNI3, TNNT1, TNNT2, TNNT3, |
| | | TTN |
| Morphology of | 11.26 | ACADM, ACTC1, ADCY6, ADRB1, ATE1, |
| cardiomyocytes | | ATP2A2, C10orf71, CABIN1, CASQ2, CAV3, |
| | | CCN2, CDH2, CSRP3, CTNNA3, DES, DTNBP1, |
| | | EDN1, FHL2, FXR1, GATA4, HDAC9, HGF, |
| | | HSPB7, IL1B, INSR, JPH2, LAMA4, LIF, LRRC10, |
| | | MB, mir-1, mir-133, mir-208, MORF4L1, MYBPC3, |
| | | MYH6, MYLK3, MYOCD, MYOZ2, Nppb, Pln, |
| | | RAF1, RPS6KA2, RYR2, SLC8A1, TAB1, TERT, |
| | | TGFBR3, TMEM38A, TNNI3, TNNT2, TRIM55, |
| | | TRIM63, TTN, VAV2, VAV3, VEGFA |
| Cardiac Hypertrophy | 6.71 | ACVR2A, ADCY5, ADCY6, ADCY9, ADRA1B, |
| Signaling (Enhanced) | | ADRA2A, ADRB1, AGTR1, AKAP13, ATP2A2, |
| | | CACNA1C, CACNA2D1, CAMK2D, EDN1, |
| | | EIF4E, EIF4EBP1, FGF13, FGF20, FGF6, FGF9, |
| | | FGFR3, FZD5, FZD9, GATA4, GHR, GNB4, |
| | | HDAC4, HDAC9, HSPB1, HSPB7, IGF1R, IL13, |
| | | IL17RA, IL1B, IL20RA, IL21R, IL9R, ITGA2B, |
| | | ITGA9, ITGAD, ITGAL, ITGAX, ITGB6, ITPR1, |
| | | LIF, MAP3K20, MAP3K4, MAPK10, MAPKAPK2, |
| | | MAPKAPK3, MEF2D, MYOCD, PDE10A, PDE2A, |
| | | PDE4B, PDE7A, PDE7B, PIK3R1, PIK3R3, |
| | | PIK3R4, PLCB2, PLCD3, PLCZ1, PRKAR1A, |
| | | PRKAR2A, PRKCE, PRKCH, PRKG1, RAF1, |
| | | RALB, RAP2A, RASD1, ROCK1, ROCK2, |
| | | RPS6KB2, RYR2, TGFB2, TGFBR2, TGFBR3, |
| | | WNT11 |
| Actin Cytoskeleton | 6.24 | ACTC1, ACTN2, ACTR3, ARHGEF7, DOCK1, |
| Signaling | | EZR, FGF13, FGF20, FGF6, FGF9, GRB2, |
| | | ITGA2B, ITGA9, ITGAD, ITGAL, ITGAX, ITGB6, |
| | | MYH6, MYH7, MYH7B, MYL2, MYL3, MYLK3, |
| | | MYO10, MYO18A, MYO18B, PDGFA, PIK3R1, |
| | | PIK3R3, PIK3R4, PPP1R12A, Ppp1r12b, RAF1, |
| | | RALB, RAP2A, RASD1, ROCK1, ROCK2, SSH2, |
| | | TLN2, TRIO, TTN, VAV2, VAV3 |
| Calcium Signaling | 5.02 | ACTC1, ASPH, ATP2A2, ATP2B1, ATP2C1, |
| | | CABIN1, CACNA1C, CACNA2D1, CAMK2D, |
| | | CASQ2, CREB1, HDAC4, HDAC9, ITPR1, |
| | | MEF2D, MYH6, MYH7, MYH7B, MYL2, MYL3, |
| | | MYO10, MYO18A, MYO18B, PRKAR1A, |
| | | PRKAR2A, RAP2A, RCAN2, RYR2, SLC8A1, |
| | | TNNC1, TNNI3, TNNT1, TNNT2, TNNT3, TP63, |
| | | Tpm1, TRPC3 |
| Factors Promoting | 4.49 | ACVR2A, BMP10, BMP2, CAMK2D, CREB1, |
| Cardiogenesis in | | FZD5, FZD9, GATA4, LRP6, MAPK10, MYH6, |
| Vertebrates | | MYH7, MYL2, MYOCD, PLCB2, PLCD3, PLCZ1, |
| | | PRKCE, PRKCH, ROCK1, ROCK2, SCN5A, TCF4, |
| | | TCF7L2, TGFB2, TGFBR2, TGFBR3, WNT11 |
| Cardiac Hypertrophy | 4.42 | ADCY5, ADCY6, ADCY9, ADRA1B, ADRA2A, |
| Signaling | | ADRB1, CACNA1C, CACNA2D1, CREB1, EIF4E, |
| | | GATA4, GNB4, GRB2, HSPB1, IGF1R, MAP3K4, |
| | | MAPK10, MAPKAPK2, MAPKAPK3, MEF2D, |
| | | MYL2, MYL3, PIK3R1, PIK3R3, PIK3R4, PLCB2, |
| | | PLCD3, PLCZ1, PRKAR1A, PRKAR2A, RAF1, |
| | | RALB, RAP2A, RASD1, RHOBTB1, RHOBTB2, |
| | | ROCK1, ROCK2, TAB1, TGFB2, TGFBR2 |
| White Adipose Tissue | 3.83 | ADCY5, ADCY6, ADCY9, CACNA1C, |
| Browning Pathway | | CACNA2D1, CAMP, CREB1, CTBP2, FGFR3, |
| | | FNDC5, ITPR1, LDHB, PPARGC1A, PRKAA2, |
| | | PRKAB1, PRKAB2, PRKAR1A, PRKAR2A, |
| | | PRKG1, RARB, RUNX1T1, RXRB, RXRG, |
| | | SLC16A1, VEGFA |
| Role of NFAT in | 3.75 | ADCY5, ADCY6, ADCY9, CABIN1, CACNA1C, |
| Cardiac Hypertrophy | | CACNA2D1, CAMK2D, GATA4, GNB4, GRB2, |
| | | HDAC4, HDAC9, IGF1R, ITPR1, LIF, MAPK10, |
| | | MEF2D, PIK3R1, PIK3R3, PIK3R4, PLCB2, |
| | | PLCD3, PLCZ1, PRKAR1A, PRKAR2A, PRKCE, |
| | | PRKCH, RAF1, RALB, RAP2A, RASD1, RCAN2, |
| | | SLC8A1, TGFB2, TGFBR2 |
| Thrombin Signaling | 3.19 | ADCY5, ADCY6, ADCY9, ARHGEF16, |
| | | ARHGEF3, CAMK2D, CREB1, F2RL2, GATA4, |
| | | GNB4, GRB2, ITPR1, MYL2, MYL3, PIK3R1, |
| | | PIK3R3, PIK3R4, PLCB2, PLCD3, PLCZ1, |
| | | PPP1R12A, Ppp1r12b, PRKCE, PRKCH, RAF1, |
| | | RALB, RAP2A, RASD1, RHOBTB1, RHOBTB2, |
| | | ROCK1, ROCK2 |
| Cardiac β-adrenergic | 2.97 | ADCY5, ADCY6, ADCY9, ADRB1, AKAP1, |
| Signaling | | AKAP13, AKAP6, ATP2A2, CACNA1C, |
| | | CACNA2D1, GNB4, PDE10A, PDE2A, PDE4B, |
| | | PDE7A, PDE7B, PKIG, PPP1R12A, PPP1R14C, |
| | | PPP1R3A, PPP1R3D, PPP2R2B, PPP2R3A, |
| | | PRKAR1A, PRKAR2A, RYR2, SLC8A1 |
| Apelin | 1.77 | ATP2A2, HIF1A, ITPR1, MAPK10, MYL2, MYL3, |
| Cardiomyocyte | | PIK3R1, PIK3R3, PIK3R4, PLCB2, PLCD3, |
| Signaling Pathway | | PLCZ1, PRKCE, PRKCH, SLC8A1 |
| Cardiomyocyte | 1.63 | BMP10, BMP2, GATA4, MYH7, MYL2 |
| Differentiation via | | |
| BMP Receptors | | |
| Nitric Oxide Signaling | 1.46 | ADRB1, ATP2A2, CACNA1C, CACNA2D1, |
| in the Cardiovascular | | ITPR1, PDE2A, PIK3R1, PIK3R3, PIK3R4, |
| System | | PRKAR1A, PRKAR2A, PRKCE, PRKCH, PRKG1, |
| | | RYR2, VEGFA |

MOUSE HEPATOCYTE

| | | |
|---|---|---|
| Concentration of lipid | 14.21 | ABCC4, ABCG8, ACLY, ADCY10, AGPAT2, |
| | | AGT, ANGPTL8, APOA1, Apoc1, APOE, |
| | | AQP12A/AQP12B, C3, CAMP, CERS2, CFB, |
| | | CIDEC, COL18A1, CPE, CRHR1, CYP17A1, |
| | | CYP8B1, DHCR24, EGFR, ELOVL2, EPAS1, |
| | | ESR2, F2, FABP1, FASN, FFAR4, FOXA2, GC, |
| | | GCGR, GCK, GDF15, GNAT1, Gucy2g, Gulo, HP, |
| | | HPN, HRH1, IRS2, KL, KLF15, LCAT, LDLRAP1, |
| | | LEPR, LPAR3, LRP1, LRP5, LSR, mir-290, mir-33, |
| | | MPC1, MRAP, NR0B2, NR1I3, NSMF, NXPH4, |
| | | OXSM, PCK1, PEMT, PLIN2, PLPP3, PNPLA3, |
| | | PON1, PRF1, PSEN2, PTPN1, RXRA, SCARB1, |
| | | SCD, SEC14L2, SERPINA12, SFTPB, SGMS1, |
| | | SLC37A4, WNT4, WWOX |
| FXR/RXR Activation | 12.50 | ABCG8, AGT, AMBP, APOA1, APOE, APOF, |
| | | APOH, C3, CYP8B1, FASN, FOXA2, GC, ITIH4, |
| | | LCAT, NR0B2, PCYOX1, PON1, RXRA, SCARB1, |
| | | SERPINF2, SLC27A5, VTN |
| Quantity of | 11.83 | ABCC4, AGPAT2, AMBP, APOA1, APOE, ATF6, |
| carbohydrate | | C3, CFB, CIDEC, CPE, EGFR, ESR2, F2, FABP1, |
| | | FFAR4, FOXA2, Foxp1, GCGR, GCK, GCKR, |
| | | GDF15, GLS2, GNMT, Gulo, HPN, IKBKB, IL6ST, |
| | | IRS2, LCAT, LEPR, LIFR, LRP1, LRP5, MRAP, |
| | | NR0B2, NR1I3, PCK1, PEMT, PER2, PLIN2, |
| | | PON1, PRF1, PSEN2, PTPN1, SCARB1, SCD, |
| | | SFTPB, Sik1, SLC23A1, SLC37A4, SPHK2, |
| | | STX1A, USF2, WWOX |
| Quantity of steroid | 11.36 | ABCC4, ABCG8, ADCY10, AGPAT2, AGT, |
| | | APOA1, Apoc1, APOE, AQP12A/AQP12B, |
| | | CRHR1, CYP17A1, CYP8B1, DHCR24, EGFR, |
| | | ESR2, FABP1, FASN, FFAR4, GC, GCGR, GCK, |
| | | GDF15, Gucy2g, Gulo, HP, HPN, IRS2, KL, LCAT, |
| | | LDLRAP1, LEPR, LRP1, LRP5, LSR, mir-33, |
| | | MPC1, MRAP, NR0B2, NR1I3, NSMF, PEMT, |
| | | PLIN2, PON1, PSEN2, PTPN1, RXRA, SCARB1, |
| | | SCD, SEC14L2, SERPINA12, SLC37A4, WNT4 |
| LXR/RXR Activation | 10.80 | ABCG8, AGT, AMBP, APOA1, APOE, APOF, |
| | | APOH, C3, FASN, GC, IL1RAP, ITIH4, LCAT, |
| | | PCYOX1, PON1, RELA, RXRA, SCD, SERPINF2, |
| | | VTN |
| Morphology of liver | 5.02 | ABCD3, AGPAT2, AGT, APOA1, APOE, BCR, C3, |
| | | CYP2E1, EGFR, ESR2, FFAR4, GCGR, GNMT, |
| | | IKBKB, IL6ST, LEPR, LRP1, MST1, MTF1, |
| | | NR0B2, NR1I3, PCK1, PEMT, RELA, RXRA, |
| | | SERPINA12, SLC37A4, TGFA, TMPRSS6, WWOX |
| Liver lesion | 4.90 | AGT, APOA1, APOE, BCL6, C3, CCN1, CDK1, |
| | | CERS2, CIDEC, CYP2E1, EGFR, FASN, FGFR2, |
| | | GADD45B, GNMT, HP, IKBKB, IL6ST, ITIH4, |
| | | LRP1, Meg3, mir-26, mir-455, MST1, MTF1, |
| | | NFKBIB, NINJ1, NR0B2, NR1I3, PEMT, PIM3, |
| | | PNPLA3, PRF1, PTPNI, RELA, RXRA, SCD, |
| | | SERPINC1, SLC26A1, SNAI1, TGFA |
| LPS/IL-1 Mediated | 4.84 | ABCC4, ABCG8, ACSL5, ALAS1, ALDH3A2, |
| Inhibition of RXR | | ALDH7A1, ALDH8A1, APOE, Cyp2d26, CYP2E1, |
| Function | | FABP1, IL1RAP, NR0B2, NR1I3, PAPSS2, RXRA, |
| | | SCARB1, SLC27A5, SULT4A1 |
| PXR/RXR Activation | 4.56 | ALAS1, ALDH3A2, NR0B2, NR1I3, PAPSS2, |
| | | PRKAG2, RELA, RXRA, SCD |
| Histidine Degradation | 4.50 | AMDHD1, FTCD, MTHFD1, UROC1 |
| III | | |
| Acute Phase Response | 4.20 | AGT, AMBP, APOA1, APOH, C3, CFB, F2, HP, |
| Signaling | | IKBKB, IL1RAP, IL6ST, ITIH4, NFKBIB, RELA, |
| | | SERPINF2 |
| Proliferation of liver | 4.06 | AGT, C3, CCN1, CERS2, EGFR, FGFR2, IKBKB, |
| cells | | IL6ST, ITIH4, LEPR, MST1, NR1I3, PIM3, RELA, |
| | | RXRA, TGFA, TOP1MT |
| Proliferation of | 3.47 | AGT, C3, CCN1, CERS2, EGFR, FGFR2, IKBKB, |
| hepatocytes | | IL6ST, ITIH4, MST1, RELA, RXRA, TGFA, |
| | | TOP1MT |
| Response of liver | 3.42 | APOA1, APOE, BCL6, C3, CCN1, CIDEC, GNMT, |
| | | HP, IKBKB, IL6ST, NFKBIB, NINJ1, NR0B2, |
| | | PRF1, RELA, RXRA, SCD, TGFA |
| Extrinsic Prothrombin | 3.16 | F12, F2, F7, SERPINC1 |
| Activation Pathway | | |
| Fatty Acid | 3.15 | FASN, OXSM |
| Biosynthesis Initiation | | |
| II | | |
| Iron homeostasis | 3.09 | ACO1, BMP1, BMP6, EGFR, EPAS1, GDF15, HP, |
| signaling pathway | | LRP1, SLC11A2, TFRC, TMPRSS6 |
| Hepatic Fibrosis/ | 2.81 | AGT, COL15A1, COL18A1, CYP2E1, ECE1, |
| Hepatic Stellate Cell | | EGFR, FGFR2, IFNGR2, IL1RAP, LEPR, PROK1, |
| Activation | | RELA, TGFA |
| Morphology of | 2.80 | CYP2E1, EGFR, IKBKB, LRP1, MST1, PEMT, |
| hepatocytes | | RELA, RXRA, TMPRSS6 |
| Complement System | 2.78 | C3, C8A, C8B, CFB, MASP2 |
| Coagulation System | 2.66 | F12, F2, F7, SERPINC1, SERPINF2 |
| Hepatic Cholestasis | 2.49 | ABCG8, ADCY10, CYP8B1, GCGR, IKBKB, |
| | | IL1RAP, NFKBIB, NR0B2, PRKAG2, PRKD3, |
| | | RELA, RXRA |
| PPARa/RXRa | 2.37 | ADCY10, APOA1, FASN, GPD1, IKBKB, IL1RAP, |
| Activation | | ITGB5, NFKBIB, NR0B2, PRKAG2, RELA, RXRA |
| TR/RXR Activation | 1.71 | FASN, HP, NCOA4, PCK1, RXRA, SCARB1 |
| Hepatic Fibrosis | 1.62 | AGT, CACNG3, COL18A1, IKBKB, IL1RAP, |
| Signaling Pathway | | IRS2, ITGB5, LEPR, LRP1, LRP5, NFKBIB, |
| | | PRKAG2, PRKD3, PROK1, RELA, SNAI1, TFRC, |
| | | WNT4 |

MOUSE LUNG EPITHELIAL

| | | |
|---|---|---|
| vasculature | 48.31 | Acvrl1, Adam15, Adamts6, Adamts9, Ahr, Aldh1a2, |
| development | | Aqp1, Arhgef15, Bcas3, Bmper, Bmpr2, Calcrl, |
| | | Casp8, Cav1, Ccbe1, Cd34, Cdh13, Cdh2, Cdh5, |
| | | Chd7, Cited2, Clic4, Col4a2, Col5a1, Ctnnb1, |
| | | Cxcl12, Cxcr4, Cyp1b1, Cyr61, Dll4, Dlx3, E2f7, |
| | | Efnb2, Egf17, Egr3, Elk3, Emcn, Eng, Epas1, Esm1, |
| | | Ets1, Fgfr2, Flt1, Flt4, Fmnl3, Fn1, Foxc2, Foxf1, |
| | | Foxo1, Fzd4, Fzd8, Gata4, Gata6, Gbx2, Gja4, |
| | | Grem1, Has2, Hdac7, Hectd1, Heg1, Hhex, Hif1a, |
| | | Immp2l, Itgb1, Itgb3, Jag1, Jun, Kdr, Lama1, Ltbp1, |
| | | Luzp1, Mcam, Mef2c, Meis1, Mkl2, Mmp2, Myh10, |
| | | Myocd, Nkx3-1, Nodal, Notch1, Nr2f2, Nr4a1, |
| | | Nrcam, Nrp1, Nrp2, Ntrk2, Osr1, Pcsk5, Pdgfb, |
| | | Pdgfra, Pecam1, Plcd3, Plg, Plpp3, Plxnd1, Pnpla6, |
| | | Prcp, Prdm1, Prickle1, Prok2, Prox1, Prrx1, Pten, |
| | | Ptk2, Ptprb, Qk, Ramp2, Rapgef1, Rapgef2, Rasip1, |
| | | Reck, Rhoj, Robo4, Rora, Rxra, S1pr1, Sema3c, |
| | | Sema5a, Slc4a7, Smad5, Smad6, Smad7, Smarca2, |
| | | Sox18, Sox4, Syk, T, Tbx1, Tbx2, Tbx3, Tek, Tgfb2, |
| | | Tgfbi, Tgfbr3, Thsd7a, Tmem100, Tmem204, |
| | | Tnfaip2, Tspan12, Ubp1, Vav3, Vegfc, Wt1, |
| | | Zfp36l1, Zfpm2, Zmiz1 |
| cardiovascular system | 47.65 | Ab12, Acvrl1, Adam15, Adamts6, Adamts9, Ahr, |
| development | | Aldh1a2, Aqp1, Arhgef15, Bcas3, Bmper, Bmpr2, |
| | | Calcrl, Casp8, Cav1, Ccbe1, Cd34, Cdh13, Cdh2, |
| | | Cdh5, Chd7, Cited2, Clic4, Col4a2, Col5a1, Ctnnb1, |
| | | Cxcl12, Cxcr4, Cyp1b1, Cyr61, Dll4, Dlx3, E2f7, |
| | | Efnb2, Egf17, Egr3, Elk3, Emcn, Eng, Epas1, Esm1, |
| | | Ets1, Fgfr2, Flt1, Flt4, Fmnl3, Fn1, Foxc2, Foxf1, |
| | | Foxo1, Foxp1, Fzd4, Fzd8, Gata4, Gata6, Gbx2, |
| | | Gja4, Grem1, Has2, Hdac7, Hectd1, Heg1, Hhex, |
| | | Hif1a, Immp2l, Itgb1, Itgb3, Jag1, Jun, Kcnj1, Kdr, |
| | | Lama1, Ltbp1, Luzp1, Mcam, Mef2c, Meis1, Mkl2, |
| | | Mmp2, Myh10, Myocd, Nkx3-1, Nodal, Notch1, |
| | | Nr2f2, Nr4a1, Nrcam, Nrp1, Nrp2, Ntrk2, Osr1, |
| | | Pcsk5, Pdgfb, Pdgfra, Pecam1, Plcd3, Plg, Plpp3, |
| | | Plxnd1, Pnpla6, Prcp, Prdm1, Prickle1, Prok2, Prox1, |
| | | Prrx1, Pten, Ptk2, Ptprb, Qk, Ramp2, Rapgef1, |
| | | Rapgef2, Rasip1, Reck, Rhoj, Robo4, Rora, Rxra, |
| | | S1pr1, Sema3c, Sema5a, Slc4a7, Smad5, Smad6, |
| | | Smad7, Smarca2, Sox18, Sox4, Syk, T, Tbx1, Tbx2, |
| | | Tbx3, Tek, Tgfb2, Tgfbi, Tgfbr3, Thsd7a, Tmem100, |
| | | Tmem204, Tnfaip2, Tspan12, Ubp1, Vav3, Vegfc, |
| | | Wt1, Zfp36l1, Zfpm2, Zmiz1 |
| blood vessel | 45.04 | Acvrl1, Adam15, Adamts6, Adamts9, Ahr, Aldh1a2, |
| development | | Aqp1, Bcas3, Bmper, Bmpr2, Calcrl, Casp8, Cav1, |
| | | Ccbe1, Cd34, Cdh13, Cdh2, Cdh5, Chd7, Cited2, |
| | | Clic4, Col4a2, Col5a1, Ctnnb1, Cxcl12, Cxcr4, |
| | | Cyplb1, Cyr61, Dll4, Dlx3, E2f7, Efnb2, Egf17, |
| | | Egr3, Elk3, Emcn, Eng, Epas1, Esm1, Ets1, Fgfr2, |
| | | Flt1, Flt4, Fmnl3, Fn1, Foxc2, Foxf1, Foxo1, Fzd4, |
| | | Fzd8, Gata4, Gata6, Gbx2, Gja4, Grem1, Has2, |
| | | Hdac7, Hectd1, Heg1, Hhex, Hif1a, Itgb1, Itgb3, |
| | | Jag1, Jun, Kdr, Lama1, Ltbp1, Luzp1, Mcam, Mef2c, |
| | | Meis1, Mkl2, Mmp2, Myh10, Myocd, Nkx3-1, |
| | | Notch1, Nr2f2, Nr4a1, Nrcam, Nrp1, Nrp2, Ntrk2, |
| | | Osr1, Pcsk5, Pdgfb, Pdgfra, Pecam1, Plcd3, Plg, |
| | | Plpp3, Plxnd1, Pnpla6, Prcp, Prdm1, Prickle1, Prok2, |
| | | Prox1, Prrx1, Pten, Ptk2, Ptprb, Qk, Ramp2, |
| | | Rapgef1, Rapgef2, Rasip1, Reck, Robo4, Rora, Rxra, |
| | | S1pr1, Sema3c, Sema5a, Smad5, Smad6, Smad7, |
| | | Sox18, Sox4, Syk, T, Tbx1, Tbx2, Tbx3, Tek, Tgfb2, |
| | | Tgfbi, Tgfbr3, Thsd7a, Tmem100, Tnfaip2, Tspan12, |
| | | Ubp1, Vav3, Vegfc, Wt1, Zfp36l1, Zfpm2, Zmiz1 |
| angiogenesis | 34.15 | Acvrl1, Adam15, Ahr, Bcas3, Bmper, Calcrl, Casp8, |
| | | Cav1, Ccbe1, Cd34, Cdh13, Clic4, Col4a2, Ctnnb1, |
| | | Cxcl12, Cxcr4, Cyp1b1, Cyr61, Dll4, E2f7, Efnb2, |
| | | Egf17, Egr3, Elk3, Emcn, Eng, Epas1, Esm1, Ets1, |
| | | Fgfr2, Flt1, Flt4, Fmnl3, Fn1, Foxc2, Fzd8, Gbx2, |
| | | Grem1, Hif1a, Itgb1, Itgb3, Jun, Kdr, Mcam, Meis1, |
| | | Mmp2, Notch1, Nr4a1, Nrcam, Nrp1, Nrp2, Pdgfra, |
| | | Pecam1, Plcd3, Plxnd1, Pnpla6, Prcp, Prok2, Pten, |
| | | Ptk2, Ptprb, Ramp2, Rasip1, Robo4, Rora, Rxra, |
| | | S1pr1, Sema5a, Smad5, Sox18, Syk, Tbx1, Tek, |
| | | Tgfbi, Thsd7a, Tmem100, Tnfaip2, Tspan12, Ubp1, |
| | | Vav3, Vegfc |
| Respiratory system | 14.13 | ADGRF5, AFF4, AHR, AP3B1, ARRB1, BMP7, |
| development | | BMPR2, CAV1, CAV2, CCBE1, CHD7, DNAAF11, |
| | | DPPA4, ECE1, ELK3, ELN, EPAS1, EPHA3, FAS, |
| | | FAT4, Fendrr, FGF7, FGFR2, FLT4, FOXA2, |
| | | FOXF1, Foxp1, GATA3, GIT1, HEGI, HTT, |
| | | IGF1R, ISL1, ITGA3, ITGA6, KDR, LAMC1, |
| | | LIPA, LRRK2, mir-126, mir-17, MMP2, NCOA2, |
| | | NFIB, NKIRAS2, NKX2-6, NODAL, PCSK5, |
| | | PECAM1, PLG, PTEN, Ptprd, RARB, RARG, |
| | | RC3H1, RIPPLY3, ROCK2, SAV1, SERPINE1, |
| | | SKI, SOX11, SPRY2, SPRY4, SYNE1, TBX1, TEK, |
| | | TNS3, TSHZ3, WT1, ZNF521 |
| Morphology of | 6.69 | ADGRF5, AFF4, ARRB1, BMP7, BMPR2, CAV1, |
| respiratory system | | CAV2, CCBE1, DNAAF11, DPPA4, ELK3, ELN, |
| | | EPAS1, EPHA3, FAS, FAT4, FGFR2, FLT4, |
| | | FOXA2, Foxp1, GIT1, GPC6, HTT, IGF1R, ITGA3, |
| | | ITGA6, LAMC1, LIPA, LRRK2, MMP2, NFIB, |
| | | NKX2-6, Nrg1, PBX1, PECAM1, PLG, PTEN, |
| | | Ptprd, RARB, RARG, RBL2, SERPINE1, SOX11, |
| | | SOX7, SPRY2, SPRY4, SYNE1, TBX1, TSHZ1, |
| | | TTLL1, ZNF521 |
| Cell death of | 6.29 | ABL2, ACVRL1, ADAM15, BCL2L11, CASP8, |
| endothelial cells | | CDH5, COL4A2, DUSP6, EPAS1, FAS, FLT1, |
| | | FLT4, HGF, KDR, LIPA, MALAT1, MMP2, |
| | | PECAM1, PLG, PPARD, PTEN, PTK2, SEMA3F, |
| | | SEMA6A, SERPINE1, SOCS1 |
| Remodeling of blood | 6.17 | ACVRL1, AHR, BCL6B, BMPR2, CHD7, DLL4, |
| vessel | | EFNB2, EPAS1, EXT1, JAG1, KCNK6, KL, |
| | | MEF2C, SEMA3C, SERPINE1, TBX1, TEK, UBR4 |
| Transcription of DNA | 6.08 | ABLIM1, ACVRL1, ADRB2, AEBP2, AFF3, AHR, |
| | | ALX1, AP3B1, APBB2, ATOH1, BACH1, BACH2, |
| | | BARX2, BCL6B, BMP7, BMPR2, CAV1, CAVIN2, |
| | | CAVIN4, CBFA2T3, CHD7, COL4A2, CSRNP1, |
| | | DACH1, DHX36, DLL4, DMRT1, E2F3, EBF1, |
| | | EGR3, ELK3, EPAS1, ESRRG, ETS1, FGF7, |
| | | FGFR2, FLI1, FOXA2, FOXF1, FOXK1, FOXL1, |
| | | Foxp1, GATA2, GATA3, GMNN, HDAC7, |
| | | HDAC9, HIVEP2, Hmga2, IGF1R, IL22, IRF1, |
| | | IRF8, ISL1, JAG1, JAK2, JUN, KLF3, KLF4, |
| | | LCOR, LPIN2, MAF, MAML3, MBD2, MDFIC, |
| | | MECOM, MEF2A, MEF2C, Meis1, mir-467, MNT, |
| | | MRTFB, NCOA2, NFATC2, NFIB, NFKB1, |
| | | NODAL, Nrg1, PBX1, PCBP3, PPARD, PRDM1, |
| | | PRMT6, PROX1, RALGAPA1, RARB, RARG, |
| | | RFX3, RIPPLY3, RUNX1, RYBP, SERTAD2, |
| | | SETD3, SKI, SLC39A8, SMAD1, SMAD3, SOX11, |
| | | SOX18, SOX7, TBX1, TBXT, TCF4, TLE4, |
| | | TRAF7, TWIST2, WT1, WWOX, WWTR1, YES1, |
| | | ZEB1, ZEB2, ZMIZ1, ZNF326, ZNF608, ZNF704 |
| Expression of RNA | 5.29 | ABLIM1, ACVRL1, ADRB2, AEBP2, AFF3, AHR, |
| | | ALX1, AMPH, Ank2, AP3B1, APBB2, ATF6B, |
| | | ATOH1, ATP1B1, BACH1, BACH2, BARX2, |
| | | BCL6B, BMP7, BMPR2, CAV1, CAVIN2, |
| | | CAVIN4, CBFA2T3, CHD7, CLDN5, COL4A2, |
| | | CSRNP1, DACHI, DHX36, DLL4, DMRT1, |
| | | DNAJC1, DUSP4, E2F3, EBF1, EDN3, EGR3, |
| | | ELK3, ELP1, EPAS1, ESRRG, ETS1, FGF7, |
| | | FGFR2, FLI1, FOXA2, FOXF1, FOXK1, FOXL1, |
| | | Foxp1, GATA2, GATA3, GMNN, HDAC7, |
| | | HDAC9, HGF, HIP1, HIVEP2, Hmga2, HTT, |
| | | IGF1R, IKBKB, IL22, IRF1, IRF8, ISL1, JAG1, |
| | | JAK2, JUN, Kdm1b, KIT, KLF3, KLF4, LCOR, let- |
| | | 7, LPIN2, LRRK2, MAF, MAML3, MBD2, MDFIC, |
| | | MECOM, MEF2A, MEF2C, Meis1, mir-146, mir-17, |
| | | mir-181, mir-467, MNT, MRTFB, NCOA2, |
| | | NFATC2, NFIB, NFKB1, NGF, NOCT, NODAL, |
| | | NR2C2, Nrg1, NTRK2, PBX1, PCBP3, PMP22, |
| | | POLR2C, PPARD, PPP1R15A, PRDM1, PRKAA2, |
| | | PRMT6, PROX1, PTEN, QKI, RAC1, RALGAPA1, |
| | | RAMP2, RAP1A, RARB, RARG, RBL2, RC3H1, |
| | | RELN, RFX3, RIPPLY3, ROCK2, RPS6KA2, |
| | | RUNX1, RYBP, SERTAD2, SETD3, SKI, |
| | | SLC39A8, SMAD1, SMAD3, SMAD6, SMARCA2, |
| | | SOCS1, SOX11, SOX18, SOX7, TBX1, TBXT, |
| | | TCF4, TLE4, TMEM135, TNC, TRAF7, TTC21B, |
| | | TWIST2, VAV3, VIM, WT1, WWOX, WWTR1, |
| | | YES1, ZEB1, ZEB2, ZMIZ1, ZNF326, ZNF608, |
| | | ZNF704 |
| Quantity of progenitor | 5.28 | AHR, ATP2B1, BCL2L11, BLK, CASP8, CAV2, |
| cells | | CBFA2T3, CD247, CSK, DLL4, DUSP6, EBF1, |
| | | EFNB2, EGR3, EPAS1, ETS1, FAS, FLI1, GATA2, |
| | | HIVEP2, IGF1R, IL1R1, IRF1, IRF8, ITPR2, JAK2, |
| | | KDR, KIT, KITLG, KL, KLF4, LAMC1, let-7, |
| | | LTBR, Ly6a (includes others), MAF, MECOM, |
| | | Meis1, mir-17, NFKB1, NR2C2, PBX1, PECAM1, |
| | | PLCG1, PLXND1, PPARD, PRKCH, PRKDC, |
| | | PTEN, RAC1, RARG, RBL2, RNASEL, RUNX1, |
| | | SCARB1, SOCS1, TCF4, TCIM, THEMIS, TLE4, |
| | | TMOD3, TNFRSF11B, VAV3, VCAM1, WIPF1, |
| | | ZEB1 |
| Angiogenesis of | 4.60 | ACVRL1, CCN1, CDH5, DLL4, FGFR2, FLT1, |
| lesion | | FLT4, HGF, IKBKB, KDR, KITLG, LTBR, mir-17, |
| | | mir-467, MMP2, PLG, S1PR1, SEMA6A, |
| | | SERPINE1, TEK, VAV3, VEGFC, WT1 |
| Quantity of airway | 4.10 | ABCG1, AP3B1, EPAS1, FGF7, LIPA, NDEL1, |
| epithelial cells | | PTEN, RARB |
| Proliferation of | 4.07 | ADAM15, BMPR2, CALCRL, CDH13, DLL4, |
| vascular cells | | E2F3, EFNB2, FLT1, HGF, IGF1R, IRF1, let-7, mir- |
| | | 126, PECAM1, PLG, PTEN, PTK2, RAC1, ROCK2, |
| | | Rps41, SEMA3F, SERPINE1, SMAD3, SOCS1, |
| | | SPRY4 |
| Morphology of germ | 4.06 | AFDN, BMPR2, DAD1, EXT2, FGF7, FOXA2, |
| layer | | FOXF1, GMNN, HIRA, HTT, ISL1, LAMA1, |
| | | MECOM, METAP2, PBX1, PLPP3, PTGIS, RAC1, |
| | | SKI, SMAD1 |
| Thrombin Signaling | 3.14 | ADCY1, ADCY9, ARHGEF15, ARHGEF3, F2RL2, |
| | | F2RL3, GATA2, GATA3, GNA14, GNAT1, GNG7, |
| | | IKBKB, ITPR2, MYL4, NFKB1, PLCD3, PLCG1, |
| | | PRKCH, PTK2, RAC1, RAP1A, RHOJ, ROCK2 |
| eNOS Signaling | 2.84 | ADCY1, ADCY9, AQP1, CASP8, CAV1, CCNA1, |
| | | CHRNA10, FLT1, FLT4, HSPA4, ITPR2, KDR, |
| | | PLCG1, PRKAA2, PRKAB1, PRKCH, SLC7A1, |
| | | VEGFC |
| Pulmonary Healing | 2.72 | BLK, BMPR2, FGF7, FGFR2, JAG1, KDR, MMP2, |
| Signaling Pathway | | NFKB1, PECAM1, PRKAA2, PRKAB1, PRKCH, |
| | | RAC1, RAP1A, SMAD1, TCF4, TNFRSF11B, |
| | | VEGFC, WNT2B, WWTR1, YES1 |
| Apelin Endothelial | 2.49 | ADCY1, ADCY9, GNA14, GNAT1, GNG7, JUN, |
| Signaling Pathway | | MEF2A, MEF2C, NFKB1, PRKAA2, PRKAB1, |
| | | PRKCH, RAP1A, SMAD3, TEK, VCAM1 |
| Cardiomyocyte | 2.46 | BMP7, BMPR2, MEF2C, SMAD1, SMAD6 |
| Differentiation via | | |
| BMP Receptors | | |
| Pulmonary Fibrosis | 2.15 | AEBP2, BMPR2, CAV1, COL15A1, COL25A1, |
| Idiopathic Signaling | | COL4A2, COL5A1, EFNB2, FGFR2, ITGA2, JAG1, |
| Pathway | | JAK2, JUN, MMP2, NFKB1, PDGFD, PLG, PTEN, |
| | | PTK2, RAP1A, ROCK2, RPS6KA2, SERPINE1, |
| | | SMAD3, TCF4, VIM, WNT2B, WWTR1 |
| Wound Healing | 2.01 | BMPR2, COL15A1, COL25A1, COL4A2, COL5A1, |
| Signaling Pathway | | FGF7, FGFR2, IKBKB, IL1R1, ITGA3, ITGA6, |
| | | JAK2, JUN, LAMA1, LAMC1, NFKB1, PDGFD, |
| | | RAC1, RAP1A, TNFRSF11B, VEGFC, VIM |
| Production of Nitric | 1.52 | ELP1, IKBKB, IRF1, IRF8, JAK2, JUN, NFKB1, |
| Oxide and Reactive | | PLCG1, PON1, PPMIJ, PPP2R2A, PRKCH, RAC1, |
| Oxygen Species in | | RAP1A, RHOJ, TNFRSF11B |
| Macrophages | | |
| Opioid Signaling | 1.50 | ADCY1, ADCY9, ARRB1, BLK, CACNA1C, |
| Pathway | | CACNA2D4, GNA14, GNAT1, GNG7, GRK5, |
| | | ITPR2, KCNJ3, NFKB1, PRKCH, RAC1, RAP1A, |
| | | RGS12, RGS3, RPS6KA2, SCN7A, TCF4, YES1 |
| Sphingosine-1- | 1.37 | ADCY1, ADCY9, CASP7, CASP8, PDGFD, |
| phosphate Signaling | | PLCD3, PLCG1, PTK2, RAC1, RHOJ, S1PR1 |

MOUSE IMMUNE

| | | |
|---|---|---|
| hematopoietic or | 5.19 | Carmil2, Cbfa2t3, Cd4, Cxcr5, Dll1, Gata2, Gfi1, |
| lymphoid organ | | H2-DMa, Mpl, Nkx2-3, Runx1, Runx3, Sema4a, |
| development | | Tgfbr3, Tmem91 |
| Abnormal | 4.96 | GFI1, GIMAP1-GIMAP5, LAG3, LMNA, NKX2-3, |
| morphology of | | NQO1, PIK3CD, RHOF, RUNX1, RUNX3, |
| lymphoid organ | | TBXA2R, TGFBR3 |
| immune system | 4.96 | Bfsp2, Clic4, Dll1, Gja1, Hoxa5, Ripor2, Tjp2 |
| development | | |
| Th2 Pathway | 4.69 | GFI1, HLA-DMB, IL12RB1, PIK3CD, RUNX3, |
| | | TGFBR3 |
| Abnormal | 4.55 | GIMAP1-GIMAP5, LAG3, LMNA, NKX2-3, |
| morphology of spleen | | NQO1, PIK3CD, RHOF, RUNX3, TBXA2R, |
| | | TGFBR3 |
| hemopoiesis | 4.41 | Cbfa2t3, Cd4, Dll1, Gata2, Gfi1, H2-DMa, Mpl, |
| | | Nkx2-3, Runx1, Runx3, Sema4a, Tgfbr3, Tmem91 |
| Th1 and Th2 | 4.11 | GFI1, HLA-DMB, IL12RB1, PIK3CD, RUNX3, |
| Activation Pathway | | TGFBR3 |
| Quantity of lymphoid | 3.42 | GFI1, GIMAP1-GIMAP5, LAG3, NKX2-3, |
| organ | | PIK3CD, RUNX1, RUNX3, TGFBR3 |
| Transcription of DNA | 3.31 | AFF3, AGAP2, ATF7IP, BHLHA15, CRY2, |
| | | GATA2, GBX2, GFI1, HOXA5, NCOR2, NKX2-3, |
| | | NR0B2, RUNX1, RUNX3, SGSM1, ZNF326 |
| Differentiation of | 3.07 | GATA2, GFI1, NKX2-3, RUNX1, RUNX3, ZNF831 |
| antigen presenting | | |
| cells | | |
| Quantity of leukocytes | 3.00 | GATA2, GFI1, GIMAP1-GIMAP5, IL12RB1, |
| | | LAG3, NEDD4L, NKX2-3, NQO1, PIK3CD, PIM3, |
| | | RHOF, RPS6KA4, RUNX1, RUNX3, SLC2A1, |
| | | TBXA2R |
| IL-23 Signaling | 2.91 | IL12RB1, PIK3CD, RUNX1 |
| Pathway | | |
| Th1 Pathway | 2.80 | HLA-DMB, IL12RB1, PIK3CD, RUNX3 |
| Cell death of | 2.79 | GATA2, GFI1, NQO1, PIK3CD, RUNX1, TBXA2R |
| hematopoietic | | |
| progenitor cells | | |
| Leukocyte | 2.77 | ARHGAP9, CTNND1, CTTN, NCF4, PIK3CD |
| Extravasation | | |
| Signaling | | |
| Quantity of myeloid | 2.76 | GATA2, GFI1, GIMAP1-GIMAP5, IL12RB1, |
| cells | | NEDD4L, NQO1, PIK3CD, PIM3, RUNX1, |
| | | RUNX3, TBXA2R |
| Leukotriene | 2.74 | ALOX5AP, GGT1 |
| Biosynthesis | | |
| Development of | 2.70 | GATA2, GFI1, INSL3, RUNX1, RUNX3, ZNF831 |
| phagocytes | | |
| Differentiation of | 2.69 | GFI1, RUNX1, ZNF831 |
| conventional dendritic | | |
| cells | | |
| Eicosanoid Signaling | 2.50 | ALOX5AP, GGT1, TBXA2R |
| Quantity of B | 2.48 | GFI1, GIMAP1-GIMAP5, NKX2-3, NQO1, |
| lymphocytes | | PIK3CD, PIM3, RHOF, SLC2A1 |
| Apoptosis of T | 2.42 | GFI1, GIMAP1-GIMAP5, NQO1, PIK3CD, |
| lymphocytes | | RUNX1, TBXA2R |
| T Cell Exhaustion | 1.66 | HLA-DMB, IL12RB1, LAG3, PIK3CD, TGFBR3 |
| Signaling Pathway | | |
| Senescence Pathway | 1.39 | CACNB2, PIK3CD, RPS6KA4, TGFBR3 |
| GM-CSF Signaling | 1.39 | PIK3CD, RUNX1 |

### REFERENCES

Barefoot M E, et al. Reference Module in Biomedical Sciences 365-378 (2020) doi:10.1016/b978-0-12-801238-3.11669-1.
Barefoot M E, et al. Detection of cell types contributing to cancer from circulating, cell-free methylated DNA. Frontiers in Genetics 12: 671057, 2021.
Barefoot M E, et al. Cell-free, methyulated DNA in blood samples reveals tissue-specific, cellular damage from radiation treatment. bioRxiv (2022) doi: https://doi.org/10.1101/2022.04.12.487966.
Barazzuol L, et al. Prevention and treatment of radiotherapy-induced side effects. Molecular Oncology 14: 1538-1554 (2020).
Brownlee Z, et al. Late complications of radiation therapy for breast cancer: evolution in techniques and risk over time. Gland Surgery 7: 371-378 (2018).
Cleuren A C A, et al. The in vivo endothelial cell translatome is highly heterogeneous across vascular beds. Proceedings of the National Academy of Sciences of the United States of America 116: 201912409 (2019).
Diehl F, et al. Circulating mutant DNA to assess tumor dynamics. Nature Medicine 14: 985-90 (2008).
Dor Y & Cedar H. Principles of DNA methylation and their implications for biology and medicine. Lancet 392: 777-786 (2018).
Feng W., et al. Single cell analysis of endothelial cells identified organ-specific molecular signatures and heart-specific cell populations and molecular features. Frontiers in Cardiovascular Medicine 6: 165 (2019).
Greenberg M V C. & Bourc'his D. The diverse roles of DNA methylation in mammalian development and disease. Nature Review Molecular Cell Biology 20: 590-607 (2019).
Hyduke D R, et al. Identifying radiation exposure biomarkers from mouse blood transcriptome. International Journal of Bioinformatics Research and Applications 9: 365 (2013).
Khan S, et al. EndoDB: a database of endothelial cell transcriptomics data. Nucleic Acids Research 47: D736-D744 (2018).
Li W, et al. CancerDetector: ultrasensitive and non-invasive cancer detection at the resolution of individual reads using cell-free DNA methylation sequencing data. Nucleic Acids Research 46: e89-e89 (2018).
Lo Y M D, et al. Quantitative analysis of fetal DNA in maternal plasma and serum: implications for noninvasive prenatal diagnosis. American Journal of Human Genetics 62: 768-775 (1998).
Loyfer N, et al. A human DNA methylation atlas reveals principles of cell-type specific methylation and identifies thousands of cell-type specific regulatory elements. Biorxiv 2022.01.24.477547 (2022) doi:10.1101/2022.01.24.477547.
Loyfer & Kaplan. wgbstools: A computational suite for DNA methylation sequencing data representation, visualization, and analysis. wgbstools.
Maggi E C, et al. Development of a method to implement whole-genome bisulfite sequencing of cfDNA from cancer patients and a mouse tumor model. Frontiers Genetics 09: 6 (2018).
Moss J, et al. Comprehensive human cell-type methylation atlas reveals origins of circulating cell-free DNA in health and disease. Nature Communications 9: 5068 (2018).
Newman A M, et al. An ultrasensitive method for quantitating circulating tumor DNA with broad patient coverage. Nature Medicine 20: 548-54 (2014).
Nolan D. J., et al. Molecular signatures of tissue-specific microvascular endothelial cell heterogeneity in organ maintenance and regeneration. Developmental Cell 26: 204-219 (2013).
Park M-T, et al. Radio-sensitivities and angiogenic signaling pathways of irradiated normal endothelial cells derived from diverse human organs. Journal of Radiation Research 53: 570-580 (2012).
Sabbagh M F, et al. Transcriptional and epigenomic landscapes of CNS and non-CNS vascular endothelial cells. Elife 7: e36187 (2018).
Scott C A, et al. Identification of cell-type specific methylation signals in bulk whole genome bisulfite sequencing data. Genome Biology 21: 156 (2020).
Shiels P G, et al. The role of epigenetics in renal ageing. Nature Reviews Nephrology 13: 471-482 (2017).
Tapio, S. Pathology and biology of radiation-induced cardiac disease. Journal of Radiation Research 57: 439-448 (2016).
Wagner J U G & Dimmeler S. Cellular cross-talks in the diseased and aging heart. Journal of Molecular & Cellular Cardiology 138: 136-146 (2019).
White J & Joiner M C. Radiation Toxicity: A Practical Guide. Cancer Treatment 128: 65-109 (2006).
Wijerathne H, et al. Mechanisms of radiation-induced endothelium damage: emerging models and technologies. Radiotherapy & Oncology 158: 21-32 (2021).

What is claimed is:

1. A method of determining if a subject has suffered tissue damage from exposure to a toxic agent, the method comprising
(a) sequencing cell-free DNA (cfDNA) in a biospecimen from the subject;
(b) determining cellular origin of the cfDNA by identifying the methylation patterns in one or more portions of the sequence of the cfDNA that contains methylation sites, wherein the cellular origin of the cfDNA is determined when the methylation pattern in the one or more portions is the same as a known cell-type specific methylation patterns;
(c) measuring the quantity of the cfDNA of the determined cellular origin, and
(d) comparing the measured quantity of the cfDNA of the determined cellular origin with a normal quantity of cfDNA of the determined cellular origin;
wherein an increase in the measured quantity of the cfDNA of the determined cellular origin over the normal quantity of cfDNA of the determined cellular origin is indicative that the subject has suffered or suffers tissue damage from the exposure.

2. A method of determining if a subject has suffered tissue damage from exposure to a toxic agent, the method comprising, at two or more time points,
(a) sequencing cell-free DNA (cfDNA) in a biospecimen from the subject;
(b) determining cellular origin of the cfDNA by identifying the methylation patterns in one or more portions of the sequence of the cfDNA that contains methylation sites, wherein the cellular origin of the cfDNA is determined when the methylation pattern in the one or more portions is the same as a known cell-type specific methylation patterns; and
(c) measuring the quantity of the cfDNA of the determined cellular origin,
wherein an increase in the measured quantity of the cfDNA of the determined cellular origin at a later time point as compared to an earlier time point is indicative that the subject has suffered or suffers tissue damage from the exposure.

3. A method of treating a subject who has suffered tissue damage from exposure to a toxic agent, the method comprising administering a treatment for the tissue damage to the subject,
wherein the subject is determined to have suffered from tissue damage by a method comprising:
(a) sequencing cell-free DNA (cfDNA) in a biospecimen from the subject;
(b) determining cellular origin of the cfDNA by identifying the methylation patterns in one or more portions of the sequence of the cfDNA that contains methylation sites, wherein the cellular origin of the cfDNA is determined when the methylation pattern in the one or more portions is the same as a known cell-type specific methylation patterns;
(c) measuring the quantity of the cfDNA of the determined cellular origin, and
(d) comparing the measured quantity of the cfDNA of the determined cellular origin with a normal quantity of cfDNA of the determined cellular origin;
wherein an increase in the measured quantity of the cfDNA of the determined cellular origin over the normal quantity of cfDNA of the determined cellular origin is indicative that the subject has suffered tissue damage.

4. A method of treating a subject who has suffered tissue damage from exposure to a toxic agent, the method comprising administering a treatment for the tissue damage to the subject,
wherein the subject is determined to have suffered from tissue damage by a method comprising, at two or more time points:
(a) sequencing cell-free DNA (cfDNA) in a biospecimen from the subject;
(b) determining cellular origin of the cfDNA by identifying the methylation patterns in one or more portions of the sequence of the cfDNA that contains methylation sites, wherein the cellular origin of the cfDNA is determined when the methylation pattern in the one or more portions is the same as a known cell-type specific methylation patterns; and
(c) measuring the quantity of the cfDNA of the determined cellular origin,
wherein an increase in the measured quantity of the cfDNA of the determined cellular origin at a later time point as compared to an earlier time point is indicative that the subject has suffered tissue damage.

5. A method of treating tissue damage in a subject, the method comprising administering a treatment for the tissue damage to the subject and monitoring the tissue damage,
wherein the monitoring comprises:
(a) sequencing cell-free DNA (cfDNA) in a biospecimen from the subject;
(b) determining cellular origin of the cfDNA by identifying the methylation patterns in one or more portions of the sequence of the cfDNA that contains methylation sites, wherein the cellular origin of the cfDNA is determined when the methylation pattern in the one or more portions is the same as a known cell-type specific methylation patterns;
(c) measuring the quantity of the cfDNA of the determined cellular origin, and
(d) comparing the measured quantity of the cfDNA of the determined cellular origin with a normal quantity of cfDNA of the determined cellular origin;
wherein a decrease in the measured quantity of the cfDNA of the determined cellular origin as compared to the normal quantity of cfDNA of the determined cellular origin is indicative that the treatment is effective, and an increase or no change in the measured quantity of the cfDNA of the determined cellular origin over the normal quantity of cfDNA of the determined cellular origin is indicative that the treatment is not effective.

6. A method of treating tissue damage in a subject, the method comprising administering a treatment for the tissue damage to the subject and monitoring the tissue damage,
wherein the monitoring comprises, at two or more time points:
(a) sequencing cell-free DNA (cfDNA) in a biospecimen from the subject;
(b) determining cellular origin of the cfDNA by identifying the methylation patterns in one or more portions of the sequence of the cfDNA that contains methylation sites, wherein the cellular origin of the cfDNA is determined when the methylation pattern in the one or more portions is the same as a known cell-type specific methylation patterns; and
(c) measuring the quantity of the cfDNA of the determined cellular origin,
wherein a decrease in the measured quantity of the cfDNA of the determined cellular origin at later time point as compared to an earlier time point is indicative that the treatment is effective, and an increase or no change in the measured quantity of the cfDNA of the determined cellular origin at a later time point as compared to an earlier time point is indicative that the treatment is not effective.

7. The method of claim 5 or 6, wherein the tissue damage is caused by exposure to a toxic agent.

8. The method of any one of claims 1-4 or 7, wherein the toxic agent comprises radiation.

9. The method of claim 8, wherein the radiation is for therapeutic purposes, accidental, or environmental.

10. The method of claim 8, wherein the radiation comprises a radioactive substance.

11. The method of claim 10, wherein the radioactive substance is ingested by the subject, inhaled by the subject, or absorbed through body surface contamination by the subject.

12. The method of any one of claims 1-4 or 7, wherein the toxic agent comprises a microorganism.

13. The method of claim 12, wherein the microorganism comprises a pathogen.

14. The method of claim 13, wherein the pathogen is selected from a bacterium and virus.

15. The method of any one of claims 1-4 or 7, wherein the toxic agent is from a synthetic chemical source or from a biological source.

16. The method of any one of claims 1-4 or 7, wherein the toxic agent comprises a pharmaceutical therapy.

17. The method of any one of claims 1-4 or 7, wherein the toxic agent comprises a chemical or biological or radioactive substance used a weapon.

18. A method of treating a subject in need thereof, the method comprising administering a treatment to the subject and monitoring whether the treatment causes tissue damage in the subject,
wherein the monitoring comprises:
(a) sequencing cell-free DNA (cfDNA) in a biospecimen from the subject;
(b) determining cellular origin of the cfDNA by identifying the methylation patterns in one or more portions of the sequence of the cfDNA that contains methylation sites, wherein the cellular origin of the cfDNA is determined when the methylation pattern in the one or more portions is the same as a known cell-type specific methylation patterns;
(c) measuring the quantity of the cfDNA of the determined cellular origin, and
(d) comparing the measured quantity of the cfDNA of the determined cellular origin with a normal quantity of cfDNA of the determined cellular origin;
wherein an increase in the measured quantity of the cfDNA of the determined cellular origin over the normal quantity of cfDNA of the determined cellular origin is indicative that the treatment is causing tissue damage.

19. A method of treating a subject in need thereof, the method comprising administering a treatment to the subject and monitoring whether the treatment causes tissue damage in the subject,
wherein the monitoring comprises, at two or more time points:
(a) sequencing cell-free DNA (cfDNA) in a biospecimen from the subject;
(b) determining cellular origin of the cfDNA by identifying the methylation patterns in one or more portions of the sequence of the cfDNA that contains methylation sites, wherein the cellular origin of the cfDNA is determined when the methylation pattern in the one or more portions is the same as a known cell-type specific methylation patterns; and
(c) measuring the quantity of the cfDNA of the determined cellular origin,
wherein an increase in the measured quantity of the cfDNA of the determined cellular origin at a later time point as compared to an earlier time point is indicative that the treatment is causing tissue damage.

20. The method of any one of claims 1-19, further comprising adjusting the treatment administered to the subject when the treatment is indicated to be not effective or causing tissue damage.

21. The method of any one of claims 5-20, wherein the normal quantity of cfDNA comprises a quantity of cfDNA for the determined cellular origin that is generated in a population of individuals who were not exposed to the toxic agent, or who were not administered the treatment.

22. A method of treating a subject having a tumor, the method comprising
(A) monitoring a response to a first treatment, an adverse reaction to the first treatment, or a combination thereof, in which the monitoring comprises:
(i) determining whether there is an adverse reaction to the first treatment, comprising
(a) sequencing circulating tumor DNA (cfDNA) in a biospecimen from the subject;
(b) determining cellular origin of the cfDNA by identifying the methylation patterns in one or more portions of the sequence of the cfDNA that contains methylation sites, wherein the cellular origin of the cfDNA is determined when the methylation pattern in the one or more portions is the same as a known cell-type specific methylation patterns;
(c) measuring the quantity of the cfDNA of the determined cellular origin, and
(d) comparing the measured quantity of the cfDNA of the determined cellular origin with a normal quantity of cfDNA of the determined cellular origin, in which an increase in the measured quantity of the cfDNA of the determined cellular origin over the normal quantity of cfDNA of the determined cellular origin is indicative of an adverse reaction;
(ii) determining whether there is a response to the first treatment, comprising:
(a) sequencing circulating tumor DNA (cfDNA) in a biospecimen from the subject,
(b) determining clonal heterogeneity of cells of the tumor by genotyping the cfDNA, in which the presence of more than one clone of the tumor cells or the presence of a tumor cell clone that has not been previously identified in the subject is indicative of an ineffective response to the first treatment; and
(B) either administering the same treatment as the first treatment when it is determined that there is no adverse reaction, that there is not an ineffective response, or a combination thereof; or administering an adjusted treatment when it is determined that there is an adverse reaction, that there is an ineffective response, or a combination thereof.

23. The method of claim 22, wherein the normal quantity of cfDNA comprises a quantity of cfDNA for the determined cellular origin that is generated in a population of individuals who do not have a tumor.

24. The method of claim 22, wherein the normal quantity of cfDNA comprises a quantity of cfDNA for the determined cellular origin that is generated in a population of individuals who did not receive the first treatment.

25. A method of treating a subject having a tumor, the method comprising
(A) monitoring a response to a first treatment, an adverse reaction to the first treatment, or a combination thereof, in which the monitoring comprises, at two or more time points,
(i) determining whether there is an adverse reaction to the first treatment, comprising
(a) sequencing cell-free (cfDNA) in a biospecimen from the subject;
(b) determining cellular origin of the cfDNA by identifying the methylation patterns in one or more portions of the sequence of the cfDNA that contains methylation sites, wherein the cellular origin of the cfDNA is determined when the methylation pattern in the one or more portions is the same as a known cell-type specific methylation patterns; and
(c) measuring the quantity of the cfDNA of the determined cellular origin, wherein an increase in the measured quantity of the cfDNA of the determined cellular origin at a later time point as compared to an earlier time point is indicative of an adverse reaction; and
(ii) determining whether there is a response to the first treatment, comprising
(a) sequencing circulating tumor (cfDNA) in a biospecimen from the subject,
(b) determining clonal heterogeneity of cells of the tumor by genotyping the cfDNA, wherein the presence of more than one clone of the tumor cells or the presence of a tumor cell clone in a subsequent time point that has not been identified at a previous time point is indicative of an ineffective response to the first treatment; and
(B) either administering the same treatment as the first treatment when it is determined that there is no adverse reaction, that there is not an ineffective response, or a combination thereof; or administering an adjusted treatment when it is determined that there is an adverse reaction, that there is an ineffective response, or a combination thereof.

27. The method of any one of claims 1-26, wherein the biospecimen comprises a biological fluid.

28. The method of claim 27, wherein the biological fluid is selected from blood, serum, plasma, cerebrospinal fluid, saliva, urine, and sputum.

29. The method of claim 27, wherein the biological fluid comprises blood, serum, or plasma.

30. The method of any one of claims 1-29, wherein the methylation pattern comprises a segment of nucleotide sequence containing at least 3 CpG dinucleotides.

31. The method of any one of claims 1-30, wherein the known methylation patterns are set forth in Table 2.
